(19) United States
(12) Patent Application Publication
(10) Pub. No.: US 2012/0202287 A1
(43) Pub. Date: Aug. 9, 2012

(54) Stem Cell Culture Methods

(76) Inventors: David Roger Adams, Edinburgh (GB); Peter Burton, Glasgow (GB); Miles Douglas Houslay, Renfrewshire (GB); Graeme Milligan, Glasgow (GB); Joanne Mountford, Glasgow (GB)

(21) Appl. No.: 13/145,316

(22) PCT Filed: Jan. 7, 2010
(86) PCT No.: PCT/GB2010/000002

§ 371 Date: Nov. 3, 2011

(30) Foreign Application Priority Data
Jan. 22, 2009 (GB) 0901069.5
Sep. 18, 2009 (GB) 0916476.5

(51) Int. Cl. C12N 5/071; C07D 473/34; C07D 487/04; C07D 473/00; C12N 5/0735; C07D 471/04

(52) U.S. Cl. 435/366; 435/377; 546/118; 544/262; 544/264; 544/277

(57) ABSTRACT

The invention provides methods for reversibly inhibiting stem cell differentiation wherein a compound of formula (I) is contacted with a stem cell. The invention further provides a method for preparing a culture medium, a culture medium supplement and a composition comprising a compound of formula (I).

The present invention relates to methods for culturing stem cells and is particularly concerned with providing methods for expanding populations of pluripotent stem cells by reversibly inhibiting differentiation of the stem cells during culturing.

Pluripotent stem cells are a primary focus of research, in particular because they are potentially useful as a source for tissue or organ replacement. Clinical and research applications of pluripotent stem cells require reproducible cell culture methods to provide adequate numbers of cells of suitable quality. However, it is generally difficult to produce large numbers of stem cells which maintain stem cell characteristics e.g. pluripotency. This is particularly the case for human embryonic stem (hES) cells. Therefore, research has focussed on optimizing the conditions for stem cell culture with a view to providing larger populations of pluripotent stem cells.

hES cells were originally derived from human blastocysts using mouse embryonic fibroblasts (mEFs) as feeder cells (Thomson et al. (1998) Science 282:1145-1147). hES cells are still commonly maintained using human or mouse embryonic fibroblasts as feeder cells, or as a source of conditioned medium, or both. The extrinsic factors required for maintaining hES cell pluripotency are still not well understood. It is important that any component used to inhibit stem cell differentiation does so in a reversible manner, such that when an appropriately sized population of pluripotent cells has been generated, the “freezing” effect can be reversed and differentiation can proceed as normal. It is known that basic fibroblast growth factor (FGF), either when used alone or in combination with other factors, supports undifferentiated growth of hES cells and hence is typically used as a component in stem cell culture media. However, FGF is expensive and hence stem cell culturing methods which involve its use are expensive. Thus, there is a need for an alternative for FGF which will inhibit stem cell differentiation in a reversible manner, thus making it possible to provide a cost effective method for producing large populations of pluripotent stem cells.

In this regard, the present inventors have surprisingly found that compounds having adenosine deaminase (ADA) inhibitory activity are effective in inhibiting stem cell differentiation in a reversible manner. Advantageously, compounds having ADA inhibitory activity are well known, are available commercially from a number of sources and are generally low cost. In particular, it has been found that the level of inhibition of differentiation is equal to or better than the level of inhibition of differentiation observed when FGF is present in the culture medium. Thus, when these compounds are used in place of FGF, a significant reduction in the costs associated with producing large populations of pluripotent stem cells is obtained.

Accordingly, the invention provides a method of inhibiting stem cell differentiation comprising contacting an ADA inhibitor with a stem cell.

The present invention also provides a method of inhibiting stem cell differentiation comprising contacting a compound of formula (I) with a stem cell:

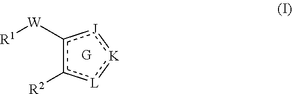

wherein

W is selected from C(Z)₂ and NZ;

each Z is independently selected from hydrogen, C₁₋₁₂ alkyl, C₂₋₁₂ alkenyl, C₂₋₁₂ alkynyl, halogen, —SR⁵, —OR⁵, —NR⁶R⁶, aryl, heteroaryl, —COR⁶, C₃₋₁₀ cycloalkyl and C₃₋₁₀ heterocycloalkyl or (Z)₂ is ═O;

J and K are each independently selected from N, NR³, NR⁴ and CR³;

L is selected from N and NR⁴, wherein if L is N, one of J or K is NR⁴;

ring G is an aromatic ring;

R¹ is selected from hydrogen, C₁₋₁₂ alkyl, C₁₋₁₂ alkenyl, C₂₋₁₂ alkynyl, halogen, —SR⁷, —OR⁷, —NR⁸R⁸, aryl, heteroaryl, —COR^{S}, C₃₋₁₂ cycloalkyl and C₃₋₁₀ heterocycloalkyl;

R² is selected from hydrogen, C₁₋₁₂ alkyl, C₂₋₁₂ alkenyl, C₂₋₁₂ alkynyl, halogen, —SR⁹, —OR⁹, aryl, heteroaryl, —COR¹⁰, C₃₋₁₀ cycloalkyl and C₃₋₁₀ heterocycloalkyl; or alternatively

R¹ and R² are joined to form a 5 to 7 membered carbocyclic ring, optionally including one, two or three unsaturated bonds, wherein optionally one or more of the carbon atoms which form the 5 to 7 membered carbocyclic ring is replaced with a heteroatom selected from N, S and O, and wherein each one of the atoms which form the 5 to 7 membered ring is independently optionally substituted with one or two R³² groups, wherein each R³² is independently selected from hydrogen, halogen, C₁₋₁₂-alkyl, C₂₋₁₂-alkenyl, C₂₋₁₂-alkynyl, aryl, heteroaryl, —OR³³, NR³⁴R³⁴, —COR³⁴, C₃₋₁₂ cycloalkyl and C₃₋₁₀ heterocycloalkyl;

R³ is selected from hydrogen, C₁₋₁₂ alkyl, C₂₋₁₂ alkenyl, C₂₋₁₂ alkynyl, halogen, —SR¹¹, —OR¹¹, NR¹²R¹², aryl, heteroaryl, —COR¹², C₃₋₁₀ cycloalkyl and C₃₋₁₀ heterocycloalkyl;

R⁴ is a group of formula (IIA) or (IIB):

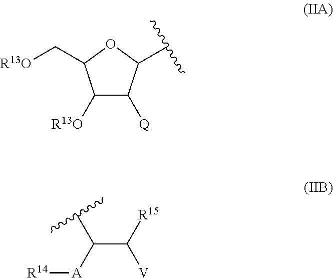

wherein Q is selected from —H and —OH;

R¹³ is selected from hydrogen, C₁₋₁₂ alkyl, C₂₋₁₂ alkenyl, C₂₋₁₂ alkynyl, aryl, heteroaryl, —COR¹⁶, C₃₋₁₀ cycloalkyl and C₃₋₁₀ heterocycloalkyl;

A is a single bond or a group of formula —O-M-, wherein M is selected from C₁₋₆ alkyl, C₂₋₆ alkenyl and C₂₋₆ alkynyl;

V is selected from hydrogen, —OR¹⁷, —SR¹⁷, NR¹⁸R¹⁸ and cyano;

R¹⁴ is selected from hydrogen, C₁₋₁₂ alkyl, C₂₋₁₂ alkenyl, C₂₋₁₂ alkynyl, halogen, —SR¹⁹, —OR¹⁹, —NR²⁰R²⁰, aryl, heteroaryl, —COR²⁰, C₃₋₁₀ cycloalkyl and C₃₋₁₀ heterocycloalkyl, wherein each of said C₁₋₁₂ alkyl, C₂₋₁₂ alkenyl, C₂₋₁₂-alkynyl, C₁₋₁₀-alkoxy, aryl, heteroaryl and C₃₋₁₀ cycloalkyl is optionally substituted with 1, 2 or 3 groups independently selected from hydrogen, halogen, C₁₋₁₂-alkyl, C₂₋₁₂-alkenyl, aryl, heteroaryl, —OR²⁵ and NR²⁵R²⁶;

R¹⁵ is selected from hydrogen, C₁₋₁₂ alkyl, C₂₋₁₂ alkenyl, C₂₋₁₂ alkynyl, halogen, —CF₃, —SR²¹, OR²¹, —NR²²R²², aryl, heteroaryl, —COR²², C₃₋₁₀ cycloalkyl and C₃₋₁₀ heterocycloalkyl;

each R⁵, R⁷, R⁹, R¹¹, R¹⁷, R¹⁹, R²¹ and R³³ is independently selected from hydrogen, C₁₋₁₂ alkyl, C₂₋₁₂ alkenyl, halogen, NR²³R²⁴, aryl, heteroaryl, C₃₋₁₀ cycloalkyl and C₃₋₁₀ heterocycloalkyl wherein each of said C₁₋₁₂ alkyl, C₂₋₁₂ alkenyl, C₂₋₁₂ alkynyl, C₁₋₁₂-alkoxy, aryl, heteroaryl and C₃₋₁₀ cycloalkyl is optionally substituted with 1, 2 or 3 groups independently selected from hydrogen, halogen, C₁₋₁₂-alkyl, C₂₋₁₂-alkenyl, aryl, heteroaryl, —OR²⁵ and NR²⁵R²⁶;

each R⁶, R⁸, R¹⁰, R¹², R¹⁶, R¹⁸, R²⁰, R²² and R³⁴ is independently selected from hydrogen, C₁₋₁₂ alkyl, C₂₋₁₂ alkenyl, —OR²⁷, halogen, NR²⁷R²⁸, —COR²⁸, aryl, heteroaryl, C₃₋₁₀ cycloalkyl and C₃₋₁₀ heterocycloalkyl wherein each of said C₁₋₁₂ alkyl, C₂₋₁₂ alkenyl, C₂₋₁₂-alkynyl, aryl, heteroaryl, C₃₋₁₀ cycloalkyl and C₃₋₁₀ heterocycloalkyl is optionally substituted with 1, 2 or 3 groups independently selected from hydrogen, halogen, —OR³⁰, C₁₋₁₂-alkyl, C₂₋₁₂ alkenyl, C₂₋₁₂ alkynyl, aryl, heteroaryl, C₁₋₁₂ alkoxy and NR³⁰R³¹; and

R²³, R²⁴, R²⁵, R²⁶, R²⁷, R²⁸, R³⁰ and R³¹ are independently selected from H and C₁₋₆ alkyl,

or a pharmaceutically acceptable salt thereof.

The methods of the present invention are typically carried out ex vivo.

The invention also provides use of an ADA inhibitor for inhibiting stem cell differentiation.

The invention also provides use of a compound of formula (I) for inhibiting stem cell differentiation.

The invention also provides use of an ADA inhibitor in the manufacture of a medicament for inhibiting stem cell differentiation.

The invention also provides use of a compound of formula (I) in the manufacture of a medicament for inhibiting stem cell differentiation.

The invention also provides an ADA inhibitor for inhibiting stem cell differentiation.

The invention also provides a compound of formula (I) for inhibiting stem cell differentiation.

The invention also provides a culture medium for expanding a population of pluripotent stem cells comprising an ADA inhibitor.

The invention also provides a culture medium for expanding a population of pluripotent stem cells comprising a compound of formula (I).

The invention further provides a method for preparing a culture medium comprising the steps of (a) obtaining a culture medium; and (b) adding an ADA inhibitor to the culture medium.

The invention further provides a method for preparing a culture medium comprising the steps of (a) obtaining a culture medium; and (b) adding a compound of formula (I) to the culture medium.

The invention also provides a culture medium supplement that comprises an ADA inhibitor.

The invention also provides a culture medium supplement that comprises a compound of formula (I).

The invention further provides a composition comprising an ADA inhibitor and stem cells.

The invention further provides a composition comprising a compound of formula (I) and stem cells.

The term “ADA inhibitor” as used herein is intended to refer to any compound which exhibits ADA inhibitory activity. Adenosine deaminase is a key enzyme in purine metabolism which irreversibly deaminates adenosine to form inosine. ADA is ubiquitous in human tissues and plays a crucial role in immune system development.

ADA inhibitors are known to be useful in the treatment of hypertension, lymphomas, ischaemic injury and leukaemia. More recently, they have also been found to be effective as anti-inflammatory drugs.

A large number of ADA inhibitors are known in the art and any of these known ADA inhibitors may be used in the method of the present invention. Examples of commercially available ADA inhibitors include 9-(2-hydroxy-3-nonyl)adenine (EHNA), available from Sigma and Calbiochem, 2-chloro-2′-deoxyadenosine (cladribine), available from Sigma, N⁶-methyl adenosine[6-(methylamino)purine 9-ribofuranoside], available from Sigma, 2-fluoroadenosine, available from Aldrich, 9-β-D-arabinofuranosyl-2-fluoroadenine (fludarabine desphosphate), available from Sigma, coformycin, available from Finechemie & Pharma Co Ltd and China Allochem Pharma Co Ltd and deoxycoformycin (pentostatin), available from Tocris Bioscience, NetQem LLC, Amfinecom Inc., 3B Scientific Corporation, AK Scientific Inc and Molcan Corporation.

Preferably, the ADA inhibitor is a compound of formula (I).

With reference to compounds of formula (I), in one embodiment, J is N, K is CR³ and L is NR⁴.

In one embodiment, J is CH, K is NR⁴ and L is N.

In one embodiment, R¹ and R² are joined to form a 5 to 7 membered carbocyclic ring optionally including one, two or three unsaturated bonds, wherein optionally one or more of the carbon atoms which form the 5 to 7 membered carbocyclic ring is replaced with a heteroatom selected from N, S and O, and wherein each one of the atoms which form the 5 to 7 membered ring is independently optionally substituted with one or two R³² groups, wherein each R³² is independently selected from hydrogen, halogen, C₁₋₁₂-alkyl, C₂₋₁₂-alkenyl, C₂₋₁₂-alkynyl, aryl, heteroaryl, —OR³³, NR³⁴R³⁴, —COR³⁴, C₃₋₁₂ cycloalkyl and C₃₋₁₀ heterocycloalkyl.

In one embodiment, J is N, K is CR³ and L is NR⁴ and R¹ and R² are joined to form a 5 to 7 membered carbocyclic ring optionally including one, two or three unsaturated bonds wherein optionally one or more of the carbon atoms which form the 5 to 7 membered carbocyclic ring is replaced with a heteroatom selected from N, S and O, and wherein each one of the atoms which form the 5 to 7 membered ring is independently optionally substituted with one or two R³² groups, wherein each R³² is independently selected from hydrogen, halogen, C₁₋₁₂-alkyl, C₂₋₁₂-alkenyl, C₂₋₁₂-alkynyl, aryl, heteroaryl, —OR³³, NR³⁴R³⁴, —COR³⁴, C₃₋₁₂ cycloalkyl and C₃₋₁₀ heterocycloalkyl.

Where R¹ and R² are joined to form a 7 membered carbocyclic ring, preferably W is NZ, wherein Z is hydrogen and one of the carbon atoms of the 7 membered ring is replaced with N.

Alternatively, where R¹ and R² are joined to form a 7 membered carbocyclic ring, preferably W is C(Z)₂, wherein one Z is hydrogen, the other Z is —OH and one of the carbon atoms of the 7 membered ring is replaced with N.

Where R¹ and R² are joined to form a 7 membered carbocyclic ring as defined in the preceding paragraph, preferably R⁴ is a group of formula IIA wherein Q is H and R¹³ is —H. Therefore, in one embodiment, J is N, K is CR³, L is NR⁴; R¹ and R² are joined to form a 7 membered carbocyclic ring, wherein each of the carbon atoms of the ring is substituted with two hydrogen.

In one embodiment, the compound of formula (I) is deoxycoformycin (pentostatin). Pentostatin is commercially available from Tocris Bioscience, NetQem LLC, Amfinecom Inc., 3B Scientific Corporation, AK Scientific Inc and Molcan Corporation.

Alternatively, R¹ and R² may be joined to form a six membered carbocyclic ring optionally including one, two or three unsaturated bonds wherein optionally one or more of the carbon atoms which form the 5 to 7 membered carbocyclic ring is replaced with a heteroatom selected from N, S and O, and wherein each one of the atoms which form the six membered ring is independently optionally substituted with one or two R³² groups, wherein each R³² is independently selected from hydrogen, halogen, C₁₋₁₂-alkyl, C₂₋₁₂-alkenyl, C₂₋₁₂-alkynyl, aryl, heteroaryl, —OR³³, NR³⁴R³⁴, —COR³⁴, C₃₋₁₂ cycloalkyl and C₃₋₁₀ heterocycloalkyl.

Where R¹ and R² are joined to form a six membered carbocyclic ring, preferably the compound of formula (I) has the formula (IA):

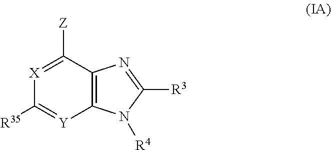

wherein X and Y are independently selected from N and CH;

R³⁵ is selected from hydrogen, halogen, C₁₋₁₂ alkyl, C₂₋₁₂ alkenyl, C₂₋₁₂ alkynyl, —SR³⁶, —OR³⁶, —NR³⁷R³⁷, aryl, heteroaryl, —COR³⁷, C₃₋₁₀ cycloalkyl, C₃₋₁₀ heterocycloalkyl;

each R³⁶ is independently selected from hydrogen, C₁₋₁₂ alkyl, C₂₋₁₂ alkenyl, C₂₋₁₂ alkynyl, C₂₋₁₂ alkoxy, halogen, NR³⁸R³⁹, aryl, heteroaryl and C₃₋₁₀ cycloalkyl, wherein each of said C₁₋₁₂ alkyl, C₂₋₁₂-alkynyl, C₂₋₁₂-alkynyl, C₁₋₁₂ alkoxy, aryl, heteroaryl and C₃₋₁₀ cycloalkyl is optionally substituted with 1, 2 or 3 groups independently selected from hydrogen, halogen, C₁₋₁₂-alkyl, C₂₋₁₂-alkenyl, aryl, heteroaryl, C₁₋₁₂ alkoxy and NR⁴⁰R⁴¹;

each R³⁷ is independently selected from hydrogen, C₁₋₁₂ alkyl, C₂₋₁₂ alkenyl, C₂₋₁₂ alkynyl, halogen, —OR⁴², NR⁴³R⁴³, aryl, heteroaryl, C₃₋₁₀ cycloalkyl and C₃₋₁₀ heterocycloalkyl wherein each of said C₁₋₁₂ alkyl, C₂₋₁₂-alkynyl, C₂₋₁₂-alkynyl, aryl, heteroaryl, C₃₋₁₀ cycloalkyl and C₃₋₁₀ heterocycloalkyl is optionally substituted with 1, 2 or 3 groups independently selected from hydrogen, halogen, C₁₋₁₂-alkyl, C₂₋₁₂-alkenyl, aryl, heteroaryl, —OR⁴⁴ and NR⁴⁵R⁴⁵, and

R³⁸, R³⁹, R⁴⁰, R⁴¹, R⁴², R⁴³ R⁴⁴ and R⁴⁵ are independently selected from H and (C₁₋₆)alkyl.

In one embodiment, both X and Y are CH. Alternatively, X is CH and Y is N. Alternatively, X is N and Y is CH. Alternatively, both X and Y are N. Preferably, both X and Y are N.

As described above, each Z is independently selected from hydrogen, C₁₋₁₂ alkyl, C₂₋₁₂ alkenyl, C₂₋₁₂ alkynyl, halogen, —SR⁵, —OR⁵, —NR⁶R⁶, aryl, heteroaryl, —COR⁶, C₃₋₁₀ cycloalkyl and C₃₋₁₀ heterocycloalkyl or (Z)₂ is ═O. Preferably Z is selected from C₁₋₁₂ alkyl, optionally substituted with 1, 2 or 3 groups independently selected from hydrogen, halogen, C₁₋₁₂-alkyl, C₂₋₁₂-alkenyl, aryl, heteroaryl, —OR⁴⁴ and NR⁴⁵R⁴⁵ and NR⁶R⁶. Preferably Z is NR⁶R⁶, wherein each R⁶ may be the same or different. Where each R⁶ is different, NR⁶R⁶ may be NHNH₂ or NHOH, preferably NHOH. Where both R⁶ are the same, preferably NR⁶R⁶ is NH₂.

R³ is selected from hydrogen, C₁₋₁₂ alkyl, C₂₋₁₂ alkenyl, C₂₋₁₂ alkynyl, halogen, —SR¹¹, —OR¹¹, NR¹²R¹², aryl, heteroaryl, —COR¹², C₃₋₁₀ cycloalkyl and C₃₋₁₀ heterocycloalkyl. In one embodiment, R³ is selected from hydrogen and C₁₋₁₂ alkyl. Preferably, R³ is hydrogen.

Where the compound of formula (I) has the structure (IA), R³⁵ is selected from hydrogen, halogen, C₁₋₁₂ alkyl, C₂₋₁₂ alkenyl, C₂₋₁₂ alkynyl, —SR³⁶, —OR³⁶, —NR³⁷R³⁷, aryl, heteroaryl, —COR³⁷, C₃₋₁₀ cycloalkyl, C₃₋₁₀ heterocycloalkyl. In one embodiment, R³⁵ is hydrogen or C₁₋₁₂ alkyl, preferably hydrogen.

Where the compound of formula (I) has the structure (IA), R⁴ is a group of formula (IIA) or formula (IIB). Preferably R⁴ is a group of formula (IIA).

In an embodiment where R⁴ is a group of formula (IIA), preferably Q is OH and R¹³ is hydrogen.

In a group of formula (IIB), A may be a single bond or a group of formula —O-M, wherein M is selected from C₁₋₆ alkyl, C₂₋₆ alkenyl and C₂₋₆ alkynyl. Preferably, A is a single bond.

In a group of formula (IIB), R¹⁴ is selected from hydrogen, C₁₋₁₂ alkyl, C₂₋₁₂ alkenyl, C₂₋₁₂ alkynyl, halogen, —SR¹⁹, —OR¹⁹, —NR²⁰R²⁰, aryl, heteroaryl, —COR²⁰, C₃₋₁₀ cycloalkyl and C₃₋₁₀ heterocycloalkyl wherein each of said C₁₋₁₂ alkyl, C₂₋₁₂ alkenyl, C₂₋₁₂-alkynyl, C₁₋₁₀-alkoxy, aryl, heteroaryl and C₃₋₁₀ cycloalkyl is optionally substituted with 1, 2 or 3 groups independently selected from hydrogen, halogen, C₁₋₁₂-alkyl, C₂₋₁₂-alkenyl, aryl, heteroaryl, —OR²⁵ and NR²⁵R²⁶. In one embodiment, R¹⁴ is C₁₋₁₂ alkyl, preferably C₃₋₈ alkyl, more preferably C₆ alkyl. In another embodiment, R¹⁴ is hydrogen.

In a group of formula (IIB), V is selected from hydrogen, —OR¹⁷, —SR¹⁷, NR¹⁸R¹⁸ and cyano. In one embodiment, V is selected from —OR¹⁷ and NR¹⁸R¹⁸, wherein each of R¹⁷ and R¹⁸ is preferably hydrogen. Preferably V is —OH.

In a group of formula (IIB), R¹⁵ is selected from hydrogen, C₁₋₁₂ alkyl, C₂₋₁₂ alkenyl, C₂₋₁₂ alkynyl, halogen, —CF₃, —SR²¹, OR²¹, —NR²²R²², aryl, heteroaryl, —COR²², C₃₋₁₀ cycloalkyl and C₃₋₁₀ heterocycloalkyl. In one embodiment, R¹⁵ is C₁₋₁₂ alkyl, preferably C₁₋₆ alkyl, preferably C₁₋₃ alkyl, more preferably C₁ alkyl. In another embodiment, R¹⁵ is C₁₋₁₂ alkyl, preferably C₂₋₁₀ alkyl, preferably C₆₋₈ alkyl, more preferably C₈ alkyl.

Where compounds as defined herein exist in one or more geometrical, optical, enantiomeric, diastereomeric and tautomeric forms, including but not limited to cis- and trans-forms, E- and Z-forms, R-, S- and meso-forms, keto-, and enol-forms, unless otherwise stated a reference to a particular compound includes all such isomeric forms, including racemic and other mixtures thereof. Where appropriate such isomers can be separated from their mixtures by the application or adaptation of known methods (e.g. chromatographic techniques and recrystallisation techniques). Where appropriate such isomers can be prepared by the application of adaptation of known methods (e.g. asymmetric synthesis).

Where R⁴ is a group of formula (IIB), there are four possible stereoisomers, (2R, 3R), (2R, 3S), (2S, 3R) and (2S, 3S).

In one embodiment, the compound of formula (I) is a compound of formula (IA), wherein both X and Y are N, Z is NR⁶R⁶, wherein each R⁶ may be the same or different and are preferably the same and are both hydrogen, R³ and R³⁵ are hydrogen and R⁴ is a group of formula (IIA), wherein Q is —OH and R¹³ is hydrogen or a pharmaceutically acceptable salt thereof.

In a further embodiment, the compound of formula (I) is a compound of formula (I), wherein J is N, K is CH, L is NR⁴, R² is H, W is carbonyl, R¹ is NHMe and R⁴ is a group of formula IIB, wherein A is a single bond, V is selected from —OR¹⁷ and NR¹⁸R¹⁸, wherein R¹⁸ is preferably hydrogen and V is preferably —OH, R¹⁴ is C₁₋₁₂ alkyl, preferably C₃₋₈ alkyl, more preferably C₆ alkyl and R¹⁵ is C₁₋₁₂ alkyl, preferably C₁₋₆ alkyl, preferably C₁₋₃ alkyl, more preferably C₁ alkyl or a pharmaceutically acceptable salt thereof.

In a further embodiment, the compound of formula (I) is a compound of formula (I), wherein J is CH, L is N, K is NR⁴, R¹ and R² are joined to form a six membered carbocyclic ring as defined above wherein two of the carbon atoms, preferably at positions 3 and 5 in the six membered ring, are replaced with a heteroatom selected form N, S and O, preferably an N atom, optionally substituted with one or two R³² groups, wherein each R³² is independently selected from hydrogen, halogen, C₁₋₁₂-alkyl, C₂₋₁₂-alkenyl, C₂₋₁₂-alkynyl, aryl, heteroaryl, —OR³³, NR³⁴R³⁴, —COR³⁴, C₃₋₁₂ cycloalkyl and C₃₋₁₀ heterocycloalkyl, the ring contains one, two or three double bonds, preferably two double bonds and R⁴ is a group of formula IIB, wherein A is a single bond, V is hydrogen, R¹⁴ is hydrogen and R¹⁵ is C₁₋₁₂ alkyl, preferably C₂₋₁₀ alkyl, preferably C₆₋₈ alkyl, more preferably C₈ alkyl or a pharmaceutically acceptable salt thereof.

In one embodiment, the compound of formula (I) is 2-decyl-2H-pyrazolo[3,4-d]pyrimidin-4-amine or a pharmaceutically acceptable salt thereof.

In one embodiment, the compound of formula (I) is 2-nonyl-2H-pyrazolo[3,4-d]pyrimidin-4-amine or a pharmaceutically acceptable salt thereof.

In one embodiment, the compound of formula (I) is 2-undecyl-2H-pyrazolo[3,4-d]pyrimidin-4-amine or a pharmaceutically acceptable salt thereof.

In one embodiment, the compound of formula (I) is 2-octyl-2H-pyrazolo[3,4-d]pyrimidin-4-amine or a pharmaceutically acceptable salt thereof.

In a further embodiment, the compound of formula (I) is a compound of formula (I), wherein J is CH, K is N, L is NR⁴, R¹ and R² are joined to form a six membered carbocyclic ring as defined above wherein two of the carbon atoms, preferably at positions 3 and 5 in the six membered ring, are replaced with a heteroatom selected form N, S and O, preferably an N atom, optionally substituted with one or two R³² groups, wherein each R³² is independently selected from hydrogen, halogen, C₁₋₁₂-alkyl, C₂₋₁₂-alkenyl, C₂₋₁₂-alkynyl, aryl, heteroaryl, —OR³³, NR³⁴R³⁴, —COR³⁴, C₃₋₁₂ cycloalkyl and C₃₋₁₀ heterocycloalkyl, the ring contains one, two or three double bonds, preferably two double bonds and R⁴ is a group of formula IIB, wherein A is a single bond, V is hydrogen, R¹⁴ is hydrogen and R¹⁵ is C₁₋₁₂ alkyl, preferably C₂₋₁₀ alkyl, preferably C₆₋₈ alkyl, more preferably C₇ alkyl or a pharmaceutically acceptable salt thereof.

In one embodiment, the compound of formula (I) is 1-nonyl-1H-pyrazolo[3,4-d]pyrimidin-4-amine or a pharmaceutically acceptable salt thereof.

In one embodiment, the compound of formula (I) is 1-octyl-1H-pyrazolo[3,4-d]pyrimidin-4-amine or a pharmaceutically acceptable salt thereof.

In a further embodiment, the compound of formula (I) is a compound of formula (I), wherein J is CH, K is CH and L is NR⁴, R¹ and R² are joined to form a six membered carbocyclic ring optionally including one, two or three unsaturated bonds, preferably three unsaturated bonds wherein two of the carbon atoms which form the 6 membered carbocyclic ring are replaced with a heteroatom selected from N, S and O, preferably N and preferably at positions 2 and 4 of the six membered ring and wherein each one of the atoms which form the 6 membered ring is independently optionally substituted with one or two R³² groups, wherein each R³² is independently selected from hydrogen, halogen, C₁₋₁₂-alkyl, C₂₋₁₂-alkenyl, C₂₋₁₂-alkynyl, aryl, heteroaryl, —OR³³, NR³⁴R³⁴, —COR³⁴, C₃₋₁₂ cycloalkyl and C₃₋₁₀ heterocycloalkyl and R⁴ is a group of formula IIB, wherein A is a single bond, V is selected from —OR¹⁷ and NR¹⁸R¹⁸, wherein R¹⁸ is preferably hydrogen and V is preferably —OH, R¹⁴ is C₁₋₁₂ alkyl, preferably C₃₋₈ alkyl, more preferably C₆ alkyl and R¹⁵ is C₁₋₁₂ alkyl, preferably C₁₋₆ alkyl, preferably C₁₋₃ alkyl, more preferably C₁ alkyl or a pharmaceutically acceptable salt thereof.

In a further embodiment, the compound of formula (I) is a compound of formula (IA), wherein X and Y are both CH, Z is NR⁶R⁶, wherein each R⁶ may be the same or different and are preferably the same and are both hydrogen, R³ and R³⁵ are hydrogen and R⁴ is a group of formula (IIB), wherein A is a single bond, V is selected from —OR¹⁷ and NR¹⁸R¹⁸, wherein each of R¹⁷ and R¹⁸ is preferably hydrogen and V is preferably —OH, R¹⁴ is C₁₋₁₂ alkyl, preferably C₃₋₈ alkyl, more preferably C₆ alkyl and R¹⁵ is C₁₋₁₂ alkyl, preferably C₁₋₆ alkyl, preferably C₁₋₃ alkyl, more preferably C₁ alkyl or a pharmaceutically acceptable salt thereof.

In a further embodiment, the compound of formula (I) is a compound of formula (IA), wherein X is CH, Y is N, Z is hydrogen or NR⁶R⁶, wherein each R⁶ may be the same or different and are preferably the same and are both hydrogen, R³ and R³⁵ are hydrogen and R⁴ is a group of formula (IIB), wherein A is a single bond, V is selected from —OR¹⁷ and NR¹⁸R¹⁸, wherein each of R¹⁷ and R¹⁸ is preferably hydrogen and V is preferably —OH, R¹⁴ is C₁₋₁₂ alkyl, preferably C₃₋₈ alkyl, more preferably C₆ alkyl and R¹⁵ is C₁₋₁₂ alkyl, preferably C₁₋₆ alkyl, preferably C₁₋₃ alkyl, more preferably C₁ alkyl or a pharmaceutically acceptable salt thereof.

In one embodiment, the compound of formula (I) is 3-(3H-imidazo[4,5-b]pyridin-3-yl)nonan-2-ol, preferably erythro-3-(3H-imidazo[4,5-b]pyridin-3-yl)nonan-2-ol or a pharmaceutically acceptable salt thereof.

In a further embodiment, the compound of formula (I) is a compound of formula (IA), wherein X is N, Y is CH, Z is hydrogen or NR⁶R⁶, wherein each R⁶ may be the same or different and are preferably the same and are both hydrogen, R³ and R³⁵ are hydrogen and R⁴ is a group of formula (IIB), wherein A is a single bond, V is selected from —OR¹⁷ and NR¹⁸R¹⁸, wherein each of R¹⁷ and R¹⁸ is preferably hydrogen and V is preferably —OH, R¹⁴ is C₁₋₁₂ alkyl, preferably C₃₋₈ alkyl, more preferably C₆ alkyl and R¹⁵ is C₁₋₁₂ alkyl, preferably C₁₋₆ alkyl, preferably C₁₋₃ alkyl, more preferably C₁ alkyl or a pharmaceutically acceptable salt thereof.

In a further embodiment, the compound of formula (I) is a compound of formula (IA), wherein both X and Y are N, Z is NR⁶R⁶, wherein each R⁶ may be the same or different and are preferably the same and are both hydrogen, R³ is hydrogen, R³⁵ is selected from hydrogen, halogen, C₁₋₁₂ alkyl, C₂₋₁₂ alkenyl, C₂₋₁₂ alkynyl, —SR³⁶, —OR³⁶, —NR³⁷R³⁷, aryl, heteroaryl, —COR³⁷, C₃₋₁₀ cycloalkyl, C₃₋₁₀ heterocycloalkyl, preferably C₁₋₁₂ alkyl or aryl, preferably aryl and R⁴ is a group of formula (IIB), wherein A is a single bond, V is selected from —OR¹⁷ and NR¹⁸R¹⁸, wherein each of R¹⁷ and R¹⁸ is preferably hydrogen and V is preferably —OH, R¹⁴ is C₁₋₁₂ alkyl, preferably C₃₋₈ alkyl, more preferably C₆ alkyl and R¹⁵ is C₁₋₁₂ alkyl, preferably C₁₋₆ alkyl, preferably C₁₋₃ alkyl, more preferably C₁ alkyl or a pharmaceutically acceptable salt thereof.

In a further embodiment, the compound of formula (I) is a compound of formula (IA), wherein both X and Y are N, Z is NR⁶R⁶, wherein each R⁶ may be the same or different and are preferably the same and are both hydrogen, R³ and R³⁵ are hydrogen and R⁴ is a group of formula (IIB), wherein A is a single bond, V is selected from —OR¹⁷ and NR¹⁸R¹⁸, wherein each of R¹⁷ and R¹⁸ is preferably hydrogen and V is preferably —OH, R¹⁴ is C₁₋₁₂ alkyl, preferably C₃₋₈ alkyl, more preferably C₆ alkyl and R¹⁵ is C₁₋₁₂ alkyl, preferably C₁₋₆ alkyl, preferably C₁₋₃ alkyl, more preferably C₁ alkyl or a pharmaceutically acceptable salt thereof.

In a further embodiment, the compound of formula (I) is a compound of formula (IA), wherein both X and Y are N, Z is NR⁶R⁶, wherein each R⁶ may be the same or different and are preferably the same and are both hydrogen, R³ and R³⁵ are hydrogen and R⁴ is a group of formula (IIB), wherein A is a single bond, V is selected from —OR¹⁷ and NR¹⁸R¹⁸, wherein each of R¹⁷ and R¹⁸ is preferably hydrogen and V is preferably —OH, R¹⁴ is C₁₋₁₂ alkyl, preferably C₁₋₆ alkyl, more preferably C₁ alkyl and R¹⁵ is C₁₋₁₂ alkyl, preferably C₁₋₆ alkyl, preferably C₁₋₃ alkyl, more preferably C₂ alkyl substituted with an aryl group or a pharmaceutically acceptable salt thereof.

In one embodiment, the compound of formula (IA) is (3S,4R)-4-(6-amino-9H-purin-9-yl)-1-phenylpentan-3-ol or a pharmaceutically acceptable salt thereof.

In a further embodiment, the compound of formula (I) is a compound of formula (IA), wherein both X and Y are N, Z is NR⁶R⁶, wherein each R⁶ may be the same or different and are preferably the same and are both hydrogen, R³ and R³⁵ are hydrogen and R⁴ is a group of formula (IIB), wherein A is a single bond, V is selected from —OR¹⁷ and NR¹⁸R¹⁸, wherein each of R¹⁷ and R¹⁸ is preferably hydrogen and V is preferably —OH, R¹⁴ is C₁₋₁₂ alkyl, preferably C₁₋₆ alkyl, more preferably C₁ alkyl and R¹⁵ is C₁₋₁₂ alkyl, preferably C₁₋₆ alkyl, preferably C₂₋₅ alkyl, more preferably C₅ alkyl or a pharmaceutically acceptable salt thereof.

In one embodiment, the compound of formula (IA) is (2R,3S)-2-(6-amino-9H-purin-9-yl)nonan-3-ol or (2S,3R)-2-(6-amino-9H-purin-9-yl)nonan-3-ol.

In a further embodiment, the compound of formula (I) is a compound of formula (IA), wherein both X and Y are N, Z is NR⁶R⁶, wherein each R⁶ may be the same or different and are preferably the same and are both hydrogen, R³ and R³⁵ are hydrogen and R⁴ is a group of formula (IIB), wherein A is a single bond, V is selected from —OR¹⁷ and NR¹⁸R¹⁸, wherein each of R¹⁷ and R¹⁸ is preferably hydrogen and V is preferably —OH, R¹⁴ is C₁₋₁₂ alkyl, preferably C₁₋₆ alkyl, more preferably C₁ alkyl and R¹⁵ is C₁₋₁₂ alkyl, preferably C₁₋₆ alkyl, preferably C₂₋₅ alkyl, more preferably C₄ alkyl or a pharmaceutically acceptable salt thereof.

In one embodiment, the compound of formula (I) is (2R,3S)-2-(6-amino-9H-purin-9-yl)octan-3-ol.

In a further embodiment, the compound of formula (I) is a compound of formula (IA), wherein both X and Y are N, Z is NR⁶R⁶, wherein each R⁶ may be the same or different and are preferably the same and are both hydrogen, R³ and R³⁵ are hydrogen and R⁴ is a group of formula (IIB), wherein A is a single bond, V is selected from —OR¹⁷ and NR¹⁸R¹⁸, wherein each of R¹⁷ and R¹⁸ is preferably hydrogen and V is preferably —OH, R¹⁴ is C₁₋₁₂ alkyl, preferably C₁₋₆ alkyl, more preferably C₁ alkyl and R¹⁵ is C₁₋₁₂ alkyl, preferably C₁₋₆ alkyl, preferably C₁₋₄ alkyl, more preferably C₂ alkyl or a pharmaceutically acceptable salt thereof.

In one embodiment, the compound of formula (I) is (2R,3S)-2-(6-amino-9H-purin-9-yl)hexan-3-ol.

In a further embodiment, the compound of formula (I) is a compound of formula (I), wherein J is N, K is N and L is NR⁴, R¹ and R² are joined to form a six membered carbocyclic ring optionally including one, two or three unsaturated bonds, preferably three unsaturated bonds wherein two of the carbon atoms which form the 6 membered carbocyclic ring are replaced with a heteroatom selected from N, S and O, preferably N and preferably at positions 2 and 4 of the six membered ring and wherein each one of the atoms which form the 6 membered ring is independently optionally substituted with one or two R³² groups, wherein each R³² is independently selected from hydrogen, halogen, C₁₋₁₂-alkyl, C₂₋₁₂-alkenyl, C₂₋₁₂-alkynyl, aryl, heteroaryl, —OR³³, NR³⁴R³⁴, —COR³⁴, C₃₋₁₂ cycloalkyl and C₃₋₁₀ heterocycloalkyl and R⁴ is a group of formula IIB, wherein A is a single bond, V is selected from —OR¹⁷ and NR¹⁸R¹⁸, wherein R¹⁸ is preferably hydrogen and V is preferably —OH, R¹⁴ is C₁₋₁₂ alkyl, preferably C₃₋₈ alkyl, more preferably C₆ alkyl and R¹⁵ is C₁₋₁₂ alkyl, preferably C₁₋₆ alkyl, preferably C₁₋₃ alkyl, more preferably C₁ alkyl or a pharmaceutically acceptable salt thereof.

In a further embodiment, the compound of formula (I) is a compound of formula (IA), wherein both X and Y are N, Z is NR⁶R⁶, wherein each R⁶ may be the same or different and are preferably the same and are both hydrogen, R³ and R³⁵ are hydrogen and R⁴ is a group of formula (IIB), wherein A is a single bond, V is —OR¹⁷ or hydrogen, R¹⁴ is hydrogen and R¹⁵ is C₁₋₁₂ alkyl, preferably C₂₋₁₀ alkyl, preferably C₆₋₈ alkyl, more preferably C₈ alkyl or a pharmaceutically acceptable salt thereof.

In a further embodiment, the compound of formula (I) is a compound of formula (IA), wherein both X and Y are N, Z is NR⁶R⁶, wherein each R⁶ may be the same or different and are preferably the same and are both hydrogen, R³ and R³⁵ are hydrogen and R⁴ is a group of formula (IIB), wherein A is a single bond, V is —OR¹⁷ or hydrogen, R¹⁴ is C₁₋₁₂ alkyl, preferably C₁₋₁₀ alkyl, preferably C₁₋₆ alkyl, more preferably C₁ alkyl and R¹⁵ is hydrogen or C₁₋₁₂ alkyl, preferably C₁₋₁₀ alkyl, preferably C₁₋₆ alkyl, preferably C₁ alkyl or a pharmaceutically acceptable salt thereof.

In one embodiment, the compound of formula (I) is 9-(nonan-3-yl)-9H-purin-6-amine.

In a preferred embodiment of the present invention, the compound of formula (I) is 3-(6-aminopurin-9-yl)nonan-2-ol (also known as 9-(2-hydroxy-3-nonyl)adenine; or EHNA). EHNA is commercially available from Sigma and Calbiochem. EHNA is generally sold in its hydrochloride salt form as a racemic mixture of the (2R, 3S) and (2S, 3R) stereoisomers.

EHNA and its analogues are well known in the art and have been identified as potent ADA inhibitors and phosphodiesterase-2 inhibitors. In this regard, EHNA has been linked to cardiovascular and cancer chemotherapy/anti-viral applications. The present inventors have surprisingly found that EHNA is particularly effective in inhibiting differentiation in stem cells, particularly embryonic stem cells, in particular, human embryonic stem cells.

Erythro-EHNA is a mixture of the (2R, 3S) and (2S, 3R) stereoisomers of EHNA. Threo-EHNA is a mixture of the (2R, 3R) and (2S, 3S) stereoisomers of EHNA. Preferably the ADA inhibitor of the present invention is erythro-EHNA. Preferably the ADA inhibitor used in the present invention is the (2S, 3R) stereoisomer of EHNA.

Any combination of the specific embodiments described above is disclosed herein.

The skilled person will recognise that the compounds of the invention may be prepared, in a known manner, in a variety of ways. The routes below are merely illustrative of some of the methods that can be employed for the synthesis of compounds of formula (I).

Where R⁴ is a group of formula (IIB), a reagent which is useful in the preparation of compounds of formula (I) is an amino alcohol. Suitable amino alcohols may be prepared from an amino substituted carboxylic acid. The person skilled in the art will be familiar with the conditions required to achieve this. An exemplary reaction scheme is illustrated in Scheme 1, wherein the carboxylic acid is first transformed into a methyl ketone by reaction with acetic anhydride in pyridine and then subsequently reduced further in the presence of potassium borohydride. Further details of this reaction scheme can be found in the publication by Schaeffer and Schwender, J. Med. Chem., 1974, vol. 17, pp 6-8.

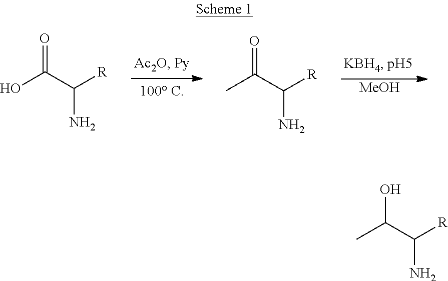

Alternatively, where the group (IIB) of formula (I) is desired to have a particular stereochemistry, a suitable amino alcohol reagent may be prepared by reaction of an epoxide precursor. Suitable epoxide precursors may be produced as shown in scheme 2 below. Further details of this reaction scheme can be found in publication by Abushanab et al, J. Org. Chem., 1988, vol. 53, pp 2598-2602.

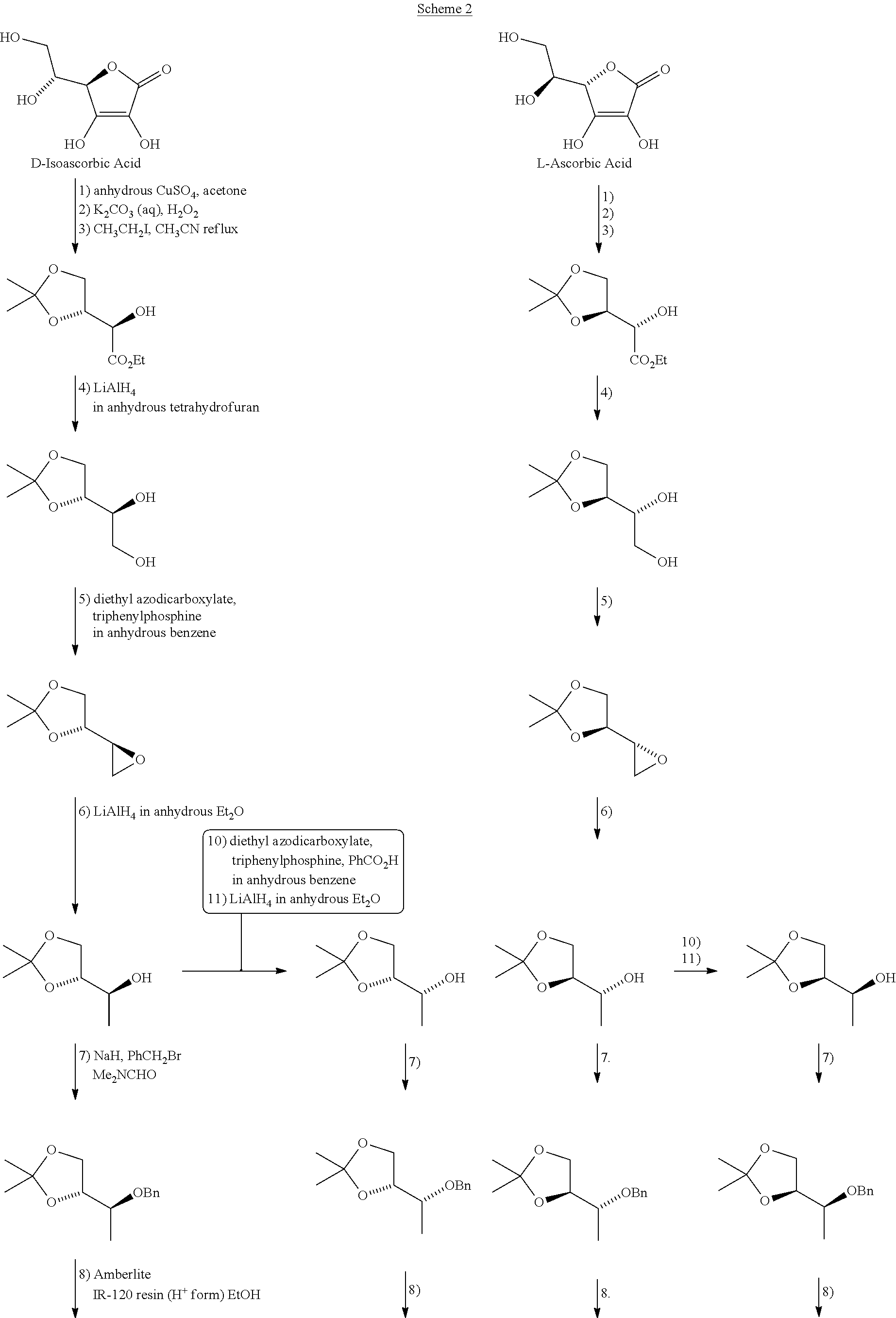

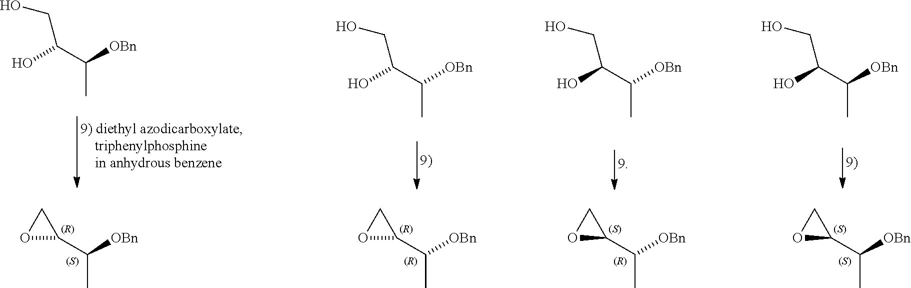

By use of an epoxide precursor, it is possible to prepare an amino alcohol having a specific stereochemistry by the route as set out in Scheme 3 below, for example, wherein an epoxide precursor is reacted with a Grignard reagent to effect opening of the ring, followed by removal of the protecting group. Such a route is well established in the chemical literature, for example, in the publication by Vargeese et al (J. Med. Chem., 1994, vol. 37, pp 3844-3849).

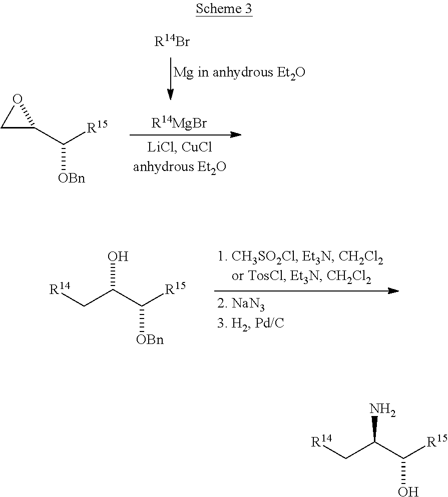

### Imidazole Analogues of EHNA

Imidazole carboxamide compounds of formula (I), e.g. compounds of formula (I), wherein J is N; K is CH; L is NR⁴; R² is H; W is carbonyl; R¹ is NR⁶R⁶, wherein each R⁶ may be the same or different and selected from hydrogen, C₁₋₁₂ alkyl, C₂₋₁₂ alkenyl, optionally substituted with 1, 2 or 3 —OH groups; and R⁴ is a group of formula IIB, wherein V is OH, R¹⁵ is Me, A is a single bond and R¹⁴ is C₁₋₁₂ alkyl may be produced by the series of reactions illustrated in Scheme 4 below and as described in Cristalli et al., J. Med. Chem. 1991, 34, 1187-1192. Thus, starting from an amino alcohol of formula R¹⁴-A-CH(NH₂)—CH(OH)—R¹⁵ sequential reactions with ethyl 2-amino-2-cyanoethanoate and triethyl orthoformate, followed by sodium nitrite and hypophosphorus acid followed by amination of the ester with an amine of formula HNR⁶R⁶ or ammonia affords the imidazole carboxamide compound.

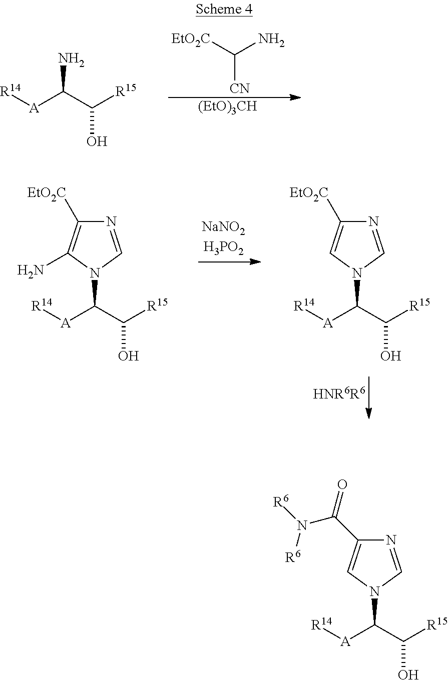

Imidazole carboxamide compounds of formula (I), e.g. compounds of formula (I), wherein J is N; K is CH; L is NR⁴; R² is H; W is carbonyl; R¹ is NR⁶R⁶, wherein each R⁶ may be the same or different and selected from H or C₁₋₃ alkyl; and R⁴ is a group of formula IIB, wherein V is OH, R¹⁵ is Me, A is —CH₂CH₂ and R¹⁴ is C₁₋₁₂ alkyl or aryl may also be produced by the series of reactions illustrated in Scheme 4b below and as described in Terasaka et al., J. Med. Chem., 2005, vol. 48, pp 4750-4753. According to this scheme the following reaction sequence is applied to a chiral 3-(tert-butyldimethylsilyloxy)-2-oxobutylphosphonate starting material (R¹⁵═CH₃): (i) sequential treatment of the phosphonate with n-butyllithium and an aldehyde to facilitate a Horner-Wadsworth-Emmons reaction; (ii) stereoselective reduction of the resulting unsaturated ketone with L-selectride; (iii) hydrogenation; (iv) transformation of the resulting alcohol into a methanesulfonate derivative; (v) alkylation of an imidazole carboxamide using the methanesulfonate from the preceding step; (vi) removal of the tert-butyldimethylsilyl protecting group. The 3-(tert-butyldimethylsilyloxy)-2-oxobutylphosphonate starting material may be prepared as described by Shapiro et al in the publication, Tetrahedron Lett., 1990, vol. 31, pp 5733-5736.

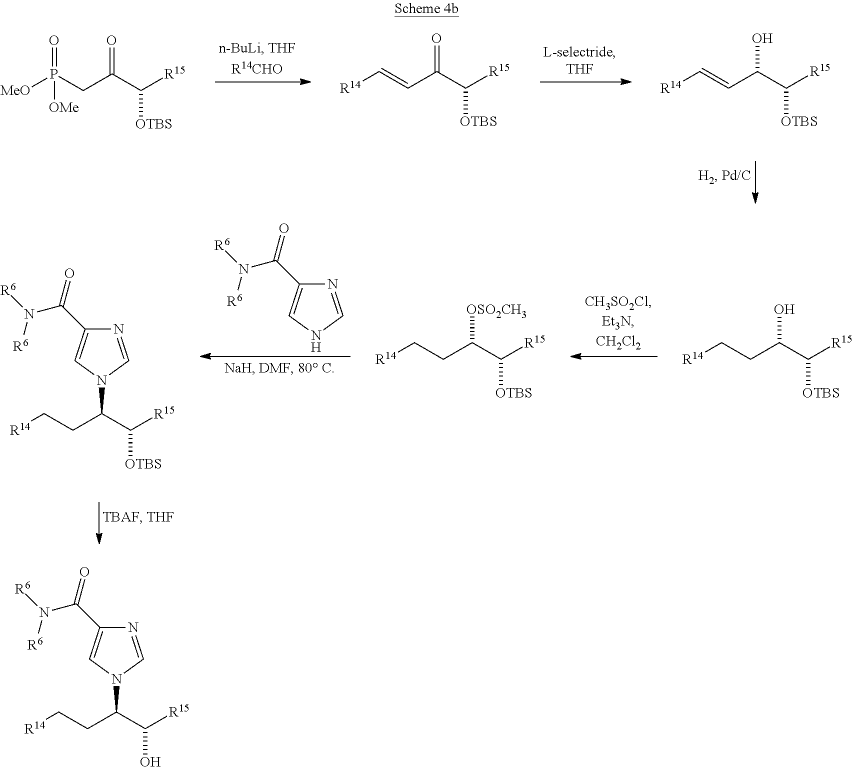

6-(hydroxymethyl)purines Derivatives

Compounds of formula (I) wherein R⁴ is a group of formula (IA) may be prepared as illustrated in Scheme 5 below and as described in detail in \check{S}ilhár et al, Organic Letters, 2004, Vol. 6, 19, 3225-3228. Preparation of the starting material is described in Gerster, J. F.; Jones, J. W.; Robins, R. K. J. Org. Chem., 1963, 28, 945-948. The first compound illustrated in Scheme 5 below is prepared by reacting inosine with acetic anhydride, followed by reaction with POCl₃

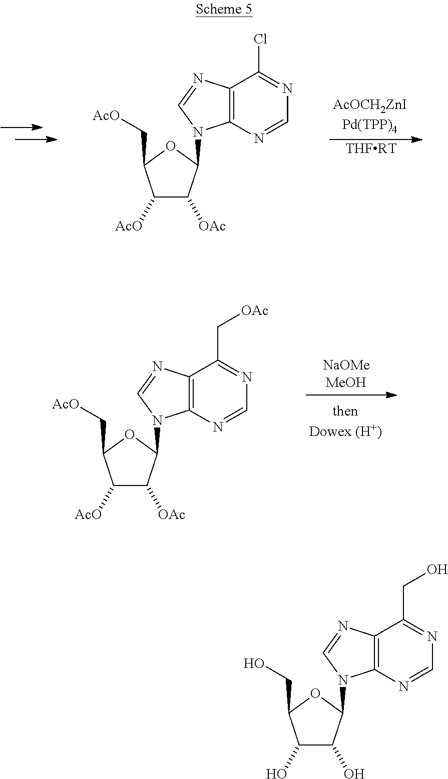

Pyrazolo(3,4-d)pyrimidines

Compounds of formula (I), wherein J is CH, L is N, K is R⁴ and R¹ and R² are joined to form a six membered carbocyclic ring wherein two of the carbon atoms (specifically at positions 3 and 5 in the six membered ring) are substituted for N atoms, the ring contains two double bonds and R⁴ is a C₁₋₁₂ alkyl group may be produced according to the series of reactions illustrated in scheme 6 below and as described in Da Settimo et al. J. Med. Chem. 2005, 48, 5162-5174.

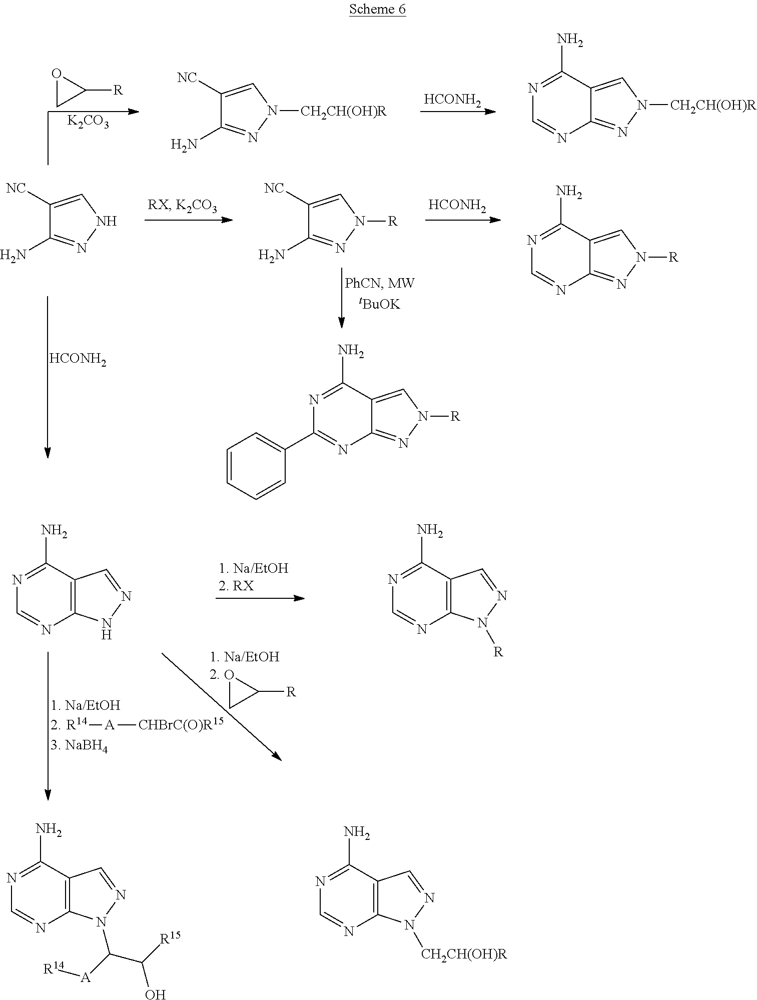

### EHNA and Analogues

As has been described previously, EHNA is a compound which is well known as an ADA inhibitor and its synthesis is therefore well documented. The reaction schemes which follow therefore illustrate one example of a synthetic route by which this compound and its analogues may be prepared.

Amino alcohol starting reagents may be prepared as described earlier. Compounds of formula (IA), wherein both X and Y are N, Z is NR⁶R⁶, wherein each R⁶ may be the same or different and are preferably the same and are both hydrogen, R³ and R³⁵ are hydrogen and R⁴ is a group of formula (IIB), wherein A is a single bond, V is selected from —OR¹⁷ and NR¹⁸R¹⁸, wherein R¹⁸ is preferably hydrogen and V is preferably —OH, R¹⁴ is C₁₋₁₂ alkyl, preferably C₃₋₈ alkyl, more preferably C₆ alkyl and R¹⁵ is C₁₋₁₂ alkyl, preferably C₁₋₆ alkyl, preferably C₁₋₃ alkyl, more preferably C₁ alkyl may be prepared using an amine alcohol starting compound by the series of reactions as illustrated in Schemes 7 and 8 below and as described in Baker et al in the publication, J. Org. Chem. 1982, 47, 2179-2184, and by Schaeffer and Schwender in the publication, J. Med. Chem., 1974, vol. 17, pp 6-8.

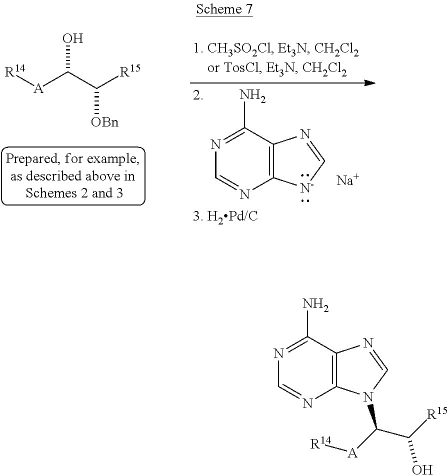

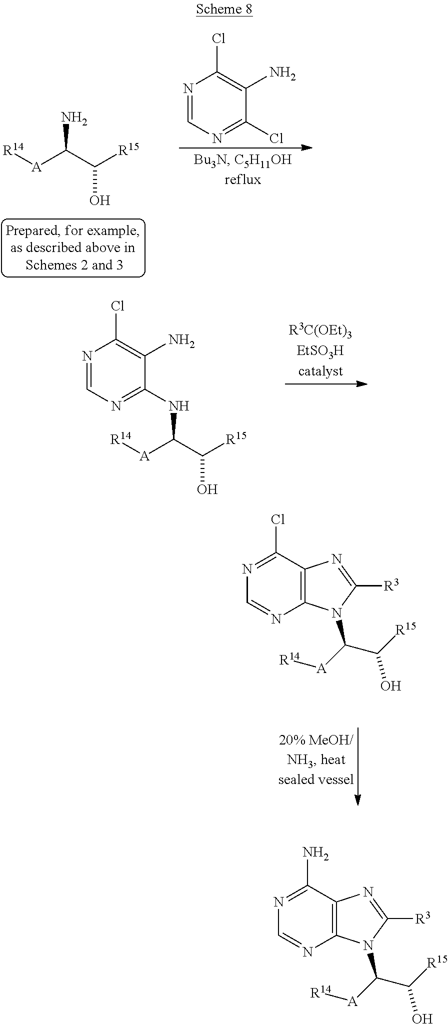

7-Deaza analogues of EHNA, specifically compounds of formula (I), wherein J is CH, K is CH and L is NR⁴, R¹ and R² are joined to form a six membered carbocylic ring optionally including one, two or three unsaturated bonds, preferably three unsaturated bonds wherein two of the carbon atoms which form the 6 membered carbocyclic ring are replaced with a heteroatom selected from N, S and O, preferably N and preferably at positions 2 and 4 of the six membered ring and wherein each one of the atoms which form the 6 membered ring is independently optionally substituted with one or two R³² groups, wherein each R³² is independently selected from hydrogen, halogen, C₁₋₁₂-alkyl, C₂₋₁₂-alkenyl, C₂₋₁₂-alkynyl, aryl, heteroaryl, —OR³³, NR³⁴R³⁴, —COR³⁴, C₃₋₁₂ cycloalkyl and C₃₋₁₀ heterocycloalkyl and R⁴ is a group of formula IIB, wherein A, V, R¹⁴ and R¹⁵ are as defined herein may be prepared according to the reactions illustrated in Scheme 9 and as described in Cristalli et al, J. Med. Chem., 1988, 31, 390-393.

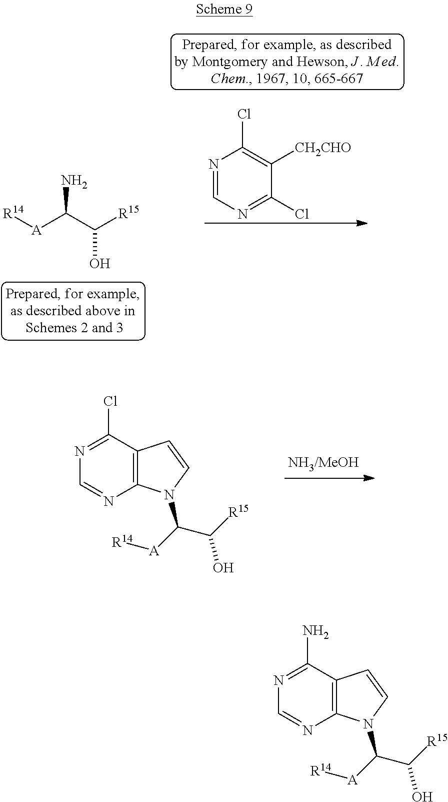

1,3-Deaza analogues of EHNA, specifically compounds of formula (IA), wherein X and Y are both CH, Z is NR⁶R⁶, wherein each R⁶ may be the same or different and are preferably the same and are both hydrogen, R³ and R³⁵ are hydrogen and R⁴ is a group of formula (IIB), wherein A, V, R¹⁴ and R¹⁵ are as defined herein, may be prepared by the series of reactions illustrated in Scheme 10 below and as described in Cristalli et al, J. Med. Chem., 1988, 31, 390-393.

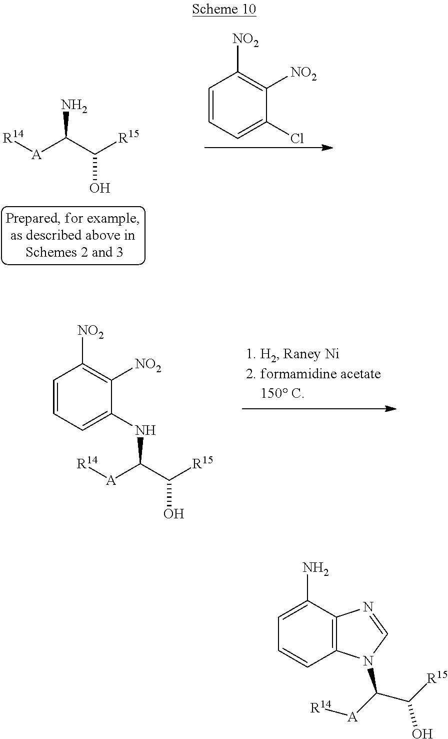

1-Deazapurine analogues of EHNA, specifically compounds of formula (IA), wherein X is CH, Y is N, Z is hydrogen or NR⁶R⁶, wherein each R⁶ may be the same or different and are preferably the same and are both hydrogen, R³ and R³⁵ are hydrogen and R⁴ is a group of formula (IIB), wherein A, V, R¹⁴ and R¹⁵ are as defined herein, may be prepared according to the series of reactions illustrated in Scheme 11 below and as described in Antonini et al., J. Med. Chem. 1984, 27, 274-278.

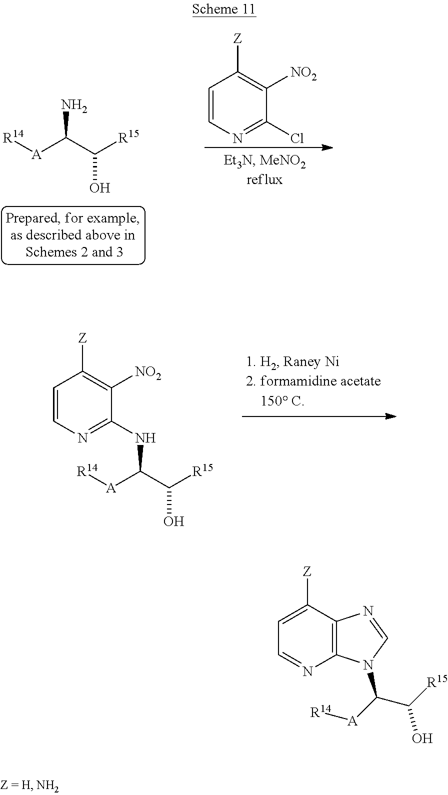

3-Deaza and 3-deazapurine analogues of EHNA, specifically compounds of formula (IA), wherein X is N, Y is CH, Z is hydrogen or NR⁶R⁶, wherein each R⁶ may be the same or different and are preferably the same and are both hydrogen, R³ and R³⁵ are hydrogen and R⁴ is a group of formula (IIB), wherein A, V, R¹⁴ and R¹⁵ are as defined herein, may be prepared by the series of reactions illustrated in scheme 12 below and as described in Antonini et al., J. Med. Chem. 1984, 27, 274-278.

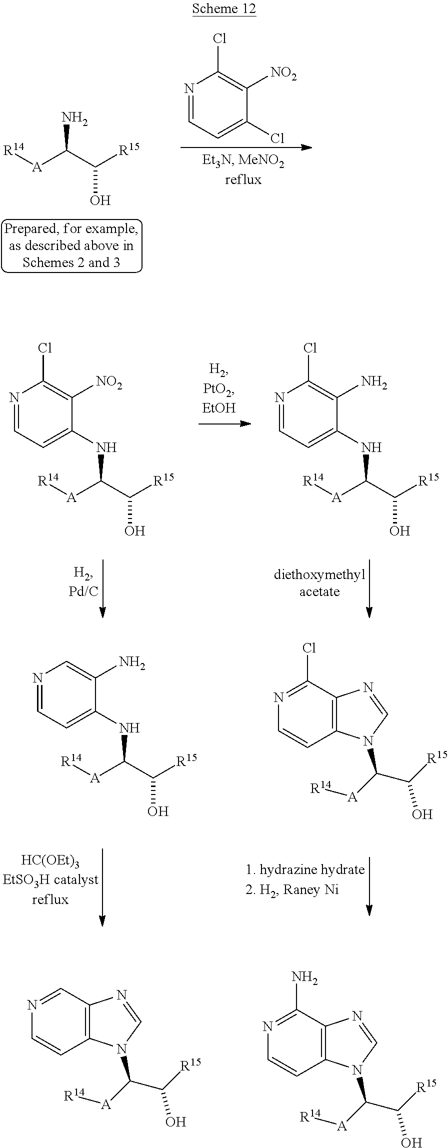

2-Phenyl adenine and 8-aza adenine analogues of EHNA, specifically compounds of formula (IA), wherein both X and Y are N, Z is NR⁶R⁶, wherein each R⁶ may be the same or different and are preferably the same and are both hydrogen, R³ is hydrogen, R³⁵ is selected from hydrogen, halogen, C₁₋₁₂ alkyl, C₂₋₁₂ alkenyl, C₂₋₁₂ alkynyl, —SR³⁶, —OR³⁶, —NR³⁷R³⁷, aryl, heteroaryl, —COR³⁷, C₃₋₁₀ cycloalkyl, C₃₋₁₀ heterocycloalkyl, preferably C₁₋₁₂ alkyl or aryl, preferably aryl and R⁴ is a group of formula (IIB), wherein A, V, R¹⁴ and R¹⁵ are as defined herein, may be prepared by the series of reactions illustrated in Scheme 13 below and as described in Biagi et al., Farmaco, 2002, 57, 221-233.

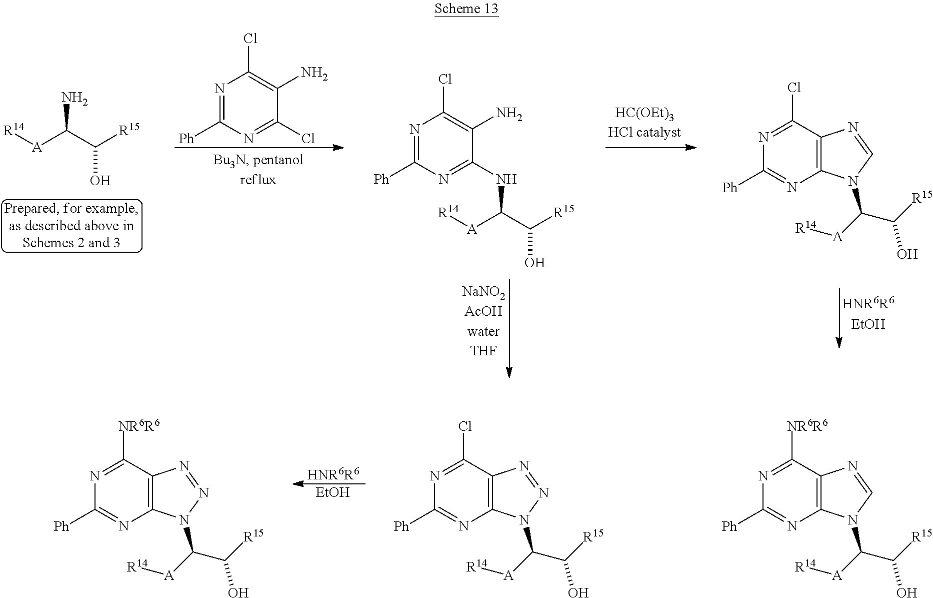

The present invention is concerned with providing a method of inhibiting stem cell differentiation during culture and thus producing large populations of stem cells, in particular pluripotent stem cells. Examples of stem cells which may be used with the present invention include pluripotent stem cells, mesenchymal stem cells, neural stem cells, hematopoietic stem cells, induced-pluripotent stem cells, adipose-derived stem cells and amniotic fluid-derived stem cells. Preferably pluripotent stem cells are used in conjunction with the present invention. Pluripotent stem cells are those that have the potential to differentiate into cells of all three germ layers (endoderm, mesoderm and ectoderm) under appropriate conditions. Pluripotent stem cells are not totipotent i.e. they cannot form an entire organism, such as a foetus. Pluripotent stem cells for use in the method of the invention can be obtained using well-known methods. It is envisaged that various types of pluripotent stem cells may be used in conjunction with the invention, whether obtained from embryonic, foetal or adult tissue. Stem cells may be cloned directly from an organism for use in the invention, but established stem cell lines will typically be used. Accordingly, in some embodiments, the initial population of stem cells are the progeny of previously isolated stem cells or are the progeny of an established stem cell line, such that the invention does not involve any use of a tissue sample.

The ADA inhibitors described herein may be used to inhibit differentiation in mammalian stem cells, particularly primate embryonic stem cells. Primate embryonic stem cells include human, Rhesus monkey and marmoset embryonic stem cells. Mouse embryonic stem cells may also be used. Preferably, the ADA inhibitors of the present invention are used to inhibit stem cell differentiation in embryonic stem cells, more preferably human embryonic stem cells.

ES cells are prepared from the inner cell mass (ICM) of a mammalian blastocyst using known techniques. For example, human ES cells can be obtained using the methods described in Thomson et al. (1998) Science 282:1145-1147, Thomson et al. (1998) Curr. Top. Dev. Biol. 38:133 and U.S. Pat. No. 5,843,780. In some embodiments where hES cells are used, the initial population of cells are the progeny of previously-isolated hES cells or are the progeny of an established line of hES cells, such that the invention does not involve any use of a human embryo. In other embodiments, the initial population of hES cells are the progeny of cells or a cell line obtained using a method that did not involve any use of a human embryo.

Commercially available hES cell lines can be used. Examples of commercially available stem cell lines that might be used in the invention include, but are not limited to: H1, H7, H9, ES01-06, Nott1 & 2, Shef1-7, NCL1-7 and RH1-7.

In the method of the invention, the ADA inhibitors as described herein are used to inhibit stem cell differentiation by contacting the ADA inhibitor with the stem cell. In this regard, the method of the present invention may generally involve the steps of providing a population of pluripotent stem cells, providing a culture medium which comprises, inter alia, an ADA inhibitor as defined herein, contacting the stem cells with the culture medium and culturing the cells under appropriate conditions. The method may include a further step, after the culturing step, of passaging the cells into a further culture medium and then further culturing the cells under appropriate conditions. These steps may be performed in any order.

The ADA inhibitor may be added to the culture medium prior to contact with the stem cells. The ADA inhibitor is added to the culture medium in an amount sufficient to inhibit the differentiation of the pluripotent cells which will be cultured thereon. In one embodiment, the ADA inhibitor is added to the culture medium in an amount in the range from about 1 nM to about 10 mM, alternatively in an amount in the range from about 1×10⁻⁸ M to about 1×10⁻³ M, alternatively in an amount in the range from about 1×10⁻⁷ M to about 1×10⁻⁴ M, alternatively in an amount in the range from about 1×10⁻⁶ M to about 1×10⁻⁵ M. The present inventors have surprisingly found that by including an ADA inhibitor in the culture medium, a high level of inhibition of differentiation can be obtained in the absence of exogenous FGF in the culture medium. Therefore, the amount of FGF in the culture medium may be reduced or eliminated.

In this regard, the present invention further provides a culture medium for expanding a population of pluripotent stem cells comprising an ADA inhibitor as defined herein. Cell culture media typically contain a large number of ingredients, which are necessary to support maintenance of cultured cells. A culture medium of the invention will therefore normally contain many other ingredients in addition to an ADA inhibitor. Suitable combinations of ingredients can readily be formulated by the skilled person. A culture medium according to the invention will generally be a nutrient solution comprising standard cell culture ingredients, such as amino acids, vitamins, inorganic salts, a carbon energy source, and a buffer.

A culture medium according to the invention may be generated by modification of an existing cell culture medium. The skilled person understands the types of culture media that might be used for pluripotent stem cell culture. Potentially suitable cell culture media are available commercially, and include Dulbecco's Modified Eagle Media (DMEM), Minimal Essential Medium (MEM), Knockout-DMEM (KO-DMEM), Glasgow. Minimal Essential Medium (G-MEM), Basal Medium Eagle (BME), DMEM/Ham's F12, Advanced DMEM/Ham's F12, Iscove's Modified Dulbecco's Media and Minimal Essential Media (MEM).

A culture medium for use in the invention may comprise one or more amino acids. The skilled person understands the appropriate types and amounts of amino acids for use in stem cell culture media. Amino acids which may be present include L-alanine, L-arginine, L-asparagine, L-aspartic acid, L-cysteine, L-cystine, L-glutamic acid, L-glutamine, glycine, L-histidine, L-isoleucine, L-leucine, L-lysine, L-methionine, L-phenyl alanine, L-proline, L-serine, L-threonine, L-tryptophan, L-tyrosine, L-valine and combinations thereof. Some culture media will contain all of these amino acids. Generally, each amino acid when present is present at about 0.001 to about 1 g/L of medium (usually at about 0.01 to about 0.15 g/L), except for L-glutamine which is present at about 0.05 to about 1 g/L (usually about 0.1 to about 0.75 g/L). The amino acids may be of synthetic origin.

A culture medium for use in the invention may comprise one or more vitamins. The skilled person understands the appropriate types and amounts of vitamins for use in stem cell culture media. Vitamins which may be present include thiamine (vitamin B1), riboflavin (vitamin B2), niacin (vitamin B3), D-calcium pantothenate (vitamin B5), pyridoxal/pyridoxamine/pyridoxine (vitamin B6), folic acid (vitamin B9), cyanocobalamin (vitamin B12), ascorbic acid (vitamin C), calciferol (vitamin D2), DL-alpha tocopherol (vitamin E), biotin (vitamin H) and menadione (vitamin K).

A culture medium for use in the invention may comprise one or more inorganic salts. The skilled person understands the appropriate types and amounts of inorganic salts for use in stem cell culture media. Inorganic salts are typically included in culture media to aid maintenance of the osmotic balance of the cells and to help regulate membrane potential. Inorganic salts which may be present include salts of calcium, copper, iron, magnesium, potassium, sodium, zinc. The salts are normally used in the form of chlorides, phosphates, sulphates, nitrates and bicarbonates. Specific salts that may be used include CaCl₂, CuSO₄-5H₂O, Fe(NO₃)-9H₂O, FeSO₄-7H₂O, MgCl, MgSO₄, KCl, NaHCO₃, NaCl, Na₂HPO₄, Na₂HPO₄—H₂O and ZnSO₄-7H₂O.

The osmolarity of the medium may be in the range from about 200 to about 400 mOsm/kg, in the range from about 290 to about 350 mOsm/kg, or in the range from about 280 to about 310 mOsm/kg. The osmolarity of the medium may be less than about 300 mOsm/kg (e.g. about 280 mOsm/kg).

A culture medium for use in the invention may comprise a carbon energy source, in the form of one or more sugars. The skilled person understands the appropriate types and amounts of sugars to use in stem cell culture media. Sugars which may be present include glucose, galactose, maltose and fructose. The sugar is preferably glucose, particularly D-glucose (dextrose). A carbon energy source will normally be present at between about 1 and about 10 g/L.

A culture medium for use in the invention may comprise a buffer. A suitable buffer can readily be selected by the skilled person. The buffer may be capable of maintaining the pH of the culture medium in the range about 6.5 to about 7.5 during normal culturing conditions, most preferably around pH 7.0. Buffers that may be used include carbonates (e.g. NaHCO₃), chlorides (e.g. CaCl₂), sulphates (e.g. MgSO₄) and phosphates (e.g. NaH₂PO₄). These buffers are generally used at about 50 to about 500 mg/l. Other buffers such as N-[2-hydroxyethyl]-piperazine-N′-[2-ethanesul-phonic acid] (HEPES) and 3-[N-morpholino]-propanesulfonic acid (MOPS) may also be used, normally at around 1000 to around 10,000 mg/l.

A culture medium of the invention may contain serum. Serum obtained from any appropriate source may be used, including fetal bovine serum (FBS), goat serum or human serum. Preferably, human serum is used. Serum may be used at between about 1% and about 30% by volume of the medium, according to conventional techniques.

In other embodiments, a culture medium of the invention may contain a serum replacement. Various different serum replacement formulations are commercially available and are known to the skilled person. Where a serum replacement is used, it may be used at between about 1% and about 30% by volume of the medium, according to conventional techniques.

In other embodiments, a culture medium of the invention may be serum-free and/or serum replacement-free. A serum-free medium is one that contains no animal serum of any type. Serum-free media may be preferred to avoid possible xeno-contamination of the stem cells. A serum replacement-free medium is one that has not been supplemented with any commercial serum replacement formulation.

A culture medium may comprise cholesterol or a cholesterol substitute. Cholesterol may be provided in the form of the HDL or LDL extract of serum. Where the HDL or LDL extract of serum is used, it is preferably the extract of human serum. The optimal amount of cholesterol or cholesterol substitute can readily be determined from the literature or by routine experimentation. A synthetic cholesterol substitute may be used rather than cholesterol derived from an animal source. For example, Synthecol™ (Sigma S5442) may be used in accordance with the manufacturer's instructions.

The culture medium may further comprise transferrin or a transferrin substitute. Transferrin may be provided in the form of recombinant transferrin or in the form of an extract from serum. Preferably, recombinant human transferrin or an extract of human serum is used. An iron chelate compound may be used as a transferrin substitute. Suitable iron chelate compounds are known to those of skill in the art, and include ferric citrate chelates and ferric sulphate chelates. The optimal amount of transferrin or transferrin substitute can readily be determined from the literature or by routine experimentation. In some embodiments, a culture medium of the invention may comprise transferrin at about 5.5 μg/ml.

The culture medium may further comprise albumin or an albumin substitute, such as bovine serum albumin (BSA), human serum albumin (HSA), a plant hydrolysate (e.g. a rice or soy hydrolysate), Albumax® I or Albumax® II. The optimal amount of albumin or albumin substitute can readily be determined from the literature or by routine experimentation. In some embodiments, a culture medium of the invention may comprise albumin at about 0.5 μg/ml.

The culture medium may further comprise insulin or an insulin substitute. Natural or recombinant insulin may be used. A zinc-containing compound may be used as an insulin substitute, e.g. zinc chloride, zinc nitrate, zinc bromide or zinc sulphate. The optimal amount of insulin or insulin substitute can readily be determined from the literature or by routine experimentation. In some embodiments, a culture medium of the invention may comprise insulin at about 10 μg/ml.

The culture medium may comprise progesterone, putrescine, and/or selenite. If selenite is present, it is preferably in the form of sodium selenite. The optimal amount of these ingredients can readily be determined from the literature or by routine experimentation.

A culture medium of the invention may comprise one or more additional nutrients or growth factors that have previously been reported to benefit pluripotent stem cell culture. For example, a culture medium may comprise transforming growth factor beta 1 (TGFβ1), leukemia inhibitor factor (LIF), ciliary neurotrophic factor (CNTF), interleukin 6 (IL-6) or stem cell factor (SCF). Antibodies or other ligands that bind to the receptors for such substances may also be used.

A culture medium for use in the invention may comprise one or more trace elements, such as ions of barium, bromium, cobalt, iodine, manganese, chromium, copper, nickel, selenium, vanadium, titanium, germanium, molybdenum, silicon, iron, fluorine, silver, rubidium, tin, zirconium, cadmium, zinc and/or aluminium.

A culture medium may further comprise phenol red as a pH indicator, to enable the status of the medium to be easily monitored (e.g. at about 5 to about 50 mg/litre).

The medium may comprise a reducing agent, such as β-mercaptoethanol at a concentration of about 0.1 mM.

The invention may be used in conjunction with a culture medium as described in GB application No. 0810304.6 filed on 5 Jun. 2008 or a culture medium as described in GB application No. 0821363.9 filed on 21 Nov. 2008. The culture media described in GB 0810304.6 and GB 0821363.9 comprise, amongst other ingredients, a farnesoid X receptor (FXR) agonist, a retinoid X receptor (RXR) or retinoic acid receptor (RAR) agonist, a peroxisome proliferator-activated receptor (PPAR) agonist, and/or a thyroid hormone receptor (THR) agonist.

The RXR or RAR agonist may be a retinoid, preferably a retinol, a retinol or retinoic acid. In one embodiment, the RXR or RAR agonist is all-trans-retinol (ATR), 13-cis retinoic acid (13cRA), 9-cis retinoic acid (9cRA), methoprene acid (MPA), 13-cis retinol (13cROL), retinyl acetate (RETACT), acitretin (ACT) or 4-hydroxyretinoic acid (4HRA).

The FXR agonist may be a cholesterol metabolite such as a bile acid selected from cholic acid (CA), deoxycholic acid (DCA), chenodeoxycholic acid (CDCA) or lithocholic acid (LCA), or a glycine or taurine conjugate thereof. Alternatively, the FXR agonist may be an arachidonic acid, a linolenic acid or a docosahexaenoic acid, specifically α-linolenic acid (ALA), γ-linolenic acid (GLA) or di-homo-γ-linolenic acid (DGLA).

The PPAR agonist may be an unsaturated fatty acid, a saturated fatty acid, a dicarboxylic fatty acid, an eicosanoid, a prostaglandin 12 analog, a leukotriene B4 analog, a leukotriene D4 antagonist, a hypolipidemic agent, a hypoglycemic agent, a hypolipidemic and hypoglycaemic agent, a nonsteroidal anti-inflammatory drug, a carnitine palmitoyl transferase I (CPT1) inhibitor, or a fatty acyl-CoA dehydrogenase inhibitor. In one embodiment, the PPAR agonist may be 5,8,11,14-eicosatetraynoic acid, bezafibrate, clofibric acid, gemfibrozil, WY14643 or tetradecylthioacetic acid.

The THR agonist may be an iodothyronine, such as a di-iodothyronine, tri-iodothyronine or tetra-iodothyronine, for example 3,5-diiodothyronine (3,5-T₂), 3,3′-diiodothyronine (3,3-T₂), 3,3′-T₂ sulphate (3,3-T₂S), 3,5-diiodo-L-tyrosine dihydrate (DLTdH), 3,5,3′-triiodo-L-thyronine (T3), 3,3′,5-T₃ sulphate (3,3′,5-T₃S), 3,5,3′,5′-tetra-iodothyronine (T4), 3,5,3′,5′-tetraiodo-L-thyronine or 3,5-diiodo-4-hydroxyphenylpropionic acid (DIHPA). The THR agonist may be 3,5-diiodo-L-thyronine.

In one embodiment, the culture medium for use in the present invention may comprise a bile acid, a retinol and a diiodothyronine, preferably cholic acid, all-trans-retinol and 3,5-diiodo-L-thyronine. In one embodiment the culture medium for use in the present invention may comprise cholic acid, all-trans-retinol, 3,5-diiodo-L-thyronine, cholesterol, transferrin, L-glutamine, progesterone, putrescine, insulin, selenite and DL-alpha-tocopherol (Vitamin E).

The present invention may be used in conjunction with a culture medium as described in Example 2 of GB 0810304.6. Alternatively, the present invention may be used in conjunction with a culture medium as described in Example 3 of GB 0810304.6 Alternatively, the present invention may be used in conjunction with a culture medium as described in Example 2 of GB 0821363.9. Alternatively, the present invention may be used in conjunction with a culture medium as described in Example 3 of GB 0821363.9. Alternatively, the present invention may be used in conjunction with a culture medium as described in Example 4 of GB 0821363.9. Alternatively, the present invention may be used in conjunction with a culture medium as described in Example 5 of GB 0821363.9.

In one embodiment, a culture medium may comprise transferrin, insulin, progesterone, putrescine, and sodium selenite.

‘N2 Supplement’ (available from Invitrogen, Carlsbad, Calif.; www.invitrogen.com; catalog no. 17502-048; and from PAA Laboratories GmbH, Pasching, Austria; www.paa.com; catalog no. F005-004; Bottenstein & Sato, PNAS, 76(1):514-517, 1979) may be used to formulate a culture medium that comprises contains transferrin, insulin, progesterone, putrescine, and sodium selenite. N2 Supplement is supplied by PAA Laboratories GmbH as a 100× liquid concentrate, containing 500 μg/ml human transferrin, 500 μg/ml bovine insulin, 0.63 μg/ml progesterone, 1611 μg/ml putrescine, and 0.52 μg/ml sodium selenite. N2 Supplement may be added to a culture medium as a concentrate or diluted before addition to a culture medium. It may be used at a 1× final concentration or at other final concentrations. Use of N2 Supplement is a convenient way to incorporate transferrin, insulin, progesterone, putrescine and sodium selenite into a culture medium for use in the invention.

In one embodiment, a culture medium may comprise biotin, cholesterol, linoleic acid, linolenic acid, progesterone, putrescine, retinol, retinyl acetate, sodium selenite, tri-iodothyronine (T3), DL-alpha tocopherol (vitamin E), albumin, insulin and transferrin.

‘B27 Supplement’ (available from Invitrogen, Carlsbad, Calif.; www.invitrogen.com; currently catalog no. 17504-044; and from PAA Laboratories GmbH, Pasching, Austria; www.paa.com; catalog no. F01-002; Brewer et al., J Neurosci Res., 35(5):567-76, 1993) may be used to formulate a culture medium that comprises biotin, cholesterol, linoleic acid, linolenic acid, progesterone, putrescine, retinol, retinyl acetate, sodium selenite, tri-iodothyronine (T3), DL-alpha tocopherol (vitamin E), albumin, insulin and transferrin. B27 Supplement is supplied by PAA Laboratories GmbH as a liquid 50× concentrate, containing amongst other ingredients biotin, cholesterol, linoleic acid, linolenic acid, progesterone, putrescine, retinol, retinyl acetate, sodium selenite, tri-iodothyronine (T3), DL-alpha tocopherol (vitamin E), albumin, insulin and transferrin. Of these ingredients at least linolenic acid, retinol, retinyl acetate and tri-iodothyronine (T3) are nuclear hormone receptor agonists as described elsewhere herein. B27 Supplement may be added to a culture medium as a concentrate or diluted before addition to a culture medium. It may be used at a 1× final concentration or at other final concentrations. Use of B27 Supplement is a convenient way to incorporate biotin, cholesterol, linoleic acid, linolenic acid, progesterone, putrescine, retinol, retinyl acetate, sodium selenite, tri-iodothyronine (T3), DL-alpha tocopherol (vitamin E), albumin, insulin and transferrin into a culture medium for use in the invention.

N2 Supplement and B27 Supplement may be used in combination in a culture medium for use in the invention.

The culture medium may be a conditioned medium. Conditioned medium is produced by culturing a population (typically of non-pluripotent) cells in a culture medium for a time sufficient to condition the medium, then harvesting the conditioned medium. Conditioned medium contains growth factors, cytokines and other nutrients secreted by the conditioning cells that support growth of stem cells. In some embodiments, the medium comprises conditioned VitroHES (VitroLife AB, Sweden).

Where a conditioned medium is used, the medium may be conditioned on mammalian cells, e.g. mouse cells or human cells. Various different types of mammalian cells may be used to produce conditioned medium suitable for pluripotent stem cell culture, including mouse embryonic fibroblasts (mEF), human foreskin cells and human fallopian epithelial cells. Preferably, mEF cells are used. Conditioned medium may be prepared by well known methods, e.g. by culturing mEFs and harvesting the culture medium after an appropriate time (e.g. ˜1 day at 37° C.). The cells used to condition a medium may be irradiated or treated with a substance (e.g. mitomycin C) to prevent their proliferation.

An appropriate culturing time to condition a medium may be estimated by the skilled person, based on known methods. Alternatively, the time required to condition the medium can be determined by assessing the effect of the conditioned medium on pluripotent stem cell growth and differentiation. The conditioning time can be altered after assessing the effect of the conditioned medium on stem cell growth and differentiation. Typically, a medium will be conditioned for between about 1 and about 72 hours, such as between about 4 hours and about 48 hours, or between about 4 hours and about 24 hours, at 37° C.

The period over which a conditioned medium can support pluripotent stem cell expansion may likewise be estimated by the skilled person, based on known methods, or may be assessed experimentally. The period before replacement or exchange of conditioned medium can therefore be altered after assessing the effect of a conditioned medium on stem cell growth and differentiation. Conditioned medium is typically used to support cell growth for between about 6 hours and about 72 hours, such as between about 12 hours and about 56 hours, e.g. for about 24-36 hours or for about 24-48 hours, before replacement or exchange with a further batch of conditioned medium.

Where a conditioned medium is used, it has surprisingly been found that a high level of inhibition of differentiation can be obtained without adding FGF to the conditioned media.

Alternatively, the culture medium may be a fresh culture medium. A fresh medium is a medium that has not been conditioned. A fresh medium may be preferred, because such a medium may be chemically defined (i.e. all of the ingredients in the medium and their concentrations may be known), in contrast to a conditioned medium (which is not fully defined because the conditioning cells alter the composition of the medium, and because of batch-to-batch variations).

Alternatively, the culture medium may be a mixture of a fresh medium and a conditioned medium. When a conditioned medium and a fresh medium are mixed, the conditioned medium and the fresh medium may be of the same type or may be of different types. By ‘different types’ is meant that the combination of ingredients in the conditioned medium prior to conditioning is different to the combination of ingredients in the fresh medium (e.g. the conditioned medium may be conditioned VitroHES and the fresh medium may be DMEM/F12). In other words, the mixed culture medium is not one that would be obtained by merely diluting a concentrated medium with an non-concentrated or diluted form of the same medium, nor is it one that would be obtained by adding to a conditioned medium more fresh medium of the same type.

The use of a mixture of a conditioned medium and a fresh medium of different types may be preferred, as it may provide a more complex nutrient mixture that is of further benefit to pluripotent stem cells in culture. Accordingly, in one aspect the invention provides a method for preparing a mixed culture medium for expanding a population of pluripotent stem cells, comprising: (a) providing a conditioned medium; (b) providing a fresh medium; (c) adding an ADA inhibitor to the fresh medium; and (d) mixing at least part of the conditioned medium with at least part of the fresh medium, thereby forming a mixed culture medium, wherein the conditioned medium and the fresh medium are of different types. The invention also provides methods, compositions and uses as described herein involving such mixed culture media.

In this regard, the culture medium may be a mixture of a conditioned medium and a fresh medium of different types, which comprises a bile acid, a retinol and a diiodothyronine. The culture medium may be a mixture of a conditioned medium and a fresh medium of different types, which comprises cholic acid, all-trans-retinol and 3,5-diiodo-L-thyronine.

In one embodiment, the culture medium may be a mixture of a conditioned medium and a fresh medium, which comprises biotin, cholesterol, linoleic acid, linolenic acid, progesterone, putrescine, retinol, retinyl acetate, sodium selenite, tri-iodothyronine (T3), DL-alpha tocopherol (vitamin E), albumin, insulin and transferrin.

B27 Supplement (available from Invitrogen, Carlsbad, Calif.; www.invitrogen.com; currently catalog no. 17504-044; and from PAA Laboratories GmbH, Pasching, Austria; www.paa.com; catalog no. F01-002; Brewer et al., J Neurosci Res., 35(5):567-76, 1993) and/or N2 Supplement (available from Invitrogen, Carlsbad, Calif.; www.invitrogen.com; catalog no. 17502-048; and from PAA Laboratories GmbH, Pasching, Austria; www.paa.com; catalog no. F005-004; Bottenstein & Sato, PNAS, 76(1):514-517, 1979) may be used to formulate such a culture medium. As noted elsewhere herein, use of B27 Supplement is a convenient way to incorporate biotin, cholesterol, linoleic acid, linolenic acid, progesterone, putrescine, retinol, retinyl acetate, sodium selenite, tri-iodothyronine (T3), DL-alpha tocopherol (vitamin E), albumin, insulin and transferrin into a culture medium for use in the invention. As noted elsewhere herein, use of N2 Supplement is a convenient way to incorporate transferrin, insulin, progesterone, putrescine and sodium selenite into a culture medium for use in the invention.

In one embodiment, the culture medium is a mixture of a conditioned medium and a fresh medium, which has been supplemented with B27 Supplement and N2 Supplement.

In one embodiment, the culture medium is a mixture of a conditioned medium and a fresh medium of different types, which comprises biotin, cholesterol, linoleic acid, linolenic acid, progesterone, putrescine, retinol, retinyl acetate, sodium selenite, tri-iodothyronine (T3), DL-alpha tocopherol (vitamin E), albumin, insulin and transferrin.

In one embodiment, the culture medium is a mixture of a conditioned medium and a fresh medium of different types, which has been supplemented with B27 Supplement and N2 Supplement. In this embodiment, the fresh medium may be DMEM/F12 (Invitrogen). The conditioned medium may be VitrohES (Vitrolife AB) conditioned on mouse embryonic fibroblast cells. For example, the culture medium may comprise a mixture of mEF-conditioned VitrohES and fresh DMEM/F12, which has been supplemented with B27 Supplement and N2 Supplement.

If a conditioned medium is mixed with a fresh medium, the conditioned medium and fresh medium may be mixed to form a mixed culture medium that comprises at least 5%, at least 10%, at least 15%, at least 20%, at least 25%, at least 30%, at least 35%, at least 40%, at least 50%, at least 60%, at least 70%, at least 80% or at least 90%, by volume (or by dry weight) conditioned medium.

If a conditioned medium is mixed with a fresh medium, the conditioned medium and fresh medium may be mixed to form a mixed culture medium that comprises at least 5%, at least 10%, at least 15%, at least 20%, at least 25%, at least 30%, at least 35%, at least 40%, at least 50%, at least 60%, at least 70%, at least 80% or at least 90%, by volume (or by dry weight) fresh medium.

A culture medium may be a 1× formulation or a concentrated formulation, e.g. a 2× to 250× concentrated medium formulation. In a 1× formulation each ingredient in the medium is at the concentration intended for cell culture. In a concentrated formulation one or more of the ingredients is present at a higher concentration than intended for cell culture. Concentrated culture media is well known in the art. Culture media can be concentrated using known methods e.g. salt precipitation or selective filtration. A concentrated medium may be diluted for use with water (preferably deionized and distilled) or any appropriate solution, e.g. an aqueous saline solution, an aqueous buffer or a culture medium.

The present invention further provides a method for preparing a culture medium as defined above comprising the steps of (a) obtaining a culture medium; and (b) adding an ADA inhibitor to the culture medium.

The present invention further provides a culture medium supplement that comprises an ADA inhibitor as defined herein.

A “culture medium supplement” is a mixture of ingredients that cannot itself support pluripotent stem cells, but which enables or improves pluripotent stem cell culture when combined with other cell culture ingredients. The supplement can therefore be used to produce functional cell culture medium of the invention by combining with other cell culture ingredients to produce appropriate medium formulation. The use of culture medium supplements is well known in the art.

A culture medium supplement may be a concentrated liquid supplement (e.g. a 2× to 250× concentrated liquid supplement) or may be a dry supplement. Both liquid and dry supplements are well known in the art. A supplement may be lyophilised.

A supplement of the invention may be sterilized prior to use to prevent contamination, e.g. by ultraviolet light, heating, irradiation or filtration. A culture medium supplement may be frozen (e.g. at −20° C. or −80° C.) for storage or transport.

The cells may be cultured in contact with an extracellular matrix material or in contact with a feeder cell layer. Feeder cell layers are often used to support a culture of pluripotent stem cells, and to inhibit their differentiation. A feeder cell layer is generally a monolayer of cells that is co-cultured with, and which provides a surface suitable for growth of, the pluripotent cells of interest. The feeder cell layer provides an environment in which the cells of interest can grow. Feeder cells are typically mitotically inactivated (e.g. by irradiation or treatment with mitomycin C) to prevent their proliferation. The person skilled in the art will be familiar with the use of a layer of feeder cells.

Alternatively, the cells may be cultured in contact with an extracellular matrix material. A variety of substances have been used as extracellular matrix materials for pluripotent stem cell culture, and an appropriate material can readily be selected by the skilled person. An extracellular matrix material may comprise fibronectin, vitronectin, laminin, collagen (particularly collagen II, collagen III or collagen IV), thrombospondin, osteonectin, secreted phosphoprotein 1, heparan sulphate, dermatan sulphate, gelatine, merosin, tenasin, decorin, entactin or a basement membrane preparation from Engelbreth-Holm-Swarm (EHS) mouse sarcoma cells (e.g. Matrigel®; Becton Dickenson). Mixtures of extracellular matrix materials may be used, if desired.

Preferably, the extracellular matrix material comprises fibronectin. Bovine fibronectin, recombinant bovine fibronectin, human fibronectin, recombinant human fibronectin, mouse fibronectin, recombinant mouse fibronectin or synthetic fibronectin may be used.

The stem cells may be cultured in an environment which is sterile and/or temperature stable.

Cells may be passaged in the methods of the invention using known methods, e.g. by incubating the cells with trypsin and EDTA for 5-15 minutes at 37° C. A trypsin substitute (e.g. TrypLE from Invitrogen) may be used, if desired. Collagenase, dispase, accutase or other known reagents may also be used to passage the cells. Passaging is typically required every 2-8 days, such as every 4-7 days, depending on the initial seeding density. In some embodiments, the cell culture methods of the invention do not comprise any step of manually selecting undifferentiated cells when the cells are passaged. In some embodiments, the passaging of the cells may be automated, i.e. without manipulation by a laboratory worker.

The pluripotent stem cells will be seeded onto a support at a density that promotes cell proliferation but which limits differentiation. Typically, a plating density of at least 15,000 cells/cm² is used. A plating density of between about 15,000 cells/cm² and about 200,000 cells/cm² may be used. Single-cell suspensions or small cluster of cells will normally be seeded, rather than large clusters of cells, as in known in the art.

In the methods of the present invention, the stem cells may be cultured in any suitable cell culture vessel as a support. Cell culture vessels of various shapes and sizes (e.g. flasks, single or multiwell plates, single or multiwell dishes, bottles, jars, vials, bags, bioreactors) and constructed from various different materials (e.g. plastic, glass) are known in the art, A suitable cell culture vessel can readily be selected by the person skilled in the art.

The present invention further provides a hermetically-sealed vessel containing a culture medium of the invention. Hermetically-sealed vessels may be preferred for transport or storage of the culture media, to prevent contamination. The vessel may be any suitable vessel, such as a flask, a plate, a bottle, a jar, a vial or a bag.

As has been described above, it has surprisingly been found that, where an ADA inhibitor is included in the culture medium upon which the stem cells are cultured, differentiation of the pluripotent cells is inhibited. In particular, it has been found that the level of inhibition of differentiation is equal to or better than the level of inhibition of differentiation observed when FGF is present in the culture medium. The person skilled in the art will be readily familiar with the various techniques and methods which can be used to identify undifferentiated, pluripotent and proliferative cells and for identifying the proportion of a population of cultured stem cells which are undifferentiated, pluripotent and proliferative.

In order to ascertain the efficacy of the compounds of the present invention, each compound was tested to determine whether it can maintain the stem cell marker NANOG and/or block the differentiation marker PAX6 in the face of differentiating conditions in a manner similar to EHNA. Cells enzymatically passaged onto matrigel-coated dishes and grown for 2 weeks in defined media with or without (control) compound addition were analysed by qRT-PCR to determine the level of NANOG and PAX6 expression. In clarifying which compounds had a full EHNA-like effect, i.e. in order to identify those compounds which have a similar effect to EHNA, on gene expression those which maintained at least 50% of the level of NANOG-expression in comparison to EHNA and those that inhibited the expression of PAX6 to 50% or less than the value of the untreated controls were considered to have an EHNA-like effect. Those which had a single effect, to these levels, on either PAX6 or NANOG, were considered to have a partial-EHNA effect.

An ADA inhibitor which inhibits stem cell differentiation is one which reduces stem cell differentiation by at least about 10%, preferably at least about 20%, preferably at least about 30%, preferably at least about 40%, preferably at least about 50%, preferably at least about 60%, preferably at least about 70%, preferably about 80%, preferably at least about 85%, preferably at least about 90%, preferably at least about 91%, preferably at least about 92%, preferably at least about 93%, preferably at least about 94%, preferably at least about 95%.

The % by which stem cell differentiation has been reduced can be readily determined by the person skilled in the art. In particular, the % by which stem cell differentiation has been reduced can be determined using staining to evaluate reduction in expression of one or more stem cells marker, such as OCT 4, SSEA, SSEA4, TRA1-60 AND TRA1-80 or increase in expression of one or more differentiation marker, such as SSEA1. A suitable protocol is described below:
(i) remove medium from cells and wash several times with phosphate buffered saline (PBS);
(ii) if staining for extracellular markers (e.g. cell surface markers SSEA, SSEA4, TRA1-60 AND TRA1-80), proceed directly to step (vii);
(iii) for intracellular markers (e.g. OCT 4), fix cells at room temperature by contacting the cells with 4% paraformaldehyde for 10 minutes;
(iv) remove paraformaldehyde and wash three times with PBS;
(v) add 100% ethanol and incubate for 2 minutes;
(vi) remove ethanol and wash three times with PBS;
(vii) incubate cells with PBS containing 10% goat serum for 1 hour;
(viii) remove the PBS/goats serum and add primary antibody diluted in PBS/10% goat serum;
(ix) incubate for 1 hour at room temperature;
(x) remove primary antibody and wash three times with PBS;
(xi) incubate cells with secondary antibody diluted in PBS/10% goats serum for 30 minutes, ensuring that the cells are kept covered;
(xii) remove secondary antibody and wash three times with PBS;
(xiii) mount cells in 4′,6-diamidino-2-phenylindole (DAPI), a fluorescent stain containing mount and cover wells using a glass coverslip;
(xiv) analyse the cells using a microscope in order to quantify the cells which are positive for the stem cell specific antibodies.

The primary and secondary antibodies which may be used in such an assay include those detailed below:

### Primary Antibodies

| Antibody | Species | Company | Cat. No. | Dilution |
|---|---|---|---|---|
| SSEA-1 | mouse IgM | Hybdridoma bank | MC-480 | 1/5 |
| | | University of IOWA | | |
| SSEA-3 | mouse IgM | Hybdridoma bank | MC-631 | 1/5 |
| | | University of IOWA | | |
| SSEA-4 | mouse IgG | Hybdridoma bank | MC-813-70 | 1/5 |
| | | University of IOWA | | |
| Oct.4 | Mouse IgG | Santa Cruz | SC5279 | 1/200 |
| Tra 1-81 | mouse IgM | Santa Cruz | SC21706 | 1/200 |
| Tra 1-60 | mouse IgM | Santa Cruz | SC21705 | 1/200 |

### Secondary Antibodies

| Antibody | Species | Company | Cat. No. | Dilution |
|---|---|---|---|---|
| Alexa fluor 488 | Goat | Invitrogen | A11029 | 1/400 |
| anti-mouse IgG | | | | |
| Anti mouse IgM | Goat | Jackson | 115-095-020 | 1/200 |

One way in which pluripotent stem cells may be identified is by their ability to differentiate into cells of all three germ layers e.g. by determining the ability of the cells to differentiate into cells showing detectable expression of markers specific for all three germ layers. Stem cells can be allowed to form embryoid bodies in vitro, then the embryoid bodies studied to identify cells of all three germ layers. Alternatively, stem cells can be allowed to form teratomas in vivo (e.g. in SCID mice), then the teratomas studied to identify cells of all three germ layers. Accordingly, by use of an ADA inhibitor as defined herein, it may be possible to produce a population of stem cells wherein at least 50%, at least 55%, at least 60%, at least 70%, at least 80%, at least 85%, at least 90%, at least 91%, at least 92%, at least 93%, at least 94% or at least 95%, of the stem cells are capable of differentiating into cells of all three germ layers in vitro or in vivo.

Alternatively or in addition, the genomic integrity of stem cells can be confirmed by karyotype analysis. Stem cells can be karyotyped using known methods. A normal karyotype is where all chromosomes are present (i.e. euploidy) with no noticeable alterations. Accordingly, by use of an ADA inhibitor as defined herein, it may be possible to produce a population of stem cells wherein at least 50%, at least 55%, at least 60%, at least 70%, at least 80%, at least 85%, at least 90%, at least 91%, at least 92%, at least 93%, at least 94% or at least 95%, of the stem cells exhibit normal karyotypes.

Alternatively or in addition, it is possible to determine whether or not differentiation of a pluripotent cell has occurred via phenotypic markers. Stem cell markers (both intracellular and extracellular) may be detected using known techniques, such as immunocytochemistry, flow cytometry (e.g. fluorescence-activated cell sorting) and reverse transcriptase-PCR (RT-PCR). Examples of markers in human embryonic stem cells which will be down-regulated under normal differentiating conditions are POU5F1 (OCT-4), NANOG, zinc finger protein 42 (ZFP42) or reduced expression protein 1 (REX 1) and (sex determining region Y)-box 2 (SOX2). In addition PAX6 is the earliest marker of neuronal progenitor differentiation. Accordingly, by use of an ADA inhibitor as defined herein, it may be possible to produce a population of stem cells wherein at least 50%, at least 55%, at least 60%, at least 70%, at least 80%, at least 85%, at least 90%, at least 91%, at least 92%, at least 93%, at least 94% or at least 95%, of the stem cells express POU5F1 (OCT-4), NANOG, zinc finger protein 42 (ZFP42) or reduced expression protein 1 (REX 1) and (sex determining region Y)-box 2 (SOX2).

It is also possible to identify undifferentiated, pluripotent and proliferative stem cells by reference to morphological characteristics. Undifferentiated, pluripotent and proliferative stem cells are readily recognisable by those skilled in the art. For example, under a normal microscope, hES cells typically have high nuclear/cytoplasmic ratios, prominent nucleoli and compact colony formation with poorly discernible cell junctions.

### DEFINITIONS

The term “reversible inhibition” as used herein means that the inhibitory effect is such that the cells remain pluripotent i.e. they maintain the ability to differentiate into all three germ layers.

The term “aromatic” is used to refer to a compound which has a conjugated system of double bonds, lone pairs or empty orbitals which exhibit a stabilization which exceeds that which would be expected as a consequence of conjugation alone. Examples of aromatic compounds include benzene, toluene, ortho-xylene, para-xylene, pyridine, imidazole, pyrazole, naphthalene and anthracene.

The term “carbocyclic ring” is used to refer to a ring system which is composed of carbon atoms.

The term ‘halogen’ includes fluorine, chlorine, bromine and iodine.

The term ‘hydrocarbyl’ includes linear, branched or cyclic monovalent groups consisting of carbon and hydrogen. Hydrocarbyl groups thus include alkyl, alkenyl and alkynyl groups, cycloalkyl (including polycycloalkyl), cycloalkenyl and aryl groups and combinations thereof, e.g. alkylcycloalkyl, alkylpolycycloalkyl, alkylaryl, alkenylaryl, cycloalkylaryl, cycloalkenylaryl, cycloalkylalkyl, polycycloalkylalkyl, arylalkyl, arylalkenyl, arylcycloalkyl and arylcycloalkenyl groups. Preferred hydrocarbyl are C₁₋₁₂ hydrocarbyl, more preferably C₁₋₈ hydrocarbyl.

The terms ‘alkyl’, ‘alkenyl’ or ‘alkynyl’ are used herein to refer to both straight and branched chain forms.

The term ‘alkyl’ includes monovalent saturated hydrocarbyl groups. Preferred alkyl are C₁₋₁₂, preferably C₁₋₁₀ alkyl, preferably C₁₋₆, preferably C₁₋₄ alkyl, such as methyl, ethyl, n-propyl, i-propyl or t-butyl groups.

The term “cycloalkyl” is used to describe cyclic alkyl groups and includes C₃₋₁₀ groups, preferably C₅₋₈ groups.

The term ‘alkenyl’ includes monovalent hydrocarbyl groups having at least one carbon-carbon double bond and preferably no carbon-carbon triple bonds. Preferred alkenyl are C₂₋₁₂ alkenyl, preferably C₂₋₁₀, alkenyl, preferably C₂₋₆ alkenyl, preferably C₂₋₄ alkenyl.

The term ‘alkynyl’ includes monovalent hydrocarbyl groups having at least one carbon-carbon triple bond and preferably no carbon-carbon double bonds. Preferred alkynyl are ₋₁₂ alkynyl, preferably C₂₋₁₀, alkynyl, preferably C₂₋₆ alkynyl, preferably C₂₋₄ alkynyl.

The term ‘alkoxy’ means alkyl-O—.

The term ‘aryl’ includes monovalent aromatic groups, such as phenyl or naphthyl. In general, the aryl groups may be monocyclic or polycyclic fused ring aromatic groups. Preferred aryl are C₆₋₁₄ aryl.

Other examples of aryl groups are monovalent derivatives of aceanthrylene, acenaphthylene, acephenanthrylene, anthracene, azulene, chrysene, coronene, fluoranthene, fluorene, as-indacene, s-indacene, indene, naphthalene, ovalene, perylene, phenalene, phenanthrene, picene, pleiadene, pyrene, pyranthrene and rubicene.

The term ‘heteroaryl’ includes aryl groups in which up to three carbon atoms, preferably up to two carbon atoms, more preferably one carbon atom, are each replaced independently by O, S, Se or N, preferably O, S or N. Preferred heteroaryl are C₅₋₁₄ heteroaryl. Examples of heteroaryl are pyridyl, pyrrolyl, thienyl or furyl.

Other examples of heteroaryl groups are monovalent derivatives of acridine, carbazole, β-carboline, chromene, cinnoline, furan, imidazole, indazole, indole, indolizine, isobenzofuran, isochromene, isoindole, isoquinoline, isothiazole, isoxazole, naphthyridine, perimidine, phenanthridine, phenanthroline, phenazine, phthalazine, purine, pyran, pyrazine, pyrazole, pyridazine, pyridine, pyrimidine, pyrrole, pyrrolizine, quinazoline, quinoline, quinolizine, quinoxaline, thiophene and xanthene. Preferred heteroaryl groups are five- and six-membered monovalent derivatives, such as the monovalent derivatives of furan, imidazole, isothiazole, isoxazole, pyran, pyrazine, pyrazole, pyridazine, pyridine, pyrimidine, pyrrole, pyrrolizine and thiophene. The five-membered monovalent derivatives are particularly preferred, i.e. the monovalent derivatives of furan, imidazole, isothiazole, isoxazole, pyrazole, pyrrole and thiophene.

The aryl or heteroaryl groups may be substituted with 1, 2 or 3 groups independently selected from hydrogen, halogen, —OR³⁰, C₁₋₁₂-alkyl, C₂₋₁₂ alkenyl, C₁₋₁₂ alkynyl, aryl, heteroaryl, C₁₋₁₂ alkoxy, NR³⁰R³¹ and NR^{a}C(O)R^{b} wherein R³⁰, R³¹ and R^{a} are independently selected from H and C₁₋₆ alkyl and R^{b} is selected from C₁₋₁₂ alkyl optionally substituted with 1, 2, or 3 groups independently selected from hydrogen, halogen, aryl and heteroaryl, wherein each of said aryl and heteroaryl may be substituted with 1, 2 or 3 groups selected from hydrogen and halogen.

The term ‘heteroalkylene’ includes alkylene groups in which up to three carbon atoms, preferably up to two carbon atoms, more preferably one carbon atom, are each replaced independently by O, S, Se or N, preferably O, S or N.

The term ‘heterocycloalkyl’ includes cycloalkyl groups in which up to three carbon atoms, preferably up to two carbon atoms, more preferably one carbon atom, are each replaced independently by O, S, Se or N, preferably O, S or N.

Where reference is made to a carbon atom of a hydrocarbyl or other group being replaced by an O, S, Se or N atom, what is intended is that:

—CH═ is replaced by —N═; or

—CH₂— is replaced by —O—, —S— or —Se—.

One or more of the C₁₋₁₂ alkyl, C₂₋₁₂ alkenyl, C₂₋₁₂-alkynyl, aryl, heteroaryl, C₃₋₁₀ cycloalkyl and C₃₋₁₀ heterocycloalkyl groups of the compound of formula (I) may be optionally substituted with 1, 2 or 3 groups independently selected from hydrogen, halogen, —OR³⁰, C₁₋₁₂-alkyl, C₂₋₁₂ alkenyl, C₂₋₁₂ alkynyl, aryl, heteroaryl, C₁₋₁₂ alkoxy and NR³⁰R³¹, wherein R³⁰ and R³¹ are independently selected from H and C₁₋₆ alkyl. The use of terms in the singular, e.g. “a cell” encompasses the plural (e.g. “cells”) unless the context requires otherwise.

The term “pharmaceutically acceptable salt” means a physiologically or toxicologically tolerable salt and includes, when appropriate, pharmaceutically acceptable base addition salts and pharmaceutically acceptable acid addition salts. For example (i) where a compound used in the invention contains one or more acidic groups, for example carboxy groups, pharmaceutically acceptable base addition salts that can be formed include sodium, potassium, calcium, magnesium and ammonium salts, or salts with organic amines, such as, diethylamine, N methyl-glucamine, diethanolamine or amino acids (e.g. lysine) and the like; (ii) where a compound used in the invention contains a basic group, such as an amino group, pharmaceutically acceptable acid addition salts that can be formed include hydrochlorides, hydrobromides, sulfates, phosphates, acetates, citrates, lactates, tartrates, mesylates, tosylates, benzenesulfonates, maleates, fumarates, xinafoates, p-acetamidobenzoates, succinates, ascorbates, oleates, bisulfates and the like.

Hemisalts of acids and bases can also be formed, for example, hemisulfate and hemicalcium salts.

A “population” of cells is any number of cells greater than 1, but is preferably at least 1×10³ cells, at least 1×10⁴ cells, at least 1×10⁵ cells, at least 1×10⁶ cells, at least 1×10⁷ cells, at least 1×10⁸ cells, or at least 1×10¹⁰ cells

The term “comprising” encompasses “including” as well as “consisting” e.g. a composition “comprising” X may consist exclusively of X or may include something additional e.g. X+Y.

The term “about” in relation to a numerical value x means, for example, x+10%.

The word “substantially” does not exclude “completely” e.g. a composition which is “substantially free” from Y may be completely free from Y. Where necessary, the word “substantially” may be omitted from the definition of the invention.

The invention will now be described further by reference to the following figures and examples which are in no way intended to be limiting on the scope of the invention.

### FIGURES

FIG. 1 illustrates a summary of normal adenosine metabolism;

FIG. 2A shows that EHNA maintains stem cells in an undifferentiated state for 8 passages;

FIG. 2B shows that EHNA maintains stem cells in an undifferentiated state for 10 passages;

FIG. 2C illustrates that in the absence of FGF and EHNA, stem cell differentiation occurs by passage 10;

FIG. 3 shows that the expression of the stem cell markers NANOG and POU5F1 is maintained in the absence of FGF by EHNA;

FIG. 4 shows TLDA analysis of stem cell markers;

FIG. 5 is an immunofluorescent image of human embryonic stem cells grown in the absence of FGF and presence of EHNA, wherein the cells were fixed with 4% paraformaldehyde and stained with POU5F1 specific antibodies;

FIG. 6 shows immunofluorescent images of labelled markers which show differentiation into all three germ layers of hESCs grown in the absence of FGF but in the presence of EHNA for 22 passages;

FIG. 7 shows the absence of differentiation marker SSEA1 (a) and presence of stem cell markers SSEA3 (b), SSEA4 (c), TRA1-60 (d), TRA1-80 (e) and POU5F1 (f) by immunofluorescence in hESCs grown in the absence of FGF and presence of EHNA for 10 passages feeder free;

FIG. 8 shows the maintenance of POU5F1 expression by EHNA in the absence of exogenous FGF in cells passaged feeder free for 7 passages but derived directly from feeders;

FIG. 9 shows a series of two graphs obtained by qRT-PCR analysis which illustrate the relative expression of the stem cell markers NANOG, POU5F1, SOX and ZFP42, and the differentiation marker PAX6, in cells treated with EHNA and PDE inhibitors (A and B);

FIG. 10 shows a series of two graphs obtained by qRT-PCR analysis which illustrate the relative expression of the stem cell marker and the differentiation marker PAX6, in cells treated with differing ADA inhibitors (A and B);

FIG. 11A shows qRT-PCR expression data for NANOG expression after 15 days of neuronal differentiation;

FIG. 11B shows qRT-PCR expression data for POU5F1 expression after 15 days of neuronal differentiation;

FIG. 11C shows qRT-PCR expression data for ZFP42 expression after 15 days of neuronal differentiation;

FIG. 11D shows qRT-PCR expression data for PAX6 expression after 15 days of neuronal differentiation;

FIG. 12A shows qRT-PCR expression data for NANOG, ZFP42 and PAX6 expression after 28 days of neuronal differentiation with and without EHNA treatment;

FIG. 12B shows the percentage of cells staining positive for POU5F1 after 28 days of neuronal differentiation with and without EHNA treatment.

FIG. 13 shows a series of two graphs obtained by qRT-PCR analysis which illustrate the relative expression of the stem cell marker NANOG and the differentiation marker PAX6, in cells treated with EHNA and compounds of formula (I), HWC6, HWC7, HWC8, HWC9, HWC10, HWC12, HWC13, HWC14, HWC15, HWC16, HWC17, HWC18, HWC21 and HWC24.

FIG. 14 shows a series of two graphs obtained by qRT-PCR analysis which illustrate the relative expression of the stem cell marker NANOG and the differentiation marker PAX6, in cells treated with EHNA and compounds of formula (I), HWC25, HWC26, HWC27, HWC28, HWC29, HWC30, HWC31, HWC33, HWC34, HWC35, HWC36 and HWC37.

FIG. 15 shows a series of two graphs obtained by qRT-PCR analysis which illustrate the relative expression of the stem cell marker NANOG and the differentiation marker PAX6, in cells treated with EHNA and compounds of formula (I), HWC40, HWC41, HWC42, HWC43, HWC44, HWC45, HWC46, HWC47, HWC48, HWC49, HWC50, HWC51, HWC52, HWC53 and HWC54.

FIG. 16 shows a series of two graphs obtained by qRT-PCR analysis which illustrate the relative expression of the stem cell marker NANOG and the differentiation marker PAX6, in cells treated with EHNA and compounds of formula (I), HWC48A, HWC57, HWC58, HWC59, HWC60 and HWC61.

FIG. 17 shows a series of two graphs obtained by qRT-PCR analysis which illustrate the relative expression of the stem cell marker NANOG and the differentiation marker PAX6, in cells treated with EHNA and compounds of formula (I), HWC62, HWC63 and HWC64.

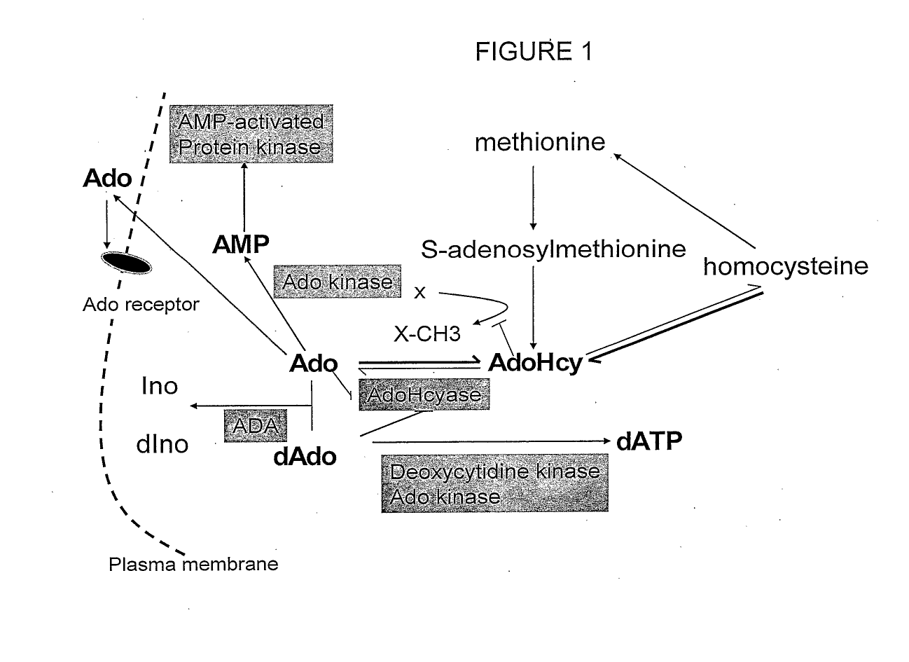

### EXAMPLES

In the following examples, the general techniques described below were employed.

### Culturing

Cells were grown on fibronectin coated dishes. A fibronectin (Calbiochem) solution (0.1 mg/ml) (diluted in PBS) is used to coat the tissue culture dishes for 15 mins at 37 C.

The fully supportive media used to culture the stem cells was a 1:1 mixture of defined media (see below) and conditioned vitrohES. vitrohES media was conditioned for 24 hours on a layer of mitotically inactivated mouse embryonic fibroblasts plated at 4×10⁵ cells/ml of conditioned media. Passaging was performed first by removing the media from the cells and washing with PBS. Trypsin (sourced from Invitrogen) was added to the cells and incubated for 1-2 mins at room temperature. Fresh media was added to neutralize the cells and then gently scraped off using a cell scraper and passaged 1 in 4 to fibronectin coated dishes.

Defined Media (all components from Carlsbad, Calif., http://www.Invitrogen.com):

### 500 ml Advanced DMEM/F12

5 ml of 100×N2 supplement
10 ml of 50× B27 supplement

### 2.5 mls 200 mM L-Glutamine

35 μl 1.43M β-mercaptoethanol

### General Differentiation

For general differentiation experiments cells were passaged with trypsin onto matrigel coated twelve-well dishes in the fully supportive media (see above). The media was then changed to defined media 24 h later and any experimental component was first added at this point. The media and experimental supplement were changed daily.

Components added:

EHNA (10 μM)-erythro-9-(2-Hydroxy-3-nonyl)adenine, HCl

### IBMX (100 μM)-isobutylmethylxanthine

### BAY-60-7550 (5-50 nM)

### HW-compounds (HWC)

Cells were routinely harvested for qRT-PCR analysis 14 days after the first addition of the experimental compound.

### RNA Isolation and Quantitative PCR

Total RNA was extracted using QIAGEN RNeasy kits (Qiagen Inc, Valencia, Calif., http://www.qiagen.com) and DNaseI treatment was performed using Turbo DNA-Free (Ambion). The absence of genomic DNA was confirmed by quantitative PCR (qPCR) (see below) using glyceraldehyde-3-phosphate dehydrogenase (GAPDH) Taqman primers and probe (MWG). cDNA was synthesized with 2 μg total RNA in 20 μl using Superscript II according to manufacturers' instructions ((Carlsbad, Calif., http://www.Invitrogen.com)). For qPCR, cDNAs were diluted 1 in 60 and 5 μl used per sample in a 15 μl total reaction volume. Platinum pPCR supermix UDG with Rox (Carlsbad, Calif., http://www.Invitrogen.com) was used according to the manufacturer's instructions using an Applied Biosystems 7300 real time PCR system (Applied Biosystems, Warrington, UK, http://europe.appliedbiosystems.com). Each reaction contained 0.6 μM of each primer and Taqman probe (MWG). A complete list of primer probe sequences is provided in Table 1 below. Biological and technical replicates were each performed in triplicate for each sample, standardised to the GAPDH housekeeping gene and relative expression values were calculated using the 7300 system SDS software (Applied Biosystems, Warrington, UK, http://europe.appliedbiosystems.com).

TABLE 1
Primer and probe sequences for qRT-PCR

| GENE | Probe | Forward Primer | Reverse Primer |
|---|---|---|---|
| NANOG | CAGCTACAAACAGGTGAAGACCTGGTTCC | GAACTCTCCAACATCCTGAAC | CGTCACACCATTGCTATTCTTC |
| | (SEQ ID No 1) | (SEQ ID No 7) | (SEQ ID No 13) |
| Pax6 | CTGTGACAACCAGAAAGGATGCCTC | AACCCCAACCAAACAAAACTC | GCGCCCCTAGTTAAAGTCTTC |
| | (SEQ ID No 2) | (SEQ ID No 8) | (SEQ ID No 14) |
| POU5F1 | CATGGCGGGACACCTGGCTTCAGATTTTGCCTTCT | CGCAAGCCCTCATTTCAC | CCAGGTCCGAGGATCAAC |
| | (SEQ ID No 3) | (SEQ ID No 9) | (SEQ ID No 15) |
| SOX-2 | CATGGAGAAAACCCGGTACGCTCA | AATGGGAGGGGTGCAAAAG | TGAGTGTGGATGGGATTGG |
| | (SEQ ID No 4) | (SEQ ID No 10) | (SEQ ID No 16) |
| ZFP42 | TAAGCCCAGGCAAGGCAAGTCAAGCCAA | GCAAAGACAAGACACCAGAAAG | CATAGCACACATAGCCATCAC |
| | (SEQ ID No 5) | (SEQ ID No 11) | (SEQ ID No 17) |
| GAPDH | TGGCATTGCCCTCAACGACCACTT | AGGTGGTCTCCTCTGACTTC | CGTTGTCATACCAGGAAATGAG |
| | (SEQ ID No 6) | (SEQ ID No 12) | (SEQ ID No 18) |

### Immunocytochemistry

After washing with PBS, cells were fixed in 4% paraformaldehyde (Sigma) for 10 mins at room temperature and washed again. Cells were then permeabilised in ethanol for 2 minutes. Blocking was performed in 10% Goat serum (sigma)/PBS (Invitrogen) for one hour at room temperature. The following primary antibodies (dilutions in brackets) were then incubated for 1 hour at room temperature diluted in blocking solution: mouse anti-POU5F1 (Santa cruz) (1:250), rabbit anti-PAX6 (Chemicon) (1:1000), mouse anti B-tubulin III (Sigma) (1:1000), mouse anti-Alpha-feta-protein (AFP) (Sigma) (1:400), mouse anti-muscle-specific actin (SMA) (Dako) (1:50), SSEA1 (Hybdridoma bank University of IOWA) (1:5), SSEA3 (Hybdridoma bank University of IOWA) (1:5), SSEA4 (Hybdridoma bank. University of IOWA) (1:5), Tra1-60 (Santa cruz) (1:200) and Tra1-80 (Santa Cruz) (1:200). The secondary antibodies (either Alexa fluor 555 goat ant-rabbit IgG (1:400) or Alexa fluor 488 Goat anti-mouse IgG (1:400) (Invitrogen)) were incubated for 30 mins at room temperature and cells were mounted using Prolong gold containing DAPI (invitrogen). All immunofluorescence was visualised and captures using Zeiss Axiovision image analysis system (http://www.ziess.com).

### Synthesis of 3-(3H-imidazo[4,5-b]pyridin-3-yl)nonan-2-ol hydrochloride (HWC5)

### 3-Amino-2-nonanone Hydrochloride

3-Amino-2-nonanone hydrochloride was prepared by adaptation of the procedure reported by Schaeffer and Schwender (J. Med. Chem., 1974, 17, 6-8).

DL-2-Aminooctanoic acid (25.0 g, 157 mmol) was suspended in pyridine (84 mL) and cooled to 0° C. Acetic anhydride (124 mL) was added to the heterogeneous mixture over a period of 25 min and the mixture was then heated at 114° C. for 3.5 h, forming an homogenous yellow solution. The reaction mixture was cooled to ambient temperature and evaporated at 20 mbar. The residual oil was diluted with ethyl acetate (100 mL) and washed with 5% sodium bicarbonate solution (280 mL) followed by saturated sodium chloride solution (50 mL). The organic layer was dried over magnesium sulfate, filtered and evaporated to give crude N-(2-oxononan-3-yl)acetamide (32.8 g) as a waxy solid. The crude N-(2-oxononan-3-yl)acetamide thus obtained was suspended in concentrated hydrochloric acid (37% w/w; 300 mL) and heated in an oil bath thermostatted at 110° C. for 10 h. TLC (ethyl acetate) indicated consumption the starting material (R_{f} 0.52) and formation of a product spot (R_{f} 0.16). The mixture was evaporated in vacuo and the residual solid dissolved in hot ethanol (50 mL), diluted with diethyl ether (100 mL) and cooled in ice. The resulting white crystalline precipitate was collected by filtration, washing with diethyl ether (100 mL), and dried over P₂O₅ in vacuo for 18 h to give 3-aminononan-2-one hydrochloride (20.6 g, 106 mmol; 68%): δ_{H} (200 MHz; DMSO-d₆) 8.44 (3H, bs), 4.16-3.98 (1H, m), 2.23 (3H, s), 1.95-1.65 (2H, m), 1.39-1.13 (8H, m), 0.83 (3H, approx. t, J 6.8); δ_{C} (50 MHz; DMSO-d₆) 205.05 (C), 58.82 (CH), 31.42 (CH₂), 29.32 (CH₂), 28.90 (CH₂), 27.27 (CH₃), 24.42 (CH₂), 22.49 (CH₂), 14.44 (CH₃).

### 3-Aminononan-2-ol

3-Aminononan-2-ol was prepared by adaptation of the procedure reported by Schaeffer and Schwender (J. Med. Chem., 1974, 17, 6-8).

3-Aminononan-2-one hydrochloride (20.5 g, 106 mmol) was dissolved in anhydrous methanol (74 mL) at ambient temperature under argon and cooled to −14° C. Potassium borohydride (11.4 g, 212 mmol) was added in portions over a period of 10 min, maintaining an internal temperature below −10° C. The pale yellow heterogeneous mixture was stirred at −14° C. for 2 h, then slowly allowed to attain ambient temperature and stirred for a further 16 h. The solvent was evaporated in vacuo to give a residue that was dissolved in water (50 mL) and extracted with chloroform (3×70 mL). The organic layer was dried over magnesium sulfate, filtered and evaporated in vacuo, giving an orange oil (16.9 g) that was subjected to Kugelrohr distillation (oven temperature 140-160° C., 2.5 mbar) to afford 3-aminononan-2-ol (11.2 g, 70.3 mmol; 66%) as a pale yellow oil. ¹H NMR analysis indicated that the product comprised a 1:4 mixture of threo- and erythro-3-aminononan-2-ol. NMR data for major erythro-product: δ_{H} (200 MHz; CDCl₃) 3.66 (1H, dq, J 3.9 and 6.4, H-2), 2.79-2.64 (1H, m, H-3), 1.33-1.18 (10H, m), 1.05 (3H, d, J 6.4, CH₃-1), 0.84 (3H, approx. t, J 6.7, CH₃-9); δ_{C} (50 MHz; CDCl₃) 69.88 (CH), 56.10 (CH), 33.16 (CH₂), 31.92 (CH₂), 29.55 (CH₂), 26.71 (CH₂), 22.74 (CH₂), 16.98 (CH₃), 14.20 (CH₃). Partial ¹H NMR data for the minor threo-product: δ_{H} (200 MHz; CDCl₃) 3.38 (1H, approx. quintet, J 6.4, H-2), 2.48-2.35 (1H, m, H-3), 1.16 (3H, d, J 6.2, CH₃-1).

### erythro-3-(3-Nitropyridin-2-ylamino)nonan-2-ol

3-(3-Nitropyridin-2-ylamino)nonan-2-ol was prepared by adaptation of the procedure reported by Antonini et al (J. Med. Chem., 1984, 27, 274-278).

A stirred mixture of 2-chloro-3-nitropyridine (400 mg, 2.52 mmol), 3-aminononan-2-ol (4:1 erythro-1 threo-mixture; 442 mg, 2.78 mmol), triethylamine (551 μL, 3.95 mmol) and nitromethane (10 mL) was heated in an oil bath thermostatted at 118° C. for 2 h. TLC (4:1 light petroleum/ethyl acetate) indicated the consumption of 2-chloro-3-nitropyridine (R_{f} 0.29) and formation of two product components at R_{f} 0.34 and 0.20. The reaction mixture was concentrated in vacuo to afford a residue that was dissolved in water (30 mL) and extracted with chloroform (3×30 mL). The organic extract was dried over sodium sulfate and evaporated to give an oily residue that was subjected to flash column chromatography (30 g silica). Elution with 5:1 light petroleum/ethyl acetate (600 mL) yielded erythro-3-(3-nitropyridin-2-ylamino)nonan-2-ol (473 mg, 1.68 mmol; 67%) as a viscous yellow oil (R_{f} 2.0): δ_{H} (200 MHz; CDCl₃) 8.41 (1H, dd, J 1.8 and 8.3, pyridine ring H-4), 8.29 (1H, dd, J 1.8 and 4.6, pyridine ring H-6), 8.14 (1H, br d, J 7.2, NH), 6.63 (1H, dd, J 4.6 and 8.3, pyridine ring H-5), 4.51-4.27 (1H, m, chain H-2), 4.06 (1H, br d, J 4.9, OH), 4.03-3.88 (1H, m, chain H-3), 1.71-1.17 (10H, m), 1.12 (3H, d, J 6.3, chain CH₃-1), 0.81 (3H, approx. t, J 6.5, chain CH₃-9); δ_{C} (50 MHz; CDCl₃) 155.43 (CH), 153.56 (C), 136.13 (CH), 128.52 (C), 112.31 (CH), 71.16 (CH), 57.45 (CH), 31.77 (CH₂), 31.50 (CH₂), 29.24 (CH₂), 26.69 (CH₂), 22.71 (CH₂), 17.85 (CH₃), 14.20 (CH₃).

### erythro-3-(3-Aminopyridin-2-ylamino)nonan-2-ol

3-(3-Aminopyridin-2-ylamino)nonan-2-ol was prepared by adaptation of the procedure reported by Antonini et al (J. Med. Chem., 1984, 27, 274-278).

A stirred solution of 3-(3-nitropyridin-2-ylamino)nonan-2-ol (443 mg, 1.58 mmol) in a mixture of ethanol (20 mL) and ethyl acetate (10 mL) was hydrogenated at ambient temperature over 10% palladium on charcoal (100 mg) under a hydrogen pressure of 1 atmosphere for 18 h. TLC (9:1 dichloromethane/methanol) indicated the consumption of starting material (R_{f} 0.74) and formation of a product component (R_{f} 0.51). The catalyst was removed by filtration under nitrogen and the filtrate evaporated to give erythro-3-(3-aminopyridin-2-ylamino)nonan-2-ol (411 mg, 1.55 mmol; 99%) as a colourless (air sensitive) oil: δ_{H} (200 MHz; CDCl₃) 7.56 (1H, dd, J 1.5 and 5.2, pyridine ring H-6), 6.84 (1H, dd, J 1.5 and 7.4, pyridine ring H-4), 6.47 (1H, dd, J 5.2 and 7.4, pyridine ring H-5), 4.28 (1H, br s), 3.99 (1H, br m), 3.92 (1H, dq, J 1.8 and 6.4, chain H-2), 3.60 (3H, br s), 1.58-1.16 (10H, m), 1.07 (3H, d, J 6.3), 0.83 (3H, approx. t, J 6.5); δ_{C} (50 MHz; CDCl₃) 151.04 (C), 138.07 (CH), 128.39 (C), 123.10 (CH), 113.58 (CH), 71.44 (CH), 58.49 (CH), 32.59 (CH₂), 31.87 (CH₂), 29.39 (CH₂), 27.17 (CH₂), 22.77 (CH₂), 17.50 (CH₃), 14.24 (CH₃).

### erythro-3-(3H-imidazo[4,5-b]pyridin-3-yl)nonan-2-ol

3-(3H-imidazo[4,5-b]pyridin-3-yl)nonan-2-ol was prepared by adaptation of the procedure reported by Antonini et al (J. Med. Chem., 1984, 27, 274-278).

Concentrated hydrochloric acid (37% w/w; 0.30 mL) was added to a stirred solution of erythro-3-(3-aminopyridin-2-ylamino)nonan-2-ol (378 mg, 1.50 mmol) in triethyl orthoformate (15 mL, 90 mmol). The reaction mixture was stirred at ambient temperature for 20 h. TLC (95:5 dichloromethane/methanol) indicated consumption of the starting material (R_{f} 0.20, brown on exposure to air) and formation of a product component (R_{f} 0.18). The reaction mixture was then neutralized with saturated sodium bicarbonate solution and extracted with dichloromethane (3×20 mL). The combined organic extract was washed with saturated sodium chloride solution (3×10 mL), dried over sodium sulfate, filtered and concentrated in vacuo to give an oily residue. The crude residue was subjected to flash column chromatography (30 g silica). Elution with dichloromethane/methanol (98:2, 250 mL; 95:5, 100 mL) afforded 3-(3H-imidazo[4,5-b]pyridin-3-yl)nonan-2-ol as a colourless oil (260 mg, 0.995 mmol; 66%): δ_{H} (200 MHz; CDCl₃) 8.29 (1H, dd, J 1.4 and 4.8, imidazopyridine H-5), 8.02 (1H, dd, J 1.4 and 8.1, imidazopyridine H-7), 8.02 (1H, s, imidazopyridine H-2), 7.19 (1H, dd, J 4.8 and 8.1, imidazopyridine H-6), 5.58 (1H, s), 4.41-4.18 (2H, m), 2.27-1.79 (2H, m), 1.29 (3H, d, J 6.5), 1.25-0.93 (8H, m), 0.77 (3H, t, J 6.5); δ_{C} (50 MHz; CDCl₃) 146.76 (C), 144.53 (CH), 143.58 (CH), 136.19 (C), 128.59 (CH), 118.50 (CH), 69.75 (CH), 63.79 (CH), 31.68 (CH₂), 29.02 (CH₂), 27.07 (CH₂), 26.49 (CH₂), 22.65 (CH₂), 20.53 (CH₃), 14.15 (CH₃).

The hydrochloride salt of erythro-3-(3H-imidazo[4,5-b]pyridin-3-yl)nonan-2-ol was prepared by addition of a saturated solution of hydrogen chloride in diethyl ether (10 mL) to a solution of erythro-3-(3H-imidazo[4,5-b]pyridin-3-yl)nonan-2-ol (130 mg) in dichloromethane (10 mL). The precipitated salt was collected by filtration and dried in vacuo P₂O₅ to afford erythro-3-(3H-imidazo[4,5-b]pyridin-3-yl)nonan-2-ol hydrochloride as a colourless powder (Found C, 60.57; H, 8.27; N, 14.31.; C₁₅H₂₄ClN₃O requires C, 60.49; H, 8.12; N, 14.11.).

### Synthesis of 1-decyl-1H-pyrazolo[3,4-d]pyrimidin-4-amine (HWC-4) and 2-decyl-2H-pyrazolo[3,4-d]pyrimidin-4-amine hydrochloride (HWC6)

### 3-Amino-1-decyl-1H-pyrazole-4-carbonitrile

3-Amino-1-decyl-1H-pyrazole-4-carbonitrile was prepared following the procedure reported by Da Settimo et al (J. Med. Chem., 2005, 48, 5162-5174):

1-Bromodecane (3.73 mL, 18.0 mmol) was added dropwise to a suspension of 3-amino-1H-pyrazole-4-carbonitrile (1.62 g, 15.0 mmol) and anhydrous potassium carbonate (2.49 g, 18.0 mmol) in N,N-dimethylformamide (15 mL). The reaction mixture was stirred at 50° C. for 20 h. TLC (2:1 light petroleum/ethyl acetate) indicated consumption of the 3-amino-1H-pyrazole-4-carbonitrile starting material (R_{f} 0.03) and formation of product (R_{f} 0.31). After cooling to ambient temperature, the inorganic material was removed by filtration and the filtrate was evaporated to dryness in vacuo. The resulting residue was subjected to flash column chromatography (70 g silica), eluting with light petroleum/ethyl acetate (3:1, 200 mL; 2:1, 200 mL; 1:1, 200 mL) to afford the product (3.22 g, 13.0 mmol; 87%), a colourless powder, as a 2.6:1 mixture of 3-amino-1-decyl-1H-pyrazole-4-carbonitrile and 5-amino-1-decyl-1H-pyrazole-4-carbonitrile isomers. Major isomer, 3-amino-1-decyl-1H-pyrazole-4-carbonitrile: δ_{H} (200 MHz; CDCl₃) 7.44 (1H, s, pyrazole H-5), 4.13 (2H, br s, NH₂), 3.85 (2H, t, J 7.1, chain CH₂-1), 1.86-1.64 (2H, m, chain CH₂-2), 1.30-1.14 (14H, m), 0.83 (3H, approx. t, J 6.5, chain CH₃-10); δ_{C} (50 MHz; CDCl₃) 156.87 (C), 140.13 (C), 133.95 (CH), 114.03 (C), 52.83 (CH₂), 31.99 (CH₂), 29.73 (CH₂), 29.61 (CH₂), 29.55 (CH₂), 29.40 (CH₂), 29.18 (CH₂), 26.55 (CH₂), 22.81 (CH₂), 14.26 (CH₃).

### 1-decyl-1H-pyrazolo[3,4-d]pyrimidin-4-amine (HWC-4) & 2-decyl-2H-pyrazolo[3,4-d]pyrimidin-4-amine (HWC-3)

1-Decyl-1H-pyrazolo[3,4-d]pyrimidin-4-amine (HWC-4) and 2-decyl-2H-pyrazolo[3,4-d]pyrimidin-4-amine (HWC-3) were prepared following the procedure reported by Da Settimo et al (J. Med. Chem., 2005, 48, 5162-5174):

A solution of isomeric pyrazolecarbonitriles, 3-amino-1-decyl-1H-pyrazole-4-carbonitrile and 5-amino-1-decyl-1H-pyrazole-4-carbonitrile (2.6:1; 500 mg, 2.013 mmol), in formamide (1.0 mL) was heated at 210° C. for 2 h. TLC (9:1 dichloromethane/methanol) indicated consumption of the starting pyrazoles (R_{f} 0.69) and formation of two product components (R_{f} 0.25 & 0.39). After cooling to ambient temperature ice-water was added to the brown reaction mixture. The precipitated solid was collected by filtration, washing with water, and then subjected to flash column chromatography (35 g silica). Elution with dichloromethane/methanol (98:2, 250 mL; 95:5, 300 mL; 9:1, 250 mL) afforded 2-decyl-2H-pyrazolo[3,4-d]pyrimidin-4-amine (245 mg, 0.890 mmol; 44%) (R_{f} 0.25) and 1-decyl-1H-pyrazolo[3,4-d]pyrimidin-4-amine (132 mg, 0.479 mmol; 24%) (R_{f} 0.39), both as colourless powders.

The identity of the major product, 2-decyl-2H-pyrazolo[3,4-d]pyrimidin-4-amine (HWC-3; R_{f} 0.25), was confirmed by acquisition of a NOESY spectrum. δ_{H} (400 MHz, DMSO-d₆) 8.28 (1H, s), 8.10 (1H, s), 7.55 (2H, br s), 4.29 (2H, t, J 7.2), 1.85-1.75 (2H, m), 1.29-1.12 (14H, m), 0.80 (3H, approx. t, J 6.7); δ_{C} (101 MHz, DMSO-d₆) 159.69 (C), 159.15 (C), 155.90 (CH), 124.44 (CH), 101.17 (C), 52.77 (CH₂), 31.25 (CH₂), 29.60 (CH₂), 28.87 (CH₂), 28.85 (CH₂), 28.62 (CH₂), 28.44 (CH₂), 25.85 (CH₂), 22.05 (CH₂), 13.91 (CH₃); (Found C, 65.29; H, 9.18; N, 25.36.; C₁₅H₂₅N₅ requires C, 65.42; H, 9.15; N, 25.43.). This compound exhibited poor solubility in 5% DMSO-water and, to facilitate biological assessment, was converted into its hydrochloride salt (HWC-6) by treatment with a saturated solution of hydrogen chloride in diethyl ether followed by evaporation.

NMR data for minor isomer, 1-decyl-1H-pyrazolo[3,4-d]pyrimidin-4-amine (HWC-4; R_{f} 0.39): δ_{H} (200 MHz; CDCl₃) 8.37 (1H, s), 7.91 (1H, s), 6.04 (2H, br s), 4.38 (2H, t, J 7.1, chain CH₂-1), 1.97-1.82 (2H, m, chain CH₂-2), 1.35-1.15 (14H, m), 0.84 (3H, approx. t, J 6.5, chain CH₃-10); δ_{C} (50 MHz; CDCl₃) 157.58 (C), 155.55 (CH), 153.34 (C), 130.36 (CH) 100.66 (C), 47.48 (CH₂), 31.99 (CH₂), 29.79 (CH₂), 29.61 (CH₂), 29.58 (CH₂), 29.39 (CH₂), 29.26 (CH₂), 26.77 (CH₂), 22.80 (CH₂), 14.25 (CH₃).

### Synthesis of racemic erythro-1-(2-hydroxynonan-3-yl)-N-methyl-1H-imidazole-4-carboxamide (HWC-7)

### Ethyl erythro-5-amino-1-(-2-hydroxynonan-3-yl)-1H-imidazole-4-carboxylate

Ethyl erythro-5-amino-1-(-2-hydroxynonan-3-yl)-1H-imidazole-4-carboxylate was prepared following the procedure of Cristalli et al (J. Med. Chem., 1991, 34, 1187-1192):

Triethyl orthoformate (2.12 mL, 12.7 mmol) was added to a solution of ethyl 2-amino-2-cyanoacetate (1.54 g, 12 mmol) in acetonitrile (15 mL) at room temperature under an atmosphere of argon. The mixture was refluxed at an external temperature of 93° C. for 1 h to give a pale yellow homogenous mixture. The mixture was allowed to cool and 3-aminononan-2-ol (preparation—vide supra; 2.00 g, 12.6 mmol) was added to give a red-orange homogenous mixture. After stirring at ambient temperature for 22 h the mixture was evaporated to give an orange oil that was subjected to flash chromatography. Elution with methanol/dichloromethane (0.4:10) afforded ethyl 5-amino-1-(-2-hydroxynonan-3-yl)-1H-imidazole-4-carboxylate (1.19 g, 3.99 mmol; 33%): δ_{H} (200 MHz; CDCl₃) 7.01 (1H, s, imidazole CH), 5.40 (2H, br s), 4.35-3.94 (3H, m), 4.29 (2H, q, J 7.1, ester CH₂), 1.66-1.94 (2H, m, chain CH₂-4), 1.35 (3H, t, J 7.1, ester CH₃), 1.35-1.11 (8H, m), 1.11 (3H, d, J 6.4, chain CH₃-1), 0.83 (3H, approx. t, J 6.5, chain CH₃-9); δ_{C} (50 MHz; CDCl₃) 164.99 (C), 147.07 (C), 130.68 (CH), 111.87 (C), 70.29 (CH), 61.00 (CH), 59.93 (CH₂), 31.63 (CH₂), 28.97 (CH₂), 26.36 (CH₂), 22.65 (CH₂), 20.80 (CH₂), 18.21 (CH₃), 14.77 (CH₃), 14.15 (CH₃).

### Ethyl erythro-1-(-2-hydroxynonan-3-yl)-1H-imidazole-4-carboxylate

Ethyl erythro-1-(-2-hydroxynonan-3-yl)-1H-imidazole-4-carboxylate was prepared following the procedure of Cristalli et al (J. Med. Chem., 1991, 34, 1187-1192):

To a stirred mixture of ethyl 5-amino-1-(2-hydroxynonan-3-yl)-1H-imidazole-4-carboxylate (625 mg, 2.10 mmol), acetonitrile (4.5 mL) and 50% w/w hypophosphorus acid (25 mL) at −20° C. was added dropwise a solution of sodium nitrite (348 mg, 5.04 mmol) in water (4.5 mL). After stirring at −20° C. for 3 h and then at ambient temperature overnight, the resulting orange mixture was neutralized with saturated sodium hydrogen carbonate solution and the yellow mixture extracted with ethyl acetate (100 mL, then 2×50 mL). TLC (1:1 light petroleum/acetone) indicated consumption of ethyl 5-amino-1-(2-hydroxynonan-3-yl)-1H-imidazole-4-carboxylate starting material (R_{f} 0.29) and formation of product (R_{f} 0.42). The red organic layer was dried over sodium sulfate, filtered, concentrated and the residue subjected to flash chromatography on silica gel (30 g). Gradient elution (1:2-1.5:2 acetone/light petroleum) afforded rac erythro-(±)-1-(2-hydroxynonan-3-yl)-N-methyl-1H-imidazole-4-carboxamide (412 mg, 1.46 mmol; 69%) as an pale yellow oil: δ_{H} (200 MHz; CDCl₃) 7.59 (1H, d, J 1.3), 7.46 (1H, d, J 1.4), 4.31 (2H, q, J 7.1), 4.09-3.81 (2H, m), 3.28 (1H, br s), 2.02-1.68 (2H, m), 1.34 (3H, t, J 7.1), 1.27-1.05 (8H, m), 1.12 (3H, d, J 6.4), 0.81 (3H, approx. t, J 6.5); δ_{C} (50 MHz; CDCl₃) 163.15 (C), 138.08 (CH), 133.59 (C), 124.47 (CH), 69.70 (CH), 64.85 (CH), 60.72 (CH₂), 31.67 (CH₂), 29.41 (CH₂), 29.02 (CH₂), 26.06 (CH₂), 22.67 (CH₂), 19.56 (CH₃), 14.59 (CH₃), 14.17 (CH₃).

### erythro-1-(2-hydroxynonan-3-yl)-N-methyl-1H-imidazole-4-carboxamide (HWC-7)

erythro-1-(2-Hydroxynonan-3-yl)-N-methyl-1H-imidazole-4-carboxamide (HWC-7) was prepared by adaptation of the procedure of Cristalli et al (J. Med. Chem., 1991, 34, 1187-1192):

A stirred solution of ethyl erythro-1-(2-hydroxynonan-3-yl)-1H-imidazole-4-carboxylate (306 mg, 1.08 mmol) in 33% w/w ethanolic methylamine (12 mL) was heated at 50° C. for 16 h in a heavy-walled sealed tube. TLC (1:1 light petroleum/acetone) indicated consumption of starting material (R_{f} 0.42) and formation of product (R_{f} 0.28). The solvent was removed in vacuo and the residue subjected to flash chromatography on silica gel (24 g). Gradient elution (1:2-1:1 acetone/light petroleum) returned starting material (76 mg) followed by erythro-1-(2-hydroxynonan-3-yl)-N-methyl-1H-imidazole-4-carboxamide (216 mg) as a colourless oil. The hydrochloride salt of erythro-1-(2-hydroxynonan-3-yl)-N-methyl-1H-imidazole-4-carboxamide was obtained as an oil by treatment with a saturated solution of hydrogen chloride in diethyl ether followed by evaporation. Consequently it was converted back into the free base form by partition between saturated sodium bicarbonate solution and dichloromethane. The organic extract was dried (sodium sulfate), evaporated and the residue subjected to flash chromatography on silica gel (10 g). Elution with hexane/acetone (1:1; 200 mL) returned erythro-1-(2-hydroxynonan-3-yl)-N-methyl-1H-imidazole-4-carboxamide (171 mg, 0.640 mmol; 59%): δ_{H} (200 MHz; CDCl₃) 7.56 (1H, d, J 1.3), 7.34 (1H, d, J 1.3), 7.20 (1H, q, J 5.0, NH), 4.64 (1H, br s, OH), 4.03-3.71 (2H, m, chain H-2 and H-3), 2.85 (3H, d, J 5.0, NCH₃), 1.98-1.58 (2H, m, chain CH₂-4), 1.30-1.04 (8H, m), 1.01 (3H, d, J 6.1, chain CH₃-1), 0.76 (3H, approx. t, J 6.5, chain CH₃-9); δ_{C} (50 MHz; CDCl₃) 163.73 (C), 136.91 (CH), 136.62 (C), 121.13 (CH), 69.47 (CH), 64.79 (CH), 31.62 (CH₂), 29.72 (CH₂), 28.95 (CH₂), 26.07 (CH₂), 25.79 (CH₃), 22.59 (CH₂), 19.42 (CH₃), 14.09 (CH₃); (found [M+H]⁺ 268.2018, calculated for C₁₄H₂₆O₂N₃: 268.2020). ¹H and ¹³C NMR spectra exhibited a minor set of signals corresponding threo-1-(2-hydroxynonan-3-yl)-N-methyl-1H-imidazole-4-carboxamide (ca. 5% contaminant) that was inseparable by flash column chromatography.

### Synthesis of racemic erythro-3-(1H-imidazo[4,5-c]pyridin-1-yl)nonan-2-ol (HWC-8)

### erythro-3-(2-Chloro-3-nitropyridin-4-ylamino)nonan-2-ol

erythro-3-(2-Chloro-3-nitropyridin-4-ylamino)nonan-2-ol was prepared by adaptation of the procedure reported by Antonini et al (J. Med. Chem., 1984, 27, 274-278):

A solution of 2,4-dichloro-3-nitropyridine (5.60 g, 29.0 mmol), erythro-3-aminononan-2-ol (5.08 g, 31.9 mmol) and triethylamine (6.56 mL, 47.0 mmol) in nitromethane (140 mL) was heated at 105° C. for 1 h. TLC (1:1 light petroleum/ethyl acetate) indicated consumption of the starting material (R_{f} 0.75) and formation of a major product component (R_{f} 0.50). After concentration in vacuo, the residue was dissolved in water and extracted with dichloromethane (3×70 mL). The combined organic layers were washed with brine (25 mL), dried over sodium sulfate, filtered and evaporated under reduced pressure to a residue, which was chromatographed on a silica gel column (60 g). Elution with light petroleum/ethyl acetate (4:1, 250 mL; 3:1 800 mL) gave erythro-3-(2-chloro-3-nitropyridin-4-ylamino)nonan-2-ol (2.09 g) as a yellow oil and mixed fractions. The mixed fractions were re-chromatographed on a silica gel column (40 g) eluting with light petroleum/ethyl acetate (4:1, 500 mL; 3:1, 400 mL; 1:1, 100 mL) to afford a further quantity of erythro-3-(2-chloro-3-nitropyridin-4-ylamino)nonan-2-ol (1.24 g). Combined yield (3.33 g, 10.5 mmol; 35%): δ_{H} (200 MHz; CDCl₃) 7.90 (1H, d, J6.2), 6.75 (1H, d, J 6.3), 6.53 (1H, d, J9.0), 4.00-3.86 (1H, m), 3.66-3.51 (1H, m), 1.73-1.43 (2H, m, chain CH₂-4)_{,} 1.44-1.21 (8H, m), 1.19 (3H, d, J 6.5, chain CH₃-1), 0.81 (3H, approx. t, J 6.7, chain CH₃-9); δ_{C} (50 MHz; CDCl₃) 149.47 (CH), 146.10 (2×C), 131.01 (C), 108.11 (CH), 69.62 (CH), 58.84 (CH), 31.73 (CH₂), 30.41 (CH₂), 29.31 (CH₂), 26.37 (CH₂), 22.69 (CH₂), 18.90 (CH₃), 14.17 (CH₃).

### erythro-3-(3-Aminopyridin-4-ylamino)nonan-2-ol

erythro-3-(3-Aminopyridin-4-ylamino)nonan-2-ol was prepared by adaptation of the procedure reported by Antonini et al (J. Med. Chem., 1984, 27, 274-278):

A solution of erythro-3-(2-chloro-3-nitropyridin-4-ylamino)nonan-2-ol (1.00 g, 3.01 mmol) in methanol (100 mL) was stirred in the presence of palladium, 10% on carbon, (1.00 g) under an atmosphere of hydrogen for 18 at 1 atm. TLC [9:1:0.3 dichloromethane/methanol/conc. NH₃ (aq)] indicated consumption of starting material (R_{f} 0.9) and formation of a product component (R_{f} 0.18). The reaction mixture was filtered and the filtrate was evaporated to give a light brown residue which was purified by silica gel column chromatography (25 g). Elution with dichloromethane/methanol/conc. NH₃ (aq) (9:1:0.3, 300 mL) gave erythro-3-(3-aminopyridin-2-ylamino)nonan-2-ol (589 mg, 2.34 mmol; 78%) as a colourless oil that solidified to a white solid upon standing (air-sensitive): δ_{H} (200 MHz; CDCl₃) 7.81 (1H, d, J5.5, pyridine H-6), 7.70 (1 H, s, pyridine H-2), 6.41 (1H, d, J 5.6, pyridine H-5), 4.32 (1H, d, J 8.8), 4.00-3.84 (1H, m), 3.42-3.30 (1H, m), 3.01 (3H, br s), 1.70-1.44 (2H, m, chain CH₂-4), 1.31-1.15 (8H, m), 1.19 (1H, d, J 6.5, chain CH₃-1), 0.81 (3H, approx. t, J 6.7, chain CH₃-9); δ_{C} (50 MHz; CDCl₃) 145.44 (CH), 143.83 (C), 138.33 (CH), 128.55 (C), 105.17 (CH), 68.63 (CH), 57.61 (CH), 31.90 (CH₂), 29.70 (CH₂), 29.54 (CH₂), 26.61 (CH₂), 22.78 (CH₂), 19.18 (CH₃), 14.24 (CH₃).

### erythro-3-(1H-imidazo[4,5-c]pyridin-1-yl)nonan-2-ol (HWC-8)

erythro-3-(1H-Imidazo[4,5-c]pyridin-1-yl)nonan-2-ol was prepared by adaptation of the procedure reported by Antonini et al (J. Med. Chem., 1984, 27, 274-278):

Concentrated hydrochloric acid (37% w/w; 0.24 mL) was added to a stirred solution of erythro-3-(3-aminopyridin-4-ylamino)nonan-2-ol (290 mg, 1.15 mmol) in triethyl orthoformate (10 mL). The reaction mixture was stirred at room temperature for 20 h. TLC (9:1:0.3 dichloromethane/methanol/conc. NH₃ (aq)) indicated consumption of starting material (R_{f} 0.18, staining brown in the air) and formation of a product component (R_{f} 0.41). The reaction mixture was neutralized with saturated sodium carbonate solution and extracted with dichloromethane (3×20 mL). The combined extract was washed with brine (3×10 mL), dried over sodium sulfate and concentrated in vacuo to give an oily residue. The latter was chromatographed on a silica gel column (30 g). Gradient elution with dichloromethane/methanol (95:5, 300 mL) and dichloromethane/methanol/conc. NH₃ (aq) (9:1:0.3, 200 mL) afforded erythro-3-(1H-imidazo[4,5-c]pyridin-1-yl)nonan-2-ol (210 mg, 0.803 mmol; 70%) as a colourless oil: δ_{H} (200 MHz; CDCl₃) 8.81 (1H, s), 8.19 (1H, d, J 5.7), 8.00 (1H, s), 7.31 (1H, d, J 5.5), 4.25-4.01 (2H, m), 3.73 (1H, br s), 2.14-1.93 (2H, m, chain CH₂-4), 1.20-1.01 (8H, m), 1.17 (3H, d, J 6.5, chain CH₃-1), 0.75 (3H, approx. t, J 6.6, chain CH₃-9); δ_{C} (50 MHz; CDCl₃) 143.86 (CH), 142.57 (CH), 141.64 (CH), 140.42 (C), 139.37 (C), 106.34 (CH), 69.23 (CH), 63.05 (CH), 31.56 (CH₂), 28.98 (CH₂), 28.69 (CH₂), 26.16 (CH₂), 22.57 (CH₂), 20.21 (CH₃), 14.07 (CH₃).

### Synthesis of racemic erythro-3-(4-amino-1H-imidazo[4,5-c]pyridin-1-yl)nonan-2-ol (HWC-9)

### erythro-3-(3-Amino-2-chloropyridin-4-ylamino)nonan-2-ol

erythro-3-(3-Amino-2-chloropyridin-4-ylamino)nonan-2-ol was prepared by adaptation of the procedure reported by Antonini et al (J. Med. Chem., 1984, 27, 274-278):

A solution of erythro-3-(2-chloro-3-nitropyridin-4-ylamino)nonan-2-ol (preparation—vide supra; 1.49 g, 4.48 mmol) in ethanol (100 mL) was stirred in the presence of platinum(IV) oxide (0.102 g) under an atmosphere of hydrogen at 1 atm for 3 h. TLC (1:1 light petroleum/ethyl acetate) indicated consumption of starting material (R_{f} 0.50) and formation of a product component (R_{f} 0.29). The reaction mixture was filtered over celite and the filtrate was evaporated to give a light brown residue which was purified by silica gel column chromatography (25 g). Gradient elution with light petroleum/ethyl acetate (3:1, 800 mL; 1:1, 500 mL) gave erythro-3-(3-amino-2-chloropyridin-4-ylamino)nonan-2-ol (1.13 g, 3.94 mmol; 88%) as a colourless oil: δ_{H} (200 MHz; CDCl₃) 7.59 (1H, d, J 5.5, pyridine H-6), 6.38 (1H, d, J 5.7, pyridine H-6), 4.37 (1H, d, J 8.7), 4.01-3.84 (1H, m), 3.54 (2H, br s), 3.43-3.29 (1H, m), 3.05 (1H, br s), 1.71-1.42 (2H, m, chain CH₂-4), 1.32-1.16 (11H, m), 0.81 (3H, approx. t, J 6.8, chain CH₃-9); δ_{C} (50 MHz; CDCl₃) 146.26 (C), 141.72 (CH), 139.44 (C), 125.02 (C), 105.05 (CH), 68.93 (CH), 58.19 (CH), 31.86 (CH₂), 29.73 (CH₂), 29.50 (CH₂), 26.54 (CH₂), 22.76 (CH₂), 19.21 (CH₃), 14.23 (CH₃).

### erythro-3-(4-Chloro-1H-imidazo[4,5-c]pyridin-1-yl)nonan-2-ol

erythro-3-(4-Chloro-1H-imidazo[4,5-e]pyridin-1-yl)nonan-2-ol was prepared by adaptation of the procedure reported by Antonini et al (J. Med. Chem., 1984, 27, 274-278):

Concentrated hydrochloric acid (37% w/w; 0.77 mL) was added to a stirred solution of erythro-3-(3-amino-2-chloropyridin-4-ylamino)nonan-2-ol (1.11 g, 3.87 mmol) in triethyl orthoformate (35 mL). The reaction mixture was stirred at room temperature for 20 h. TLC (96:4 dichloromethane/methanol) indicated consumption of starting material (R_{f} 0.18, staining brown in the air) and formation of a product component (R_{f} 0.11) and a minor component (R_{f} 0.34). The reaction mixture was neutralized with saturated sodium carbonate solution and extracted with dichloromethane (3×30 mL), dried over sodium sulfate and concentrated in vacuo to give a pale yellow oily residue. The latter was chromatographed on a silica gel column (30 g). Gradient elution with dichloromethane/methanol (98:2, 250 mL; 96:4, 250 mL) gave erythro-3-(4-chloro-1H-imidazo[4,5-c]pyridin-1-yl)nonan-2-ol (768 mg, 2.60 mmol; 67%) as a colourless oil: δ_{H} (200 MHz; CDCl₃) 8.05 (1H, d, J3.4), 8.03 (1H, s), 7.29 (1H, d), 4.30-4.10 (2H, m), 4.00 (1H, s), 2.12-1.97 (2H, m, chain CH₂-4), 1.26-1.07 (11H, m), 0.75 (3H, approx. t, J 7.0, chain CH₃-9); δ_{C} (50 MHz; CDCl₃) 143.90 (CH), 142.61 (C), 141.32 (CH), 140.60 (C), 137.24 (C), 106.23 (CH), 69.19 (CH), 63.43 (CH), 31.56 (CH₂), 28.98 (CH₂), 28.29 (CH₂), 26.10 (CH₂), 22.59 (CH₂), 20.19 (CH₃), 14.09 (CH₃).

### erythro-3-(4-Amino-1H-imidazo[4,5-c]pyridin-1-yl)nonan-2-ol hydrochloride (HWC-9)

erythro-3-(4-Amino-1H-imidazo[4,5-c]pyridin-1-yl)nonan-2-ol was prepared by adaptation of the procedure reported by Antonini et al (J. Med. Chem., 1984, 27, 274-278):

A solution of erythro-3-(4-chloro-1H-imidazo[4,5-c]pyridin-1-yl)nonan-2-ol (693 mg, 2.34 mmol) in hydrazine hydrate (14.0 mL) was refluxed for 2 h. TLC (dichloromethane/methanol 96:4) indicated consumption of starting material (R_{f} 0.11) and formation of a product component (baseline). The reaction mixture was evaporated under reduced pressure to dryness. Oxygen-free water (30 mL) and Raney nickel (1.82 g, wet weight) were added and the mixture was refluxed for 1 h. TLC (9:1 dichloromethane/methanol) indicated formation of a product component (R_{f} 0.18). The catalyst was removed by filtration through a celite, washing with hot water and hot dichloromethane. The aqueous layer was extracted with dichloromethane (3×30 mL) and the combined organic extracts were dried over sodium sulfate and evaporated. The resulting residue was chromatographed on a silica gel column (20 g). Gradient elution with dichloromethane/methanol (95:5, 500 mL; 9:1, 200 mL) gave erythro-3-(4-amino-1H-imidazo[4,5-c]pyridin-1-yl)nonan-2-ol (217 mg, 0.785 mmol; 33%) as an off-white solid: δ_{H} (200 MHz; 5% CD₃OD/CDCl₃) 7.82 (1H, s), 7.64 (1H, d, J 6.1), 6.66 (1H, d, J 6.1), 4.04-3.89 (2H, m), 2.09-1.79 (2H, m, chain CH₂-4), 1.33-1.05 (8H, m), 1.01 (3H, d, J 6.5, chain CH₃-1), 0.71 (3H, approx. t, J6.9, chain CH₃-9); δ_{C} (50 MHz; 5% CD₃OD/CDCl₃) 151.61 (C), 140.55 (CH), 139.75 (CH), 139.33 (C), 126.40 (C), 98.04 (CH), 69.07 (CH), 63.16 (CH), 31.44 (CH₂), 29.09 (CH₂), 28.78 (CH₂), 25.96 (CH₂), 22.41 (CH₂), 19.83 (CH₃), 13.82 (CH₃). erythro-3-(4-Amino-1H-imidazo[4,5-c]pyridin-1-yl)nonan-2-ol was converted into its hydrochloride salt (HWC-9) by treatment with a solution of hydrogen chloride in ether followed by evaporation.

### Synthesis of racemic erythro-3-(6-chloro-9H-purin-9-yl)nonan-2-ol (HWC-10)

### erythro-3-(6-Chloro-5-nitropyrimidin-4-ylamino)nonan-2-ol

erythro-3-(6-Chloro-5-nitropyrimidin-4-ylamino)nonan-2-ol was prepared by adaptation of procedures reported by Schaeffer and Schwender (J. Med. Chem., 1974, 17, 6-8 and by Zhang et al (Bioorg. Med. Chem., 2006, 14, 8314-8322):

An ice-cooled 250 mL round bottom flask was charged with erythro-3-aminononan-2-ol (4.10 g, 25.7 mmol), ethanol (110 mL) and triethylamine (18.5 mL, 133 mmol). To the pale yellow solution was added 4,6-dichloro-5-nitropyrimidine (5.42 g, 27.9 mmol). The orange mixture was maintained at 0° C. for 3 h and the solvent was then evaporated at room temperature. The residue was dissolved in ethyl acetate (300 mL), washed with ice-cold water (3×25 mL), dried over magnesium sulfate and concentrated in vacuo to give a dense brown oil (9.85 g). The crude material was chromatographed on a silica gel column (200 g). Gradient elution with light petroleum/ethyl acetate (9:1, 1 L; 7:1, 1.6 L; 3:1, 1.2 L) gave erythro-3-(6-chloro-5-nitropyrimidin-4-ylamino)nonan-2-ol (4.13 g; 51%) as a yellow oil: δ_{H} (200 MHz; CDCl₃) 8.30 (1H, s), 7.53 (1H, d, J 8.6), 4.50-4.34 (1H, m), 4.01-3.88 (1H, m), 2.51 (1H, s), 1.68-1.44 (2H, m, chain CH₂-4), 1.43-1.19 (8H, m), 1.17 (3H, d, J6.5, chain CH₃-1), 0.80 (3H, approx. t, J6.6, chain CH₃-9); δ_{C} (50 MHz; CDCl₃) 158.07 (CH), 156.37 (C), 155.51 (C), 107.80 (C), 69.87 (CH), 57.19 (CH), 31.74 (CH₂), 29.50 (CH₂), 29.22 (CH₂), 26.28 (CH₂), 22.69 (CH₂), 18.79 (CH₃), 14.17 (CH₃).

### erythro-3-(5-Amino-6-chloropyrimidin-4-ylamino)nonan-2-ol

A mixture of erythro-3-(6-chloro-5-nitropyrimidin-4-ylamino)nonan-2-ol (1.02 g, 3.22 mmol) and tin(II) chloride dihydrate (3.63 g, 16.1 mmol) in ethanol (32 mL) was stirred at 80° C. for 10 minutes. TLC (1:1 light petroleum/ethyl acetate) indicated consumption of starting material (R_{f} 0.70) and formation of a product component (R_{f} 0.20). The solvent was removed in vacuo and the residue was dissolved in dichloromethane (120 mL) and washed with saturated sodium bicarbonate solution (2×40 mL). The organic layer was dried over magnesium sulfate and concentrated in vacuo. The crude residue was purified by a silica gel column (40 g). Elution with light petroleum/ethyl acetate (1:1, 1000 mL) gave erythro-3-(5-amino-6-chloropyrimidin-4-ylamino)nonan-2-ol (0.58 g; 63%) as a dense yellow oil that solidified upon standing: δ_{H} (200 MHz; CDCl₃) 7.98 (1H, s), 4.96 (1H, d, J 7.1, NH), 4.21-4.08 (1H, m, chain H-3), 3.97 (1H, qd, J 6.4 and 2.5, chain H-2), 3.60-3.40 (3H, br s), 1.62-1.46 (2H, m, chain CH₂-4), 1.31-1.22 (8H, m), 1.13 (3H, d, J 6.4, chain CH₃-1), 0.84 (3H, approx. t, J 6.8, chain CH₃-9); δ_{C} (50 MHz; CDCl₃) 155.41 (C), 148.99 (CH), 143.03 (C), 122.17 (C), 70.87 (CH), 57.39 (CH), 31.84 (CH₂), 30.59 (CH₂), 29.33 (CH₂), 26.72 (CH₂), 22.75 (CH₂), 18.27 (CH₃), 14.21 (CH₃).

### erythro-3-(6-Chloro-9H-purin-9-yl)nonan-2-ol (HWC-10)

Erythro-3-(6-Chloro-9H-purin-9-yl)nonan-2-ol was prepared by adaptation of the procedure reported Schaeffer et al (J. Med. Chem., 1974, 17, 6-8):

Ethanesulfonic acid (8 μL) was added to a stirred suspension of erythro-3-(5-amino-6-chloropyrimidin-4-ylamino)nonan-2-ol (144 mg, 0.502 mmol) in triethyl orthoformate (2.1 mL) and chloroform (0.7 mL). The solid dissolved immediately, forming a pale yellow solution. The reaction mixture was stirred at room temperature for 1 h. TLC (95:5 dichloromethane/methanol) indicated consumption of starting material (R_{f} 0.18, staining yellow in air) and formation of a product component (R_{f} 0.26) and a minor product (R_{f} 0.66). The reaction mixture was diluted with dichloromethane (25 mL) and washed with saturated sodium bicarbonate solution solution (2×20 mL). The organic layer was dried over sodium sulfate, filtered and concentrated in vacuo to give a pale yellow oil that was chromatographed on a silica gel column (20 g). Elution with dichloromethane/methanol (98:2, 300 mL) gave erythro-3-(6-chloro-9H-purin-9-yl)nonan-2-ol (100 mg, 0.337 mmol; 67%) as a colourless oil: δ_{H} (200 MHz; CDCl₃) 8.64 (1H, s), 8.22 (1H, s), 4.57-4.44 (1H, chain H-3), 4.29-4.17 (1H, m, chain H-2), 4.05 (1H, br s), 2.15-1.91 (2H, m, chain CH₂-4), 1.25 (3H, d, J 6.4, chain CH₃-1), 1.20-1.02 (8H, m), 0.74 (3H, approx. t, J 6.6, chain CH₃-9); δ_{C} (50 MHz; CDCl₃) 152.00 (C), 151.73 (CH), 151.37 (C), 145.39 (CH), 131.89 (C), 69.57 (CH), 62.87 (CH), 31.62 (CH₂), 28.92 (CH₂), 27.58 (CH₂), 26.22 (CH₂), 22.63 (CH₂), 20.46 (CH₃), 14.13 (CH₃).

### Synthesis of racemic erythro-3-(9H-purin-9-yl)nonan-2-ol (HWC-12)

erythro-3-(9H-Purin-9-yl)nonan-2-ol was prepared following the procedure of Antonini et al (J. Med. Chem., 1984, 27, 274-278):

A solution of erythro-3-(6-chloro-9H-purin-9-yl)nonan-2-ol (150 mg, 0.505 mmol) in ethanol (10 mL) was stirred in the presence of palladium, 10% on carbon, (54 mg) under an atmosphere of hydrogen (1 atm) at room temperature for 5 h. TLC (9:1 dichloromethane/methanol) indicated consumption of starting material (R_{f} 0.53) and formation of a product component (R_{f} 0.31). The solvent was evaporated to give a light brown residue that was purified chromatographically on a silica gel column (25 g). Gradient elution with dichloromethane/methanol (98:2, 250 mL; 96:4, 300 mL) gave erythro-3-(9H-purin-9-yl)nonan-2-ol (60 mg, 0.229 mmol; 45%) as a colourless oil: δ_{H} (200 MHz; CDCl₃) 8.99 (1H, s), 8.84 (1H, s), 8.16 (1H, s), 4.55 (1H, br s), 4.52-4.42 (1H, chain H-3), 4.24-4.11 (1H, chain H-2), 2.20-1.90 (2H, m, chain CH₂-4), 1.24 (3H, d, J6.5, chain CH₃-1), 1.21-0.93 (8H, m), 0.73 (3H, approx. t, J6.6, chain CH₃-9); δ_{C} (50 MHz; CDCl₃) 152.19 (CH), 151.49 (C), 148.56 (CH), 145.45 (CH), 134.11 (C), 69.31 (CH), 62.17 (CH), 31.55 (CH₂), 28.87 (CH₂), 27.55 (CH₂), 26.17 (CH₂), 22.54 (CH₂), 20.40 (CH₃), 14.05 (CH₃).

### Synthesis of racemic erythro-3-(6-mercapto-9H-purin-9-yl)nonan-2-ol hydrochloride (HWC-13)

A solution of erythro-3-(6-chloro-9H-purin-9-yl)nonan-2-ol (208 mg, 0.701 mmol) and thiourea (107 mg, 1.40 mmol) in ethanol (7 mL) was heated at 85° C. for 2 h. TLC (9:1 dichloromethane/methanol) indicated consumption of starting material (R_{f} 0.53) and formation of a product component (R_{f} 0.38) (thiourea R_{f} 0.21). The clear reaction mixture was cooled and the solvent was evaporated. The residue was purified was purified chromatographically on a silica gel column (25 g). Gradient elution with dichloromethane/methanol (98:2, 650 mL; 96:4, 200 mL) gave erythro-3-(6-mercapto-9H-purin-9-yl)nonan-2-ol (134 mg, 0.455 mmol; 65%) as a white powder: δ_{H} (200 MHz; CDCl₃/CD₃OD) 8.25 (1H, s), 8.12 (1H, s), 4.47-4.35 (1H, m, chain H-3), 4.07 (1H, quintet, J 6.1, chain H-2), 2.08 (2H, q, J 7.3), 1.45-1.12 (8H, m), 1.10 (3H, d, J 6.4, chain CH₃-1), 0.80 (3H, approx. t, J 6.8, chain CH₃-9); δ_{C} (50 MHz; CDCl₃/CD₃OD) 177.95 (C), 145.74 (CH), 145.45 (C), 143.26 (CH), 136.23 (C), 70.02 (CH), 63.02 (CH), 32.62 (CH₂), 29.72 (2×CH₂), 27.00 (CH₂), 23.49 (CH₂), 20.34 (CH₃), 14.43 (CH₃). erythro-3-(6-Mercapto-9H-purin-9-yl)nonan-2-ol was converted into its hydrochloride salt (HWC-13) by treatment with a solution of hydrogen chloride in ether followed by evaporation.

### Synthesis of racemic erythro-9-(2-hydroxynonan-3-yl)-9H-purin-6-ol hydrochloride (HWC-14)

A solution of erythro-3-(6-chloro-9H-purin-9-yl)nonan-2-ol (158 mg, 0.532 mmol) in 1.2 M hydrochloric acid (10 mL) was heated under at 110° C. for 2.5 h. TLC (1:2 light petroleum/ethyl acetate) indicated the transformation of starting material (R_{f} 0.58) into product (R_{f} 0.01). Evaporation at reduced pressure gave the crude product as a pale yellow solid that was recrystallised from methanol/diethyl ether to give erythro-9-(2-hydroxynonan-3-yl)-9H-purin-6-ol hydrochloride (132 mg; 79%) as a white solid: δ_{H} (200 MHz; CD₃OD) 9.48 (1H, s), 8.30 (1H, s), 4.83-4.71 (1H, m), 4.15-4.01 (1H, m), 2.22-2.09 (2H, m, chain CH₂-4), 1.28-1.20 (11H, m), 0.85 (3H, approx. t, J 6.7, chain CH₃-9); δ_{C} (50 MHz; CD₃OD) 154.87 (C), 150.25 (CH), 149.23 (C), 140.32 (CH), 117.72 (C), 69.27 (CH), 64.82 (CH), 32.76 (CH₂)_{,} 29.90 (CH₂), 28.66 (CH₂), 26.95 (CH₂), 23.68 (CH₂), 19.88 (CH₃), 14.46 (CH₃).

### Procedure for preparation of racemic erythro-3-(6-methoxy-9H-purin-9-yl)nonan-2-ol hydrochloride (HWC-15)

A solution of erythro-3-(6-chloro-9H-purin-9-yl)nonan-2-ol (150 mg, 0.505 mmol) and sodium methoxide (0.084 mL, 2.02 mmol) in anhydrous methanol (10 mL) was heated at 70° C. for 2 h. TLC (1:2 light petroleum/ethyl acetate) indicated the transformation of starting material (R_{f} 0.58) into product (R_{f} 0.42). The reaction mixture was cooled, adjusted to pH 6 with acetic acid and evaporated. The residual solid was dissolved in water (10 mL) and extracted with dichloromethane (20 mL) and ethyl acetate (20 mL). The combined organic layers were dried over sodium sulfate, filtered and concentrated in vacuo to give a crude oil that was purified on a silica gel column (20 g). Elution with dichloromethane/methanol (96:4, 150 mL) gave erythro-3-(6-methoxy-9H-purin-9-yl)nonan-2-ol (142 mg, 0.486 mmol; 96%) as a pale yellow oil: δ_{H} (200 MHz; CDCl₃) 8.44 (1H, s), 7.89 (1H, s), 4.78 (1H, br d, J 3.6), 4.38-4.19 (2H, m, chain H-2 and H-3), 4.12 (3H, s, OMe), 2.10-1.87 (2H, m, chain CH₂-4), 1.27 (3H, d, J6.5, chain CH₃-1), 1.24-1.00 (8H, m), 0.77 (3H, approx. t, J 6.6, chain CH₃-9); δ_{C} (50 MHz; CDCl₃) 161.16 (C), 151.76 (C), 151.64 (CH), 142.44 (CH), 124.24 (C), 69.41 (CH), 63.30 (CH), 54.42 (OCH₃), 31.65 (CH₂), 28.96 (CH₂), 27.24 (CH₂), 26.28 (CH₂), 22.63 (CH₂), 20.37 (CH₃), 14.13 (CH₃). erythro-3-(6-Methoxy-9H-purin-9-yl)nonan-2-ol was converted into its hydrochloride salt (HWC-15) by treatment with a solution of hydrogen chloride in ether followed by evaporation.

### Synthesis of racemic erythro-3-[6-(methylamino)-9H-purin-9-yl]nonan-2-ol (HWC-16)

A stirred mixture of erythro-3-(6-chloro-9H-purin-9-yl)nonan-2-ol (208 mg, 0.701 mmol) and methylamine (33% w/w in ethanol; 14.0 mL) was heated at 100° C. in a heavy-walled sealed flask for 17 h. TLC (9:1 dichloromethane/methanol) indicated consumption of starting material (R_{f} 0.53) and formation of a product component (R_{f} 0.40). The reaction mixture was evaporated and the residue chromatographed on a silica gel column (25 g). Gradient elution with dichloromethane/methanol (98:2, 600 mL; 96:4, 100 mL) gave erythro-3-[6-(methylamino)-9H-purin-9-yl]nonan-2-ol (191 mg) as a pale yellow oil. ¹H NMR indicated the presence of minor impurities and the crude material was re-chromatographed on a silica gel column (20 g). Elution with light petroleum:acetone (2:1, 300 mL) gave erythro-3-(6-(methylamino)-9H-purin-9-yl)nonan-2-ol (151 mg, 0.518 mmol; 74%): δ_{H} (200 MHz; CDCl₃) 8.32 (1H, s), 7.67 (1H, s), 6.17 (1H, br q, NH), 5.48 (1H, br s, NH), 4.25-4.13 (2H, m, chain H-2 and H-3), 3.18 (3H, br d, NMe), 2.09-1.77 (2H, m, chain CH₂-4), 1.25 (3H, d, J 6.5, chain CH₃-1), 1.23-1.04 (8H, m), 0.79 (3H, approx. t, J 6.5, chain CH₃-9); δ_{C} (50 MHz; CDCl₃) 155.83 (C), 152.67 (C), 152.67 (CH), 140.03 (CH), 124.04 (C), 69.78 (CH), 63.93 (CH), 31.72 (CH₂), 29.01 (CH₂), ˜28 (br, NMe), 27.43 (CH₂), 26.47 (CH₂), 22.66 (CH₂), 20.49 (CH₃), 14.16 (CH₃).

### Procedure for preparation of racemic erythro-3-[6-(dimethylamino)-9H-purin-9-yl]nonan-2-ol hydrochloride (HWC-17)

Dimethylamine hydrochloride (3.0 g, 36.8 mmol) was dissolved in water (9 mL) and cooled in an ice-bath. Sodium hydroxide (1.47 g, 36.8 mmol) was added in portions with stirring. The resulting aqueous dimethylamine solution was added to a solution of erythro-3-(6-chloro-9H-purin-9-yl)nonan-2-ol (215 mg, 0.724 mmol) in ethanol (5 mL) and the mixture heated at 100° C. for 17 h in a heavy-walled sealed flask. TLC (1:1 light petroleum/ethyl acetate) indicated transformation of starting material (R_{f} 0.28) into product (R_{f} 0.15). The reaction mixture was concentrated in vacuo; the residue diluted with water (10 mL) and extracted with ethyl acetate (50 mL) and dichloromethane (2×15 mL). The combined extract was dried over sodium sulfate, filtered and evaporated to give an oily residue that was chromatographed on a silica gel column (25 g). Elution with dichloromethane/methanol (96:4, 250 mL) gave erythro-3-[6-(dimethylamino)-9H-purin-9-yl]nonan-2-ol (220 mg, 0.720 mmol; 99%) as a colourless oil: δ_{H} (200 MHz; CDCl₃) 8.23 (1H, s), 7.63 (1H, s), 5.65 (1H, br s), 4.29-4.11 (2H, m, chain H-2 and H-3), 3.51 (6H, br s), 2.12-1.75 (2H, m, chain CH₂-4), 1.25 (3H, d, J 6.5, chain CH₃-1), 1.22-1.00 (8H, m), 0.79 (3H, approx. t, J 6.5, chain CH₃-9); δ_{C} (50 MHz; CDCl₃) 155.23 (C), 151.71 (CH), 150.06 (C), 138.78 (CH), 120.88 (C), 69.89 (CH), 64.20 (CH), 38.79 (br, 2×CH₃), 31.74 (CH₂), 29.05 (CH₂), 27.16 (CH₂), 26.53 (CH₂), 22.69 (CH₂), 20.54 (CH₃), 14.18 (CH₃). erythro-3-[6-(Dimethylamino)-9H-purin-9-yl]nonan-2-ol was converted into its hydrochloride salt (HWC-17) by treatment with a solution of hydrogen chloride in ether followed by evaporation.

### Procedure for preparation of racemic erythro-3-(6-amino-8-methyl-9H-purin-9-yl)nonan-2-ol hydrochloride (HWC-24)

### erythro-3-(6-Chloro-8-methyl-9H-purin-9-yl)nonan-2-ol

Ethanesulfonic acid (8.00 μL, 0.098 mmol) was added to a stirred solution of erythro-3-(5-amino-6-chloropyrimidin-4-ylamino)nonan-2-ol (preparation—vide supra; 192 mg, 0.669 mmol) in triethyl orthoacetate (3.0 mL) and chloroform (1.0 mL). The reaction mixture was stirred at room temperature for 1 h. TLC (1:1 light petroleum/ethyl acetate) indicated consumption of starting material (R_{f} 0.35, staining yellow in air) and formation of a product component (R_{f} 0.30) and a minor component (R_{f} 0.56). The reaction mixture was diluted with dichloromethane (50 mL) and washed with saturated sodium bicarbonate solution (3×10 mL). The organic layer was dried over sodium sulfate, filtered and concentrated in vacuo to give a pale yellow oily which was chromatographed on a silica gel column (25 g). Gradient elution with light petroleum/ethyl acetate (3:1, 400 mL; 2:1, 300 mL; 1:1, 200 mL) gave erythro-3-(6-chloro-8-methyl-9H-purin-9-yl)nonan-2-ol (R_{f} 0.30) (102 mg, 0.328 mmol; 49%) as a dense colourless oil: δ_{H} (200 MHz; CDCl₃) 8.57 (1H, s), 4.66 (1H, s), 4.45-4.30 (1H, m), 4.18-4.04 (1H, m), 2.64 (3H, s), 2.40-1.91 (2H, m, chain CH₂-4), 1.18 (3H, d, J6.4, chain CH₃-1), 1.15-0.98 (8H, m), 0.74 (3H, approx. t, J 6.5, chain CH₃-9); δ_{C} (50 MHz; CDCl₃) 156.04 (C), 152.55 (C), 150.40 (CH), 149.66 (C), 131.23 (C), 69.40 (CH), 64.78 (CH), 31.63 (CH₂), 29.08 (CH₂), 27.72 (CH₂), 26.58 (CH₂), 22.57 (CH₂), 21.26 (CH₃), 15.60 (CH₃), 14.07 (CH₃).

### erythro-3-(6-Amino-8-methyl-9H-purin-9-yl)nonan-2-ol hydrochloride (HWC-24)

A solution of erythro-3-(6-chloro-8-methyl-9H-purin-9-yl)nonan-2-ol (97 mg, 0.31 mmol) in ammonia 7 N in methanol (10 mL) was stirred at 120° C. in a heavy-walled sealed tube for 18 h. TLC (95:5 dichloromethane/methanol) indicated the starting material (R_{f} 0.44) was transformed into a minor product (R_{f} 0.26) and a major product at (R_{f} 0.12). The reaction mixture was concentrated in vacuo to give a light brown residue that was chromatographed on a silica gel column (20 g). Gradient elution with dichloromethane/methanol (98:2, 200 mL; 96:4, 200 mL) gave erythro-3-(6-amino-8-methyl-9H-purin-9-yl)nonan-2-ol (R_{f} 0.12) (81 mg, 0.278 mmol; 89%) as a white solid: δ_{H} (200 MHz; CDCl₃) 8.14 (1H, s, H-2), 6.25 (1H, br s, OH), 4.26 (1H, qd, J 6.5 and 3.0, chain H-2), 3.97 (1H, dt, J 11.1 and 3.0, chain H-3), 2.51 (3H, s, 8-Me), 2.30-1.81 (2H, m, chain CH₂-4), 1.21 (3H, d, J6.5, chain CH₃-1), 1.22-1.06 (8H, m), 0.76 (3H, approx. t, J 6.5, chain CH₃-9); δ_{C} (50 MHz; CDCl₃) 154.78 (C), 154.73 (C), 151.22 (CH), 150.02 (C), 149.85 (C), 69.35 (CH), 64.19 (CH), 31.59 (CH₂), 29.08 (CH₂), 27.37 (CH₂), 26.55 (CH₂), 22.52 (CH₂), 21.02 (CH₃), 14.91 (CH₃), 13.98 (CH₃). The minor product component (R_{f} 0.26; ˜10 mg) was identified as 3-(6-methoxy-8-methyl-9H-purin-9-yl)nonan-2-ol: δ_{H} (200 MHz; CDCl₃) 8.39 (1H, s, H-2), 5.67 (1H, br s, OH), 4.32 (1H, qd, J 6.5 and 2.3, chain H-2), 4.16 (3H, s, OMe), 4.06 (1H, dt, J 11.1 and 2.7, chain H-3), 2.59 (3H, s, 8-Me), 2.31-1.84 (2H, m, chain CH₂-4), 1.27 (3H, d, J 6.4, chain CH₃-1), 1.22-1.06 (8H, m), 0.76 (3H, approx. t, J 6.8, chain CH₃-9); δ_{C} (50 MHz; CDCl₃) 160.49 (C), 152.44 (C), 152.05 (C), 150.49 (CH), 121.00 (C), 69.92 (CH), 64.46 (CH), 54.37 (OCH₃), 31.70 (CH₂), 29.23 (CH₂), 27.39 (CH₂), 26.65 (CH₂), 22.66 (CH₂), 21.43 (CH₃), 15.22 (CH₃), 14.14 (CH₃). erythro-3-(6-Amino-8-methyl-9H-purin-9-yl)nonan-2-ol was converted into its hydrochloride salt (HWC-24) by treatment with a solution of hydrogen chloride in ether followed by evaporation.

### Synthesis of racemic erythro-3-[6-(methylthio)-9H-purin-9-yl]nonan-2-ol (HWC-25)

Sodium thiomethoxide (71 mg, 1.0 mmol) was added to a stirred solution of erythro-3-(6-chloro-9H-purin-9-yl)nonan-2-ol (150 mg, 0.505 mmol) in a mixture of DMF (3 mL) and water (1 mL) at 0° C. The mixture was allowed to attain room temperature and stirred for 3 h. TLC (9:1 dichloromethane/methanol) indicated consumption of starting material (R_{f} 0.53) and formation of product (R_{f} 0.60). The reaction mixture was diluted with ethyl acetate (50 mL) and then washed successively with water (15 mL), saturated sodium bicarbonate solution (15 mL) and brine (15 mL). The organic phase was dried over sodium sulfate, filtered and evaporated to give a dense colourless oil that was chromatographed on a silica gel column (10 g). Gradient elution with dichloromethane/methanol (99:1, 100 mL; 98:2, 100 mL) gave partially purified product (152 mg) that was re-chromatographed on a silica gel column (10 g). Gradient elution with dichloromethane/methanol (99:1, 100 mL, 98:2, 100 mL) gave erythro-3-[6-(methylthio)-9H-purin-9-yl]nonan-2-ol (146 mg, 0.473 mmol; 94%): δ_{H} (200 MHz; CDCl₃) 8.62 (1H, s), 7.98 (1H, s), 4.72 (1H, br d, J4.1, OH), 4.38 (1H, dt, J 10.7 and 3.5, chain H-3), 4.22 (1H, ˜qt, J6.4 and 3.3, chain H-2), 2.62 (3H, s, SMe), 2.17-1.85 (2H, m, chain CH₂-4), 1.25 (3H, d, J 6.5, chain CH₃-1), 1.20-0.95 (8H, m), 0.76 (3H, approx. t, J 6.5, chain CH₃-9); δ_{C} (50 MHz; CDCl₃) 161.84 (C), 151.47 (CH), 148.06 (C), 142.72 (CH), 131.42 (C), 69.28 (CH), 62.70 (CH), 31.58 (CH₂), 28.90 (CH₂), 27.30 (CH₂), 26.17 (CH₂), 22.56 (CH₂), 20.34 (CH₃), 14.06 (CH₃), 11.86 (CH₃).

### Synthesis of racemic erythro-3-(7-amino-3H-[1,2,3]triazolo[4,5-d]pyrimidin-3-yl)nonan-2-ol (HWC-26)

Sodium nitrite (64 mg, 0.93 mmol) in water (3 mL) was added slowly to a mixture of erythro-3-(5-amino-6-chloropyrimidin-4-ylamino)nonan-2-ol (220 mg, 0.767 mmol) in ethanol (5 mL) and hydrochloric acid 1 M (2 mL) at 0° C. The mixture was stirred at 0° C. for 30 minutes, treated with conc. ammonium hydroxide (5.0 mL) and refluxed for 30 minutes. The water was reduced by azeotropic distillation of the reaction mixture with toluene and ethanol to give a crude solid (300 mg) that was chromatographed on a silica gel column (20 g). Elution with dichloromethane/methanol (95:5, 200 mL) gave erythro-3-(7-amino-3H-[1,2,3]triazolo[4,5-d]pyrimidin-3-yl)nonan-2-ol (185 mg, 0.665 mmol; 87%) as a pale yellow solid: δ_{H} (400 MHz; CDCl₃) 8.45 (1H, s), 7.20 (2H, br s, NH₂), 4.89 (1H, dt, J 11.2 and 3.3, chain H-3), 4.58 (1H, br d, J 2.2, OH), 4.37-4.30 (1H, m, chain H-2), 2.32-2.19 (1H, m), 2.08-1.99 (1H, m), 1.32 (3H, d, J 6.4, chain CH₃-1), 1.30-1.13 (7H, m), 1.05-0.93 (1H, m), 0.84 (3H, approx. t, J 6.5, chain CH₃-9); δ_{C} (101 MHz; CDCl₃) 156.26 (C), 156.25 (CH), 149.16 (C), 124.61 (C), 69.57 (CH), 66.23 (CH), 31.49 (CH₂), 28.74 (CH₂), 28.02 (CH₂), 26.05 (CH₂), 22.46 (CH₂), 19.75 (CH₃), 13.97 (CH₃).

### Procedure for preparation of racemic erythro-3-(6-hydrazinyl-9H-purin-9-yl)nonan-2-ol (HWC-27)

A mixture of erythro-3-(6-chloro-9H-purin-9-yl)nonan-2-ol (210 mg, 0.708 mmol) and hydrazine hydrate (0.344 mL) in ethanol (7.0 mL) was refluxed at 85° C. for 20 h. TLC (9:1 dichloromethane/methanol) indicated consumption of starting material (R_{f} 0.53) and formation of a product component (R_{f} 0.20, air sensitive). The reaction mixture was evaporated and the residue was chromatographed on a silica gel column (25 g). Gradient elution with dichloromethane/methanol (98:2, 200 mL; 95:5, 200 mL; 9:1, 100 mL) gave erythro-3-(6-hydrazinyl-9H-purin-9-yl)nonan-2-ol (137 mg, 0.469 mmol; 66%) as a white solid: δ_{H} (400 MHz; DMSO-d₆) 8.87 (1H, br s, NH), 8.22 (1H, br s), 8.16 (1H, s), 5.15 (1H, d, J5.5, OH), 4.57 (2H, br s, NH₂), 4.28-4.15 (1H, m, chain H-3), 4.24 (1H, ˜sextet, J 6.2, chain H-3), 2.07-2.02 (2H, m, chain CH₂-4), 1.22-1.02 (8H, m), 0.89 (3H, d, J 6.3, chain CH₃-1), 0.78 (3H, approx. t, J 6.5, chain CH₃-9); δ_{C} (101 MHz; DMSO-d₆) 155.91 (C), 152.44 (CH), 149.69 (C), 140.30 (CH), 118.25 (C), 68.42 (CH), 61.28 (CH), 31.42 (CH₂), 29.21 (CH₂), 28.49 (CH₂), 25.92 (CH₂), 22.33 (CH₂), 20.86 (CH₃), 14.27 (CH₃).

### Procedure for preparation of 1-(2-morpholinoethyl)-1H-pyrazolo[3,4-d]pyrimidin-4-amine (HWC-28) and 2-(2-morpholinoethyl)-2H-pyrazolo[3,4-d]pyrimidin-4-amine (HWC-29)

### 3-Amino-1-(2-morpholinoethyl)-1H-pyrazole-4-carbonitrile and 5-amino-1-(2-morpholinoethyl)-1H-pyrazole-4-carbonitrile

4-(2-Chloroethyl)morpholine hydrochloride (3.72 g, 20.0 mmol) was added in portions to a suspension of 3-amino-1H-pyrazole-4-carbonitrile (1.08 g, 9.99 mmol) and anhydrous potassium carbonate (3.31 g, 20.0 mmol) in DMF (12 mL) at room temperature and the reaction mixture was stirred at 60° C. for 36 h. TLC (95:5 dichloromethane/methanol) indicated consumption of starting material 3-amino-1H-pyrazole-4-carbonitrile (R_{f} 0.08) and formation of a product component (R_{f} 0.16). After cooling, the inorganic material was filtered off and the filtrate was evaporated to dryness under reduced pressure. The residue was chromatographed on a silica gel column (50 g). Gradient elution with dichloromethane I methanol (99:1, 200 mL; 98:2, 100 mL; 95:5, 250 mL) gave a 1:1 mixture of 3-amino-1-(2-morpholinoethyl)-1H-pyrazole-4-carbonitrile and 5-amino-1-(2-morpholinoethyl)-1H-pyrazole-4-carbo-nitrile (2.07 g, 9.34 mmol; 93%) as a white solid: δ_{H} (400 MHz; CDCl₃) 7.64 (one isomer 1H, s, pyrazole H), 7.43 (one isomer 1H, s, pyrazole H), 5.85 (one isomer 2H, br s, NH₂), 4.16 (one isomer 2H, br s, NH₂), 4.14-4.12 (one isomer 2H, m, chain CH₂), 4.02 (one isomer 2H, t, J 6.2, chain CH₂), 3.74-3.69 (both isomers 4H, m, morpholine OCH₂), 2.78-2.74 (both isomers 2H, m, chain CH₂), 2.61-2.58 (one isomer 4H, m, morpholine NCH₂), 2.49-2.46 (one isomer 4H, m, morpholine NCH₂); δ_{C} (101 MHz; CDCl₃) 156.55 & 151.66 (C), 139.99 & 134.68 (CH), 114.59 & 113.80 (C), 78.77 & 76.06 (C), 66.90 (both isomers, 2×CH₂), 59.12 & 57.33 (CH₂), 53.88 (isomer-2, 2×CH₂), 53.58 (isomer-1, 2×CH₂), 49.77 & 47.48 (CH₂).

### 1-(2-Morpholinoethyl)-1H-pyrazolo[3,4-d]pyrimidin-4-amine (HWC-28) and 2-(2-morpholinoethyl)-2H-pyrazolo[3,4-d]pyrimidin-4-amine (HWC-29)

Formamide (1.0 mL, 25.1 mmol) was added to a 1:1 mixture of 5-amino-1-(2-morpholinoethyl)-1H-pyrazole-4-carbonitrile and 3-amino-1-(2-morpholinoethyl)-1H-pyrazole-4-carbonitrile (664 mg, 3.00 mmol) and the reaction mixture heated at 210° C. for 1 h. TLC (9:1 dichloromethane/methanol) indicated consumption of starting material (R_{f} 0.30) and formation of a product component (R_{f} 0.20) and a minor product (R_{f} 0.09). After cooling, the brown reaction mixture was diluted with methanol, mixed with silica gel and slowly allowed to evaporate at room temperature for 18 h. The mixture was dry loaded on a silica gel column (25 g) and subjected to chromatography. Gradient elution with dichloromethane/methanol (95:5, 250 mL; 9:1, 850 mL) gave partially purified 1-(2-morpholinoethyl)-1H-pyrazolo[3,4-d]pyrimidin-4-amine (385 mg) (R_{f} 0.20) and pure 2-(2-morpholinoethyl)-2H-pyrazolo[3,4-d]pyrimidin-4-amine (R_{f} 0.09) (136 mg, 0.548 mmol; 37%). The partially purified 1-(2-morpholinoethyl)-1H-pyrazolo[3,4-d]pyrimidin-4-amine (385 mg) was re-chromatographed on a silica gel column (20 g). Gradient elution with dichloromethane/methanol (98:2, 250 mL; 95:5, 100 mL; 9:1, 400 mL) gave pure 1-(2-morpholinoethyl)-1H-pyrazolo[3,4-d]pyrimidin-4-amine (237 mg, 0.955 mmol; 64%) (R_{f} 0.20).

1-(2-Morpholinoethyl)-1H-pyrazolo[3,4-d]pyrimidin-4-amine (HWC-28): δ_{H} (400 MHz; CDCl₃/CD₃OD) 8.23 (1H, s), 7.95 (1H, s), 4.47 (2H, t, J 6.8), 3.61-3.58 (4H, m, morpholine OCH₂), 2.84 (2H, t, J 6.8), 2.50-2.46 (4H, m, morpholine NCH₂); δ_{C} (101 MHz; CDCl₃/CD₃OD) 157.89 (C): 155.39 (CH), 153.17 (C), 131.60 (CH), 100.55 (C), 66.72 (2×CH₂, morpholine OCH₂), 57.16 (CH₂), 53.31 (2×CH₂, morpholine NCH₂), 44.18 (CH₂).

2-(2-Morpholinoethyl)-2H-pyrazolo[3,4-d]pyrimidin-4-amine (HWC-29): δ_{H} (400 MHz; CDCl₃/CD₃OD) 8.22 (1H, s), 8.21 (1H, s), 4.39 (2H, t, J 6.3), 3.65-3.62 (4H, m, morpholine OCH₂), 2.88 (2H, t, J 6.3), 2.48-2.43 (4H, m, morpholine NCH₂); δ_{C} (101 MHz; CDCl₃/CD₃OD) 159.08 (C), 158.91 (C), 154.92 (CH), 125.55 (CH), 101.48 (C), 66.62 (2×CH₂, morpholine OCH₂), 57.57 (CH₂), 53.41 (2×CH₂, morpholine NCH₂), 50.92 (CH₂).

### Procedure for preparation of 2-nonyl-2H-pyrazolo[3,4-d]pyrimidin-4-amine hydrochloride (HWC-30) and 1-nonyl-1H-pyrazolo[3,4-d]pyrimidin-4-amine hydrochloride (HWC-31)

### 3-Amino-1-nonyl-1H-pyrazole-4-carbonitrile and 5-amino-1-nonyl-1H-pyrazole-4-carbonitrile

1-Bromononane (2.29 mL, 12.0 mmol) was added dropwise to a suspension of 3-amino-1H-pyrazole-4-carbonitrile (1.08 g, 10.0 mmol) and anhydrous potassium carbonate (1.66 g, 12.0 mmol) in DMF (10 mL) at room temperature. The heterogeneous reaction mixture was heated to 50° C. and stirred for 20 h. TLC (2:1 light petroleum/ethyl acetate) indicated partial consumption of starting material 3-amino-1H-pyrazole-4-carbonitrile (R_{f} 0.03) with a new product (R_{f} 0.39). The reaction mixture was stirred at 70° C. for a further 3 h to consume all of the starting material. After cooling, the inorganic material was filtered off and the filtrate was evaporated to dryness under reduced pressure. The residue was chromatographed on a silica gel column (50 g). Gradient elution with light petroleum/ethyl acetate (9:1, 100 mL; 7:1, 240 mL; 2:1, 150; 1:1, 200 mL) gave a 5:1 mixture of 3-amino-1-nonyl-1H-pyrazole-4-carbonitrile and 5-amino-1-nonyl-1H-pyrazole-4-carbonitrile (1.84 g, 7.81 mmol; 78%) as a white solid. Major product: δ_{H} (400 MHz; CDCl₃/CD₃OD) 7.46 (1H, s), 3.87 (2H, t, J 7.1), 1.77 (2H, quintet, J 7.1), 1.32-1.18 (12H, m), 0.89 (3H, approx. t, J 6.8); δ_{C} (101 MHz; CDCl₃/CD₃OD) 156.70 (C), 133.75 (CH), 113.83 (C), 78.38 (C), 52.66 (CH₂), 31.78 (CH₂), 29.56 (CH₂), 29.34 (CH₂), 29.15 (CH₂), 29.01 (CH₂), 26.40 (CH₂), 22.61 (CH₂), 14.07 (CH₃).

### 2-Nonyl-2H-pyrazolo[3,4-d]pyrimidin-4-amine hydrochloride (HWC-30) and 1-nonyl-1H-pyrazolo[3,4-d]pyrimidin-4-amine hydrochloride (HWC-31)

Formamide (1.80 mL, 45.2 mmol) was added to a mixture of 3-amino-1-nonyl-1H-pyrazole-4-carbonitrile and 5-amino-1-nonyl-1H-pyrazole-4-carbonitrile (5:1; 700 mg, 3.00 mmol) and the reaction mixture was heated at 190° C. for 3 h to give a black slurry. TLC (9:1 dichloromethane/methanol) indicated consumption of starting material (R_{f} 0.66) and formation of two products (R_{f} 0.29 & 0.39) with some trace impurities. After cooling, water was added to the brown reaction mixture and the separated solid was collected by filtration, washing with water, and chromatographed on a silica gel column (35 g). Gradient elution with dichloromethane/methanol (98:2, 400 ml; 96:4, 300 ml; 9:1, 150 ml) gave partially purified 1-nonyl-1H-pyrazolo[3,4-d]pyrimidin-4-amine and pure 2-nonyl-2H-pyrazolo[3,4-d]pyrimidin-4-amine (R_{f} 0.29) (196 mg, 0.750 mmol; 30%). The partially purified 1-nonyl-1H-pyrazolo[3,4-d]pyrimidin-4-amine was re-chromatographed on a silica gel column (20 g). Elution with light petroleum/ethyl acetate (1:1.5, 400 mL) gave 1-nonyl-1H-pyrazolo[3,4-d]pyrimidin-4-amine (R_{f}. 0.39) (104 mg, 0.398 mmol; 80%).

2-Nonyl-2H-pyrazolo[3,4-d]pyrimidin-4-amine: δ_{H} (400 MHz; CDCl₃/CD₃OD) 8.29 (1H, s), 8.09 (1H, s), 4.28 (2H, t, J 7.2), 1.94 (2H, quintet, J 7.2), 1.29-1.20 (12H, m), 0.84 (3H, approx. t, J 6.9); δ_{C} (101 MHz; CDCl₃/CD₃OD) 159.63 (C), 159.17 (C), 155.81 (CH), 123.77 (CH), 101.54 (C), 54.04 (CH₂), 31.74 (CH₂), 30.11 (CH₂), 29.32 (CH₂), 29.12 (CH₂), 29.01 (CH₂), 26.48 (CH₂), 22.56 (CH₂), 13.98 (CH₃). 2-Nonyl-2H-pyrazolo[3,4-d]pyrimidin-4-amine was converted into its hydrochloride salt (HWC-30) by treatment with a saturated solution of hydrogen chloride in diethyl ether followed by evaporation.

1-Nonyl-1H-pyrazolo[3,4-d]pyrimidin-4-amine: δ_{H} (400 MHz; CDCl₃) 8.40 (1H, s), 7.93 (1H, s), 5.91 (2H, br s), 4.41 (2H, t, J 7.2), 1.94 (2H, quintet, J 7.3), 1.34-1.22 (12H, m), 0.88 (3H, t, J 6.9); δ_{C} (101 MHz; CDCl₃) 157.53 (C), 155.57 (CH), 153.27 (C), 130.14 (CH), 100.56 (C), 47.34 (CH₂), 31.80 (CH₂), 29.65 (CH₂), 29.40 (CH₂), 29.17 (CH₂), 29.13 (CH₂), 26.64 (CH₂), 22.63 (CH₂), 14.08 (CH₃). 1-Nonyl-1H-pyrazolo[3,4-d]pyrimidin-4-amine was converted into its hydrochloride salt (HWC-31) by treatment with a saturated solution of hydrogen chloride in diethyl ether followed by evaporation.

### Synthesis of 2-undecyl-2H-pyrazolo[3,4-d]pyrimidin-4-amine hydrochloride (HWC-32) and 1-undecyl-1H-pyrazolo[3,4-d]pyrimidin-4-amine hydrochloride (HWC-33)

### 3-Amino-1-undecyl-1H-pyrazole-4-carbonitrile and 5-amino-1-undecyl-1H-pyrazole-4-carbonitrile

1-Bromoundecane (1.25 mL, 5.59 mmol) was added dropwise to a suspension of 3-amino-1H-pyrazole-4-carbonitrile (504 mg, 4.66 mmol) and anhydrous potassium carbonate (0.773 g, 5.59 mmol) in DMF (5.0 mL) and the reaction mixture was stirred at 50° C. for 48 h. TLC (2:1 light petroleum/ethyl acetate) indicated consumption of starting material 3-amino-1H-pyrazole-4-carbonitrile (R_{f} 0.03) and formation of a product component (R_{f} 0.39). After cooling, the inorganic material was filtered off and the filtrate was evaporated to dryness under reduced pressure. The crude residue was chromatographed on a silica gel column (30 g). Gradient elution with light petroleum/ethyl acetate (7:1, 200 mL; 3:1, 200; 1:1, 100 mL) gave a 2.25:1 mixture of 3-amino-1-undecyl-1H-pyrazole-4-carbonitrile and 5-amino-1-undecyl-1H-pyrazole-4-carbonitrile (1.12 g, 4.25 mmol; 91%) as a white solid. Major isomer: δ_{H} (400 MHz; CDCl₃) 7.45 (1H, s), 4.05 (2H, br s), 3.85 (2H, t, J 7.2), 1.78 (2H, quintet, J 7.2), 1.30-1.17 (16H, d, J 10.8), 0.85 (3H, approx. t, J 6.9); δ_{C} (101 MHz; CDCl₃) 156.65 (C), 133.73 (CH), 113.80 (C), 78.41 (C), 52.69 (CH₂), 31.88 (CH₂), 29.54 (CH₂), 29.40 (CH₂), 29.29 (CH₂), 29.12 (CH₂), 29.03 (CH₂), 28.88 (CH₂), 26.42 (CH₂), 22.67 (CH₂), 14.10 (CH₃).

### 1-Undecyl-1H-pyrazolo[3,4-d]pyrimidin-4-amine hydrochloride (HWC-33) and 2-undecyl-2H-pyrazolo[3,4-d]pyrimidin-4-amine hydrochloride (HWC-32)

Formamide (1.8 mL, 45.2 mmol) was added to a mixture of 3-amino-1-undecyl-1H-pyrazole-4-carbonitrile and 5-amino-1-undecyl-1H-pyrazole-4-carbonitrile (2.25/1; 780 mg, 3.00 mmol) and the reaction mixture was heated at 210° C. for 1.5 h. TLC (9:1 dichloromethane/methanol) indicated consumption of starting material (R_{f} 0.58) and formation of two product components (R_{f} 0.36 & 0.27) with some trace impurities. After cooling, water was added to the brown reaction mixture and the separated solid was collected by filtration, washing with water, and chromatographed on a silica gel column (35 g). Gradient elution with dichloromethane/methanol (98:2, 400 mL; 95:5, 250 mL; 92:8, 200 mL) gave partially purified 1-undecyl-1H-pyrazolo[3,4-d]pyrimidin-4-amine (206 mg) and pure 2-undecyl-2H-pyrazolo[3,4-d]pyrimidin-4-amine (416 mg, 1.44 mmol; 69%) (R_{f} 0.27). The partially purified 1-undecyl-1H-pyrazolo[3,4-d]pyrimidin-4-amine (R_{f}. 0.36) was re-chromatographed on a silica gel column (20 g). Elution with light petroleum/ethyl acetate (1:1.5, 400 mL) gave 1-undecyl-1H-pyrazolo[3,4-d]pyrimidin-4-amine (138 mg, 0.477 mmol; 52%).

2-Undecyl-2H-pyrazolo[3,4-d]pyrimidin-4-amine: δ_{H} (400 MHz; CDCl₃/CD₃OD) 8.26 (1H, s), 8.09 (1H, s), 4.26 (2H, t, J 7.2), 1.92 (2H, quintet, J 7.2), 1.28-1.17 (16H, m), 0.84 (3H, approx. t, J 6.9); δ_{C} (101 MHz; CDCl₃/CD₃OD) 159.54 (C), 159.22 (C), 155.76 (CH), 123.94 (CH), 101.53 (C), 54.01 (CH₂), 31.80 (CH₂), 30.10 (CH₂), 29.47 (CH₂), 29.47 (CH₂), 29.36 (CH₂), 29.22 (CH₂), 29.01 (CH₂), 26.47 (CH₂), 22.58 (CH₂), 13.98 (CH₃). 2-Undecyl-2H-pyrazolo[3,4-d]pyrimidin-4-amine was converted into its hydrochloride salt (HWC-32) by treatment with a saturated solution of hydrogen chloride in diethyl ether followed by evaporation.

1-Undecyl-1H-pyrazolo[3,4-d]pyrimidin-4-amine: δ_{H} (400 MHz; CDCl₃/CD₃OD) 8.25 (1H, s), 7.96 (1H, s), 4.33 (2H, t, J 7.2), 1.85 (2H, quintet, J 7.2), 1.35-1.15 (16H, m), 0.83 (3H, approx. t, J6.9); δ_{C} (101 MHz; CDCl₃/CD₃OD) 157.88 (C), 155.29 (CH), 152.71 (C), 131.21 (CH), 100.51 (C), 47.26 (CH₂), 31.81 (CH₂), 29.60 (CH₂), 29.46 (2×CH₂), 29.37 (CH₂), 29.22 (CH₂), 29.08 (CH₂), 26.56 (CH₂), 22.58 (CH₂), 13.98 (CH₃). 1-Undecyl-1H-pyrazolo[3,4-d]pyrimidin-4-amine was converted into its hydrochloride salt (HWC-33) by treatment with a saturated solution of hydrogen chloride in diethyl ether followed by evaporation.

### Synthesis of 1-[2-(2-methoxyethoxy)ethyl]-1H-pyrazolo[3,4-d]pyrimidin-4-amine (HWC-34) and 2-[2-(2-methoxyethoxy)ethyl]-2H-pyrazolo[3,4-d]pyrimidin-4-amine (HWC-35)

### 3-Amino-1-[2-(2-methoxyethoxy)ethyl]-1H-pyrazole-4-carbonitrile and 5-amino-1-1-[2-(2-methoxyethoxy)ethyl]-1H-pyrazole-4-carbonitrile

1-Bromo-2-(2-methoxyethoxy)ethane (753 μL, 5.59 mmol) was added dropwise to a suspension of 3-amino-1H-pyrazole-4-carbonitrile (504 mg, 4.66 mmol) and anhydrous potassium carbonate (0.773 g, 5.59 mmol) in DMF (5.0 mL) and the reaction mixture was stirred at 50° C. for 20 h. TLC (dichloromethane/methanol, 9:1) indicated consumption of starting material 3-amino-1H-pyrazole-4-carbonitrile (R_{f} 0.29) and formation of two product components (R_{f} 0.47 and 0.42). After cooling, the inorganic material was filtered off and the solution was evaporated to dryness under reduced pressure. The residue was chromatographed on a silica gel column (25 g). Gradient elution with dichloromethane/methanol (99:1, 100 mL; 98:2, 200 mL; 95:5, 200 mL) gave 5-amino-1-[2-(2-methoxyethoxy)ethyl]-1H-pyrazole-4-carbonitrile (225 mg, 1.07 mmol; 23%) (R_{f} 0.47), 3-amino-1-[2-(2-methoxyethoxy)ethyl]-1H-pyrazole-4-carbonitrile (329 mg, 1.56 mmol; 34%) (R_{f} 0.42) and a 1.8:1 mixture of the two isomers (424 mg; 43%) as colourless oils.

5-Amino-1-[2-(2-methoxyethoxy)ethyl]-1H-pyrazole-4-carbonitrile: δ_{H} (400 MHz; CDCl₃) 7.41 (1H, s, pyrazole H), 4.98 (2H, br s, NH₂), 4.15-4.11 (2H, m), 3.78-3.75 (2H, m), 3.60-3.56 (2H, m), 3.49-3.46 (2H, m), 3.32 (3H, s); δ_{C} (101 MHz; CDCl₃) 151.93 (C), 140.09 (CH), 114.61 (C), 76.20 (C), 71.43 (CH₂), 70.85 (CH₂), 70.29 (CH₂), 58.87 (OCH₃), 49.60 (NCH₂).

3-Amino-1-[2-(2-methoxyethoxy)ethyl]-1H-pyrazole-4-carbonitrile: δ_{H} (400 MHz; CDCl₃) 7.66 (1H, s, pyrazole H), 4.18 (2H, br s, NH₂), 4.12-4.09 (2H, m), 3.80-3.7 (2H, m), 3.60-3.57 (2H, m), 3.52-3.49 (2H, m), 3.38 (3H, s); δ_{C} (101 MHz; CDCl₃) 156.65 (C), 135.38 (CH), 113.82 (C), 78.89 (C), 71.74 (CH₂), 70.56 (CH₂), 68.83 (CH₂), 59.02 (OCH₃), 52.46 49.60 (NCH₂).

### 1-[2-(2-Methoxyethoxy)ethyl]-1-pyrazolo[3,4-d]pyrimidin-4-amine (HWC-34)

Formamide (0.500 mL, 12.5 mmol) was added to 5-amino-1-[2-(2-methoxyethoxy)ethyl]-1H-pyrazole-4-carbonitrile (196 mg, 0.932 mmol) and the reaction mixture was heated at 210° C. for 1.5 h. TLC (9:1 dichloromethane/methanol) indicated consumption of starting material (R_{f} 0.47) and formation of a product component (R_{f} 0.35) with some trace impurities. After cooling, the brown solution was diluted with methanol, mixed with silica gel and allowed to evaporate for 18 h. The crude sample (dry loaded) was chromatographed on a silica gel column (20 g). Gradient elution with dichloromethane/methanol (98:2, 250 mL; 95:5, 250 mL) gave an oil (600 mg). The partially purified oil was dissolved in ethyl acetate (100 mL) and washed with water (3×15). The organic phase was dried over sodium sulfate, filtered and concentrated in vacuo to give 1-[2-(2-methoxyethoxy)ethyl]-1H-pyrazolo[3,4-d]pyrimidin-4-amine (165 mg, 0.695 mmol; 75%) as a white solid: δ_{H} (400 MHz; CDCl₃) 8.34 (1H, s), 8.10 (1H, s), 6.69 (2H, br s, NH₂), 4.61 (2H, t, J 5.7), 4.00 (2H, t, J5.7), 3.65-3.60 (2H, m), 3.51-3.46 (2H, m), 3.31 (3H, s, OMe); δ_{C} (101 MHz; CDCl₃) 156.36 (C), 153.21 (CH), 153.14 (C), 132.14 (CH), 100.36 (C), 71.77 (CH₂), 70.15 (CH₂), 69.02 (CH₂), 58.89 (OMe), 46.90 (NCH₂).

### 2-[2-(2-Methoxyethoxy)ethyl]-2H-pyrazolo[3,4-d]pyrimidin-4-amine (HWC-35)

Formamide (0.700 mL, 17.6 mmol) was added to 3-amino-1-[2-(2-methoxyethoxy)ethyl]-1H-pyrazole-4-carbonitrile (304 mg, 1.45 mmol) and the reaction mixture was heated at 210° C. for 1.5 h. TLC (9:1 dichloromethane/methanol) indicated consumption of starting material (R_{f} 0.42) and formation of a product component (R_{f} 0.25) with some trace impurities. After cooling, the brown mixture was diluted with methanol, mixed with silica gel and allowed to evaporate at room temperature for 18 h. The crude material (dry loaded) was chromatographed on a silica gel column (20 g). Gradient elution with dichloromethane/methanol (98:2, 250 mL; 95:5, 250 mL; 9:1, 250 mL) gave 2-[2-(2-methoxyethoxy)ethyl]-2H-pyrazolo[3,4-d]pyrimidin-4-amine (196 mg, 0.826 mmol; 57%) as a light brown solid: δ_{H} (400 MHz; CDCl₃/CD₃OD) 8.26 (1H, s), 8.23 (1H, s), 4.49-4.45 (2H, m), 3.92-3.88 (2H, m), 3.55-3.52 (2H, m), 3.47-3.44 (2H, m), 3.29 (3H, s, OMe); δ_{C} (101 MHz; CDCl₃/CD₃OD) 159.36 (C), 159.18 (C), 155.51 (CH), 125.87 (CH), 101.64 (C), 71.53 (CH₂), 70:11 (CH₂), 69.00 (CH₂), 58.67 (OMe), 53.74 (NCH₂).

### Synthesis of racemic erythro-1-(2-hydroxynonan-3-yl)-1H-imidazole-4-carboxamide (HWC-36)

Ethyl erythro-1-(2-hydroxynonan-3-yl)-1H-imidazole-4-carboxylate (preparation—vide supra; 0.090 g, 0.319 mmol) was suspended in conc. ammonia (aq) (5 mL) in a heavy-walled sealed vessel and heated at 90° C. for 18 hours. TLC (10:0.7 dichloromethane to methanol) indicated consumption of starting material (R_{f} 0.36) and formation of product (R_{f} 0.20). The mixture was allowed to cool, diluted with water (10 mL) and extracted with dichloromethane (2×15 mL). The combined organics were washed with saturated sodium chloride solution (10 mL), dried over magnesium sulfate, filtered and concentrated under reduced pressure to give a crude oil. The crude oil was chromatographed on a silica gel column (15 g). Elution with dichloromethane/methanol (10:0.7) gave 1-(2-hydroxynonan-3-yl)-1H-imidazole-4-carboxamide as a colourless oil (48 mg; 59%): δ_{H} (400 MHz; CDCl₃) 7.66 (1H, d, J 1.2), 7.42 (1H, d, J 1.2), 7.12 (1H, br s), 5.98 (1H, br s), 3.98-3.86 (2H, m, chain H-2 and H-3), 2.56 (1H, br s), 1.93-1.71 (2H, m, chain CH₂-4), 1.30-1.04 (8H, m), 1.06 (3H, d, J 6.1, chain CH₃-1), 0.80 (3H, approx. t, J 6.6, chain CH₃-9); δ_{C} (101 MHz; CDCl₃) 165.51 (CONH₂), 137.17 (CH), 136.13 (CH), 122.17 (C), 69.64 (CH), 64.87 (CH), 31.69 (CH₂), 29.69 (CH₂); 29.03 (CH₂), 26.14 (CH₂), 22.67 (CH₂), 19.43 (CH₃), 14.15 (CH₃).

### Synthesis of 1-octyl-1H-pyrazolo[3,4-d]pyrimidin-4-amine hydrochloride (HWC-40) and 2-octyl-2H-pyrazolo[3,4-d]pyrimidin-4-amine hydrochloride (HWC-41)

### 3-Amino-1-octyl-1H-pyrazole-4-carbonitrile and 5-amino-1-octyl-1H-pyrazole-4-carbonitrile

1-Bromooctane (1.04 mL, 5.99 mmol) was added dropwise to a suspension of 3-amino-1H-pyrazole-4-carbonitrile (540 mg, 5.00 mmol) and anhydrous potassium carbonate (828 mg, 5.99 mmol) in DMF (5.0 mL) at room temperature and the reaction mixture was stirred at 50° C. for 18 h. TLC (2:1 light petroleum/ethyl acetate) indicated consumption of starting material 3-amino-1H-pyrazole-4-carbonitrile (R_{f} 0.03) and formation of product (R_{f} 0.30). After cooling, the inorganic material was filtered off and the solution was evaporated to dryness under reduced pressure. The residue was chromatographed on a silica gel column (20 g). Gradient elution with light petroleum/ethyl acetate (7:1, 200 mL; 3:1, 200; 1:1, 100 mL) gave a 2.4:1 mixture of 3-amino-1-octyl-1H-pyrazole-4-carbonitrile and 5-amino-1-octyl-1H-pyrazole-4-carbonitrile (684 mg, 3.11 mmol; 63%). Major product: δ_{H} (400 MHz; CDCl₃) 7.49 (1H, s), 4.29 (2H, br s), 3.90 (2H, t, J 7.2), 1.82 (2H, quintet, J 7.1), 1.30-1.17 (10H, m), 0.85 (3H, approx. t, J 6.6); δ_{C} (101 MHz; CDCl₃) 156.69 (C), 133.76 (CH), 113.84 (C), 78.37 (C) 52.68 (CH₂), 31.70 (CH₂), 29.57 (CH₂), 29.06 (CH₂), 28.98 (CH₂), 26.41 (CH₂), 22.58 (CH₂), 14.05 (CH₃).

### 1-Octyl-1H-pyrazolo[3,4-d]pyrimidin-4-amine hydrochloride (HWC-40) and 2-octyl-2H-pyrazolo[3,4-d]pyrimidin-4-amine hydrochloride (HWC-41)

Formamide (0.800 mL, 20.1 mmol) was added to a mixture of 5-amino-1-octyl-1H-pyrazole-4-carbonitrile (184 mg, 0.835 mmol) and 3-amino-1-octyl-1H-pyrazole-4-carbonitrile (1:2.35; 618 mg, 2.81 mmol) and the reaction mixture was heated at 210° C. for 1 h. TLC (9:1 dichloromethane/methanol) indicated consumption of starting material (R_{f} 0.65) and formation of two product components (R_{f} 0.31 & 0.24) with some trace impurities. After cooling, water was added to the brown reaction mixture and the separated solid was collected by filtration, washing with water, and dried over P₂O₅ in vacuo. The crude material was chromatographed on a silica gel column (25 g). Gradient elution with dichloromethane/methanol (98:2, 400 mL; 95:5, 250 mL; 9:1, 175 mL) gave partially purified 1-octyl-1H-pyrazolo[3,4-d]pyrimidin-4-amine (106 mg) and pure 2-octyl-2H-pyrazolo[3,4-d]pyrimidin-4-amine (365 mg, 1.48 mmol; 75%) (R_{f} 0.24). The partially purified 1-octyl-1H-pyrazolo[3,4-d]pyrimidin-4-amine (R_{f} 0.31) was re-chromatographed on a silica gel column (20 g). Elution with light petroleum/ethyl acetate (1:1.5, 400 mL) gave 1-octyl-1H-pyrazolo[3,4-d]pyrimidin-4-amine (91 mg, 0.37 mmol; 44%).

1-Octyl-1H-pyrazolo[3,4-d]pyrimidin-4-amine: δ_{H} (400 MHz; CDCl₃) 8.39 (1H, s), 7.92 (1H, s), 6.14 (2H, br s, NH₂), 4.40 (2H, t, J 7.2), 1.93 (2H, quintet, J 7.3), 1.33-1.23 (10H, m), 0.86 (3H, approx. t, J 6.9); δ_{C} (101 MHz; CDCl₃) 157.69 (C), 155.52 (CH), 153.23 (C), 130.23 (CH), 100.58 (C), 47.33 (CH₂), 31.71 (CH₂), 29.65 (CH₂), 29.09 (2×CH₂), 26.64 (CH₂), 22.58 (CH₂), 14.05 (CH₃). 1-Octyl-1H-pyrazolo[3,4-d]pyrimidin-4-amine was converted into its hydrochloride salt (HWC-40) by treatment with a saturated solution of hydrogen chloride in diethyl ether followed by evaporation.

2-Octyl-2H-pyrazolo[3,4-d]pyrimidin-4-amine: δ_{H} (400 MHz; CDCl₃/CD₃OD) 8.29 (1H, s), 8.18 (1H, s), 4.26 (2H, t, J 7.2), 1.97-1.87 (2H, m), 1.27-1.18 (10H, m), 0.82 (3H, approx. t, J 6.9); δ_{C} (101 MHz; CDCl₃/CD₃OD) 159.67 (C), 159.36 (C), 155.90 (CH), 124.09 (CH), 101.61 (C), 54.00 (CH₂), 31.65 (CH₂), 30.13 (CH₂), 29.01 (CH₂), 28.97 (CH₂), 26.48 (CH₂), 22.51 (CH₂), 13.95 (CH₃). 2-Octyl-2H-pyrazolo[3,4-d]pyrimidin-4-amine was converted into its hydrochloride salt (HWC-41) by treatment with a saturated solution of hydrogen chloride in diethyl ether followed by evaporation.

### Synthesis of (3S,4R)-4-(6-amino-9H-purin-9-yl)-1-phenylpentan-3-ol hydrochloride (HWC-42), (3S,4R)-4-(6-methoxy-9H-purin-9-yl)-1-phenylpentan-3-ol hydrochloride (HWC-43), (2S,3R)-3-(6-amino-9H-purin-9-yl)-5-phenylpentan-2-ol hydrochloride (HWC-44), (2S,3R)-3-(6-methoxy-9H-purin-9-yl)-5-phenylpentan-2-ol hydrochloride (HWC-45)

### (S)-Methyl 2-(tert-butyldimethylsilyloxy)propanoate

(S)-Methyl 2-(tert-butyldimethylsilyloxy)propanoate was prepared by adaption of the procedure reported by Jaunzeme and Jirgensons (Tetrahedron, 2008, 64, 5794-5799):

(S)-Methyl 2-hydroxypropanoate (3.25 g, 31.2 mmol) was dissolved in dichloromethane (40 mL) and cooled in an ice bath to 0° C. Imidazole (4.25 g, 62.5 mmol) was added and stirred for 5 minutes. To this mixture tert-butyldimethylsilyl chloride (4.78 g, 31.7 mmol) was added over a period of 10 minutes and the mixture was stirred for 18 h at room temperature. The reaction was quenched by the addition of water (60 mL) and the organic layer was separated and washed with hydrochloric acid (1 N, 30 mL), water (2×30 mL). The aqueous layer was back extracted with dichloromethane (2×20 mL) and the combined organic layers were washed with brine (30 mL), dried with sodium sulfate, filtered and evaporated in vacuo to give a colourless oil that was chromatographed on a silica gel column. Elution with 2% ethyl acetate/light petroleum gave (S)-methyl 2-(tert-butyldimethylsilyloxy)propanoate (4.86 g, 22.2 mmol; 71%) as a viscous colourless oil (R_{f} 0.71 in 5% ethyl acetate/light petroleum): δ_{H} (400 MHz; CDCl₃) 4.31 (1H, q, J6.8), 3.70 (3H, s), 1.37 (3H, d, J 6.8), 0.88 (9H, s), 0.08 (3H, s), 0.06 (3H, s); δ_{C} (101 MHz; CDCl₃) 174.44 (CO), 68.33 (CH), 51.76 (OCH₃), 25.66 (3×CH₃), 21.29 (CH₃), 18.25 (C), −5.04 (SiCH₃), −5.34 (SiCH₃).

### (S)-Dimethyl 3-(tert-butyldimethylsilyloxy)-2-oxobutylphosphonate

(S)-Dimethyl 3-(tert-butyldimethylsilyloxy)-2-oxobutylphosphonate was prepared by the adaptation of the procedure reported by Shapiro et al. (Tetrahedron Let., 1990, 31, 5674-5816):

n-Butyllithium (2.5 M in hexane, 30.6 mL, 76.5 mmol) was added to a solution of dimethyl methylphosphonate (8.16 mL, 75.3 mmol) in tetrahydrofuran (40 mL) at −78° C. under an atmosphere of argon over a period of 15 minutes. The mixture was stirred for a further 20 minutes followed by the addition (S)-methyl 2-(tert-butyldimethyl-silyloxy)propanoate (4.17 g, 19.1 mmol) in dry tetrahydrofuran (30 mL). The reaction mixture was allowed to warm to room temperature and stirred for 18 h. TLC (50% ethyl acetate/light petroleum) indicated consumption of starting material and formation of two new components (R_{f} 0.82 and R_{f} 0.22). The reaction was quenched by the addition of a saturated solution of ammonium chloride (30 mL) and extracted with ethyl acetate (3×20 mL). The combined organics were washed with brine (2×20 mL), dried with sodium sulfate, filtered and concentrated in vacuo to give a crude oil. The crude material was chromatographed on a silica gel column. Elution with 20% ethyl acetate/petroleum gave (S)-dimethyl 3-(tert-butyldimethylsilyloxy)-2-oxobutylphosphonate (4.50 g, 14.5 mmol; 76%) as a colourless oil: δ_{H} (400 MHz; CDCl₃) 4.22 (1H, q, J 6.8), 3.773 (3H, d, ³J_{HP} 11.2, OMe), 3.765 (3H, d, ³J_{HP} 11.2, OMe), 3.34 (1H, ABX m, ²J_{HP} 21.9, ²J_{HH} 14.9, H-1a), 3.23 (1H, ABX m, ²J_{HP} 21.9, ²J_{HH} 14.9, H-1b), 1.29 (3H, d, J 6.8), 0.90 (9H, s), 0.08 (3H, s), 0.07 (3H, s); δ_{C} (101 MHz; CDCl₃) 205.00 (C-2, d, ²J_{CP} 5.7), 74.71 (CH-3, d, ³J_{CP} 2.8), 52.87 (OCH₃, d, ²J_{CP} 6.4), 52.79 (OCH₃, d, ²J_{CP} 6.4), 34.62 (CH₂-1, d, ¹J_{CP} 134), 25.67 (3×CH₃), 20.15 (CH₃-4), 17.96 (C), −4.72 (SiCH₃), −5.10 (SiCH₃).

### (S,E)-4-(tert-Butyldimethylsilyloxy)-1-phenylpent-1-en-3-one

n-Butyllithium (2.5 M in hexane; 8.81 mL, 22.0 mmol) was added dropwise to a solution of (S)-dimethyl 3-(tert-butyldimethylsilyloxy)-2-oxobutylphosphonate (6.84 g, 22.0 mmol; Ref: Shapiro et al. Tetrahedron Lett., 1990, 31, 5733-5736) in tetrahydrofuran (100 mL) at −78° C. After 25 minutes, benzaldehyde (2.03 mL, 20.0 mmol) was added and the reaction mixture was slowly allowed to attain to room temperature and stirred for 18 h. TLC (light petroleum/ethyl acetate 95:5) indicated the presence of the starting materials benzaldehyde (R_{f} 0.31) and phosphonate (R_{f} 0.05) and a new component (R_{f} 0.44). The reaction mixture was warmed to 40° C. for 16 h after which period TLC indicated the complete consumption of starting material. The reaction mixture was quenched by the slow addition of saturated sodium bicarbonate solution (50 mL) and extracted with dichloromethane (150 mL). The organic extract was washed with brine (2×30 mL), dried with sodium sulfate, filtered and evaporated to give a crude material (6.08 g) as a pale yellow oil. The crude material was chromatographed on a silica gel column (60 g). Elution with light petroleum/ethyl acetate (95:5, 600 mL) gave (S,E)-4-(tert-butyldimethylsilyloxy)-1-phenylpent-1-en-3-one (5.03 g, 17.3 mmol; 86%): δ_{H} (200 MHz; CDCl₃) 7.71 (1H, d, J 16.1), 7.60-7.53 (2H, m), 7.41-7.35 (3H, m), 7.27 (1H, d, J 16.1), 4.32 (1H, q, J6.8), 1.35 (3H, d, J 6.8), 0.92 (9H, s), 0.08 (3H, s), 0.07 (3H, s); δ_{C} (50 MHz; CDCl₃) 202.37 (0=0), 144.05 (CH), 135.03 (C), 130.71 (CH), 129.13 (2×CH), 128.63 (2×CH), 120.33 (CH), 74.88 (CH), 25.97 (3×CH₃), 21.46 (CH₃), 18.37 (C), −4.59 (CH₃), −4.75 (CH₃).

### (3S,4S,E)-4-(tert-Butyldimethylsilyloxy)-1-phenylpent-1-en-3-ol and (2S,3S,E)-3-(tert-butyldimethylsilyloxy)-5-phenylpent-4-en-2-ol

L-Selectride (1 M in tetrahydrofuran; 20.4 mL, 20.4 mmol) was added dropwise over 30 minutes to a solution of (S,E)-4-(tert-butyldimethylsilyloxy)-1-phenylpent-1-en-3-one (4.94 g, 17.0 mmol) in tetrahydrofuran (100 mL) at −78° C. and the reaction mixture was stirred for 3 h. TLC (light petroleum/ethyl acetate 95:5) indicated the consumption of starting material (R_{f} 0.44) and the presence of two new compounds (R_{f} 0.24 and 0.18). The reaction mixture was partitioned with a mixture of ethyl acetate and water (1:1, 30 mL) and the phases then separated. The organic layer was diluted with ethyl acetate (100 mL), washed with brine, dried with sodium sulfate, filtered and evaporated to give a mixture 1:1.16 of (2S,3S,E)-3-(tert-butyldimethylsilyloxy)-5-phenylpent-4-en-2-ol and (3S,4S,E)-4-(tert-butyldimethyl-silyloxy)-1-phenylpent-1-en-3-ol (7.69 g) as a colourless oil.

### (3S,4S)-4-(tert-Butyldimethylsilyloxy)-1-phenylpentan-3-ol and (2S,3S)-3-(tert-butyl-dimethylsilyloxy)-5-phenylpentan-2-ol

A solution of (2S,3S,E)-3-(tert-butyldimethylsilyloxy)-5-phenylpent-4-en-2-ol and (3S,4S,E)-4-(tert-butyldimethylsilyloxy)-1-phenylpent-1-en-3-ol (1:1.16 mixture; 4.97 g, 17.0 mmol) in ethanol (100 mL) was hydrogenated over 10% palladium on carbon (0.892 g) under hydrogen (1 atm) at 20° C. for 20 h. TLC (light petroleum/ethyl acetate 95:5) indicated consumption of the starting materials and the presence of two new compounds (R_{f} 0.28 and 0.20). The reaction mixture was flushed with nitrogen and filtered through celite, washing with ethyl acetate. The filtrate was concentrated in vacuo and the crude residue chromatographed on a silica gel column (80 g). Gradient elution with light petroleum/ethyl acetate (99:1˜90:1) gave (3S,4S)-4-(tert-butyldimethylsilyloxy)-1-phenylpentan-3-ol (R_{f} 0.28) (2.01 g, 6.83 mmol; 87%) and (2S,3S)-3-(tert-butyldimethylsilyloxy)-5-phenylpentan-2-ol (R_{f} 0.20) (2.46 g, 8.35 mmol; 91%).

(3S,4S)-4-(tert-Butyldimethylsilyloxy)-1-phenylpentan-3-ol: δ_{H} (400 MHz; CDCl₃) 7.31-7.26 (2H, m), 7.23-7.17 (3H, m), 3.66 (1H, qd, J6.2 and 5.2, H-4), 3.33-3.29 (1H, m, H-3), 2.91-2.84 (1H, m, H-1a), 2.74-2.66 (1H, m, H-1b), 2.39 (1H, br s), 1.78-1.66 (2H, m, CH₂-2), 1.18 (3H, d, J 6.2), 0.95 (9H, s), 0.13 (6H, s); δ_{C} (101 MHz; CDCl₃) 142.31 (C), 128.49 (2×CH), 128.35 (2×CH), 125.74 (CH), 75.04 (CH), 71.84 (CH), 35.35 (CH₂), 32.13 (CH₂), 25.85 (3×CH₃), 20.25 (CH₃), 18.04 (C), −4.12 (SiCH₃), −4.81 (SiCH₃).

(2S,3S)-3-(tert-Butyldimethylsilyloxy)-5-phenylpentan-2-ol: δ_{H} (400 MHz; CDCl₃) 7.35-7.30 (2H, m), 7.25-7.20 (3H, m), 3.79 (1H, qd, J 6.3 and 4.8, H-4), 3.56 (1H, dt, J 6.0 and 4.8, H-3), 2.77-2.63 (2H, m, CH₂-1), 2.17 (1H, br s, OH), 1.98 (1H, ddt, J 13.9, 10.4 and 5.9, H-2a), 1.79 (1H, dddd, J4.8, 6.3, 11.0 and 13.9, H-2b), 1.21 (3H, d, J 6.4), 0.97 (9H, s), 0.14 (3H, s), 0.13 (3H, s); δ_{C} (101 MHz; CDCl₃) 142.22 (C), 128.43 (2×CH), 128.28 (2×CH), 125.84 (CH), 76.19 (CH), 68.98 (CH), 35.55 (CH₂), 31.22 (CH₂), 25.92 (3×CH₃), 19.56 (CH₃), 18.15 (C), −4.13 (SiCH₃), −4.57 (SiCH₃).

### 9-[(2R,3S)-3-(tert-Butyldimethylsilyloxy)-5-phenylpentan-2-yl]-6-chloro-9H-purine

Diisopropyl azodicarboxylate (2.60 mL, 13.2 mmol) was added to a mixture of (2S,3S)-3-(tert-butyldimethylsilyloxy)-5-phenylpentan-2-ol (1.27 g, 4.32 mmol), 6-chloro-9H-purine (667 mg, 4.32 mmol) and triphenylphosphine (2.48 g, 9.44 mmol) in tetrahydrofuran (100 mL) at room temperature and stirred for 18 h. TLC (3:1 light petroleum/ethyl acetate) indicated that the starting materials (baseline and R_{f} 0.71) had been transformed in to a new component (R_{f} 0.45). The reaction mixture was filtered through a short silica gel column, washing with light petroleum/ethyl acetate (3:1, 100 mL). The filtrate was evaporated in vacuo to give a crude orange oil (4.25 g) that was chromatographed on a silica gel column (80 g). Gradient elution with light petroleum/ethyl acetate (9:1, 1 L; 7:1, 800 mL; 5:1, 600 mL) gave 9-[(2R,3S)-3-(tert-butyldimethylsilyloxy)-5-phenylpentan-2-yl]-6-chloro-9H-purine (634 mg, 1.47 mmol; 34%) as a pale yellow oil: δ_{H} (200 MHz; CDCl₃) 8.74 (1H, s), 8.14 (1H, s), 7.34-7.15 (5H, m), 4.95 (1H, qd, J 7.1 & 3.4, chain H-4), 4.04 (1H, ddd, J 3.3, 4.9 & 8.0, chain H-3), 2.83-2.67 (2H, m, chain CH₂-1), 1.99-1.69 (2H, m, chain CH₂-2), 1.66 (3H, d, J 7.1), 0.86 (9H, s), −0.09 (3H, s), −0.49 (3H, s); δ_{C} (50 MHz; CDCl₃) 151.88 (CH), 151.64 (C), 151.10 (C), 144.68 (CH), 141.26 (C), 131.74 (C), 128.77 (2×CH), 128.51 (2×CH), 126.42 (CH), 73.02 (CH), 54.26 (CH), 36.39 (CH₂), 31.63 (CH₂), 25.97 (3×CH₃), 18.08 (C), 13.32 (CH₃), −4.15 (SiCH₃), −5.18 (SiCH₃).

### 9-[(2R,3S)-3-(tert-Butyldimethylsilyloxy)-5-phenylpentan-2-yl]-9H-purin-6-amine and 9-[(2R,3S)-3-(tert-butyldimethylsilyloxy)-5-phenylpentan-2-yl]-6-methoxy-9H-purine

A mixture of 9-[(2R,3S)-3-(tert-butyldimethylsilyloxy)-5-phenylpentan-2-yl]-6-chloro-9H-purine (600 mg, 1.39 mmol) and ammonia (7 N in methanol; 4.50 mL, 31.5 mmol) was heated at 80° C. in a sealed pressure tube for 18 h. TLC, (95:5 dichloromethane/methanol), indicated that the starting material (R_{f} 0.79) was transformed into a mixture of two new compounds, minor (R_{f} 0.68) and major (R_{f} 0.38). The reaction mixture was evaporated in vacuo to give a crude white solid that was chromatographed on a silica gel column (40 g). Gradient elution with dichloromethane/methanol (99:1, 150 mL; 92:2, 200 mL; 95:5, 150 mL) gave 9-[(2R,3S)-3-(tert-butyldimethylsilyloxy)-5-phenylpentan-2-yl]-9H-purin-6-amine (R_{f} 0.38) (460 mg, 1.12 mmol; 80%) as a white solid and 9-[(2R,3S)-3-(tert-butyldimethylsilyloxy)-5-phenylpentan-2-yl]-6-methoxy-9H-purine (Rf 0.68) (82 mg, 0.19 mmol; 14%) as a pale yellow dense oil.

9-[(2R,3S)-3-(tert-Butyldimethylsilyloxy)-5-phenylpentan-2-yl]-9H-purin-6-amine: δ_{H} (200 MHz; CDCl₃): δ 8.38 (1H, s), 7.86 (1H, s), 7.32-7.23 (2H, m), 7.22-7.13 (3H, m), 6.15 (2H, s), 4.86 (1H, qd, J 7.0 & 3.8, chain H-2), 4.09 (1H, ddd, J 7.5, 4.9 & 4.0, chain H-3), 2.88-2.62 (2H, m, CH₂-1), 1.99-1.65 (2H, m, CH₂-2), 1.60 (3H, d, J 7.1, CH₃-5), 0.85 (9H, s), −0.10 (3H, s), −0.46 (3H, s); δ_{C} (50 MHz; CDCl₃) 155.79 (C), 152.93 (CH), 149.91 (C), 141.72 (C), 139.80 (CH), 128.67 (2×CH), 128.51 (2×CH), 126.21 (CH), 119.70 (C), 73.25 (CH), 53.43 (CH), 36.72 (CH₂), 31.57 (CH₂), 26.02 (3×CH₃), 18.12 (C), 13.59 (CH₃), −4.12 (SiCH₃), −5.27 (SiCH₃).

9-[(2R,3S)-3-(tert-Butyldimethylsilyloxy)-5-phenylpentan-2-yl]-6-methoxy-9H-purine: δ_{H} (200 MHz; CDCl₃) 8.55 (1H, s), 7.97 (1H, s), 7.31-7.12 (5H, m), 4.89 (1H, qd, J 7.1 & 3.9, chain H-2), 4.18 (3H, s), 4.08 (1H, ddd, J 3.9, 4.8 & 7.5, chain H-3), 2.86-2.62 (2H, m), 1.93-1.71 (2H, m), 1.63 (3H, d, J 7.1), 0.86 (9H, s), −0.10 (3H, s), −0.47 (3H, s); δ_{C} (50 MHz; CDCl₃) 161.11 (C), 151.85 (CH), 151.77 (C), 141.50 (C), 141.41 (CH), 128.58 (2×CH), 128.40 (2×CH), 126.15 (CH), 121.49 (C), 73.10 (CH), 54.24 (CH), 53.68 (OCH₃), 36.51 (CH₂), 31.44 (CH₂), 25.90 (3×CH₃), 17.99 (C), 13.50 (CH₃), −4.25 (SiCH₃), −5.32 (SiCH₃).

### (3S,4R)-4-(6-Amino-9H-purin-9-yl)-1-phenylpentan-3-ol hydrochloride (HWC-42)

Tetrabutylammonium fluoride (1 M tetrahydrofuran solution; 1.70 mL, 1.70 mmol) was added to a solution of 9-[(2R,3S)-3-(tert-butyldimethylsilyloxy)-5-phenylpentan-2-yl]-9H-purin-6-amine (350 mg, 0.850 mmol) in tetrahydrofuran (17 mL) and the reaction mixture was stirred at room temperature for 18 h. TLC (95:5 dichloromethane/methanol) indicated consumption of starting material (R_{f} 0.38) and formation of a product component (R_{f} 0.24). The reaction mixture was evaporated at reduced pressure and the residue was dissolved in ethyl acetate (50 mL), washed with brine (3×10 mL), dried over sodium sulfate, filtered and concentrated in vacuo to give a white solid that was chromatographed on a silica gel column (15 g). Gradient elution with dichloromethane/methanol (98:2, 150 mL; 95:5, 200 mL; 9:1, 150 mL) gave (3S,4R)-4-(6-amino-9H-purin-9-yl)-1-phenylpentan-3-ol (R_{f} 0.24) (216 mg, 0.726 mmol; 85%) as a white solid: δ_{H} (200 MHz; CDCl₃/CD₃OD) 8.12 (1H, s), 7.85 (1H, s), 7.20-7.00 (5H, m), 4.51 (1H, qd, J 7.0 and 3.1, chain H-4), 3.81-3.76 (1H, m, chain H-3), 2.79 (1H, ddd, J 14.0, 8.4 & 6.0, chain H-1a), 2.56 (1H, dt, J 13.7 and 8.1, chain H-1b), 1.84-1.60 (2H, m, chain CH₂-2), 1.45 (3H, d, J 7.1, chain CH₃-5); δ_{C} (50 MHz; CDCl₃/CD₃OD) 155.46 (C), 152.08 (CH), 148.84 (C), 141.41 (C), 139.86 (CH), 128.36 (2×CH), 128.33 (2×CH), 125.90 (CH), 118.81 (C), 72.14 (CH), 56.18 (CH), 35.82 (CH₂), 32.26 (CH₂), 14.22 (CH₃). (2S,3R)-3-(6-Amino-9H-purin-9-yl)-5-phenylpentan-2-ol was converted into its hydrochloride salt (HWC-42) by treatment with a saturated solution of hydrogen chloride in diethyl ether followed by evaporation.

### (3S,4R)-4-(6-Methoxy-9H-purin-9-yl)-1-phenylpentan-3-ol (HWC-43)

Tetrabutylammonium fluoride (1 M in tetrahydrofuran; 0.366 mL, 0.366 mmol) was added to a solution of 9-[(2R,3S)-3-(tert-butyldimethylsilyloxy)-5-phenylpentan-2-yl]-6-methoxy-9H-purine (78.0 mg, 0.183 mmol) in tetrahydrofuran (3.6 mL) at room temperature and the reaction mixture was stirred for 18 h. TLC (95:5 dichloromethane/methanol) indicated consumption of starting material (R_{f} 0.68) and formation of a product component (R_{f} 0.39). The reaction mixture was evaporated and the residue was dissolved in ethyl acetate (20 mL), washed with brine (3×5 mL), dried over sodium sulfate, filtered and concentrated in vacuo to give a crude white solid. The crude material was chromatographed on a silica gel column (10 g). Gradient elution with dichloromethane/methanol (99:1, 100 mL; 98:2, 250 mL) gave (3S,4R)-4-(6-methoxy-9H-purin-9-yl)-1-phenylpentan-3-ol (R_{f} 0.39) (46.0 mg, 0.147 mmol; 81%): δ_{H} (200 MHz; CDCl₃/CD₃OD) 8.43 (1H, s), 8.05 (1H, s), 7.33-7.14 (5H, m), 4.73 (1H, qd, J 7.1 and 2.7, chain H-4), 4.15-4.02 (1H, m, chain H-3), 4.03 (3H, s, OMe), 3.06-2.91 (1H, m, chain H-1a), 2.73 (1H, dt, J 13.9 and 8.2, chain H-1b), 1.97-1.77 (2H, m, chain CH₂-2), 1.59 (3H, d, J 7.1, chain CH₃-5); δ_{C} (50 MHz; CDCl₃/CD₃OD) 160.34 (C), 151.38 (CH), 150.86 (C), 141.83 (C), 141.68 (CH), 128.50 (4×CH), 126.01 (CH), 120.46 (C), 71.34 (CH), 55.91 (CH), 54.14 (OCH₃), 35.75 (CH₂), 32.58 (CH₂), 13.18 (CH₃). (3S,4R)-4-(6-Methoxy-9H-purin-9-yl)-1-phenylpentan-3-ol was converted into its hydrochloride salt (HWC-43) by treatment with a saturated solution of hydrogen chloride in diethyl ether followed by evaporation.

### 9-[(3R,4S)-4-(tert-Butyldimethylsilyloxy)-1-phenylpentan-3-yl]-6-chloro-9H-purine

Diisopropyl azodicarboxylate (2.52 mL, 12.8 mmol) was added to a stirred solution of (3S,4S)-4-(tert-butyldimethylsilyloxy)-1-phenylpentan-3-ol (1.26 g, 4.27 mmol), 6-chloro-9H-purine (667 mg, 4.32 mmol) and triphenylphosphine (2.24 g, 8.54 mmol) in tetrahydrofuran (100 mL) at room temperature and the mixture was stirred at room temperature for 48 h. TLC (3:1 light petroleum/ethyl acetate) indicated almost complete consumption of starting materials (R_{f} 0.74 and baseline) and formation of product (R_{f} 0.47). The reaction mixture was filtered through a short silica gel column, washing with light petroleum/ethyl acetate (2:1, 150 mL), and the filtrate was concentrated in vacuo to give an orange oil (4.16 g) that was chromatographed on a silica gel column (80 g). Gradient elution with light petroleum/ethyl acetate (9:1, 500 mL; 7:1, 800; 5:1, 540 mL) gave 9-[(3R,4S)-4-(tert-butyldimethylsilyloxy)-1-phenylpentan-3-yl]-6-chloro-9H-purine (402 mg, 0.933 mmol; 22%) as a white solid: δ_{H} (200 MHz; CDCl₃) 8.70 (1H, s), 8.13 (1H, s), 7.34-7.19 (3H, m), 7.01-6.91 (2H, m), 4.59-4.49 (1H, m, chain H-3), 4.19 (1H, qd, J 6.3 and 4.4, chain H-4), 2.58-2.32 (4H, m, chain CH₂-1 and CH₂-2), 1.11 (3H, d, J6.3, chain CH₃-5), 0.84 (9H, s), −0.04 (3H, s), −0.19 (3H, s); δ_{C} (50 MHz; CDCl₃) 152.15 (C), 151.88 (CH), 151.13 (C), 145.19 (CH), 140.05 (C), 131.77 (C), 128.73 (2×CH), 128.34 (2×CH), 126.57 (CH), 70.07 (CH), 61.89 (CH), 32.32 (CH₂), 29.10 (CH₂), 25.95 (3×CH₃), 20.68 (CH₃), 18.06 (C), −4.17 (SiCH₃), −4.90 (SiCH₃).

### 9-[(3R,4S)-4-(tert-Butyldimethylsilyloxy]-1-phenylpentan-3-yl)-9H-purin-6-amine and 9-[(3R,4S)-4-(tert-butyldimethylsilyloxy]-1-phenylpentan-3-yl)-6-methoxy-9H-purine

A mixture of 9-[(3R,4S)-4-(tert-butyldimethylsilyloxy)-1-phenylpentan-3-yl]-6-chloro-9H-purine (354 mg, 0.821 mmol) and ammonia (7 N in methanol; 4.50 mL, 31.5 mmol) was heated at 80° C. in a sealed pressure tube for 18 h. TLC (95:5 dichloromethane/methanol) indicated consumption of starting material (R_{f} 0.81) and formation of two product components, minor (R_{f} 0.70) and major (R_{f} 0.40). The reaction mixture was evaporated to give a crude white solid that was chromatographed on a silica gel column (40 g). Gradient elution with dichloromethane/methanol (99:1, 150 mL; 92:2, 200 mL; 95:5, 150 mL) gave 9-[(3R,4S)-4-(tert-butyldimethylsilyloxy]-1-phenylpentan-3-yl)-9H-purin-6-amine (R_{f} 0.40) (228 mg, 0.554 mmol; 67%) as a white solid and 9-[(3R,4S)-4-(tert-butyldimethylsilyloxy)-1-phenylpentan-3-yl]-6-methoxy-9H-purine (R_{f} 0.70) (84 mg, 0.177 mmol; 22%) as a pale yellow dense oil.

9-[(3R,4S)-4-(tert-Butyldimethylsilyloxy)-1-phenylpentan-3-yl]-9H-purin-6-amine: δ_{H} (200 MHz; CDCl₃) 8.34 (1H, s), 7.85 (1H, s), 7.27-7.15 (3H, m), 7.07-6.97 (2H, m), 5.99 (2H, br s, NH₂), 4.44-4.34 (1H, m, chain H-3), 4.13 (1H, qd, J6.2 and 4.6, chain H-4), 2.52-2.33 (4H, m, chain CH₂-1 and CH₂-2), 1.10 (3H, d, J 6.2, chain CH₃-5), 0.85 (9H, s), −0.05 (3H, s), −0.20 (3H, s); δ_{C} (50 MHz; CDCl₃) 155.68 (C), 152.93 (CH), 150.42 (C), 140.71 (CH), 140.39 (C), 128.69 (2×CH), 128.47 (2×CH), 126.40 (CH), 120.13 (C), 70.21 (CH), 61.03 (CH), 32.33 (CH₂), 29.28 (CH₂), 25.99 (3×CH₃), 20.94 (CH₃), 17.84 (C), −4.16 (SiCH₃), −4.95 (SiCH₃).

9-[(3R,4S)-4-(tert-Butyldimethylsilyloxy)-1-phenylpentan-3-yl]-6-methoxy-9H-purine: δ_{H} (200 MHz; CDCl₃) 8.52 (1H, s), 7.97 (1H, br s), 7.23-7.12 (3H, m), 7.02-6.91 (2H, m), 4.49-4.35 (1H, m, chain H-3), 4.18 (3H, s, OMe), 4.12 (1H, qd, J 6.2 and 4.7, chain H-4), 2.55-2.26 (4H, m, chain CH₂-1 and CH₂-2), 1.07 (3H, d, J 6.2, chain CH₃-5), 0.83 (9H, s), −0.07 (3H, s), −0.21 (3H, s); δ_{C} (50 MHz; CDCl₃) 161.19 (C), 151.97 (CH), 149.39 (C), 141.98 (CH), 140.43 (C), 128.63 (2×CH), 128.37 (2×CH), 126.36 (CH), 121.73 (C), 70.08 (CH), 61.27 (CH), 54.31 (OCH₃), 32.20 (CH₂), 29.27 (CH₂), 25.91 (3×CH₃), 20.77 (CH₃), 17.99 (C), −4.26 (SiCH₃), −5.00 (SiCH₃).

### (2S,3R)-3-(6-Amino-9H-purin-9-yl)-5-phenylpentan-2-ol hydrochloride

Tetrabutylammonium fluoride (1 M in tetrahydrofuran; 1.07 mL, 1.07 mmol) was added to a solution of 9-[(3R,4S)-4-(tert-butyldimethylsilyloxy)-1-phenylpentan-3-yl]-9H-purin-6-amine (220 mg, 0.534 mmol) in tetrahydrofuran (15 mL) at room temperature and stirred for 18 h. TLC (95:5 dichloromethane/methanol) indicated consumption of starting material (R_{f} 0.40) and formation of a product component (R_{f} 0.26). The reaction mixture was evaporated and the residue was dissolved in ethyl acetate (50 mL), washed with brine (3×10 mL), dried over sodium sulfate, filtered and concentrated in vacuo to give a crude white solid. The crude material was chromatographed on a silica gel column (15 g). Gradient elution with dichloromethane/methanol (98:2, 150 mL; 95:5, 200 mL; 9:1, 150 mL) gave (2S,3R)-3-(6-amino-9H-purin-9-yl)-5-phenylpentan-2-ol (R_{f} 0.26) (150 mg, 0.504 mmol; 94%) as a dense colourless oil that solidified upon standing: δ_{H} (200 MHz; CDCl₃) 8.26 (1H, br s), 7.76 (1H, br s), 7.23-7.12 (3H, m), 7.02 (2H, ˜d, J 7.6), 6.50 (2H, s, NH₂), 5.55 (1H, br s, OH), 4.31-4.13 (2H, m, chain H-2 and H-3), 2.56-2.21 (4H, m, chain CH₂-4 and CH₂-5), 1.22 (3H, d, J 6.3, chain CH₃-1); δ_{C} (50 MHz; CDCl₃) 156.02 (C), 152.54 (CH), 149.77 (C), 140.71 (CH), 140.28 (C), 128.72 (2×CH), 128.41 (2×CH), 126.47 (CH), 120.19 (C), 69.61 (CH), 62.45 (CH), 32.21 (CH₂), 28.72 (CH₂), 20.32 (CH₃). (2S,3R)-3-(6-Amino-9H-purin-9-yl)-5-phenylpentan-2-ol was converted into its hydrochloride salt (HWC-44) by treatment with a saturated solution of hydrogen chloride in diethyl ether followed by evaporation.

### (2S,3R)-3-(6-Methoxy-9H-purin-9-yl)-5-phenylpentan-2-ol

Tetrabutylammonium fluoride (1 M in tetrahydrofuran; 0.338 mL, 0.338 mmol) was added to a solution of 9-[(3R,4S)-4-(tert-butyldimethylsilyloxy)-1-phenylpentan-3-yl]-6-methoxy-9H-purine (80.0 mg, 0.169 mmol) in tetrahydrofuran (3.5 mL) at room temperature and the reaction mixture was stirred for 18 h. TLC (95:5 dichloromethane/methanol) indicated consumption of starting material (R_{f} 0.70) and formation of a product component (R_{f} 0.40). The reaction mixture was evaporated and the residue was dissolved in ethyl acetate (20 mL), washed with brine (3×5 mL), dried over sodium sulfate, filtered and concentrated in vacuo to give a crude oil. The crude oil was chromatographed on a silica gel column (8 g). Gradient elution with dichloromethane/methanol (99:1, 100 mL; 98:2, 250 mL) gave (2S,3R)-3-(6-methoxy-9H-purin-9-yl)-5-phenylpentan-2-ol (R_{f} 0.40) (41 mg, 0.131 mmol; 78%) as a colourless oil: δ_{H} (200 MHz; CDCl₃) 8.44 (1H, s), 7.92 (1H, br s), 7.22-7.11 (3H, m), 7.02-6.91 (2H, m), 5.14 (1H, br s, OH), 4.42-4.31 (1H, m, chain H-2), 4.28 (1H, qd, J 6.5 and 4.8, chain H-3), 4.10 (3H, s, OMe), 2.54-2.31 (4H, m, chain CH₂-4 and CH₂-5), 1.25 (3H, d, J 6.4, chain CH₃-1); δ_{C} (50 MHz; CDCl₃) 160.83 (C), 151.65 (CH), 151.65 (C), 142.32 (CH), 140.16 (C), 128.69 (2×CH), 128.34 (2×CH), 126.46 (CH), 121.39 (C), 68.90 (CH), 61.95 (CH), 54.35 (OCH₃), 32.06 (CH₂), 28.13 (CH₂), 20.17 (CH₃). (2S,3R)-3-(6-Methoxy-9H-purin-9-yl)-5-phenylpentan-2-ol was converted into its hydrochloride salt (HWC-45) by treatment with a saturated solution of hydrogen chloride in diethyl ether followed by evaporation.

### Synthesis of (2S,3R)-3-(6-amino-9H-purin-9-yl)nonan-2-ol (HWC-46)

### (S,E)-2-(tert-Butyldimethylsilyloxy)non-4-en-3-one

(S,E)-2-(tert-Butyldimethylsilyloxy)non-4-en-3-one was prepared by the adaption of the procedure reported by Taddei et al (J. Org. Chem., 2006, 71, 103-107):

Lithium chloride (0.769 g, 18.1 mmol) was added to a solution of (S)-dimethyl 3-(tert-butyldimethylsilyloxy)-2-oxobutylphosphonate (5.62 g, 18.1 mmol) in acetonitrile (75 mL) under an atmosphere of argon. The cloudy reaction mixture was stirred for 5 minutes and N-ethyldiisopropylamine (2.62 mL, 15.0 mmol) was then added dropwise. The reaction mixture became very viscous and additional acetonitrile (5 mL) was added, stirring the mixture for a further for 2 h. Pentanal (1.60 mL, 15.0 mmol) was added and the reaction mixture was stirred for a further 92 h. TLC (10% ethyl acetate/light petroleum) indicated a new compound (R_{f} 0.58). The reaction mixture was quenched with brine and extracted with ethyl acetate. The combined organics were dried over sodium sulfate and evaporated in vacuo to give a crude colourless oil. The crude material was chromatographed on a silica gel column. Elution with 3% ethyl acetate/light petroleum gave (S,E)-2-(tert-butyldimethylsilyloxy)non-4-en-3-one (2.90 g, 10.7 mmol; 59%): δ_{H} (400 MHz; CDCl₃) 6.98 (1H, dt, J 15.7 & 6.9), 6.55 (1H, dt, J 15.7 & 1.6), 4.21 (1H, q, J 6.8), 2.20 (2H, qd, J 7.2 & 1.6), 1.47-1.39 (2H, m), 1.37-1.28 (2H, m), 1.28 (6H, d, J 6.80), 0.91-0.84 (12H, m), 0.043 (3H, s), 0.036 (3H, s); δ_{C} (101 MHz; CDCl₃) 201.90 (CO), 149.07 (CH), 124.07 (CH), 74.40 (CH), 32.35 (CH₂), 30.10 (CH₂), 25.73 (3×CH₃), 22.21 (CH₂), 21.13 (CH₃), 18.14 (C), 13.80 (CH₃), −4.85 (SiCH₃) −5.00 (SiCH₃).

### (2S,3S,E)-3-(tert-Butyldimethylsilyloxy)non-4-en-2-ol and (2S,3S,E)-2-(tert-butyldimethyl-silyloxy)non-4-en-3-ol

(2S,3S,E)-3-(tert-Butyldimethylsilyloxy)non-4-en-3-ol and (2S,3S,E)-2-(tert-butyl-dimethylsilyloxy)non-4-en-3-ol were prepared by the adaption of the procedure reported by Terasaka et al. (J. Med. Chem., 2005, 48, 4750-4753):

Lithium tri-sec-butylborohydride (1 M tetrahydrofuran solution; 12.8 mL, 12.8 mmol) was added dropwise to a solution of (S,E)-2-(tert-butyldimethylsilyloxy)non-4-en-3-one (2.90 g, 10.7 mmol) in tetrahydrofuran (5 mL) under an atmosphere of argon at −78° C. over a period of 15 minutes and stirred for a further 4 h. TLC (15% ethyl acetate/light petroleum) indicated a new component (R_{f} 0.57) and the reaction was quenched by the slow addition of a mixture of ethyl acetate/water (1:1, 20 mL). The organic layer was washed with brine, dried with magnesium sulfate, filtered and evaporated in vacuo to give a crude colourless oil. The crude material was chromatographed on silica gel column. Elution with ethyl acetate/light petroleum gave (2S,3S,E)-3-(tert-butyldimethylsilyloxy)non-4-en-2-ol and (2S,3S,E)-2-(tert-butyldimethylsilyloxy)non-4-en-3-ol (2.40 g, 8.79 mmol; 82%) as a (1:1) mixture.

### (2S,3S)-2-(tert-Butyldimethylsilyloxy)nonan-3-ol and (2S,3S)-3-(tert-butyldimethylsilyloxy)-nonan-2-ol

Pd (10% on carbon; 100 mg, 0.940 mmol) was added to a 1:1 mixture of (2S,3S,E)-2-(tert-butyldimethylsilyloxy)non-4-en-3-ol and (2S,3S,E)-3-(tert-butyldimethyl-silyloxy)non-4-en-2-ol (2.90 g, 10.6 mmol) in ethanol (100 mL) and stirred under an atmosphere of hydrogen for 18 h. TLC (3% ethyl acetate/light petroleum) indicated new compounds (R_{f} 0.40, 0.32 and 0.25). The reaction mixture was filtered and the filtrate was evaporated to give a crude colourless oil. The crude material was repeatedly chromatographed on silica gel columns to give (2S,3S)-2-(tert-butyldimethyl-silyloxy)nonan-3-ol (1.80 g, 6.23 mmol; 62%) and (2S,3S)-3-(tert-butyldimethylsilyloxy)nonan-2-ol (1.10 g, 4.01 mmol; 37%).

(2S,3S)-2-(tert-Butyldimethylsilyloxy)nonan-3-ol: δ_{H} (400 MHz; CDCl₃) 3.65 (1H, dq, J 5.2 & 6.2), 3.28-3.22 (1H, br m), 2.38 (1H, br d, J 4.7, OH), 1.67-1.22 (10H, m), 1.17 (3H, d, J 5.9), 0.98-0.82 (12H, m), 0.07 (3H, s), 0.06 (3H, s).

(2S,3S)-3-(tert-Butyldimethylsilyloxy)nonan-2-ol: δ_{H} (400 MHz; CDCl₃) 3.73-3.59 (1H, m), 3.44 (1H, dt, J6.3 & 4.7), 2.25 (1H, d, J5.8, OH), 1.65-1.22 (10H, m), 1.14 (3H, d, J 6.3), 0.96-0.82 (12H, m), 0.15-0.03 (6H, 2×s)

### 9-[(2S,3R)-2-(tert-Butyldimethylsilyloxy)nonan-3-yl]-6-chloro-9H-purine

Diisopropyl azodicarboxylate (1.42 mL, 7.21 mmol) was added to a mixture of (2S,3S)-2-(tert-butyldimethylsilyloxy)nonan-3-ol (1.33 g, 4.86 mmol), 6-chloro-9H-purine (940 mg, 6.08 mmol) and triphenylphosphine (1.72 g, 6.57 mmol) in tetrahydrofuran (50 mL) at room temperature and stirred for 48 h. TLC (9:1 light petroleum/ethyl acetate) indicated consumption of starting materials (R_{f} 0.58 and baseline) and formation of a product component (R_{f} 0.16). The reaction mixture was filtered through a short silica gel column, washing with light petroleum/ethyl acetate (3:1, 120 mL) and evaporation of the filtrate gave a crude orange oil (4.42 g). The crude material was chromatographed on a silica gel column (80 g). Gradient elution with light petroleum/ethyl acetate (98:2, 1 L; 95:5, 500 mL; 9:1, 1000 mL; 7:1, 200 mL) gave 9-[(2S,3R)-2-(tert-butyldimethylsilyloxy)nonan-3-yl]-6-chloro-9H-purine (435 mg, 1.06 mmol; 22%) as a dense colourless oil: δ_{H} (200 MHz; CDCl₃) 8.70 (1H, s), 8.15 (1H, s), 4.53 (1H, dt, J 10.8 & 4.4, chain H-3), 4.09 (1H, qd, J6.3 & 4.4, chain H-2), 2.22-1.92 (2H, m), 1.29-1.02 (11H, m), 0.84 (9H, s), 0.79 (3H, t, J 7.1), −0.02 (3H, s), −0.20 (3H, s); δ_{C} (50 MHz; CDCl₃) 152.27 (C), 151.90 (CH), 151.06 (C), 145.04 (CH), 131.66 (C), 70.12 (CH), 62.14 (CH), 31.62 (CH₂), 28.91 (CH₂), 27.48 (CH₂), 25.95 (3×CH₃), 25.95 (CH₂), 22.62 (CH₂), 20.71 (CH₃), 18.07 (C), 14.14 (CH₃), −4.17 (SiCH₃), −4.93 (SiCH₃).

### 9-[(2S,3R)-2-(tert-Butyldimethylsilyloxy)nonan-3-yl]-9H-purin-6-amine and 9-[(2S,3R)-2-(tert-butyldimethylsilyloxy)nonan-3-yl]-6-methoxy-9H-purine

A mixture of 9-[(2S,3R)-2-(tert-butyldimethylsilyloxy)nonan-3-yl]-6-chloro-9H-purine (404 mg, 0.983 mmol) and ammonia (7 N in methanol; 4.50 mL, 31.5 mmol) was heated at 75° C. in a sealed pressure tube for a 48 h. TLC (95:5 dichloromethane/methanol) indicated starting material (R_{f} 0.86) was transformed into two new components, minor (R_{f} 0.71) and major (R_{f} 0.27). The reaction mixture was evaporated in vacuo to give a crude white solid that was chromatographed on a silica gel column (10 g). Gradient elution with dichloromethane/methanol (99:1, 100 mL; 92:2, 250 mL) gave 9-[(2S,3R)-2-(tert-butyldimethylsilyloxy)nonan-3-yl]-9H-purin-6-amine (R_{f} 0.27) (292 mg, 0.746 mmol; 76%) as a white solid and 9-[(2S,3R)-2-(tert-butyldimethylsilyloxy)nonan-3-yl]-6-methoxy-9H-purine (R_{f} 0.71) (81 mg, 0.20 mmol; 20%) as a dense colourless oil:

9-[(2S,3R)-2-(tert-Butyldimethylsilyloxy)nonan-3-yl]-9H-purin-6-amine: δ_{H} (200 MHz; CDCl₃) 8.31 (1H, s), 7.83 (1H, s), 6.17 (2H, br s), 4.41 (1H, dt, J 10.1 & 4.9, chain H-3), 4.09 (1H, qd, J6.2 & 4.5, chain H-2), 2.10-1.94 (2H, m), 1.30-0.96 (11H, m), 0.84 (9H, s), 0.78 (3H, t, J 6.6), −0.04 (3H, s), −0.21 (3H, s); δ_{C} (50 MHz; CDCl₃): δ 155.79 (C), 152.91 (CH), 150.40 (C), 140.16 (CH), 119.68 (C), 70.31 (CH), 61.19 (CH), 31.69 (CH₂), 29.00 (CH₂), 27.48 (CH₂), 25.98 (3×CH₃), 25.98 (CH₂), 22.64 (CH₂), 20.97 (CH₃), 18.08 (C), 14.16 (CH₃), −4.19 (SiCH₃), −5.00 (SiCH₃).

9-[(2S,3R)-2-(tert-Butyldimethylsilyloxy]nonan-3-yl]-6-methoxy-9H-purine: δ_{H} (200 MHz; CDCl₃): δ 8.49 (1H, s), 7.93 (1H, s), 4.44 (1H, dt, J 10.1 & 5.0, chain H-3), 4.15 (3H, s), 4.07 (1H, qd, J 6.3 & 4.6, chain H-2), 2.12-1.89 (2H, m), 1.25-1.00 (11H, m), 0.83 (9H, s), 0.77 (3H, t, J 6.5), −0.06 (3H, s), −0.23 (3H, s); δ_{C} (50 MHz; CDCl₃): δ 161.17 (C), 152.36 (C), 151.92 (CH), 141.83 (CH), 121.51 (C), 70.23 (CH), 61.53 (CH), 54.27 (OCH₃), 31.62 (CH₂), 28.90 (CH₂), 27.59 (CH₂), 25.93 (3×CH₃), 25.93 (CH₂), 22.57 (CH₂), 20.84 (CH₃), 18.02 (C), 14.10 (CH₃), −4.25 (SiCH₃), −5.02 (SiCH₃).

### (2S,3R)-3-(6-Amino-9H-purin-9-yl)nonan-2-ol

Tetrabutylammonium fluoride (1 M tetrahydrofuran solution; 1.44 mL, 1.44 mmol) was added to a solution of 9-[(2S,3R)-2-(tert-butyldimethylsilyloxy)nonan-3-yl]-9H-purin-6-amine (282 mg, 0.720 mmol) in tetrahydrofuran (15 mL) at room temperature and the reaction mixture was stirred for 18 h. TLC (95:5 dichloromethane/methanol) indicated consumption of starting material (R_{f} 0.27) and formation of a product component (R_{f} 0.12). The reaction mixture was evaporated and the residue was dissolved in ethyl acetate (50 mL), washed with brine (3×10 mL), dried over sodium sulfate, filtered and concentrated in vacuo to give a crude oil. The crude oil was chromatographed on a silica gel column (15 g). Gradient elution with dichloromethane/methanol (95:5, 200 mL; 9:1, 150 mL) gave (2S,3R)-3-(6-amino-9H-purin-9-yl)nonan-2-ol (R_{f} 0.12) (182 mg, 0.66 mmol; 91%) as a dense colourless oil that solidified upon standing: δ_{H} (200 MHz; CDCl₃) 8.26 (1H, s), 7.77 (1H, s), 6.18 (2H, br s), 4.34-4.11 (2H, m), 2.20-1.74 (2H, m), 1.26 (3H, d, J 6.5), 1.24-0.93 (8H, m), 0.79 (3H, t, J 6.5); δ_{C} (50 MHz; CDCl₃) 155.91 (C), 152.48 (CH), 149.76 (C), 140.80 (CH), 120.07 (C), 69.71 (CH), 63.67 (CH), 31.71 (CH₂), 29.02 (CH₂), 27.47 (CH₂), 26.44 (CH₂), 22.68 (CH₂), 20.43 (CH₃), 14.18 (CH₃). (2S,3R)-3-(6-Amino-9H-purin-9-yl)nonan-2-ol was converted into its hydrochloride salt (HWC-46) by treatment with a saturated solution of hydrogen chloride in diethyl ether followed by evaporation.

### Synthesis of (2S,3R)-3-(6-methoxy-9H-purin-9-yl)nonan-2-ol (HWC-47)

### Preparation of (2S,3R)-3-(6-methoxy-9H-purin-9-yl)nonan-2-ol

Tetrabutylammonium fluoride (1 M tetrahydrofuran solution; 0.39 mL, 0.39 mmol) was added to a solution of 9-[(2S,3R)-2-(tert-butyldimethylsilyloxy)nonan-3-yl]-6-methoxy-9H-purine (80 mg, 0.20 mmol) in tetrahydrofuran (4 mL at room temperature and the reaction mixture was stirred for 18 h. TLC (95:5 dichloromethane/methanol) indicated consumption of starting material (R_{f} 0.71) and formation of a product component (R_{f} 0.30). The reaction mixture was evaporated and the residue was dissolved in ethyl acetate (20 mL), washed with brine (3×5 mL), dried with sodium sulfate, filtered and concentrated in vacuo to give a crude oil. The crude material was chromatographed on a silica gel column (10 g). Gradient elution with dichloromethane/methanol (98:2, 150 mL; 95:5, 200 mL) gave (2S,3R)-3-(6-methoxy-9H-purin-9-yl)nonan-2-ol (R_{f} 0.30) (54 mg, 0.185 mmol; 94%) as a dense colourless oil: δ_{H} (200 MHz; CDCl₃) 8.39 (1H, s), 7.89 (1H, s), 5.07 (1H, br s, OH) 4.39 (1H, dt, J 10.9 & 3.3, chain H-3), 4.28 (1H, qd, J 6.5 & 2.8, chain H-2), 4.04 (3H, s), 2.18-1.83 (2H, m), 1.27 (3H, d, J 6.5), 1.27-0.88 (8H, m), 0.74 (3H, t, J 6.5); δ_{C} (50 MHz; CDCl₃) 160.69 (C), 151.56 (CH), 151.56 (C), 142.23 (CH), 121.16 (C), 68.73 (CH), 62.55 (CH), 54.28 (OCH₃), 31.58 (CH₂), 28.90 (CH₂), 26.66 (CH₂), 26.08 (CH₂), 22.56 (CH₂), 20.14 (CH₃), 14.08 (CH₃). (2S,3R)-3-(6-Methoxy-9H-purin-9-yl)nonan-2-ol was converted into its hydrochloride salt (HWC-47) by treatment with a saturated solution of hydrogen chloride in diethyl ether followed by evaporation.

### Synthesis of (2R,3S)-2-(6-amino-9H-purin-9-yl)nonan-3-ol (HWC-48)

### 9-[(2R,3S)-3-(tert-Butyldimethylsilyloxy)nonan-2-yl]-6-chloro-9H-purine

Diisopropyl azodicarboxylate (1.11 g, 5.46 mmol) was added to a mixture of (2S,3S)-3-(tert-butyldimethylsilyloxy)nonan-2-ol (1.50 g, 5.46 mmol), 6-chloro-9H-purine (1.06 g, 6.85 mmol) and triphenylphosphine (1.94 g, 7.39 mmol) in tetrahydrofuran (50 mL) and stirred at room temperature for 18 h. TLC (9:1 light petroleum/ethyl acetate) indicated consumption of starting material (R_{f} 0.48) and formation of a product component (R_{f} 0.14). The reaction mixture was filtered through a short silica gel column, washing with light petroleum/ethyl acetate (3:1, 150 mL) and the filtrate was evaporated to give a crude yellow solid (5.22 g) that was chromatographed on a silica gel column (80 g). Gradient elution with light petroleum/ethyl acetate (98:2, 500; 95:5, 500 mL; 9:1, 1 L; 7:1, 600 mL) gave 9-[(2R,3S)-3-(tert-butyldimethylsilyloxy)nonan-2-yl]-6-chloro-9H-purine (550 mg, 1.34 mmol; 25%) as a dense colourless oil that solidified upon standing: δ_{H} (200 MHz; CDCl₃) 8.70 (1H, s), 8.16 (1H, s), 4.88 (1H, qd, J 7.1 & 3.1, chain H-2), 3.96 (1H, ddd, J 7.9, 5.0 & 3.0, chain H-3), 1.61 (3H, d, J 7.1), 1.50-1.18 (10H, m), 0.86 (3H, t, J 6.5), 0.81 (9H, s), −0.11 (3H, s), −0.53 (3H, s); δ_{C} (50 MHz; CDCl₃) 151.84 (CH), 151.63 (C), 151.00 (C), 144.80 (CH), 131.72 (C), 73.33 (CH), 54.17 (CH), 34.60 (CH₂), 31.85 (CH₂), 29.52 (CH₂), 25.97 (3×CH₃), 25.27 (CH₂), 22.74 (CH₂), 18.06 (C), 14.24 (CH₃), 12.83 (CH₃), −4.16 (SiCH₃), −5.22 (SiCH₃).

### 9-[(2R,3S)-3-(tert-Butyldimethylsilyloxy)nonan-2-yl]-9H-purin-6-amine and 9-[(2R,3S)-3-(tert-butyldimethylsilyloxy)nonan-2-yl]-6-methoxy-9H-purine

A mixture of 9-[(2R,3S)-3-(tert-butyldimethylsilyloxy)nonan-2-yl]-6-chloro-9H-purine (450 mg, 1.10 mmol) and ammonia (7 N in methanol; 4.5 mL, 31 mmol) was heated at 75° C. in a 5 mL sealed pressure tube for 18 h. TLC (95:5 dichloromethane/methanol) indicated transformation of starting material (R_{f} 0.77) into two new compounds, minor (R_{f} 0.63) and major (R_{f} 0.23). The reaction mixture was evaporated to give a crude white residue that was chromatographed on a silica gel column (10 g). Gradient elution with dichloromethane/methanol (99:1, 100 mL; 92:2, 250 mL) gave 9-[(2R,3S)-3-(tert-butyldimethylsilyloxy)nonan-2-yl]-9H-purin-6-amine (R_{f} 0.23) (321 mg, 0.820 mmol; 75%) as a white solid and 9-[(2R,3S)-3-(tert-butyldimethylsilyloxy)nonan-2-yl]-6-methoxy-9H-purine (R_{f} 0.63) (88 mg, 0.216 mmol; 20%) as a dense colourless oil.

9-[(2R,3S)-3-(tert-Butyldimethylsilyloxy)nonan-2-yl]-9H-purin-6-amine: δ_{H} (200 MHz; CDCl₃) 8.32 (1H, s), 7.85 (1H, s), 6.06 (2H, br s), 4.76 (1H, qd, J 7.0 & 3.2, chain H-2), 4.06-3.83 (1H, m, chain H-3), 1.53 (3H, d, J 7.1), 1.51-1.18 (10H, m), 0.87 (3H, t, J 5.0), 0.82 (9H, s), −0.12 (3H, s), −0.50 (3H, s); δ_{C} (50 MHz; CDCl₃) 155.73 (C), 152.85 (CH), 149.94 (C), 140.03 (CH), 119.61 (C), 73.48 (CH), 53.35 (CH), 34.84 (CH₂), 31.85 (CH₂), 29.65 (CH₂), 26.03 (3×CH₃), 25.26 (CH₂), 22.76 (CH₂), 18.12 (C), 14.25 (CH₃), 12.99 (CH₃), −4.15 (SiCH₃), −5.30 (SiCH₃).

9-[(2R,3S)-3-(tert-Butyldimethylsilyloxy)nonan-2-yl]-6-methoxy-9H-purine: δ_{H} (200 MHz; CDCl₃) 8.48 (1H, s), 7.94 (1H, s), 4.77 (1H, qd, J 6.8 & 3.2, chain H-2), 4.14 (3H, s), 4.03-3.82 (1H, m, chain H-3), 1.54 (3H, d, J 7.0), 1.43-1.20 (10H, m), 0.84 (3H, t, J6.0), 0.80 (9H, s), −0.14 (3H, s), −0.54 (3H, s); δ_{C} (50 MHz; CDCl₃) 161.10 (C), 151.70 (CH), 151.64 (C), 141.58 (CH), 121.49 (C), 73.37 (CH), 54.21 (CH), 53.59 (OCH₃), 34.67 (CH₂), 31.76 (CH₂), 29.53 (CH₂), 25.94 (3×CH₂), 25.14 (CH₂), 22.67 (CH₂), 18.00 (C), 14.16 (CH₃), 12.95 (CH₃), −4.27 (SiCH₃), −5.35 (SiCH₃).

### (2R,3S)-2-(6-amino-9H-purin-9-yl)nonan-3-ol

Tetrabutylammonium fluoride (1 M tetrahydrofuran solution; 1.48 mL, 1.48 mmol) was added to a solution of 9-[(2R,3S)-3-(tert-butyldimethylsilyloxy)nonan-2-yl]-9H-purin-6-amine (290 mg, 0.741 mmol) in tetrahydrofuran (15 mL) at room temperature and stirred for 18 h. TLC (95:5 dichloromethane/methanol), showed consumption of starting material (R_{f} 0.23) and formation of a product component (R_{f} 0.10). The reaction mixture was evaporated and the crude residue was dissolved in ethyl acetate (50 mL), washed with brine (3×10 mL), dried over sodium sulfate, filtered and concentrated in vacuo to give a crude solid. The crude material was chromatographed on a silica gel column (15 g). Elution with dichloromethane/methanol (95:5, 250 mL) gave (2R,3S)-2-(6-amino-9H-purin-9-yl)nonan-3-ol (R_{f} 0.10) (143 mg, 0.516 mmol; 70%) as a white solid: δ_{H} (200 MHz; CDCl₃/CD₃OD) 8.16 (1H, s), 7.89 (1H, s), 4.58 (1H, qd, J 7.0 & 2.6, chain H-2), 3.88-3.79 (1H, m, chain H-3), 1.47 (3H, d, J 7.1), 1.45-1.39 (2H, m), 1.39-1.07 (8H, m), 0.78 (3H, t, J 6.4); δ_{C} (50 MHz; CDCl₃/CD₃OD) 155.60 (C), 152.27 (CH), 149.03 (C), 140.09 (CH), 119.07 (C), 72.94 (CH), 56.36 (CH), 34.09 (CH₂), 31.81 (CH₂), 29.26 (CH₂), 26.15 (CH₂), 22.65 (CH₂), 14.10 (2×CH₃). (2R,3S)-2-(6-amino-9H-purin-9-yl)nonan-3-ol was converted into its hydrochloride salt (HWC-48) by treatment with a saturated solution of hydrogen chloride in diethyl ether followed by evaporation.

### Synthesis of (2R,3S)-2-(6-methoxy-9H-purin-9-yl)nonan-3-ol (HWC-49)

Tetrabutylammonium fluoride (1 M tetrahydrofuran solution; 0.42 mL, 0.42 mmol) was added to a solution of 9-[(2R,3S)-3-(tert-butyldimethylsilyloxy)nonan-2-yl]-6-methoxy-9H-purine (85 mg, 0.21 mmol) in tetrahydrofuran (50 mL) at room temperature and stirred for 18 h. TLC (95:5 dichloromethane/methanol), showed consumption of starting material (R_{f} 0.63) and formation of a product component (R_{f} 0.25). The reaction mixture was evaporated and the residue was dissolved in ethyl acetate (20 mL), washed with brine (3×5 mL), dried with sodium sulfate, filtered and concentrated in vacuo to give a crude solid. The crude material was chromatographed on a silica gel column (10 g). Elution with light petroleum/ethyl acetate (1:7, 240 mL) gave (2R,3S)-2-(6-methoxy-9H-purin-9-yl)nonan-3-ol (R_{f} 0.25). (45 mg, 0.14 mmol; 67%) as a white solid: δ_{H} (200 MHz; CDCl₃) 8.33 (1H, s), 7.84 (1H, s), 4.65 (1H, qd, J 7.0 & 2.6, chain H-2), 4.21-4.13 (1H, m, chain H-3), 3.96 (3H, s), 1.67-1.26 (13H, m), 0.85 (3H, t, J6.4); δ_{C} (50 MHz; CDCl₃) 159.85 (C), 151.27 (CH), 150.76 (C), 141.73 (CH), 120.31 (C), 71.31 (CH), 56.06 (CH), 54.02 (OCH₃), 33.94 (CH₂), 32.02 (CH₂), 29.44 (CH₂), 26.71 (CH₂), 22.81 (CH₂), 14.27 (CH₃), 12.41 (CH₃). (2R,3S)-2-(6-methoxy-9H-purin-9-yl)nonan-3-ol was converted into its hydrochloride salt (HWC-49) by treatment with a saturated solution of hydrogen chloride in diethyl ether followed by evaporation.

### Synthesis of (2S,3R)-2-(6-amino-9H-purin-9-yl)nonan-3-ol (HWC-50)

### (R)-Methyl 2-(tert-butyldimethylsilyloxy)propanoate

tert-Butyldimethylsilyl chloride (21.2 mL, 123 mmol) was added slowly to a heterogenous mixture of (R)-methyl 2-hydroxypropanoate (9.0 mL, 94 mmol) and imidazole (9.62 g, 141 mmol) in dichloromethane (300 mL) at 0° C. under an atmosphere of argon. The mixture was slowly allowed to attain room temperature and stirred for 48 h. The mixture was diluted with brine (50 mL), diluted with water (50 mL) and the layers were separated. The organics were washed with brine (50 mL), dried with magnesium sulfate, filtered and evaporated at reduced pressure (25° C. 30 mmHg) to give a crude colourless liquid. The crude material was chromatographed on a silica gel column (250 g). Gradient elution with light petroleum/ethyl acetate (10:1) gave (R)-methyl 2-(tert-butyldimethylsilyloxy)propanoate as a colourless oil (18.5 g, 85.0 mmol; 90%): δ_{H} (200 MHz; CDCl₃) 4.22 (1H, q, J6.7), 3.61 (3H, s), 1.28 (3H, d, J6.7), 0.79 (9H, s), 0.00 (6H, 2×s); δ_{C} (50 MHz; CDCl₃): 174.52 (CO), 68.39 (CH), 51.83 (OMe), 25.74 (3×CH₃), 21.37 (CH₃), 18.33 (C), −4.97 (SiCH₃), −5.29 (SiCH₃).

### (R)-Dimethyl 3-(tert-butyldimethylsilyloxy)-2-oxobutylphosphonate

(R)-Dimethyl 3-(tert-butyldimethylsilyloxy)-2-oxobutylphosphonate was prepared by the adaptation of the procedure reported by Shapiro et al. (Tetrahedron Lett., 1990, 31, 5674-5816):

n-Butyllithium (2.5 M in hexane; 28.3 mL, 70.7 mmol) was added to a solution of dimethyl methylphosphonate (7.66 mL, 70.7 mmol) in tetrahydrofuran (40 mL) under and atmosphere of argon at −78° C. over a period of 15 minutes. The mixture was stirred for further 20 minutes followed by the addition of (R)-methyl 2-(tert-butyldimethylsilyloxy)propanoate (10.2 g, 47.1 mmol) in tetrahydrofuran (60 mL). The mixture was allowed to attain room temperature and stirred for 18 h. TLC (40% ethyl acetate/light petroleum) indicated consumption of starting material and formation of a product component (R_{f} 0.25). The reaction was quenched by the addition of a saturated solution of ammonium chloride (60 mL) and extracted with ethyl acetate (3×20 mL). The combined organic extracts were washed with brine (2×20 mL), dried with sodium sulfate, filtered and concentrated in vacuo to give a crude oil. The crude material was chromatographed on a silica gel column. Elution with 20% ethyl acetate/light petroleum gave (R)-dimethyl 3-(tert-butyldimethylsilyloxy)-2-oxobutylphosphonate (13.0 g, 41.9 mmol; 89%) as a colourless oil: δ_{H} (200 MHz; CDCl₃) 4.12 (1H, q, J6.8), 3.77-3.51 (6H, m), 3.40-2.96 (2H, m), 1.19 (3H, d, J 6.8), 0.87-0.66 (9H, m), −0.02 (6H, 2×s); δ_{C} (50 MHz; CDCl₃) 205.00 (C-2, d, ²J_{CP} 6.8), 74.77 (CH-3, d, ³J_{CP} 5.0), 52.86 (OCH₃, d, ²J_{CP} 5.0), 52.74 (OCH₃, d, ²J_{CP} 6.8), 34.56 (CH₂-1, d, ¹J_{CP} 135), 25.70 (3×CH₃), 20.20 (CH₃-4), 17.99 (C), −4.69 (SiCH₃), −5.08 (SiCH₃).

### (R,E)-2-(tert-Butyldimethylsilyloxy)non-4-en-3-one

(R,E)-2-(tert-Butyldimethylsilyloxy)non-4-en-3-one was prepared by the adaption of the procedure reported by Taddei et al (J. Org. Chem., 2006, 71, 103-107):

Lithium chloride (1.26 g, 29.8 mmol) was added to a solution of (R)-dimethyl 3-(tert-butyldimethylsilyloxy)-2-oxobutylphosphonate (9.25 g, 29.8 mmol) in acetonitrile (500 mL) under argon to give a viscous mixture. N-Ethyldiisopropylamine (4.31 mL, 24.7 mmol) was then added dropwise to give a cloudy mixture that was stirred for 2 h. Valeraldehyde (2.63 mL, 24.7 mmol) was added and the reaction mixture was stirred for 72 h. TLC (5% ethyl acetate/light petroleum) indicated formation of a new product component (R_{f} 0.38). The reaction mixture was quenched with brine (30 mL), extracted with ethyl acetate (3×40 mL), and the extract dried with sodium sulfate and evaporated in vacuo to give a colourless crude oil. The crude material was chromatographed on a silica gel column (40 g). Elution with 3% ethyl acetate/light petroleum gave (R,E)-2-(tert-butyldimethylsilyloxy)non-4-en-3-one (5.80 g, 21.4 mmol; 72% yield corrected for starting material recovery): δ_{H} (200 MHz; CDCl₃) 6.99 (1H, dt, J 15.7 & 6.9), 6.56 (1H, dt, J 15.7 & 1.5), 4.22 (1H, q, J 6.8), 2.21 (2H, qd, J6.9 & 1.5), 1.53-1.11 (7H, m), 0.94-0.76 (12H, m), 0.04 (6H, 2×s); δ_{C} (50 MHz; CDCl₃) 202.13 (CO), 149.29 (CH), 124.16 (CH), 74.53 (CH), 32.50 (CH₂), 30.22 (CH₂), 25.86 (3×CH₃), 22.36 (CH₂), 21.28 (CH₃), 18.28 (C), 13.95 (CH₃), −4.72 (SiCH₃), −4.87 (SiCH₃).

### (2R,3R,E)-3-(tert-Butyldimethylsilyloxy)non-4-en-2-ol and (2R,3R,E)-2-(tert-butyl-dimethylsilyloxy)non-4-en-3-ol

(2R,3R,E)-3-(tert-Butyldimethylsilyloxy)non-4-en-2-ol and (2R,3R,E)-2-(tert-butyldimethylsilyloxy)non-4-en-3-ol were prepared by the adaption of the procedure reported by Terasaka (J. Med. Chem., 2005, 48, 4750-4753):

Lithium tri-sec-butylborohydride (1 M tetrahydrofuran solution; 29.7 mL, 29.7 mmol) was added dropwise to a solution of (R,E)-2-(tert-butyldimethylsilyloxy)non-4-en-3-one (5.36 g, 19.8 mmol) in tetrahydrofuran (80 mL) at 0° C. under an atmosphere of argon over a period of 25 minutes. The reaction mixture was stirred for 4 h at room temperature. TLC (15% ethyl acetate/light petroleum) indicated formation of a new product component (R_{f} 0.55). The reaction was quenched by the slow addition of a mixture of ethyl acetate/water (1:1, 50 mL). The organic layer was washed with brine (2×20 mL), dried with magnesium sulfate, filtered and evaporated in vacuo to give a mixture of (2R,3R,E)-3-(tert-butyldimethylsilyloxy)non-4-en-2-ol and (2R,3R,E)-2-(tert-butyl-dimethylsilyl-oxy)non-4-en-3-ol as a crude colourless oil (3.85 g, 14.1 mmol; 71%).

### (2R,3R)-2-(tert-Butyldimethylsilyloxy)nonan-3-ol and (2R,3R)-3-(tert-butyldimethylsilyl-oxy)nonan-2-ol

Crude (2R,3R,E)-3-(tert-butyldimethylsilyloxy)non-4-en-2-ol and (2R,3R,E)-2-(tert-butyldimethylsilyloxy)non-4-en-3-ol (3.85 g, 14.1 mmol) were dissolved in ethyl acetate/ethanol (1.5:1, 25 mL). Palladium 10% on carbon (300 mg, 0.28 mmol) was added and the mixture stirred for 18 h under an atmosphere of hydrogen. TLC (5% ethyl acetate/light petroleum) indicated formation of two product components and consumption of starting material. The reaction mixture was filtered and washed with a mixture of ethyl acetate/ethanol (1:1, 100 mL). The filtrate was collected and evaporated in vacuo to give a crude material (3.75 g). The crude material was subjected to repeated chromatography on silica gel columns. Elution with 2% ethylacetate/light petroleum gave (2R,3R)-3-(tert-butyldimethylsilyloxy)nonan-2-ol (1.38 g, 5.03 mmol; 36%), (2R,3R)-2-(tert-butyldimethylsilyloxy)nonan-3-ol (1.10 g, 4.01 mmol; 28%) and a mixture of the two compounds (1.30 g).

(2R,3R)-2-(tert-Butyldimethylsilyloxy)nonan-3-ol: δ_{H} (200 MHz; CDCl₃) 3.61 (1H, dq, J 5.5 & 6.2), 3.32-3.20 (1H, br m), 2.38 (1H, br d, J 4.9, OH), 1.67-1.10 (10H, m), 1.14 (3H, d, J 5.9), 0.97-0.75 (12H, m), 0.08 (3H, s), 0.07 (3H, s).

(2R,3R)-3-(tert-Butyldimethylsilyloxy)nonan-2-ol: δ_{H} (400 MHz; CDCl₃): 3.64 (1H, qd, J 6.3 & 4.6), 3.42 (1H, dt, J 6.2 & 4.7), 2.15 (1H, br s, OH), 1.66-1.23 (10H, m), 1.12 (3H, d, J6.4), 0.96-0.86 (12H, m), 0.079 (3H, s), 0.076 (3H, s).

### 9-[(2S,3R)-3-(tert-Butyldimethylsilyloxy)nonan-2-yl]-6-chloro-9H-purine

Diisopropyl azodicarboxylate (1.98 mL, 10.2 mmol) was added to a mixture of (2R,3R)-3-(tert-butyldimethylsilyloxy)nonan-2-ol (1.38 g, 5.04 mmol), 6-chloro-9H-purine (1.00 g, 6.47 mmol) and triphenylphosphine (1.98 g, 7.55 mmol) in tetrahydrofuran (50 mL) at room temperature and stirred for 18 h. TLC (9:1 light petroleum/ethyl acetate) indicated consumption of starting material (R_{f} 0.48) and formation of a product component (R_{f} 0.14). The reaction mixture was filtered through a short silica gel column, washing with light petroleum/ethyl acetate (3:1, 150 mL) and the filtrate was evaporated to give a crude yellow solid (5.22 g) that was chromatographed on a silica gel column (80 g). Gradient elution with light petroleum/ethyl acetate (98:2, 500 mL; 95:5, 500 mL; 9:1, 1 L; 7:1, 600 mL) gave 9-[(2S,3R)-3-(tert-butyldimethylsilyloxy)nonan-2-yl]-6-chloro-9H-purine (608 mg, 1.48 mmol; 29%) as a dense colourless oil that solidified upon standing: δ_{H} (200 MHz; CDCl₃) 8.70 (1H, s), 8.16 (1H, s), 4.87 (1H, qd, J 7.0 & 3.0, chain H-2), 3.95 (1H, ddd, J 7.9, 5.0 & 3.0, chain H-3), 1.60 (3H, d, J 7.1), 1.49-1.25 (10H, m), 0.87 (3H, t, J6.0), 0.81 (9H, s), −0.12 (3H, s), −0.54 (3H, s); δ_{C} (50 MHz; CDCl₃) 151.82 (CH), 151.61 (C), 150.98 (C), 144.79 (CH), 131.71 (C), 73.31 (CH), 54.16 (CH), 34.59 (CH₂), 31.84 (CH₂), 29.51 (CH₂), 25.96 (3×CH₃), 25.26 (CH₂), 22.73 (CH₂), 18.05 (C), 14.23 (CH₃), 12.82 (CH₃), −4.17 (SiCH₃), −5.23 (SiCH₃).

### 9-[(2S,3R)-3-(tert-Butyldimethylsilyloxy)nonan-2-yl]-9H-purin-6-amine and 9-[(2S,3R)-3-(tert-butyldimethylsilyloxy)nonan-2-yl]-6-methoxy-9H-purine

A mixture of 9-[(2S,3R)-3-(tert-butyldimethylsilyloxy)nonan-2-yl]-6-chloro-9H-purine (570 mg, 1.39 mmol) and ammonia (7 N in methanol; 4.5 mL, 32 mmol) was heated at 75° C. in a 5 mL sealed pressure tube for 18 h. TLC (95:5 dichloromethane/methanol) indicated consumption of starting material (R_{f} 0.77) and formation of two new product components, minor (R_{f} 0.63) and major (R_{f} 0.23). The reaction mixture was evaporated to give a crude white solid that was chromatographed on a silica gel column (10 g). Gradient elution with dichloromethane/methanol (99:1, 100 mL; 92:2, 250 mL) gave 9-[(2S,3R)-3-(tert-butyldimethylsilyloxy)nonan-2-yl]-9H-purin-6-amine (R_{f} 0.23) (390 mg, 1.00 mmol; 72%) as a white solid and 9-[(2S,3R)-3-(tert-butyldimethylsilyloxy)nonan-2-yl]-6-methoxy-9H-purine (R_{f} 0.63) (85 mg, 0.21 mmol; 15%) as a dense colourless oil.

9-[(2S,3R)-3-(tert-Butyldimethylsilyloxy)nonan-2-yl]-9H-purin-6-amine: δ_{H} (200 MHz; CDCl₃) 8.32 (1H, s), 7.85 (1H, s), 6.14 (2H, br s), 4.76 (1H, qd, J 7.0 & 3.2, chain H-2), 4.06-3.83 (1H, m, chain H-3), 1.53 (3H, d, J 7.1), 1.50-1.17 (10H, m), 0.87 (3H, t, J 5.1), 0.82 (9H, s), −0.12 (3H, s), −0.51 (3H, s); δ_{C} (50 MHz; CDCl₃) 155.75 (C), 152.84 (CH), 149.87 (C), 139.99 (CH), 119.68 (C), 73.45 (CH), 53.33 (CH), 34.84 (CH₂), 31.84 (CH₂), 29.65 (CH₂), 26.03 (3×CH₂), 25.25 (CH₂), 22.76 (CH₂), 18.11 (C), 14.25 (CH₃), 12.97 (CH₃), −4.16 (SiCH₃), −5.31 (SiCH₃).

9-[(2S,3R)-3-(tert-Butyldimethylsilyloxy)nonan-2-yl]-6-methoxy-9H-purine: δ_{H} (200 MHz; CDCl₃) 8.48 (1H, s), 7.94 (1H, s), 4.77 (1H, qd, J 7.0 & 3.3, chain H-2), 4.13 (3H, s), 4.03-3.82 (1H, m; chain H-3), 1.54 (3H, d, J 7.1), 1.49-1.18 (10H, m), 0.84 (3H, t, J 6.0), 0.80 (9H, s), −0.15 (3H, s), −0.55 (3H, s); δ_{C} (50 MHz; CDCl₃): 161.07 (C), 151.78 (CH), 151.74 (C), 141.56 (CH), 121.46 (C), 73.32 (CH), 54.20 (CH), 53.55 (OCH₃), 34.65 (CH₂), 31.74 (CH₂), 29.51 (CH₂), 25.91 (3×CH₃), 25.12 (CH₂), 22.65 (CH₂), 17.98 (C), 14.15 (CH₃), 12.90 (CH₃), −4.29 (SiCH₃), −5.39 (SiCH₃).

### (2S,3R)-2-(6-Amino-9H-purin-9-yl)nonan-3-ol

Tetrabutylammonium fluoride (1 M tetrahydrofuran solution; 1.79 mL, 1.79 mmol) was added to a solution of 9-[(2S,3R)-3-(tert-butyldimethylsilyloxy)nonan-2-yl]-9H-purin-6-amine (350 mg, 0.894 mmol) in tetrahydrofuran (15 mL) at room temperature and stirred for 18 h. TLC (95:5 dichloromethane/methanol), indicated consumption of starting material (R_{f} 0.23) and formation of a new product component (R_{f} 0.10). The reaction mixture was evaporated to give a crude residue that was dissolved in ethyl acetate (50 mL), washed with brine (3×10 mL), dried with sodium sulfate, filtered and concentrated in vacuo to give a crude solid. The crude material was chromatographed on a silica gel column (15 g). Elution with dichloromethane/methanol (95:5, 250 mL) gave (2S,3R)-2-(6-amino-9H-purin-9-yl)nonan-3-ol (R_{f} 0.10) (235 mg, 0.847 mmol; 95%) as a white solid: δ_{H} (200 MHz; CDCl₃/CD₃OD) 8.16 (1H, s), 7.87 (1H, s), 4.55 (1H, qd, J 6.9 & 2.3, chain H-2), 3.88-3.79 (1H, m, chain H-3), 1.48 (3H, d, J 7.1), 1.46-1.10 (10H, m), 0.79 (3H, t, J 6.4); δ_{C} (50 MHz; CDCl₃/CD₃OD) 155.69 (C), 152.34 (CH), 149.14 (C), 140.07 (CH), 119.24 (C), 72.96 (CH), 56.54 (CH), 34.16 (CH₂), 31.84 (CH₂), 29.30 (CH₂), 26.23 (CH₂), 22.68 (CH₂), 14.14 (CH₃), 14.06 (CH₃). (2S,3R)-2-(6-Amino-9H-purin-9-yl)nonan-3-ol was converted into its hydrochloride salt (HWC-50) by treatment with a saturated solution of hydrogen chloride in diethyl ether followed by evaporation.

### Synthesis of (2S,3R)-2-(6-methoxy-9H-purin-9-yl)nonan-3-ol (HWC-51)

Tetrabutylammonium fluoride (1 M tetrahydrofuran solution; 0.39 mL, 0.39 mmol) was added to a solution of 9-[(2S,3R)-3-(tert-butyldimethylsilyloxy)nonan-2-yl]-6-methoxy-9H-purine (80 mg, 0.20 mmol) in tetrahydrofuran (4 mL) at room temperature and stirred for 18 h. TLC (95:5 dichloromethane/methanol), indicated consumption of starting material (R_{f} 0.63) and formation of a product component (R_{f} 0.25). The reaction mixture was evaporated to give a crude residue that was dissolved in ethyl acetate (20 mL), washed with brine (3×5 mL), dried over sodium sulfate, filtered and concentrated in vacuo to give a crude solid. The crude material was chromatographed on a silica gel column (10 g). Elution with light petroleum/ethyl acetate (1:7, 240 mL) gave (2S,3R)-2-(6-methoxy-9H-purin-9-yl)nonan-3-ol (R_{f} 0.25) (54 mg, 0.19 mmol; 94%) as a white solid: δ_{H} (200 MHz; CDCl₃) 8.38 (1H, s), 7.89 (1H, s), 4.66 (1H, qd, J 7.1 & 2.1, chain H-2), 4.21-4.12 (1H, m, chain H-3), 4.01 (3H, s), 1.63-1.26 (13H, m), 0.86 (3H, t, J 6.4); δ_{C} (50 MHz; CDCl₃) 159.97 (C), 151.26 (CH), 150.79 (C), 141.72 (CH), 120.45 (C), 71.49 (CH), 56.19 (CH), 54.02 (OCH₃), 33.95 (CH₂), 31.97 (CH₂), 29.39 (CH₂), 26.63 (CH₂), 22.76 (CH₂), 14.22 (CH₃), 12.57 (CH₃). (2S,3R)-2-(6-Methoxy-9H-purin-9-yl)nonan-3-ol was converted into its hydrochloride salt (HWC-51) by treatment with a saturated solution of hydrogen chloride in diethyl ether followed by evaporation.

### Synthesis of (2R,3S)-3-(6-amino-9H-purin-9-yl)nonan-2-ol (HWC-52)

### 9-[(2R,3S)-2-(tert-Butyldimethylsilyloxy)nonan-3-yl]-6-chloro-9H-purine

Diisopropyl azodicarboxylate (1.62 mL, 8.31 mmol) was added to a mixture of (2R,3R)-2-(tert-butyldimethylsilyloxy)nonan-3-ol (1.52 g, 2.77 mmol), 6-chloro-9H-purine (0.60 g, 3.88 mmol) and triphenylphosphine (1.45 g, 5.54 mmol) in tetrahydrofuran (50 mL) at room temperature and stirred for 48 h. TLC (9:1 light petroleum/ethyl acetate) indicated starting material (R_{f} 0.58) and formation of a product component (R_{f} 0.16). The reaction mixture was filtered through a short silica gel column, washing with light petroleum/ethyl acetate (3:1, 150 mL) and the filtrate was evaporated to give an orange oil (4.42 g) that was chromatographed on a silica gel column (80 g). Gradient elution with light petroleum/ethyl acetate (98:2, 500 mL; 95:5, 500; 9:1, 1000 mL; 7:1, 250 mL) gave 9-[(2R,3S)-2-(tert-butyldimethyl-silyloxy)nonan-3-yl]-6-chloro-9H-purine (296 mg, 0.720 mmol; 26%) as a dense colourless oil: δ_{H} (200 MHz; CDCl₃) 8.70 (1H, s), 8.15 (1H, s), 4.52 (1H, dt, J 10.8 & 4.4, chain H-3), 4.09 (1H, qd, J 6.3 & 4.4, chain H-2), 2.22-1.92 (2H, m), 1.29-0.97 (11H, m), 0.84 (9H, s), 0.79 (3H, t, J 6.8), −0.03 (3H, s), −0.21 (3H, s); δ_{C} (50 MHz; CDCl₃) 152.16 (C), 151.78 (CH), 150.95 (C), 144.94 (CH), 131.57 (C), 70.03 (CH), 62.04 (CH), 31.52 (CH₂), 28.81 (CH₂), 27.39 (CH₂), 25.86 (CH₂), 25.86 (3×CH₃), 22.52 (CH₂), 20.61 (CH₃), 17.97 (C), 14.04 (CH₃), −4.27 (SiCH₃), −5.02 (SiCH₃).

### 9-[(2R,3S)-2-(tert-Butyldimethylsilyloxy)nonan-3-yl]-9H-purin-6-amine and 9-[(2R,3S)-2-(tert-butyldimethylsilyloxy)nonan-3-yl]-6-methoxy-9H-purine

A mixture of 9-[(2R,3S)-2-(tert-butyldimethylsilyloxy)nonan-3-yl]-6-chloro-9H-purine (292 mg, 0.710 mmol) and ammonia (7 N in methanol; 4.0 mL, 28.0 mmol) was heated at 75° C. in a 5 mL sealed pressure tube for 48 h. TLC (95:5 dichloromethane/methanol) indicated consumption of starting material (R_{f} 0.86) and formation of two new product components, minor (R_{f} 0.71) and major (R_{f} 0.27). The reaction mixture was evaporated to give a crude white solid that was chromatographed on a silica gel column (10 g). Gradient elution with dichloromethane/methanol (99:1, 100 mL; 92:2, 250 mL) gave 9-[(2R,3S)-2-(tert-butyldimethylsilyloxy)nonan-3-yl]-9H-purin-6-amine (R_{f} 0.27) (200 mg, 0.511 mmol; 72%) as a white solid and 9-[2R,3S)-2-(tert-butyldimethylsilyloxy)nonan-3-yl]-6-methoxy-9H-purine (R_{f} 0.71) (75 mg, 0.18 mmol; 26%) as a dense colourless oil.

9-[(2R,3S)-2-(tert-Butyldimethylsilyloxy)nonan-3-yl]-9H-purin-6-amine: δ_{H} (200 MHz; CDCl₃) 8.30 (1H, s), 7.82 (1H, s), 6.32 (2H, s), 4.40 (1H, dt, J 10.0 & 4.9, chain H-3), 4.08 (1H, qd, J 6.2 & 4.5, chain H-2), 2.10-1.94 (2H, m), 1.30-0.96 (11H, m), 0.84 (9H, s), 0.78 (3H, t, J 6.6), −0.05 (3H, s), −0.22 (3H, s); δ_{C} (50 MHz; CDCl₃) 155.86 (C), 152.89 (CH), 150.36 (C), 140.10 (CH), 119.65 (C), 70.30 (CH), 61.16 (CH), 31.67 (CH₂), 28.99 (CH₂), 27.46 (CH₂), 25.97 (3×CH₃), 25.97 (CH₂), 22.63 (CH₂), 20.96 (CH₃), 18.06 (C), 14.15 (CH₃), −4.20 (SiCH₃), −5.01 (SiCH₃).

9-[(2R,3S)-2-(tert-Butyldimethylsilyloxy)nonan-3-yl]-6-methoxy-9H-purine: δ_{H} (200 MHz; CDCl₃) 8.51 (1H, s), 7.96 (1H, s), 4.45 (1H, dt, J 10.1 & 5.0, chain H-3), 4.17 (3H, s), 4.09 (1H, qd, J6.3 & 4.6, chain H-2), 2.12-1.89 (2H, m), 1.25-1.00 (11H, m), 0.851 (9H, s), 0.79 (3H, t, J 6.6), −0.04 (3H, s), −0.21 (3H, s); δ_{C} (50 MHz; CDCl₃) 161.22 (C), 152.43 (C), 151.97 (CH), 141.85 (CH), 121.64 (C), 70.25 (CH), 61.58 (CH), 54.32 (OCH₃), 31.66 (CH₂), 28.94 (CH₂), 27.61 (CH₂), 25.97 (CH₂), 25.97 (3×CH₃), 22.61 (CH₂), 20.88 (CH₃), 18.07 (C), 14.14 (CH₃), −4.20 (SiCH₃), −4.98 (SiCH₃).

### (2R,3S)-3-(6-Amino-9H-purin-9-yl)nonan-2-ol (HWC-52)

Tetrabutylammonium fluoride (1 M tetrahydrofuran solution; 0.97 mL, 0.97 mmol) was added to a solution of 9-[(2R,3S)-2-(tert-butyldimethylsilyloxy)nonan-3-yl]-9H-purin-6-amine (190 mg, 0.485 mmol) in tetrahydrofuran (15 mL) at room temperature and stirred for 18 h. TLC (95:5 dichloromethane/methanol), indicated consumption of starting material (R_{f} 0.27) and formation of a product component (R_{f} 0.12). The reaction mixture was evaporated and the residue was dissolved in ethyl acetate (50 mL), washed with brine (3×10 mL), dried with sodium sulfate, filtered and concentrated in vacuo to give a crude dense oil. The crude material was chromatographed on a silica gel column (15 g). Elution with dichloromethane/methanol (95:5, 250 mL) gave (2R,3S)-3-(6-amino-9H-purin-9-yl)nonan-2-ol (120 mg, 0.433 mmol; 89%) as a dense colourless oil that solidified upon standing: δ_{H} (200 MHz; CDCl₃) 8.25 (1H, s), 7.78 (1H, s), 6.33 (2H, br s), 4.36-4.10 (2H, m), 2.17-1.79 (2H, m), 1.25 (3H, d, J 6.4), 1.24-0.95 (8H, m), 0.78 (3H, t, J 6.5); δ_{C} (50 MHz; CDCl₃) 155.97 (C), 152.51 (CH), 149.78 (C), 140.68 (CH), 119.97 (C), 69.64 (CH), 63.45 (CH), 31.69 (CH₂), 29.00 (CH₂), 27.51 (CH₂), 26.40 (CH₂), 22.66 (CH₂), 20.38 (CH₃), 14.17 (CH₃). (2R,3S)-3-(6-Amino-9H-purin-9-yl)nonan-2-ol was converted into its hydrochloride salt (HWC-52) by treatment with a saturated solution of hydrogen chloride in diethyl ether followed by evaporation.

### Synthesis of (2R,3S)-3-(6-methoxy-9H-purin-9-yl)nonan-2-ol (HWC-53)

Tetrabutylammonium fluoride (1 M tetrahydrofuran solution; 0.34 mL, 0.34 mmol) was added to a solution of 9-[(2R,3S)-2-(tert-butyldimethylsilyloxy)nonan-3-yl]-6-methoxy-9H-purine (70 mg, 0.17 mmol) in tetrahydrofuran (4 mLat room temperature and stirred for 18 h. TLC (95:5 dichloromethane/methanol), indicated consumption of starting material (R_{f} 0.71) and formation of a product component (R_{f} 0.30). The reaction mixture was evaporated to give a residue that was dissolved in ethyl acetate (20 mL), washed with brine (3×5 mL), dried with sodium sulfate, filtered and concentrated in vacuo to give a crude oil. The crude material was chromatographed on a silica gel column (10 g). Gradient elution with dichloromethane/methanol (98:2, 100 mL; 95:5, 100 mL) gave (2R,3S)-3-(6-methoxy-9H-purin-9-yl)nonan-2-ol (R_{f} 0.30) (47 mg, 0.16 mmol; 93%) as a dense colourless oil: δ_{H} (200 MHz; CDCl₃) 8.41 (1H, s), 7.89 (1H, s), 5.10 (1H, br s, OH), 4.38 (1H, dt, J 10.9 & 3.3, chain H-3), 4.28 (1H, qd, J 6.5 & 2.8, chain H-2), 4.06 (3H, s), 2.17-1.84 (2H, m), 1.27 (3H, d, J 6.5), 1.24-0.97 (8H, m), 0.75 (3H, t, J 6.5); δ_{C} (50 MHz; CDCl₃) 160.80 (C), 151.58 (CH), 151.58 (C), 142.29 (CH), 121.32 (C), 68.89 (CH), 62.77 (CH), 54.33 (OCH₃), 31.61 (CH₂), 28.93 (CH₂), 26.77 (CH₂), 26.14 (CH₂), 22.59 (CH₂), 20.20 (CH₃), 14.11 (CH₃). (2R,3S)-3-(6-Methoxy-9H-purin-9-yl)nonan-2-ol was converted into its hydrochloride salt (HWC-53) by treatment with a saturated solution of hydrogen chloride in diethyl ether followed by evaporation.

### Synthesis of (rac)-erythro-3-(1H-[1,2,3]triazolo[4,5-c]pyridin-1-yl)nonan-2-ol (HWC-54)

Sodium nitrite (91 mg, 1.32 mmol) in water (3.6 mL) was added dropwise to an ice-cold mixture of 3-(3-aminopyridin-4-ylamino)nonan-2-ol (276 mg, 1.10 mmol) in water (3.6 mL), acetic acid (1.6 mL) and tetrahydrofuran (7.0 mL). The reaction mixture was stirred at 0° C. for 3 h, allowed to attain room temperature and stirred for a further 2 h. TLC (9:1 dichloromethane/methanol) indicated consumption of starting material (R_{f} 0.05) and formation of a product component (R_{f} 0.50). The reaction mixture was evaporated, diluted with water and extracted with dichloromethane (3×10 mL). The combined organics were dried with sodium sulfate, filtered and concentrated in vacuo to give a light brown residue that was chromatographed on a silica gel column (20 g). Elution with hexane/ethyl acetate (1:3, 500 mL) gave 3-(1H-[1,2,3]triazolo[4,5-c]pyridin-1-yl)nonan-2-ol (248 mg) as a colourless oil (95% purity, yield 82%): δ_{H} (200 MHz; CDCl₃) 9.32 (1H, d, J 1.2), 8.40 (1H, d, J 5.9), 7.51 (1H, dd, J 1.2, 6.0), 4.73-4.60 (1H, m), 4.40-4.23 (1H, m), 3.31 (1H, bs), 2.33 (1H, s), 2.21-2.02 (1H, m), 1.25-1.08 (8H, m), 1.18 (3H, d, J 6.8), 0.76 (3H, t, J 6.6); δ_{C} (50 MHz; CDCl₃): 144.71 (CH), 144.44 (CH), 143.08 (C), 137.71 (C), 105.57 (CH), 70.23 (CH), 66.56 (CH), 31.58 (CH₂), 29.36 (CH₂), 28.93 (CH₂), 26.32 (CH₂), 22.59 (CH₂), 19.77 (CH₃), 14.10 (CH₃). 3-(1H-[1,2,3]Triazolo[4,5-c]pyridin-1-yl)nonan-2-ol was converted into its oxalate salt (HWC-54) by treatment with a solution of oxalic acid dihydrate (103 mg, 0.90 mmol) in water (3 mL) and methanol (4 mL), to give a white solid precipitate that was recrystallised from methanol. The white crystals were collected by filtration, washed with water and dried over P₂O₅ in vacuo.

### Synthesis of (rac)-9-(nonan-3-3-yl)-9H-purin-6-amine (HWC-57)

### (rac)-6-Chloro-9-(nonan-3-yl)-9H-purine

Diisopropyl azodicarboxylate (2.33 mL, 12.0 mmol) was added to a mixture of nonan-3-ol (1.26 mL, 7.20 mmol), 6-chloro-9H-purine (0.93 g, 6.00 mmol) and triphenylphosphine (2.36 g, 9.00 mmol) in tetrahydrofuran (50 mL) at room temperature and stirred for 48 h. TLC (4:1 light petroleum/ethyl acetate) indicated consumption of nonan-3-ol (R_{f} 0.59) and formation of a product component (R_{f} 0.28). The reaction mixture was filtered through a short silica gel column, washing with light petroleum/ethyl acetate (1:1, 100 mL). Evaporation of the filtrate gave a crude yellow oil that was chromatographed on a silica gel column (80 g). Gradient elution with light petroleum/ethyl acetate (9:1, 1 L; 7:1, 800 mL; 5:1, 840 mL) gave 6-chloro-9-(nonan-3-yl)-9H-purine (1.30 g, 4.63 mmol; 77%) as a dense pale yellow oil: δ_{H} (200 MHz; CDCl₃): 8.70 (1H, s), 8.08 (1H, s), 4.47 (1H, m), 2.07-1.88 (4H, m), 1.27-1.10 (8H, m), 0.83-0.72 (6H, m); δ_{C} (50 MHz; CDCl₃): 152.25 (C), 151.88 (CH), 151.14 (C), 144.15 (CH), 131.91 (C), 59.01 (CH), 34.75 (CH₂), 31.65 (CH₂), 28.90 (CH₂), 28.29 (CH₂), 26.23 (CH₂), 22.63 (CH₂), 14.15 (CH₃), 10.84 (CH₃).

### (rac)-9-(Nonan-3-yl)-9H-purin-6-amine (HWC-57)

A mixture of 6-chloro-9-(nonan-3-yl)-9H-purine (560 mg, 1.99 mmol) and ammonia (7 N in methanol; 4.5 mL, 31 mmol) was heated at 80° C. in a 5 mL sealed pressure tube for 18 h. TLC (1:1 light petroleum/ethyl acetate) indicated remaining starting material (R_{f} 0.66) and two new product components, minor (R_{f} 0.41) and major (R_{f} 0.06). The reaction mixture was evaporated to give a crude white solid that was chromatographed on a silica gel column (20 g). Gradient elution with light petroleum/ethyl acetate (4:1, 400 mL; 1:1, 200 mL) followed by dichloromethane/methanol (9:1, 100 mL) gave starting material 6-chloro-9-(nonan-3-yl)-9H-purine (287 mg) and 9-(nonan-3-yl)-9H-purin-6-amine (R_{f} 0.06) (145 mg, 0.56 mmol; 28%) as a white solid: δ_{H} (200 MHz; CDCl₃) 8.30 (1H, s), 7.76 (1H, s), 6.33 (2H, br s), 4.59-4.41 (1H, m), 2.02-1.80 (4H, m), 1.27-1.03 (8H, m), 0.86-0.69 (6H, m); δ_{C} (50 MHz; CDCl₃) 155.89 (C), 152.84 (CH), 150.47 (C), 139.21 (CH), 119.87 (C), 57.88 (CH), 34.94 (CH₂), 31.70 (CH₂), 28.98 (CH₂), 28.45 (CH₂), 26.22 (CH₂), 22.64 (CH₂), 14.16 (CH₃), 10.81 (CH₃). 9-(Nonan-3-yl)-9H-purin-6-amine was converted into its oxalate salt (HWC-57) by treatment with a solution of oxalic acid dihydrate (103 mg, 0.90 mmol) in water (3 mL) in methanol (4 mL), to give a white solid precipitate that was re-crystallised from methanol. The white crystals were collected by filtration, washed with water and dried over P₂O₅ in vacuo.

### Synthesis of (2R,3S)-2-(6-amino-9H-purin-9-yl)octan-3-ol (HWC-58)

### (S,E)-2-(tert-Butyldimethylsilyloxy)oct-4-en-3-one

Lithium chloride (0.690 g, 16.3 mmol) was added to a solution of (S)-dimethyl 3-(tert-butyldimethylsilyloxy)-2-oxobutylphosphonate (5.06 g, 16.3 mmol) dissolved in acetonitrile (100 mL) under argon and stirred for a 2 minutes. N,N-Diisopropylethylamine (2.36 mL, 13.5 mmol) was added whereupon the reaction became viscous and was stirred for 2 h. Butyraldehyde (1.58 mL, 13.5 mmol) was added and the mixture was stirred for 92 h at room temperature. The reaction mixture was quenched with brine (35 mL) and extracted with ethyl acetate (3×30 mL). The combined organic layers were washed with brine (20 mL), dried with sodium sulfate and evaporated under reduced pressure to give a crude colourless oil. The crude oil was chromatographed on a silica gel column. Elution with 2% ethyl acetate/light petroleum (200 mL) gave a colourless oil (S,E)-2-(tert-butyldimethylsilyloxy)oct-4-en-3-one (2.20 g, 8.58 mmol; 53%): δ_{H} (200 MHz; CDCl₃) 6.96 (1H, dt, J 15.7 & 6.9), 6.54 (1H, dt, J 15.7 & 1.4), 4.19 (1H, q, J 6.8), 2.16 (2H, qd, J 7.1 & 1.4), 1.45 (2H, sextet, J 7.4), 1.24 (3H, d, J 6.8), 0.90 (3H, t, J 7.3), 0.85 (9H, s), 0.01 (3H, s), −0.01 (3H, s); δ_{C} (50 MHz; CDCl₃) 202.00 (CO), 148.95 (CH), 124.29 (CH), 74.51 (CH), 34.83 (CH₂), 25.84 (3×CH₃), 21.36 (CH₂), 21.25 (CH₃), 18.26 (C), 13.81 (CH₃), −4.75 (SiCH₃), −4.89 (SiCH₃).

### (2S,3S,E)-2-(tert-Butyldimethylsilyloxy)oct-4-en-3-ol and (2S,3S,E)-3-(tert-butyldimethyl-silyloxy)oct-4-en-2-ol

Lithium tri-sec-butylborohydride (1 M tetrahydrofuran solution; 10.5 mL, 10.5 mmol) was added dropwise over a period of 10 minutes to solution of (S,E)-2-(tert-butyldimethylsilyloxy)oct-4-en-3-one (1.79 g, 6.98 mmol) in tetrahydrofuran (40 mL) at 0° C. under an atmosphere of argon. The reaction mixture was stirred for 3 h, quenched by the addition of a mixture of ethyl acetate/water (1:1) and separated. The aqueous phase was extracted twice with ethyl acetate and the combined organics were washed with brine, dried with sodium sulfate, filtered and evaporated under reduced pressure. The crude material was chromatographed on a silica gel column. Gradient elution with ethyl acetate/light petroleum (0-2%) gave a mixture of (2S,3S,E)-2-(tert-butyldimethylsilyloxy)oct-4-en-3-ol and (2S,3S,E)-3-(tert-butyldimethylsilyloxy)oct-4-en-2-ol (1.73 g, 6.70 mmol) that was taken forward in the hydrogenation reaction detailed below.

### (2S,3S)-2-(tert-Butyldimethylsilyloxy)octan-3-ol and (2S,3S)-3-(tert-butyldimethyl-silyloxy)octan-2-ol

Palladium 10% on carbon (0.70 g) was added to a mixture of (2S,3S,E)-2-(tert-butyldimethyl yloxy)oct-4-en-3-ol and (2S,3S,E)-3-(tert-butyldimethyl-silyloxy)oct-4-en-2-ol (1.70 g, 6.58 mmol) dissolved in a mixture of ethanol and ethyl acetate (2:1, 30 mL). The substrates were hydrogenated under hydrogen (1 atm) for 12 h. TLC (5% ethyl acetate/light petroleum) indicated conversion of substrates to product products (R_{f} 0.42). The reaction mixture was filtered over a pad of Celite, washing with ethyl acetate (3×20 mL), and the filtrate was evaporated under reduced pressure to give a crude mixture of (2S,3S)-2-(tert-butyldimethylsilyloxy)octan-3-ol and (2S,3S)-3-(tert-butyldimethylsilyloxy)octan-2-ol (1.33 g) that was taken forward into the Mitsunobu coupling step detailed below.

### 9-[(2R,3S)-3-(tert-Butyldimethylsilyloxy)octan-2-yl]-6-chloro-9H-purine and 9-[(2S,3R)-2-(tert-butyldimethylsilyloxy)octan-3-yl]-6-chloro-9H-purine

Procedure for the preparation was adapted from Hikishima et al (Bioorg. Med. Chem., 2006, 14, 1660-1670):

Diisopropyl azodicarboxylate (2.30 mL, 12.0 mmol) was added dropwise to a mixture of (2S,3S)-2-(tert-butyldimethylsilyloxy)octan-3-ol and (2S,3S)-3-(tert-butyldimethylsilyloxy)octan-2-ol (1.56 g, 6.00 mmol), 6-chloro-9H-purine (1.21 g, 7.80 mmol) and triphenylphosphine (2.36 g, 9.00 mmol) in tetrahydrofuran (50 mL) under an atmosphere of argon. The reaction mixture was stirred at room temperature for 18 h and then filtered over silica gel, washing with (3:1 petroleum/ethyl acetate, 200 mL). The filtrate was evaporated under reduced pressure to give a viscous dark yellow liquid that was chromatographed on a silica gel column. Elution with 2%. petroleum/ethyl acetate (200 mL) gave 9-[(2S,3R)-2-(tert-butyldimethylsilyloxy)octan-3-yl]-6-chloro-9H-purine (155 mg, 0.39 mmol; 7%) and 9-[(2R,3S)-3-(tert-butyldimethylsilyloxy)octan-2-yl]-6-chloro-9H-purine (387 mg, 0.98 mmol; 16%).

9-[(2S,3R)-2-(tert-butyldimethylsilyloxy)octan-3-yl]-6-chloro-9H-purine: δ_{H} (200 MHz; CDCl₃) 8.66 (1H, s), 8.13 (1H, s), 4.49 (1H, dt, J 10.9 & 4.4, chain H-3), 4.06 (1H, qd, J6.3 & 4.4, chain H-2), 2.15-1.88 (2H, m), 1.30-0.93 (9H, m), 0.80 (9H, s), 0.73 (3H, t, J 6.8), −0.07 (3H, s), −0.24 (3H, s).

9-[(2R,3S)-3-(tert-butyldimethylsilyloxy)octan-2-yl]-6-chloro-9H-purine: δ_{H} (200 MHz; CDCl₃) 8.67 (1H, s), 8.14 (1H, s), 4.84 (1H, qd, J 7.1 & 3.1, chain H-2), 3.96-3.88 (1H, m, chain H-3), 1.57 (3H, d J 7.1), 1.57-1.13 (8H, m), 0.85 (3H, t, J6.5), 0.79 (9H, s), −0.14 (3H, s), −0.55 (3H, s); δ_{C} (101 MHz; CDCl₃) 151.64 (CH), 151.50 (C), 150.82 (C), 144.62 (CH), 131.58 (C), 73.25 (CH), 54.11 (CH), 34.43 (CH₂), 31.90 (CH₂), 25.83 (3×CH₃), 24.82 (CH₂), 22.54 (CH₂), 17.92 (C), 14.02 (CH₃), 12.77 (CH₃), −4.31 (SiCH₃), −5.32 (SiCH₃).

### 9-[(2R,3S)-3-(tert-Butyldimethylsilyloxy)octan-2-yl]-9H-purin-6-amine

A mixture of 9-[(2R,3S)-3-(tert-butyldimethylsilyloxy)octan-2-yl]-6-chloro-9H-purine (377 mg, 0.95 mmol) in aqueous ammonia (SG 0.880; 4.0 mL) was heated at 80° C. in a 5 mL sealed pressure tube for 18 h. TLC (95:5 dichloromethane/methanol) indicated consumption of starting material (R_{f} 0.80) and formation of two new product components, minor (R_{f} 0.75) and major (R_{f} 0.30). The reaction mixture was evaporated to give a crude white solid that was chromatographed on a silica gel column (10 g). Gradient elution with dichloromethane/methanol (99:1, 250 mL; 98:2, 200 mL; 9:1, 120 mL) gave 9-[(2R,3S)-3-(tert-butyldimethylsilyloxy)octan-2-yl]-9H-purin-6-amine (R_{f} 0.30) (243 mg, 0.64 mmol; 68%) as a white solid: δ_{H} (200 MHz; CDCl₃) 8.34 (1H, s), 7.87 (1H, s), 5.93 (2H, br s), 4.76 (1H, qd, J 7.1 & 3.3, chain H-2), 4.02-3.94 (1H, m, chain H-3), 1.56 (3H, d, J 7.1), 1.55-1.12 (8H, m), 0.89 (3H, t, J 6.5), 0.85 (9H, s), −0.09 (3H, s), −0.48 (3H, s); δ_{C} (50 MHz; CDCl₃): 155.67 (C), 152.89 (CH), 149.92 (C), 140.06 (CH), 119.71 (C), 73.50 (CH), 53.37 (CH), 34.82 (CH₂), 32.21 (CH₂), 26.04 (3×CH₃), 24.98 (CH₂), 22.70 (CH₂), 18.12 (C), 14.23 (CH₃), 13.01 (CH₃), −4.15 (CH₃), −5.29 (CH₃).

### (2R,3S)-2-(6-Amino-9H-purin-9-yl)octan-3-ol (HWC-58)

Tetrabutylammonium fluoride (1 M tetrahydrofuran solution; 0.39 mL, 0.39 mmol) was added to a solution of 9-[(2R,3S)-3-(tert-butyldimethylsilyloxy)octan-2-yl]-9H-purin-6-amine (223 mg, 0.59 mmol) in tetrahydrofuran (4 mL at room temperature and stirred for 18 h. TLC (95:5 dichloromethane/methanol), indicated consumption of starting material (R_{f} 0.30) and formation of a product component (R_{f} 0.13). The reaction mixture was evaporated to give a crude residue that was dissolved in ethyl acetate (50 mL), washed with brine (3×10 mL), dried with sodium sulfate, filtered and concentrated in vacuo to give a crude white solid. The crude material was chromatographed on a silica gel column (10 g). Gradient elution with dichloromethane/methanol (95:5, 100 mL; 9:1, 100 mL) gave (2R,3S)-2-(6-amino-9H-purin-9-yl)octan-3-ol (R_{f} 0.13) (146 mg, 0.55 mmol; 94%) as a white solid: δ_{H} (200 MHz; CDCl₃/CD₃OD): 8.16 (1H, s), 7.92 (1H, s), 4.53 (1H, qd, J 7.1 & 2.8, chain H-2), 3.84-3.76 (1H, m, chain H-3), 1.46 (3H, d, J 7.1), 1.42-1.32 (2H, m), 1.36-1.07 (6H, m), 0.79 (3H, t, J 6.5); δ_{C} (50 MHz; CDCl₃/CD₃OD): 155.53 (C), 152.18 (CH), 148.95 (C), 140.01 (CH), 118.91 (C), 72.83 (CH), 56.06 (CH), 33.96 (CH₂), 31.66 (CH₂), 25.71 (CH₂), 22.51 (CH₂), 13.96 (CH₃), 13.91 (CH₃).

### Synthesis of (2S,3R)-3-(6-amino-9H-purin-9-yl)octan-2-ol (HWC-59)

### 9-[(2S,3R)-2-(tert-Butyldimethylsilyloxy)octan-3-yl]-9H-purin-6-amine

A mixture of 9-[(2S,3R)-2-(tert-butyldimethylsilyloxy)octan-3-yl]-6-chloro-9H-purine (150 mg, 0.38 mmol) in ammonia (7 N in methanol; 4 mL, 28 mmol) was heated at 80° C. in a sealed tube for 12 h. TLC of the reaction mixture (2% methanol/dichloromethane) indicated new product components (R_{f} 0.29 and R_{f} 0.09). The reaction mixture was evaporated and the crude residue was chromatographed on a silica gel column (20 g). Gradient elution with dichloromethane (50 mL) followed by dichloromethane/methanol (99:1, 50 mL; 98:2, 50 mL; 97:3, 50 mL; 96:4, 50 mL) gave 9-[(2S,3R)-2-(tert-butyldimethylsilyloxy)octan-3-yl]-9H-purin-6-amine (91 mg, 0.24 mmol; 64%): δ_{H} (200 MHz; CDCl₃) 8.33 (1H, s), 7.85 (1H, s), 6.17 (2H, br s), 4.41 (1H, dt, J 10.1 & 4.9, chain H-3), 4.07 (1H, qd, J 6.2 & 4.5, chain H-2), 2.10-1.92 (2H, m), 1.15 (9H, m), 0.94-0.61 (12H, m), −0.04 (3H, s), −0.21 (3H, s); δ_{C} (50 MHz; CDCl₃): 155.81 (C), 152.92 (CH), 150.39 (C), 140.14 (CH), 119.67 (C), 70.30 (CH), 61.18 (CH), 31.52 (CH₂), 27.42 (CH₂), 25.98 (3×CH₃), 25.68 (CH₂), 22.54 (CH₂), 20.97 (CH₃), 18.08 (C), 14.11 (CH₃), −4.19 (SiCH₃), −5.01 (SiCH₃).

### (2S,3R)-3-(6-amino-9H-purin-9-yl)octan-2-ol (HWC-59)

Tetrabutylammonium fluoride (1 M tetrahydrofuran solution; 0.46 mL, 0.46 mmol) was added to a solution of 9-[(2S,3R)-2-(tert-butyldimethylsilyloxy)octan-3-yl]-9H-purin-6-amine (86 mg, 0.23 mmol) in tetrahydrofuran (4.0 mL) at room temperature and stirred for 18 h. TLC (9:1 dichloromethane/methanol) indicated consumption of starting material (R_{f} 0.58) and formation of a product component (R_{f} 0.38). The reaction mixture was evaporated to give a crude residue that was dissolved in ethyl acetate (50 mL), washed with brine (4×10 mL), dried with sodium sulfate, filtered and concentrated in vacuo to give a crude oil. The crude material was chromatographed on a silica gel column (15 g). Elution with dichloromethane/methanol (95:5, 350 mL) gave semi-pure (2S,3R)-3-(6-amino-9H-purin-9-yl)octan-2-ol (59 mg) as a colourless oil (R_{f} 0.38). The semi-pure material was re-chromatographed on a silica gel column (10 g). Elution with ethyl acetate/acetone (1.5:1, 400 mL) gave (2S,3R)-3-(6-amino-9H-purin-9-yl)octan-2-ol (50 mg, 0.19 mmol; 83%): δ_{H} (200 MHz; CDCl₃) 8.24 (1H, s), 7.83 (1H, s), 6.71 (2H, br s), 5.69 (1H, br s), 4.32 (1H, dt, J 10.7 & 3.1, chain H-3), 4.20 (1H, qd, J 6.5 & 2.7, chain H-2), 2.14-1.81 (2H, m), 1.24 (3H, d, J 6.6), 1.24-0.93 (6H, m), 0.76 (3H, t, J 6.3); δ_{C} (50 MHz; CDCl₃) 156.08 (C), 152.52 (CH), 149.82 (C), 140.43 (CH), 119.74 (C), 69.46 (CH), 62.92 (CH), 31.44 (CH₂), 27.56 (CH₂), 25.99 (CH₂), 22.49 (CH₂), 20.27 (CH₃), 14.04 (CH₃).

### Synthesis of (2S,3S)-3-(6-amino-9H-purin-9-yl)hexan-2-ol (HWC-60)

### (S,E)-2-(tert-Butyldimethylsilyloxy)hex-4-en-3-one

Prepared by the adaptation of a procedure reported by Taddei et al (J. Org. Chem., 2006, 71, 103-107):

Lithium chloride (0.69 g, 16.2 mmol) was added to a solution of (S)-dimethyl-3-(tert-butyldimethylsilyloxy)-2-oxobutylphosphonate (5.03 g, 16.2 mmol) in acetonitrile (100 mL) under argon at room temperature. N,N-Diisoproplyethylamine (2.41 mL, 13.4 mmol) was added and the reaction mixture was stirred for 2 h to give a viscous mixture. Acetaldehyde (0.99 mL, 17.2 mmol) was added and the reaction mixture was stirred for a further 92 h. TLC (10% ethyl acetate/light petroleum) indicated a new component (R_{f} 0.52). The reaction mixture was quenched with brine (50 mL), extracted with ethyl acetate (3×40 mL), dried with sodium sulfate and evaporated to give a crude colourless oil (2 g). The crude material was chromatographed on a silica gel column (40 g). Elution with 2% ethyl acetate/light petroleum gave (S,E)-2-(tert-butyldimethylsilyloxy)hex-4-en-3-one (1.92 g, 8.40 mmol; 52%): δ_{H} (200 MHz; CDCl₃) 6.99 (1H, dq, J 15.6 & 6.9), 6.57 (1H, dt, J 15.5 & 1.6), 4.22 (1H, q, J 6.8), 1.89 (3H, dd, J 6.8 & 1.6), 1.27 (3H, d, J 6.8), 0.88 (9H, s), 0.04 (6H, s); δ_{C} (50 MHz; CDCl₃) 201.91 (CO), 144.35 (CH), 125.88 (CH), 74.50 (CH), 25.91 (3×CH₃), 21.30 (CH₃), 18.70 (CH₃), 18.33 (C), −4.69 (SiCH₃), −4.83 (SiCH₃).

### (2S,3S,E)-2-(tert-Butyldimethylsilyloxy)hex-4-en-3-ol and (2S,3S,E)-3-(tert-butyldimethylsilyloxy)hex-4-en-2-ol

Prepared by adaptation of a procedure reported by Terasaka et al. (J. Med. Chem., 2005, 48, 4750-4753):

Lithium tri-sec-butylborohydride (1 M tetrahydrofuran solution; 11.2 mL, 11.2 mmol) was added dropwise to a solution of (S,E)-2-(tert-butyldimethylsilyloxy)hex-4-en-3-one (1.71 g, 7.50 mmol) in tetrahydrofuran (40 mL) under argon at 0° C. over a period of 15 minutes and the reaction mixture was stirred for a further 3 h. TLC (5% ethyl acetate/light petroleum) indicated formation of a new component (R_{f} 0.29). The reaction mixture was quenched by the slow addition of a mixture of ethyl acetate/water (1:1, 20 mL). The organic layer was washed with brine (2×10 mL), dried with sodium sulfate and evaporated to give a pale brown oil. The crude oil was chromatographed on a silica gel column (30 g). Elution with 2% ethyl acetate/light petroleum gave a mixture of (2S,3S,E)-2-(tert-butyldimethylsilyloxy)hex-4-en-3-ol and (2S,3S,E)-3-(tert-butyldimethylsilyloxy)-hex-4-en-2-ol (1.70 g, 7.38 mmol; 98%) that was taken forward in the hydrogenation step detailed below.

### (2S,3S)-2-(tert-Butyldimethylsilyloxy)hexan-3-ol and (2S,3S)-3-(tert-butyldimethylsilyloxy)hexan-2-ol

10% Palladium on charcoal (100 mg) was added to a mixture of (2S,3S,E)-2-(tert-butyldimethylsilyloxy)hex-4-en-3-ol and (2S,3S,E)-3-(tert-butyldimethylsilyloxy)hex-4-en-2-ol (1.68 g, 7.29 mmol) dissolved in ethanol (20 mL). The mixture was hydrogenated for 18 h at room temperature under hydrogen (1 atm). TLC (5% ethyl acetate/light petroleum) indicated consumption of starting material and the reaction mixture was filtered, washing with ethanol (2×30 mL). The filtrate was concentrated in vacuo to give a mixture of crude (2S,3S)-2-(tert-butyldimethylsilyloxy)hexan-3-ol and (2S,3S)-3-(tert-butyldimethylsilyloxy)hexan-2-ol (1.43 g): δ_{H} (200 MHz; CDCl₃) that was taken forward in the Mitsunobu coupling step detailed below.

### 9-[(2S,3R)-2-(tert-Butyldimethylsilyloxy)hexan-3-yl]-6-chloro-9H-purine and 9-[(2R,3S)-3-(tert-butyldimethylsilyloxy)hexan-2-yl]-6-chloro-9H-purine

Procedure adapted from Hikishima (Bioorg. Med. Chem., 2006, 14, 1660-1670):

Diisopropyl azodicarboxylate (2.51 mL, 12.9 mmol) was added to a mixture of (2S,3S)-2-(tert-butyldimethylsilyloxy)hexan-3-ol and (2S,3S)-3-(tert-butyldimethyl-silyloxy)hexan-2-ol (1.50 g, 6.45 mmol), triphenylphosphine (2.54 g, 9.68 mmol) and 6-chloro-9H-purine (1.30 g, 8.39 mmol) dissolved in tetrahydrofuran (50 mL) under an atmosphere of argon and stirred for 18 h at room temperature. The reaction mixture was filtered through a short silica pad, washing with petroleum/ethyl acetate (3:1, 100 mL). The filtrate was evaporated at reduced pressure to give a crude oil that was chromatographed on a silica gel column (80 g). Gradient elution with light petroleum (100 mL) followed by light petroleum/ethyl acetate (98:2, 200 mL; 96:4, 100 mL; 94:6, 100 mL; 92:8, 100 mL) gave 9-[(2S,3R)-2-(tert-butyldimethylsilyloxy)hexan-3-yl]-6-chloro-9H-purine (251 mg, 0.68 mmol; 11%) and 9-[(2S,3S)-3-(tert-butyldimethylsilyloxy)hexan-2-yl]-6-chloro-9H-purine (171 mg, 0.46 mmol; 7%).

9-[(2S,3R)-2-(tert-Butyldimethylsilyloxy)hexan-3-yl]-6-chloro-9H-purine: δ_{H} (200 MHz; CDCl₃) 8.71 (1H, s), 8.17 (1H, s), 4.56 (1H, dt, J 11.4 & 4.0, chain H-3), 4.10 (1H, qd, J 6.2 & 4.1, chain H-2), 2.24-1.89 (2H, m), 1.21-0.99 (2H, m), 1.18 (3H, d, J 6.3), 0.87 (3H, t, J 6.5), 0.86 (9H, s), −0.02 (3H, s), −0.19 (3H, s).

9-[(2R,3S)-3-(tert-Butyldimethylsilyloxy)hexan-2-yl]-6-chloro-9H-purine: δ_{H} (200 MHz; CDCl₃) 8.73 (1H, s), 8.18 (1H, s), 4.88 (1H, qd, J 7.1 & 3.1, chain H-2), 4.02-3.94 (1H, m, chain H-3), 1.61 (3H, d, J 7.1), 1.61-0.13 (4H, m), 0.98 (3H, t, J 6.5), 0.84 (9H, s), −0.09 (3H, s), −0.51 (3H, s).

### 9-[(2S,3R)-2-(tert-Butyldimethylsilyloxy)hexan-3-yl]-9H-purin-6-amine

A mixture of 9-[(2S,3R)-2-(tert-butyldimethylsilyloxy)hexan-3-yl]-6-chloro-9H-purine (251 mg, 0.68 mmol) in aqueous ammonia (SG 0.880; 3.0 mL) was heated at 100° C. in a sealed tube for 18 h. TLC (5% methanol/dichloromethane) indicated formation of a new component (R_{f} 0.24). The reaction mixture was cooled and extracted with ethyl acetate (3×20 mL). The combined organic extracts were washed with brine (15 mL), dried with sodium sulfate and evaporated under reduced pressure to give a crude white solid that was chromatographed on a silica gel column (20 g). Gradient elution with dichloromethane (100 mL); followed by dichloromethane/methanol (99:1, 100 mL; 98:2, 100 mL; 97:3, 100 mL) gave 9-[(2S,3R)-2-(tert-butyldimethylsilyloxy)hexan-3-yl]-9H-purin-6-amine (186 mg, 0.53 mmol; 78%): δ_{H} (200 MHz; CDCl₃) 8.35 (1H, s), 7.86 (1H, s), 5.69 (2H, br s), 4.45 (1H, dt, J 11.2 & 4.3, chain H-3), 4.11 (1H, qd, J 6.3 & 4.1, chain H-2), 2.20-1.87 (2H, m), 1.19-1.04 (2H, m), 1.17 (3H, d, J6.3), 0.88 (3H, t, J 6.5), 0.88 (9H, s), −0.02 (3H, s), −0.19 (3H, s).

### (2S,3R)-3-(6-Amino-9H-purin-9-yl)hexan-2-ol (HWC-60)

Tetrabutylammonium fluoride (1 M tetrahydrofuran solution; 1.03 mL, 1.03 mmol) was added to a solution of 9-[(2S,3R)-2-(tert-butyldimethylsilyloxy)hexan-3-yl]-9H-purin-6-amine (180 mg, 0.52 mmol) in tetrahydrofuran (10 mL) at room temperature and stirred for 18 h. TLC (1:1 ethyl acetate/acetone) indicated consumption of starting material (R_{f} 0.64) and formation of a product component (R_{f} 0.26). The reaction mixture was evaporated to give a crude residue that was dissolved in ethyl acetate (50 mL), washed with brine (3×10 mL), dried with sodium sulfate, filtered and concentrated in vacuo to give a crude oil. The crude material was chromatographed on a silica gel column (15 g). Gradient elution with dichloromethane/methanol (98:2, 250 mL; 95:5, 300 mL) gave partially purified (2S,3R)-3-(6-amino-9H-purin-9-yl)hexan-2-ol (R_{f} 0.26, 116 mg) as a colourless oil. The partially purified material was re-chromatographed on a silica gel column (15 g). Gradient elution with ethyl acetate/acetone (1.5:1, 250 mL; 1:1, 200 mL) gave (2S,3R)-3-(6-amino-9H-purin-9-yl)hexan-2-ol (R_{f} 0.26) (67 mg, 0.29 mmol; 55%) as a white amorphous solid: δ_{H} (200 MHz; CDCl₃) 8.22 (1H, s), 7.83 (1H, s), 6.81 (2H, br s), 5.72 (1H, br s), 4.35 (1H, dt, J 11.0 & 3.2), 4.18 (1H, qd, J 6.5 & 2.9, chain H-2), 2.15-1.77 (2H, m), 1.22 (3H, d, J 7.1), 1.22-0.98 (2H, m), 0.81 (3H, t, J 7.2); δ_{C} (50 MHz; CDCl₃) 156.10 (C), 152.51 (CH), 149.84 (C), 140.35 (CH), 119.64 (C), 69.37 (CH), 62.39 (CH), 29.71 (CH₂), 20.23 (CH₃), 19.48 (CH₂), 13.71 (CH₃).

### Synthesis of (2R,3S)-2-(6-amino-9H-purin-9-yl)hexan-3-ol (HWC-61)

### 9-[(2R,3S)-3-(tert-Butyldimethylsilyloxy)hexan-2-yl]-9H-purin-6-amine

A mixture of 9-[(2R,3S)-3-(tert-butyldimethylsilyloxy)hexan-2-yl]-6-chloro-9H-purine (165 mg, 0.45 mmol) and aqueous ammonia (SG 0.880; 3 mL) was heated at 100° C. in a sealed tube for 18 h. TLC (2% methanol/dichloromethane) indicated formation of a new component (R_{f} 0.15). The reaction mixture was cooled and extracted with ethyl acetate (2×15 mL). The combined organic layers were washed with brine (10 mL) and dried with sodium sulfate. The filtrate was evaporated under reduced pressure to give a crude white solid that was chromatographed on silica gel. Gradient elution with dichloromethane/methanol (99:1, 100 mL; 98:2, 100 mL; 97:3, 100 mL) gave 9-[(2R,3S)-3-(tert-butyldimethylsilyloxy)hexan-2-yl]-9H-purin-6-amine (125 mg, 0.36 mmol; 80%): δ_{H} (200 MHz; CDCl₃) 8.33 (1H, s), 7.86 (1H, s), 6.18 (2H, br.s), 4.75 (1H, qd, J 7.1 & 3.3, chain H-2), 4.03-3.95 (1H, m, chain H-3), 1.59-1.32 (7H, m), 0.94 (3H, t, J 7.1), 0.84 (9H, s), 0.10 (3H, s), −0.491 (3H, s); δ_{C} (50 MHz; CDCl₃) 155.80 (C), 152.86 (CH), 149.85 (C), 139.92 (CH), 119.64 (C), 73.23 (CH), 53.34 (CH), 36.97 (CH₂), 26.01 (3×CH₃), 18.68 (CH₂), 18.09 (C), 14.47 (CH₃), 12.94 (CH₃), −4.19 (SiCH₃), −5.32 (SiCH₃).

### (2R,3S)-2-(6-Amino-9H-purin-9-yl)hexan-3-ol (HWC-61)

Tetrabutylammonium fluoride (1 M tetrahydrofuran solution; 0.57 mL, 0.57 mmol) was added to a solution of 9-[(2R,3S)-3-(tert-butyldimethylsilyloxy)hexan-2-yl]-9H-purin-6-amine (125 mg, 0.286 mmol) in tetrahydrofuran (5.0 mL) at room temperature and stirred for 18 h. TLC (ethyl acetate/acetone 1:1), indicated consumption of starting material (R_{f} 0.44) and formation of a product component (R_{f} 0.14). The reaction mixture was evaporated to give a crude residue that was dissolved in ethyl acetate (50 mL), washed with brine (3×10 mL), dried with sodium sulfate, filtered and concentrated in vacuo to give a crude oil. The crude material was chromatographed on a silica gel column (5 g). Gradient elution with dichloromethane/methanol (95:5, 100 mL; 9:1, 100 mL) gave a crude oil that was re-chromatographed on a silica gel column (15 g). Elution with ethyl acetate/acetone (1:1, 550 mL) gave (2R,3S)-2-(6-amino-9H-purin-9-yl)hexan-3-ol (R_{f} 0.14) (46 mg, 0.20 mmol; 68%) as a white solid: δ_{H} (200 MHz; CDCl₃) 8.16 (1H, s), 7.90 (1H, s), 6.43 (2H, br s), 5.25 (1H, br s), 4.53 (1H, qd, J 7.1 & 2.7, chain H-2), 3.89-3.81 (1H, m, chain H-3), 1.46 (3H, d, J 7.1), 1.45-1.24 (4H, m), 0.84 (3H, t, J 7.0); δ_{C} (50 MHz; CDCl₃/CD₃OD) 155.66 (C), 152.30 (CH), 149.08 (C), 140.03 (CH), 119.08 (C), 72.59 (CH), 56.26 (CH), 36.15 (CH₂), 19.34 (CH₂), 13.95 (CH₃), 13.88 (CH₃).

### Synthesis of (rac)-9-(octan-3-yl)-9H-purin-6-amine (HWC-62)

### (rac)-6-Chloro-9-(octan-3-yl)-9H-purine

Diisopropyl azodicarboxylate (1.50 mL, 8.00 mmol) was added to a stirred mixture of octan-3-ol (0.76 mL, 4.8 mmol), 6-chloro-9H-purine (618 mg, 4.00 mmol) and triphenylphosphine (1.60 g, 6.00 mmol) in tetrahydrofuran (30 mL) at room temperature. After 18 h TLC (80% light petroleum/ethyl acetate) indicated formation of a product component (R_{f} 0.35). The reaction mixture was concentrated in vacuo and filtered through a short silica gel column, washing with light petroleum/ethyl acetate (1:1). Evaporation of the filtrate gave a crude yellow oil that was chromatographed on a silica gel column (60 g). Elution with light petroleum/ethyl acetate (9:1, 1 L; 7:1, 800 mL) gave 6-chloro-9-(decan-3-yl)-9H-purine (825 mg, 3.10 mmol; 77%) as a dense yellow oil: δ_{H} (200 MHz; CDCl₃) 8.71 (1H, s), 8.09 (1H, s), 4.56-4.41 (1H, m), 2.11-1.81 (4H, m), 1.33-0.95 (6H, m), 0.82-0.75 (6H, m); δ_{C} (50 MHz; CDCl₃) 152.19 (C), 151.80 (CH), 151.08 (C), 144.08 (CH), 131.85 (C), 59.96 (CH), 34.61 (CH₂), 31.30 (CH₂), 28.19 (CH₂), 25.86 (CH₂), 22.42 (CH₂), 13.96 (CH₃), 10.73 (CH₃).

### (rac)-9-(Octan-3-yl)-9H-purin-6-amine (HWC-62)

A mixture of 6-chloro-9-(octan-3-yl)-9H-purine (323 mg, 1.21 mmol) and aqueous ammonia (SG 0.880; 4.5 mL) was heated at 100° C. in a 5 mL sealed pressure tube for 18 h. TLC (95:5 dichloromethane/methanol) indicated conversion of starting material (R_{f} 0.72) into product (R_{f} 0.22). The reaction mixture was evaporated to give a crude waxy solid that was chromatographed on a silica gel column (15 g). Elution with dichloromethane/methanol (95:5, 100 mL) gave 9-(octan-3-yl)-9H-purin-6-amine (R_{f} 0.22) (286 mg, 1.16 mmol; 96%) as a pale yellow solid: δ_{H} (200 MHz; CDCl₃) 8.31 (1H, s), 7.62 (1H, s), 6.51 (2H, br s), 4.45-4.31 (1H, m), 2.04-1.75 (4H, m), 1.29-0.95 (6H, m), 0.77 (6H, t, J 7.3); δ_{C} (50 MHz; CDCl₃) 155.95 (C), 152.79 (CH), 150.39 (C), 139.06 (CH), 119.83 (C), 57.76 (CH), 34.77 (CH₂), 31.37 (CH₂), 28.32 (CH₂), 25.83 (CH₂), 22.44 (CH₂), 13.97 (CH₃), 10.69 (CH₃).

9-(Octan-3-yl)-9H-purin-6-amine was converted into its hydrochloride salt (HWC-62) by treatment with a saturated solution of hydrogen chloride in diethyl ether followed by evaporation.

### Synthesis of (rac)-9-(decan-3-yl)-9H-purin-6-amine (HWC-63)

### (rac)-6-Chloro-9-(decan-3-yl)-9H-purine

Diisopropyl azodicarboxylate (1.49 mL, 7.66 mmol) was added to a mixture of decan-3-ol (600 mg, 3.83 mmol), 6-chloro-9H-purine (650 mg, 4.21 mmol) and triphenylphosphine (1.50 g, 5.74 mmol) in tetrahydrofuran (40 mL) at room temperature and stirred for 24 h. TLC (70% light petroleum/ethyl acetate) indicated consumption of decan-3-ol (R_{f} 0.65) and formation of a product component (R_{f} 0.20). The reaction mixture was concentrated in vacuo and filtered through a short silica gel column, washing with light petroleum/ethyl acetate (1:1, 50 mL). Evaporation of the filtrate gave a crude yellow oil that was chromatographed on a silica gel column (40 g). Elution with light petroleum/ethyl acetate (95:5, 100 mL) gave 6-chloro-9-(decan-3-yl)-9H-purine (57 mg, 0.193 mmol; 5%) as a white solid: δ_{H} (200 MHz; CDCl₃): 8.85 (1H, s), 8.32 (1H, s), 5.10-4.88 (1H, m), 2.13-1.79 (4H, m), 1.39-1.05 (10H, m), 0.85 (3H, t, J 7.3), 0.81 (3H, ˜t, J 6.9).

### (rac)-9-(Decan-3-yl)-9H-purin-6-amine (HWC-63)

A mixture of 6-chloro-9-(decan-3-yl)-9H-purine (54 mg, 0.18 mmol) and aqueous ammonia (SG 0.880; 3 mL) was heated at 100° C. in a 5 mL sealed pressure tube for 18 h. TLC (3% methanol/dichloromethane) indicated conversion of starting material to a product component (R_{f} 0.29). The reaction mixture was evaporated to give a crude white solid that was chromatographed on a silica gel column. Elution with 2% methanol/dichloromethane gave 9-(decan-3-yl)-9H-purin-6-amine (35 mg, 0.13 mmol; 69%) as a white solid: δ_{H} (200 MHz; CDCl₃) 8.35 (1H, s), 7.93 (1H, s), 5.84 (2H, br s), 4.25 (1H, quintet, J6.7), 2.03-1.70 (4H, m), 1.26-0.97 (10H, m), 0.77 (3H, t, J 7.3), 0.71 (3H, ˜t, J 6.9); δ_{C} (50 MHz; CDCl₃) 160.34 (C), 152.73 (CH), 151.09 (C), 143.38 (CH), 112.34 (C), 60.99 (CH), 35.63 (CH₂), 31.72 (CH₂), 29.33 (CH₂), 29.27 (CH₂), 29.07 (CH₂), 25.99 (CH₂), 22.62 (CH₂), 14.12 (CH₃), 10.33 (CH₃).

9-(Decan-3-yl)-9H-purin-6-amine was converted into its hydrochloride salt (HWC-63) by treatment with a saturated solution of hydrogen chloride in diethyl ether followed by evaporation.

### Synthesis of 9-(decan-1-yl)-9H-purin-6-amine (HWC-64)

### 6-Chloro-9-(decan-1-yl)-9H-purine

Diisopropyl azodicarboxylate (1.8 mL, 9.3 mmol) was added to a stirred solution of decan-1-ol (1.1 g, 6.9 mmol), 6-chloro-9H-purine (720 mg, 4.66 mmol) and triphenylphosphine (1.8 g, 6.9 mmol) in tetrahydrofuran (30 mL) at ambient temperature. After 18 h TLC (9:1 light petroleum/ethyl acetate) indicated formation of a product component (R_{f} 0.2). The reaction mixture was filtered through a short silica gel column, washing with light petroleum/ethyl acetate (1:1). The filtrate was evaporated and the resulting red oil was chromatographed on flash silica gel (gradient elution with 15-20% ethyl acetate/light petroleum) to give 6-chloro-9-(decan-1-yl)-9H-purine (1.30 g, 4.41 mmol; 96%) as a pale brown powder: δ_{H} 8.74 (1H, s), 8.11 (1H, s), 4.28 (2H, t, J 7.2), 1.92 (2H, ˜quintet, J 7.0), 1.41-1.13 (14H, m), 0.6 (3H, ˜t, J 6.4).

### 9-(Decan-1-yl)-9H-purin-6-amine (HWC-64)

A mixture of 6-chloro-9-(decan-1-yl)-9H-purine (300 mg, 1.02 mmol) and aqueous ammonia (SG 0.880; 3 mL) was heated at 100° C. in a 5 mL sealed pressure tube for 18 h. TLC (2% methanol/dichloromethane) indicated conversion of starting material (R_{f} 0.45) to a product component (R_{f} 0.1). The reaction mixture was extracted with ethyl acetate; the extract was washed with saturated brine, dried (sodium sulfate) and evaporated. The resulting crude white solid (282 mg) was chromatographed on a silica gel column. Elution with 2% methanol/dichloromethane gave 9-(decan-1-yl)-9H-purin-6-amine (203 mg, 0.737 mmol; 72%) as a white solid: δ_{H} (200 MHz; CDCl₃) 8.35 (1H, s), 7.78 (1H, s), 6.32 (2H, br s), 4.16 (2H, t, J 7.1), 1.86 (2H, ˜quintet, J 6.5), 1.40-1.15 (14H, m), 0.88 (3H, ˜t, J 6.9); δ_{C} (101 MHz, CDCl₃) 155.73 (C), 152.91 (CH), 150.07 (C), 140.32 (CH), 119.68 (C), 43.95 (CH₂), 31.82 (CH₂), 30.08 (CH₂), 29.43 (CH₂), 29.40 (CH₂), 29.21 (CH₂), 29.03 (CH₂), 26.65 (CH₂), 22.63 (CH₂), 14.08 (CH₃).

9-(Decan-1-yl)-9H-purin-6-amine was converted into its hydrochloride salt (HWC-64) by treatment with a saturated solution of hydrogen chloride in diethyl ether followed by evaporation.

### Example 1

hESCs (SA121) were placed into standard feeder free conditions without exogenous FGF (including conditioned media made without addition of exogenous FGF) but supplemented with 10 μM EHNA. Cells were initially seeded from a trypsin passage of a standard, FGF containing, feeder free culture (passage 33 post feeder free; p100 total).

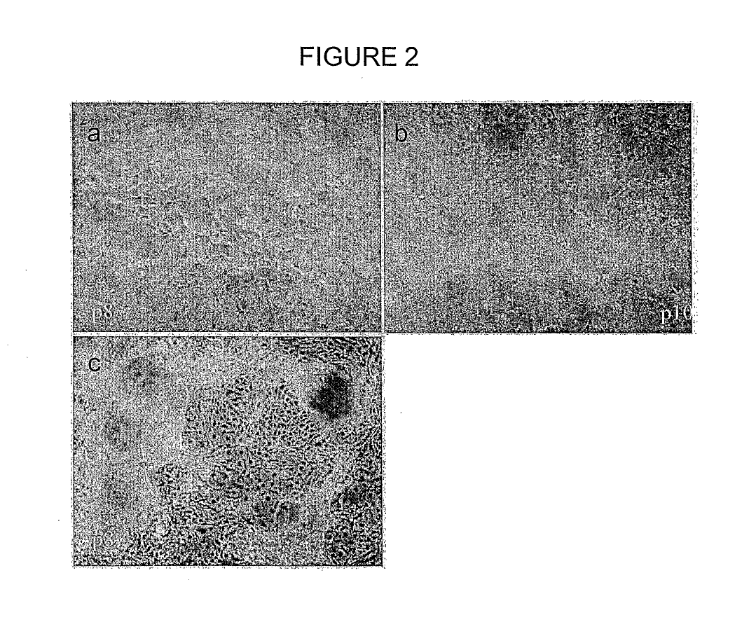

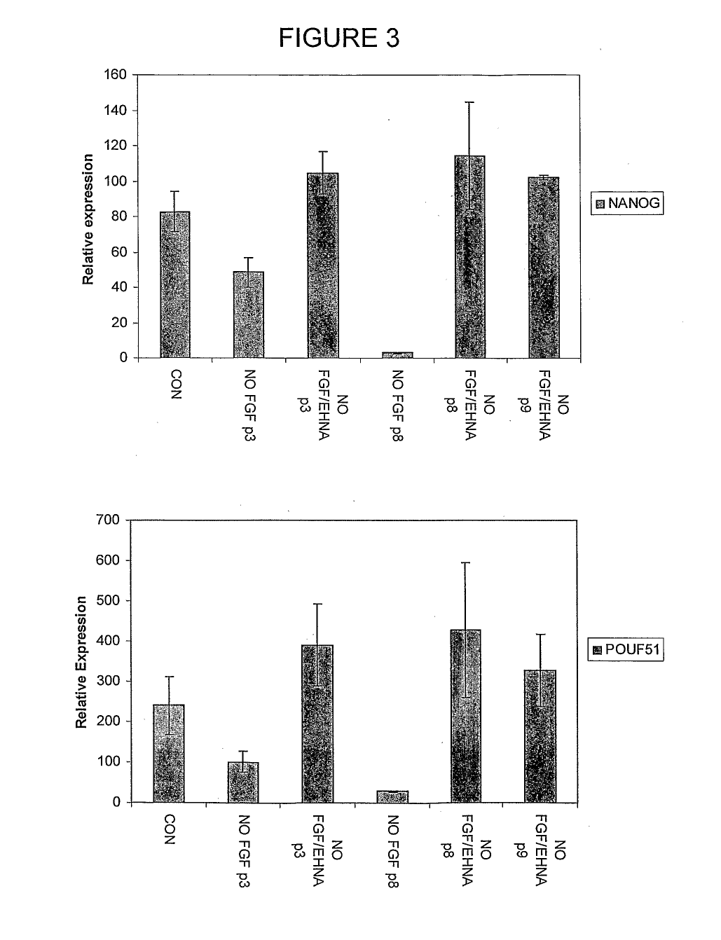

Controls were also set up without FGF or EHNA. Three lines were set up to grow independently for each condition and these were routinely passaged using trypsin as normal. Passaging was usually a 1 in 4 split approximately every 4 days. The results obtained demonstrate that hESCs can be maintained in an undifferentiated state in the absence of FGF if EHNA is present (FIGS. 2-4). In the absence of FGF and EHNA, a reduction in the expression of stem cells markers POU5F1 and NANOG can be seen as early as passage 3 (FIG. 3) and large amounts of differentiation can be seen by passage 8 (FIGS. 2 and 3).

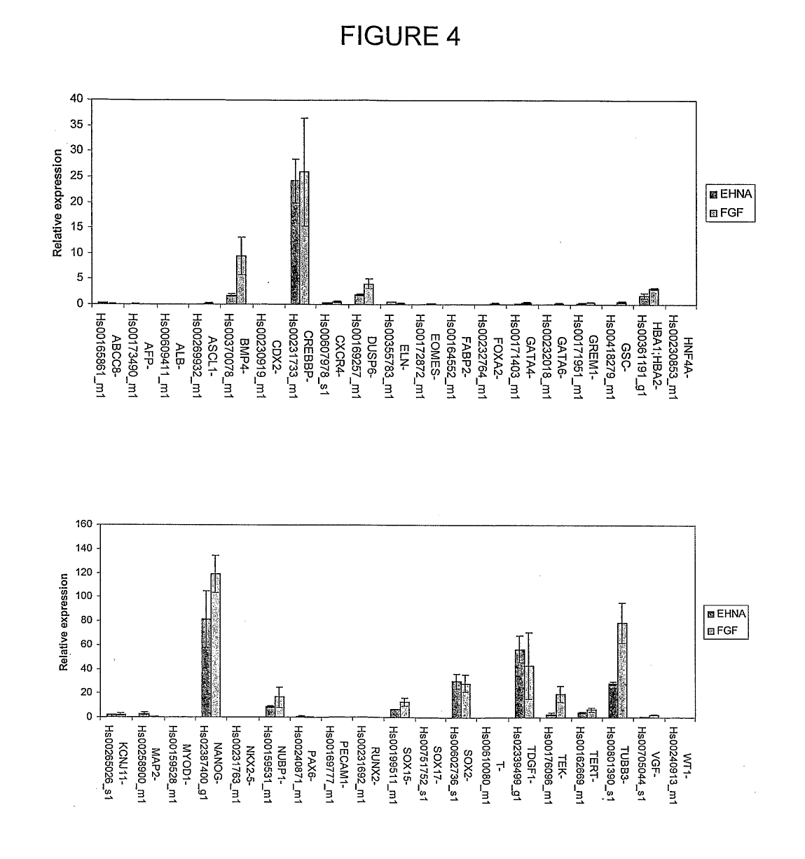

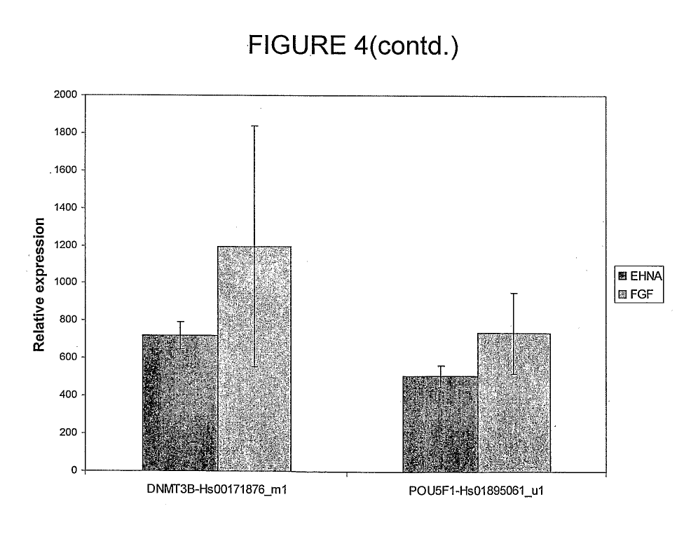

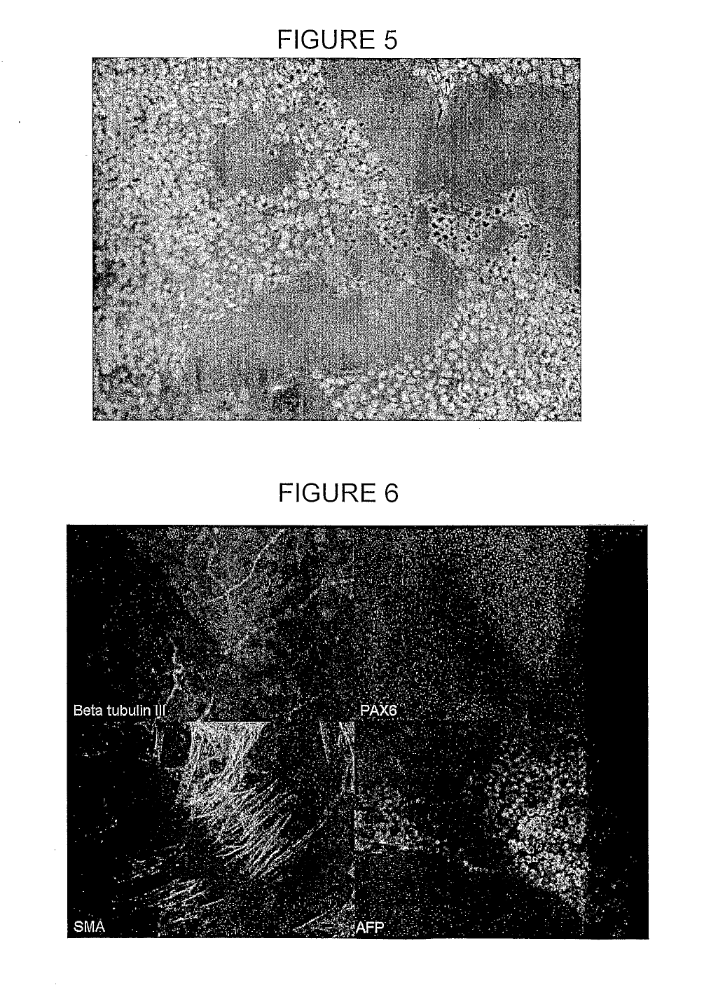

It has surprisingly been found that hESCs can be enzymatically passaged in feeder free conditions without exogenous FGF but supplemented with EHNA for at least 30 passages from feeder free culture. The cells show appropriate hESC gene expression (TLDA) at passage 30 in comparison to hESCs grown in the absence of EHNA but the presence of FGF (FIG. 4) and are all positive for POU5F1 (green) at passage 21 (FIG. 5).

Cells grown for at least 22 passages in the absence of FGF but the presence of EHNA can differentiate to all three germ layers. Cells were differentiated either passively by removing EHNA and replacing the fibronectin support with gelatin, in a monolayer with 20% serum or through EB formation. Cells were stained with pax6 (ectoderm), beta-tubulin III (ectoderm), alpha-feta protein (AFP) (endoderm) and smooth muscle Actin (SMA) (mesoderm) in order to detect differentiation in to all three germ layers (FIG. 6).

At least one replicate line was karyotypically normal at passage 21.

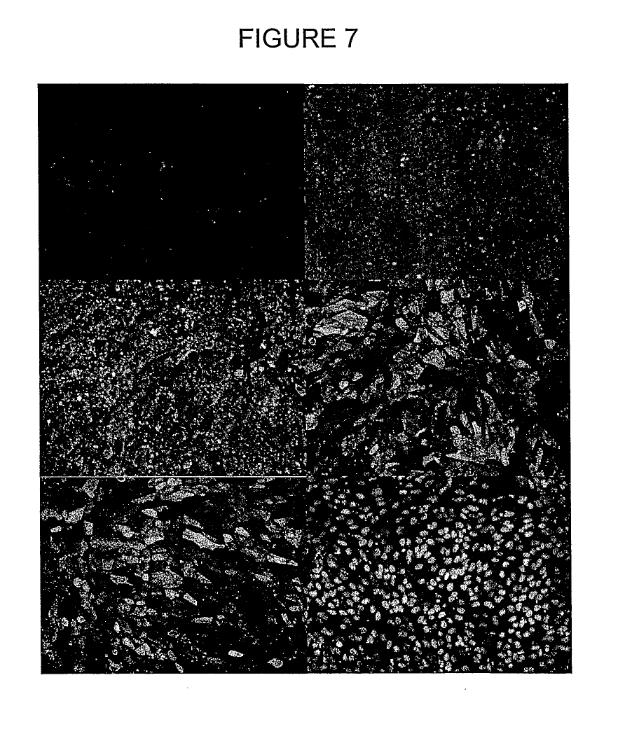

A further culture of SA121 was transferred to standard feeder free conditions without exogenous FGF (including conditioned media made without addition of exogenous FGF) but supplemented with 10 μM EHNA. Cells were initially seeded from a trypsin passage of a standard, FGF containing, feeder free culture (passage 7 post feeder free; p 48 total). These cells were grown for 10 passages and shown to negative for the differentiation marker SSEA1 (FIG. 7a) and positive for the stem-cell markers SSEA3, SSEA4, TRA-160, TRA-180 and OCT4 (FIG. 7 b-f respectively).

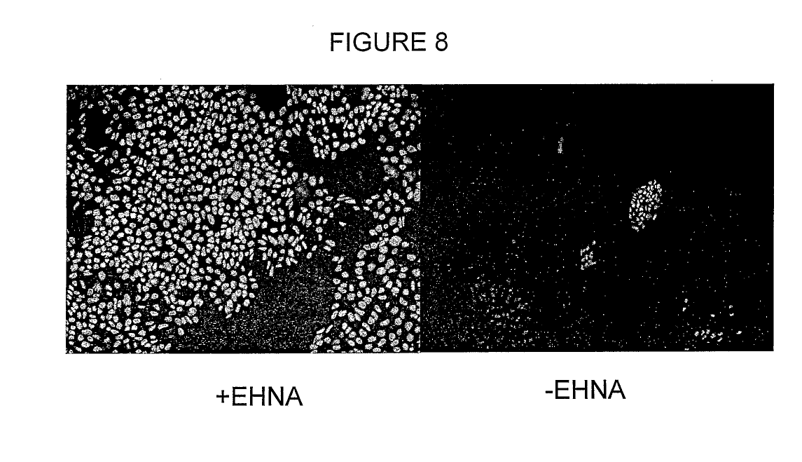

A culture of the hESC line SA461 was transferred from a supportive MEF feeder layer directly, using manual dissection, to standard feeder free conditions in the absence of exogenous FGF but supplemented with 10 uM EHNA. A control was also grown with neither FGF nor EHNA present. Cells were subsequently passaged by the standard trypsin feeder free technique. It was determined by passage 7 that the EHNA containing cultures were almost 100% positive for POU5F1 whereas positive staining in the cultures absent for FGF and EHNA was minimal (FIG. 8).

On the basis of these results, it is clear that EHNA, which is an example of an ADA inhibitor according to the present invention, can be used to effectively inhibit stem cell differentiation.

### Example 2

The aim of this experiment was to deconvolute the role of EHNA in stem cell marker maintenance during differentiation.

The results obtained demonstrate that it is the inhibition of adenosine deaminase that prolongs the expression of stem cell markers and inhibits the expression of neuronal marker PAX6 during monolayer differentiation.

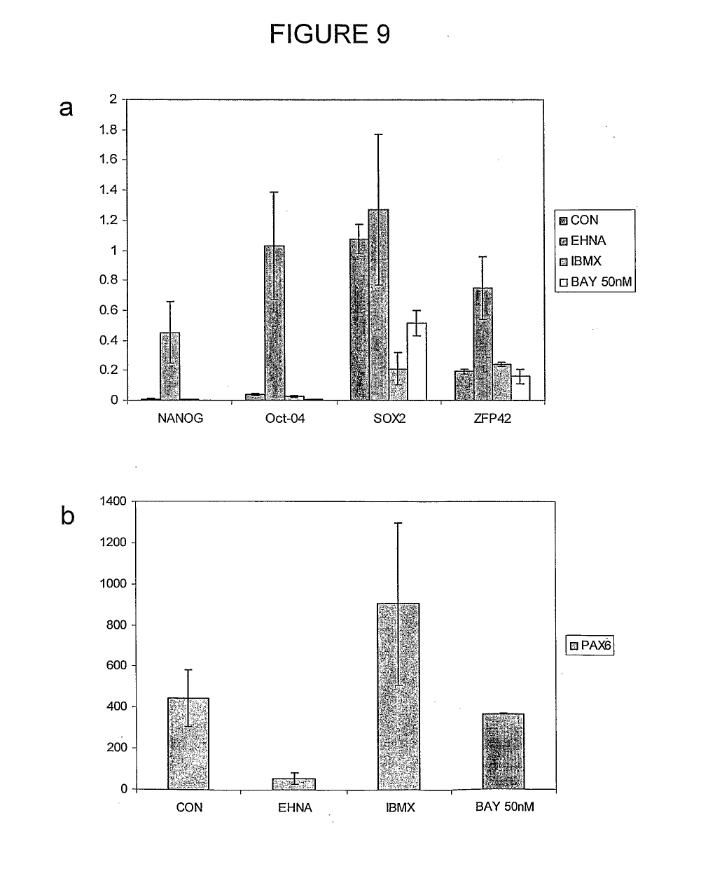

Cells were enzymatically passaged onto matrigel-coated dishes and grown for 2 weeks in defined media. After 14 days, qRT-PCR was utilised to analyse the expression of a variety of markers of pluripotency and differentiation. In the graphs in FIG. 9 the value 1 was set to the expression level of the gene in undifferentiated SA121 hESCs. In normal differentiating conditions POU5F1, NANOG and ZFP42 were down-regulated markedly indicating differentiation (FIG. 9a). Sox2 expression remained comparable to an undifferentiated hESC level but this gene is also associated with differentiated neuronal cell types (Wegner and Stolt, Trends in Neurosciences, 28, 11, November 2005, 583 to 588)

PAX6 is the earliest marker of neuronal progenitor differentiation so far identified, occurring as early as 6 days post plating (Pankratz et al., Stem Cells 2007; 25:1511-1520), this marker was seen to be induced in these differentiating conditions indicating a level of neuronal differentiation (FIG. 9b).

The addition of EHNA distinctly inhibited the down-regulation of the stem cell markers NANOG, ZFP42 and POU5F1 and in the case of SOX2 maintained transcription at the same level as seen in undifferentiated hESCs and the untreated samples (FIG. 9a). Treatment with EHNA also inhibited the rise of PAX6 expression levels seen in untreated cells indicating that neuronal differentiation has also been repressed (FIG. 9b). Whether the inhibition of differentiation marker expression extends to markers for other cell types is unknown. All markers tested (AFP, CD34, Brachyury, PECAM1, CDX2 and FOXA2) were not sufficiently expressed in the untreated cells for inhibition to be detected in the EHNA treated cells so PAX6 was utilised as the differentiation marker in further experiments (data not shown). No induction of these markers, however, was seen in the EHNA treated cells.

### Example 3

EHNA is also known to inhibit phosphodiesterase 2 (PDE2). In order to establish whether the inhibition of stem cell differentiation by EHNA was caused by PDE2 inhibition, the specific PDE2 inhibitor BAY-60-7550 (BAY) was also added to the cells as was the pan-PDE inhibitor 3-Isobutyl-1-methylxanthine (IBMX). Neither BAY nor IBMX maintained stem cell marker expression in differentiating conditions (FIG. 9a), nor did they inhibit PAX6 induction (FIG. 9b) indicating ADA inhibition as the cause of the gene expression alterations.

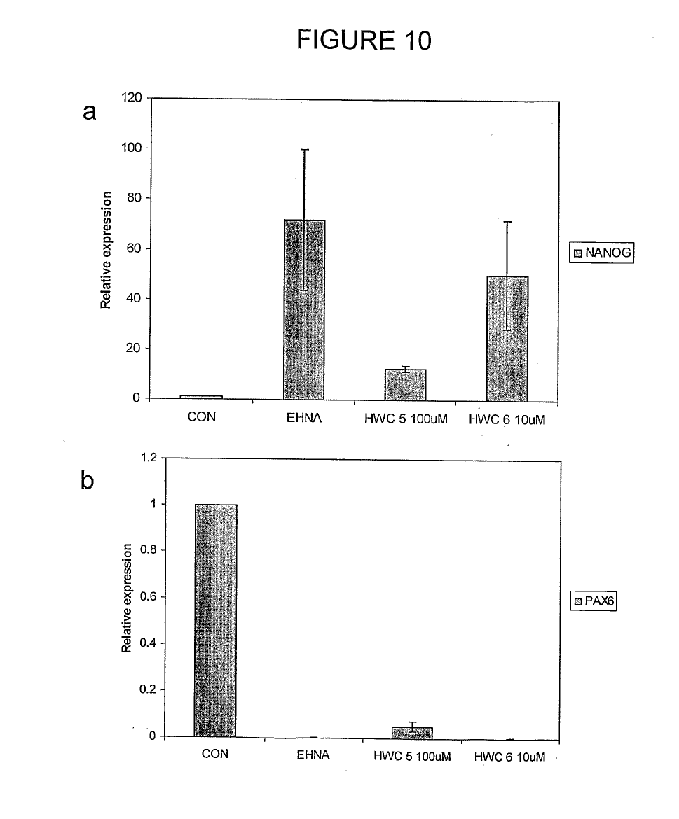

In order to further clarify the role of ADA in the inhibition of differentiation a further 2 ADA inhibitors were used: HWC-5 (erythro-3-(3H-imidazo[4,5-b]pyridin-3-yl)nonan-2-ol) is a 6-deamino-1-deaza EHNA analogue with a reported IC; 550 nM for ADA inhibition (Antonini et al., 1984), and HWC-6 (2-decyl-2H-pyrazolo[3,4-d]pyrimidin-4-amine hydrochloride) which is of the 2-alkylpyrazolo[3,4-d]pyrimidin-4-amine ADA inhibitor class with a reported Kᵢ 0.13 nM value for ADA inhibition (Settimo et al., 2005). Both of these ADA inhibitors are capable of maintaining NANOG and inhibiting PAX6 expression in differentiating conditions (FIG. 10). In FIG. 10, the value 1 was set to the expression level in the untreated control samples.

These data indicate HWC-5 and HWC-6 can prevent differentiation of hESCs.

### Example 4

The present inventors have found that EHNA delays the onset and reduces the amount of neuronal marker expression during early neuronal differentiation.

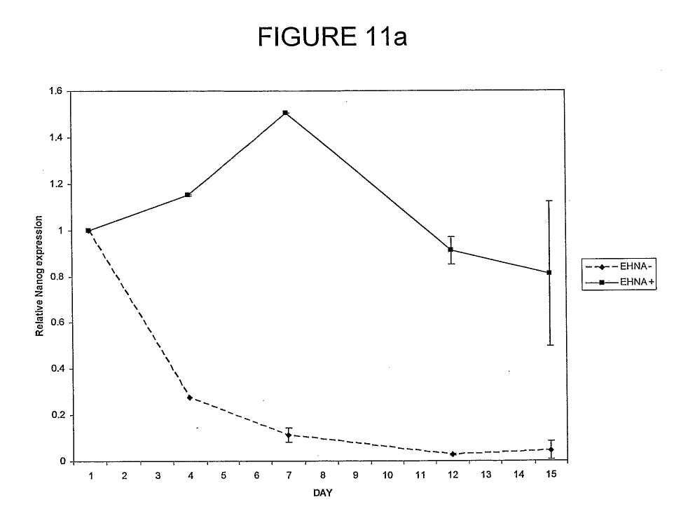

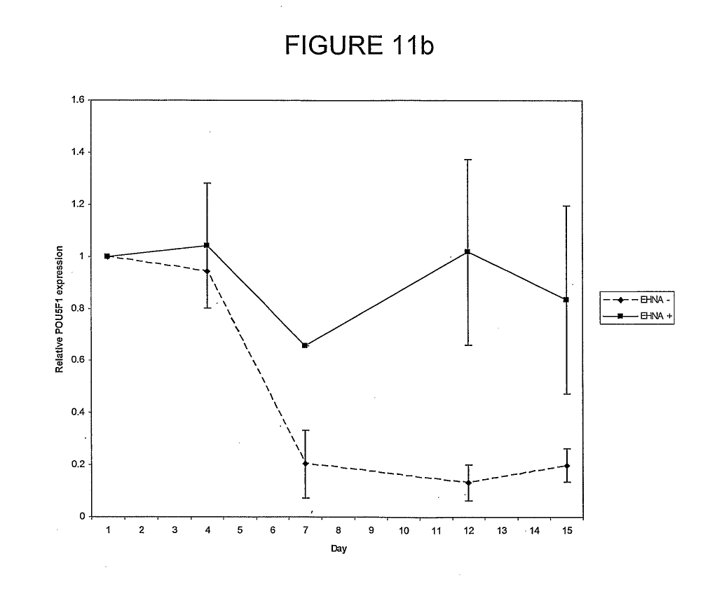

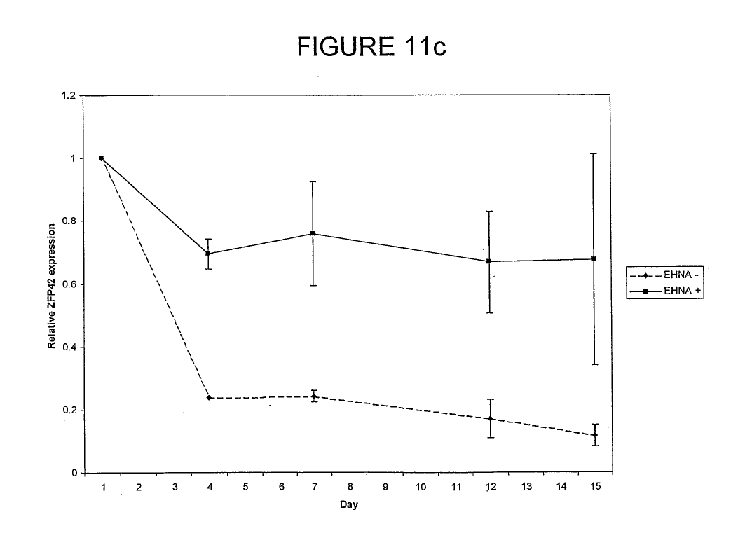

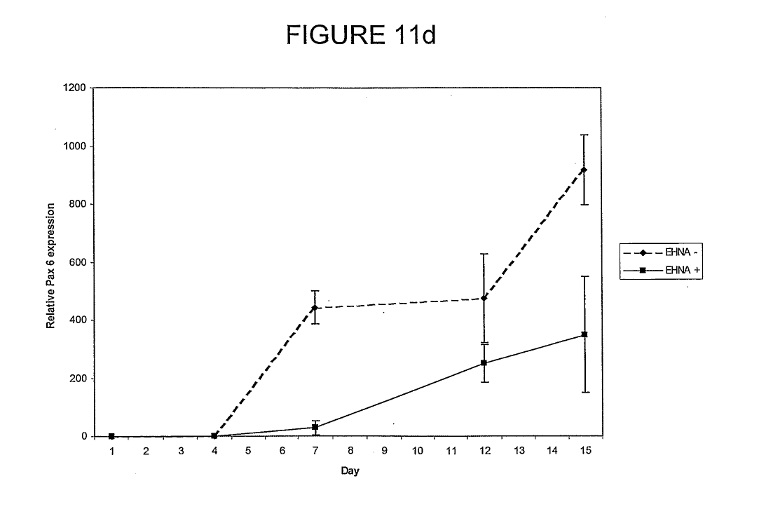

hESCs were put through the first 2 weeks of a directed monolayer neuronal differentiation with and without EHNA. RNA samples were taken at various time intervals and a variety of marker gene expression was measured using qRT-PCR. Expression levels of the stem cell genes POU5F1, NANOG and ZFP42 were maintained largely at undifferentiated levels in the EHNA treated samples throughout the experiment, whereas expression levels in the untreated samples dropped to at least 20% of hESC level in the first 4 to 7 days (FIGS. 11a-c).

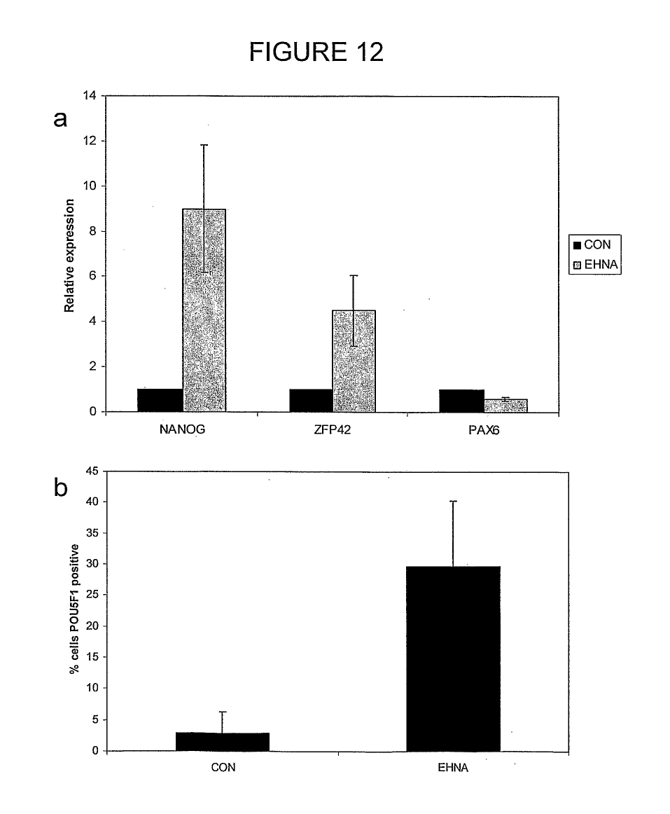

In the control neuronal differentiation PAX6 expression was massively upregulated/induced at day 7 and continued to rise during the rest of the differentiation (FIG. 11d). The effect of EHNA on PAX6 expression is not to inhibit its activation/upregulation but rather to delay or reduce the level of expression. In cells allowed to differentiate for 4 weeks (including a passage at 2 weeks), the PAX6 levels in the EHNA treated cells had reached in excess of 50% of the control differentiation but were still expressing considerably higher levels of the stem cells markers NANOG and ZFP42 (FIG. 12e). At least 10 times more cells still stained positive for POU5F1 expression in the EHNA treated differentiation samples indicating that the cells are capable of differentiating but inhibition of ADA seems to reduce the capacity to do so (FIG. 11b).

In FIGS. 11a-d, the value of 1 was set to the level of expression in undifferentiated hES cells. In FIG. 12a, the value of 1 was set to the expression level in the untreated control samples.

### Example 5

The aim of this experiment was to test the role of the HWC compounds in maintaining the stem cell marker NANOG and blocking the differentiation marker PAX6 in the face of differentiating conditions. This is in order to functionally test the properties of the compounds required for stem cell marker maintenance and differentiation blocking.

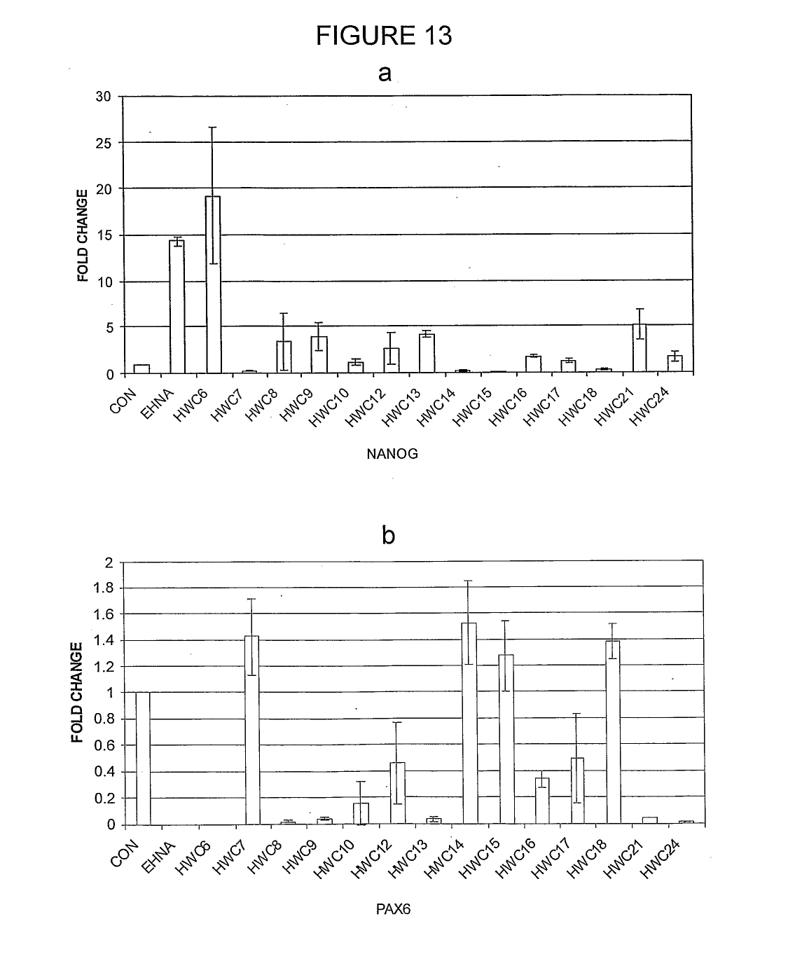

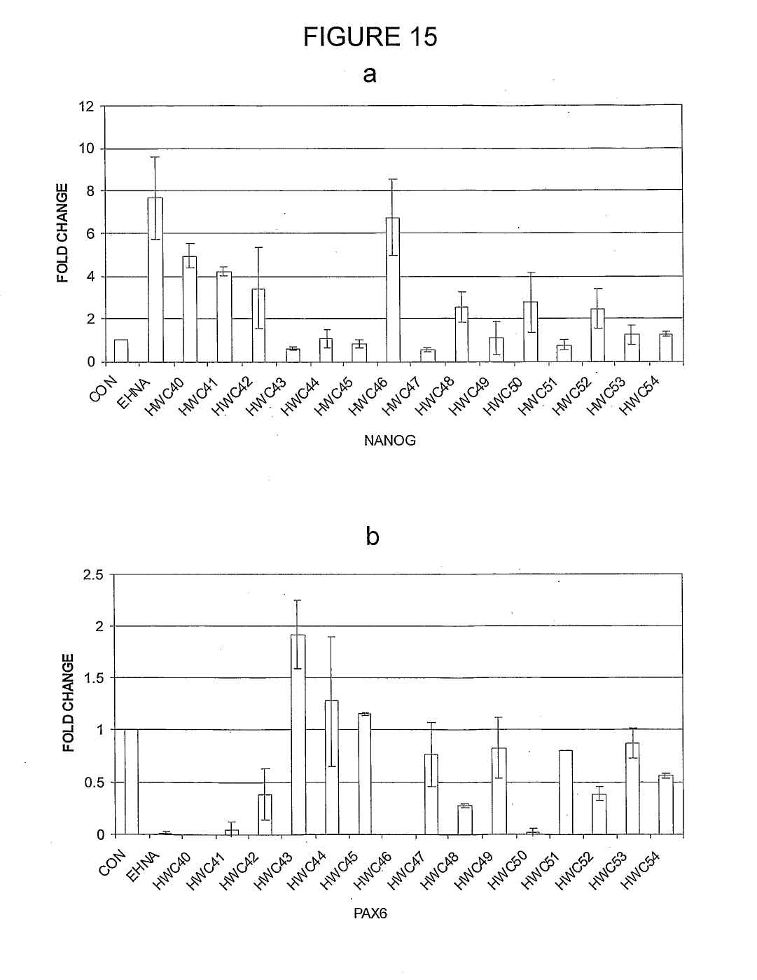

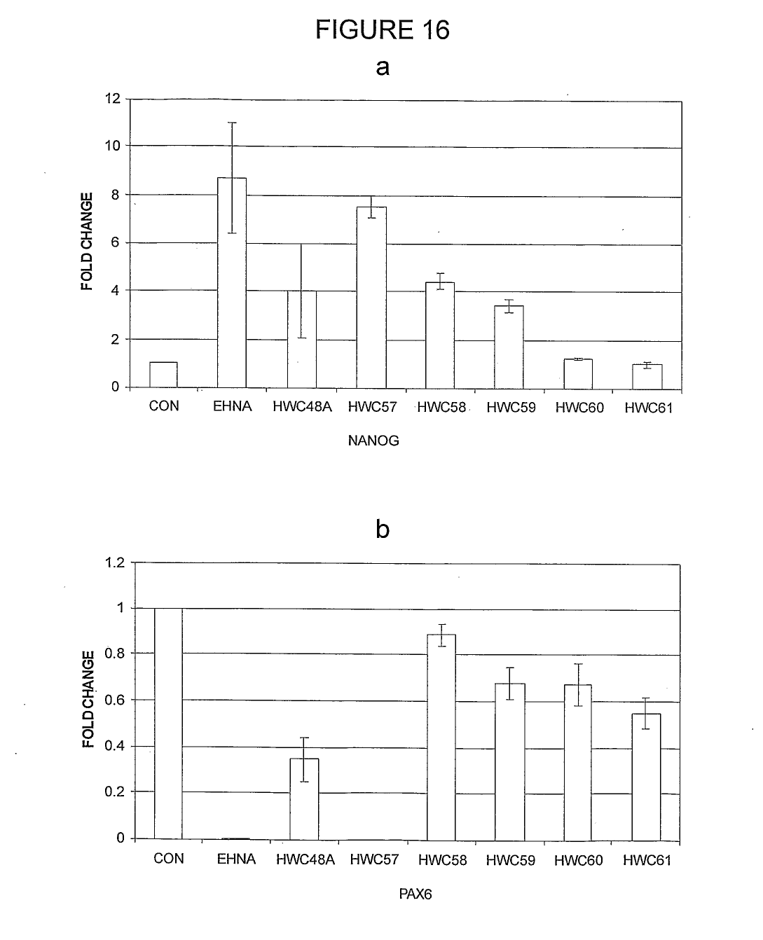

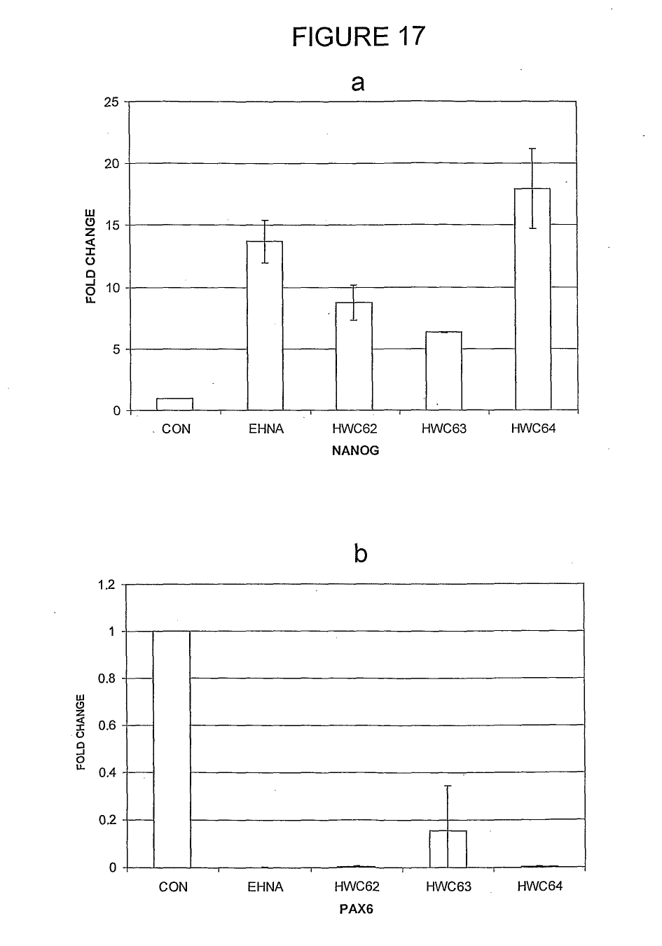

Cells were enzymatically passaged onto matrigel-coated dishes and grown for 2 weeks in defined media (1× confluent T25 tissue culture flask was passaged to 2×12 well dishes) with or without (control) compound addition. All compounds were added at 10 uM and each was performed in triplicate. Media and compounds were changed every 48 hours. After 14 days RNA was isolated and qRT-PCR was utilised to analyse the expression of NANOG and PAX6. The results are illustrated in FIGS. 13 to 17.

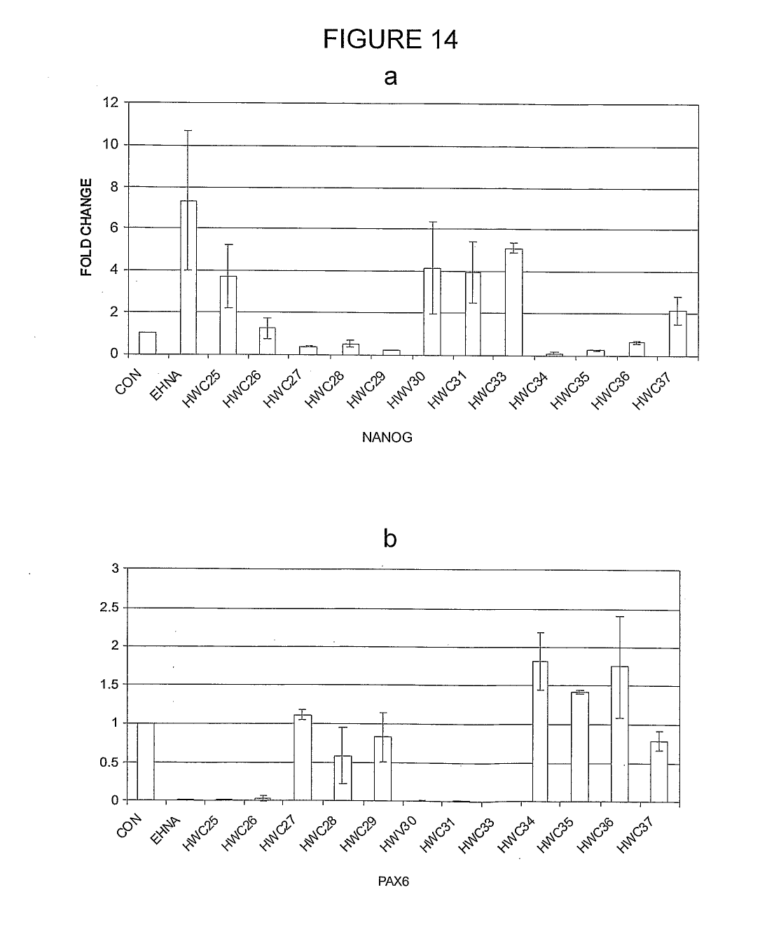

In the graphs in FIGS. 13, 14 15, 16 and 17 the value 1 was set relative to the expression level of the gene in the control (no compound) samples. These data show that EHNA clearly maintains a higher level of NANOG than the untreated control and also inhibits the onset of the differentiation marker PAX6 seen in the control. This experiment allowed the effect of the EHNA-related compounds to be measured directly in comparison to EHNA. In clarifying which compounds had an EHNA-like effect on gene expression those which maintained at least 50% of the level of NANOG-expression in comparison to EHNA and those that inhibited the expression of PAX6 to 50% or less than the value untreated controls were considered to have an EHNA-like effect. Those which had on effect, to these levels, on either PAX6 or NANOG were considered to have a partial-EHNA effect. The effect of each compound can be seen in FIGS. 13-17 and the results are summarised in Table 2 below.

TABLE 2

| | | PARTIAL | PARTIAL | |
|---|---|---|---|---|
| | Full | EHNA | EHNA | NO |
| | EHNA | EFFECT | EFFECT | EHNA |
| | effect | (NANOG) | (PAX6) | EFFECT |
| EHNA | X | | | |
| HWC25 | X | | | |
| HWC31 | X | | | |
| HWC33 | X | | | |
| HWC40 | X | | | |
| HWC41 | X | | | |
| HWC46 | X | | | |
| HWC57 | X | | | |
| HWC6 | X | | | |
| HWV30 | X | | | |
| HWC62 | X | | | |
| HWC64 | X | | | |
| HWC58 | | X | | |
| HWC10 | | | X | |
| HWC12 | | | X | |
| HWC13 | | | X | |
| HWC16 | | | X | |
| HWC17 | | | X | |
| HWC21 | | | X | |
| HWC24 | | | X | |
| HWC26 | | | X | |
| HWC27 | | | X | |
| HWC28 | | | X | |
| HWC29 | | | X | |
| HWC34 | | | X | |
| HWC35 | | | X | |
| HWC37 | | | X | |
| HWC42 | | | X | |
| HWC48 | | | X | |
| HWC48A | | | X | |
| HWC50 | | | X | |
| HWC52 | | | X | |
| HWC8 | | | X | |
| HWC9 | | | X | |
| HWC63 | | | X | |
| HWC14 | | | | X |
| HWC15 | | | | X |
| HWC18 | | | | X |
| HWC36 | | | | X |
| HWC43 | | | | X |
| HWC44 | | | | X |
| HWC45 | | | | X |
| HWC47 | | | | X |
| HWC49 | | | | X |
| HWC51 | | | | X |
| HWC53 | | | | X |
| HWC54 | | | | X |
| HWC59 | | | | X |
| HWC60 | | | | X |
| HWC61 | | | | X |
| HWC7 | | | | X |

1. A method of inhibiting stem cell differentiation comprising contacting a compound of formula (I) with a stem cell: 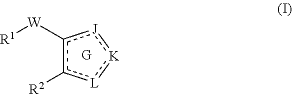
wherein
W is selected from C(Z)₂ and NZ;
each Z is independently selected from hydrogen, C₁₋₁₂ alkyl, C₂₋₁₂ alkenyl, C₂₋₁₂ alkynyl, halogen, —SR⁵, —OR⁵, —NR⁶R⁶, aryl, heteroaryl, —COR^{E}, C₃₋₁₀ cycloalkyl and C₃₋₁₀ heterocycloalkyl or (Z)₂ is ═O;
J and K are each independently selected from N, NR³, NR⁴ and CR³;
L is selected from N and NR⁴, wherein if L is N, one of J or K is NR⁴;
ring G is an aromatic ring;
R¹ is selected from hydrogen, C₁₋₁₂ alkyl, C₁₋₁₂ alkenyl, C₂₋₁₂ alkynyl, halogen, —SR⁷, —OR⁷, —NR⁸R⁸, aryl, heteroaryl, —COR⁸, C₃₋₁₂ cycloalkyl and C₃₋₁₀ heterocycloalkyl;
R² is selected from hydrogen, C₁₋₁₂ alkyl, C₂₋₁₂ alkenyl, C₂₋₁₂ alkynyl, halogen, —SR⁹, —OR⁹, —NR¹⁰R¹⁰, aryl, heteroaryl, —COR¹⁰, C₃₋₁₀ cycloalkyl and C₃₋₁₀ heterocycloalkyl; or alternatively
R¹ and R² are joined to form a 5 to 7 membered carbocyclic ring, optionally including one, two or three unsaturated bonds, wherein optionally one or more of the carbon atoms which form the 5 to 7 membered carbocyclic ring is replaced with a heteroatom selected from N, S and O, and wherein each one of the atoms which form the 5 to 7 membered ring is independently optionally substituted with one or two R³² groups, wherein each R³² is independently selected from hydrogen, halogen, C₁₋₁₂-alkyl, C₂₋₁₂-alkenyl, C₂₋₁₂-alkynyl, aryl, heteroaryl, —OR³³, NR³⁴R³⁴, —COR³⁴, C₃₋₁₂ cycloalkyl and C₃₋₁₀ heterocycloalkyl;
R³ is selected from hydrogen, C₁₋₁₂ alkyl, C₂₋₁₂ alkenyl, C₂₋₁₂ alkynyl, halogen, —SR¹¹, —OR¹¹, NR¹²R¹², aryl, heteroaryl, —COR¹², C₃₋₁₀ cycloalkyl and C₃₋₁₀ heterocycloalkyl;
R⁴ is a group of formula (IIA) or (IIB): 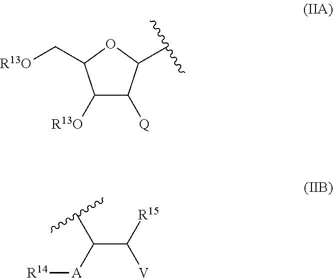
wherein Q is selected from —H and —OH;
R¹³ is selected from hydrogen, C₁₋₁₂ alkyl, C₂₋₁₂ alkenyl, C₂₋₁₂ alkynyl, aryl, heteroaryl, —COR¹⁶, C₃₋₁₀ cycloalkyl and C₃₋₁₀ heterocycloalkyl;
A is a single bond or a group of formula —O-M-, wherein M is selected from C₁₋₆ alkyl, C₂₋₆ alkenyl and C₂₋₆ alkynyl;
V is selected from hydrogen, —OR¹⁷, —SR¹⁷, NR¹⁸R¹⁸ and cyano;
R¹⁴ is selected from hydrogen, C₁₋₁₂ alkyl, C₂₋₁₂ alkenyl, C₂₋₁₂ alkynyl, halogen, —SR¹⁹, —OR¹⁹, —NR²⁰R²⁰, aryl, heteroaryl, —COR²⁰, C₃₋₁₀ cycloalkyl and C₃₋₁₀ heterocycloalkyl wherein each of said C₁₋₁₂ alkyl, C₂₋₁₂ alkenyl, C₂₋₁₂-alkynyl, C₁₋₁₀-alkoxy, aryl, heteroaryl and C₃₋₁₀ cycloalkyl is optionally substituted with 1, 2 or 3 groups independently selected from hydrogen, halogen, C₁₋₁₂-alkyl, C₂₋₁₂-alkenyl, aryl, heteroaryl, —OR²⁵ and NR²⁵R²⁶;
R¹⁵ is selected from hydrogen, C₁₋₁₂ alkyl, C₂₋₁₂ alkenyl, C₂₋₁₂ alkynyl, halogen, —CF₃, —SR²¹, OR²¹, NR²²R²², aryl, heteroaryl, —COR²², C₃₋₁₀ cycloalkyl and C₃₋₁₀ heterocycloalkyl;
each R⁵, R⁷, R⁹, R¹¹, R¹⁷, R¹⁹, R²¹ and R³³ is independently selected from hydrogen, C₁₋₁₂ alkyl, C₂₋₁₂ alkenyl, halogen, NR²³R²⁴, aryl, heteroaryl, C₃₋₁₀ cycloalkyl and C₃₋₁₀ heterocycloalkyl wherein each of said C₁₋₁₂ alkyl, C₂₋₁₂ alkenyl, C₂₋₁₂-alkynyl, C₁₋₁₀-alkoxy, aryl, heteroaryl and C₃₋₁₀ cycloalkyl is optionally substituted with 1, 2 or 3 groups independently selected from hydrogen, halogen, C₁₋₁₂-alkyl, C₂₋₁₂-alkenyl, aryl, heteroaryl, —OR²⁵ and NR²⁵R²⁶;
each R⁶, R⁸, R¹⁰, R¹², R¹⁶, R¹⁸, R²⁰, R²² and R³⁴ is independently selected from hydrogen, C₁₋₁₂ alkyl, C₂₋₁₂ alkenyl, —OR²⁷, halogen, NR²⁷R²⁸, —COR²⁸, aryl, heteroaryl, C₃₋₁₀ cycloalkyl and C₃₋₁₀ heterocycloalkyl wherein each of said C₁₋₁₂ alkyl, C₂₋₁₂ alkenyl, C₂₋₁₂-alkynyl, aryl, heteroaryl, C₃₋₁₀ cycloalkyl and C₃₋₁₀ heterocycloalkyl is optionally substituted with 1, 2 or 3 groups independently selected from hydrogen, halogen, —OR³⁰, C₁₋₁₂-alkyl, C₂₋₁₂ alkenyl, C₂₋₁₂ alkynyl, aryl, heteroaryl, C₁₋₁₂ alkoxy and NR³⁰R³¹; and
R²³, R²⁴, R²⁵, R²⁶, R²⁷, R²⁸, R³⁰ and R³¹ are independently selected from H and C₁₋₆ alkyl or a pharmaceutically acceptable salt thereof.

2. A method according to claim 1, wherein J is N, K is CR³ and L is NR⁴.

3. A method according to claim 1 or claim 2, wherein R¹ and R² are joined to form a 6 membered carbocyclic ring wherein optionally one or more of the carbon atoms which form the 5 to 7 membered carbocyclic ring is replaced with a heteroatom selected from N, S and O, and wherein each one of the atoms which form the 5 to 7 membered ring is independently optionally substituted with one or two R³² groups, wherein each R³² is independently selected from hydrogen, halogen, C₁₋₁₂-alkyl, C₂₋₁₂-alkenyl, C₂₋₁₂-alkynyl, aryl, heteroaryl, —OR³³, NR³⁴R³⁴, —COR³⁴, C₃₋₁₂ cycloalkyl and C₃₋₁₀ heterocycloalkyl.

4. A method according to claim 3, wherein the compound has formula (IA): 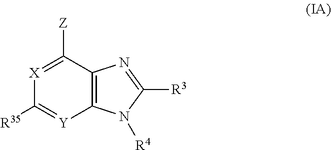
wherein X and Y are independently selected from N and CH;
R³⁵ is selected from hydrogen, halogen, C₁₋₁₂ alkyl, C₂₋₁₂ alkenyl, C₂₋₄₂ alkynyl, —SR³⁶, —OR³⁶, —NR³⁷R³⁷, aryl, heteroaryl, —COR³⁷, C₃₋₁₀ cycloalkyl, C₃₋₁₀ heterocycloalkyl;
each R³⁶ is independently selected from hydrogen, C₁₋₁₂ alkyl, C₂₋₁₂ alkenyl, C₂₋₁₂ alkynyl, C₂₋₁₂ alkoxy, halogen, NR³⁸R³⁹, aryl, heteroaryl and C₃₋₁₀ cycloalkyl, wherein each of said C₁₋₁₂ alkyl, C₂₋₁₂-alkynyl, C₂₋₁₂-alkynyl, C₁₋₁₂ alkoxy, aryl, heteroaryl and C₃₋₁₀ cycloalkyl is optionally substituted with 1, 2 or 3 groups independently selected from hydrogen, halogen, C₁₋₁₂-alkyl, C₂₋₁₂-alkenyl, aryl, heteroaryl, C₁₋₁₂ alkoxy and NR⁴⁰R⁴¹;
each R³⁷ is independently selected from hydrogen, C₁₋₁₂ alkyl, C₂₋₁₂ alkenyl, C₂₋₁₂ alkynyl, halogen, —OR⁴², NR⁴³R⁴³, aryl, heteroaryl, C₃₋₁₀ cycloalkyl and C₃₋₁₀ heterocycloalkyl wherein each of said C₁₋₁₂ alkyl, C₂₋₁₂-alkynyl, C₂₋₁₂-alkynyl, aryl, heteroaryl, C₃₋₁₀ cycloalkyl and C₃₋₁₀ heterocycloalkyl is optionally substituted with 1, 2 or 3 groups independently selected from hydrogen, halogen, C₁₋₁₂-alkyl, C₂₋₁₂-alkenyl, aryl, heteroaryl, —OR⁴⁴ and NR⁴⁵R⁴⁵; and
R³⁸, R³⁹, R⁴⁰, R⁴¹, R⁴², R⁴³ R⁴⁴ and R⁴⁵ are independently selected from H and (C₁₋₆)alkyl.

5. A method according to claim 4, where X and Y are both N.

6. A method according to any preceding claim, wherein R⁴ is a group of formula (IIB).

7. A method according to claim 6, wherein V is —OH.

8. A method according to claim 6 or claim 7, wherein A is a single bond and R¹⁴ is C₁₋₁₀ alkyl.

9. A method according to any one of claims 6 to 8, wherein R¹⁵ is C₁₋₁₀ alkyl.

10. A method according to any preceding claim, wherein Z is —NR⁶R⁶.

11. A method according to claim 10, wherein each R⁶ is hydrogen.

12. A method according to any preceding claim, wherein the compound of formula (I) is an ADA inhibitor.

13. A method according to any preceding claim, wherein the compound is 3-(6-aminopurin-9-yl)nonan-2-ol or a pharmaceutically acceptable salt thereof.

14. A method according to claim 13, wherein the compound is erythro-3-(6-aminopurin-9-yl)nonan-2-ol or a pharmaceutically acceptable salt thereof.

15. A method according to any one of claims 1 to 12, wherein the compound is erythro-3-(3H-imidazo[4,5-b]pyridin-3-yl) nonan-2-ol or a pharmaceutically acceptable salt thereof.

16. A method according to any one of claims 1 to 12, wherein the compound is 2-decyl-2H-pyrazolo[3,4-d]pyrimidin-4-amine or a pharmaceutically acceptable salt thereof.

17. A method of inhibiting stem cell differentiation comprising contacting an ADA inhibitor with a stem cell.

18. A method according to any preceding claim, wherein the stem cells are embryonic stem cells.

19. A method according to any preceding claim, wherein the stem cells are human stem cells.

20. Use of a compound of formula (I) as defined in any one of claims 1 to 16, for inhibiting stem cell differentiation.

21. Use of an ADA inhibitor for inhibiting stem cell differentiation.

22. Use according to claim 20 or claim 21, wherein the stem cells are embryonic stem cells.

23. Use according to any one of claims 20 to 22, wherein the stem cells are human stem cells.

24. Use of a compound of formula (I) as defined in any one of claims 1 to 16, in the manufacture of a medicament for inhibiting stem cell differentiation.

25. Use of an ADA inhibitor in the manufacture of a medicament for inhibiting stem cell differentiation.

26. Use according to claim 24 or claim 25, wherein the stem cells are embryonic stem cells.

27. Use according to any one of claims 24 to 26, wherein the stem cells are human stem cells.

28. A compound of formula (I) as defined in any one of claims 1 to 16 for inhibiting stem cell differentiation.

29. An ADA inhibitor for inhibiting stem cell differentiation.

30. A compound according to claim 28 or an ADA inhibitor according to claim 29, wherein the stem cells are embryonic stem cells.

31. A compound according to claim 28 or an ADA inhibitor according to claim 29, wherein the stem cells are human stem cells.

32. A culture medium for expanding a population of pluripotent stem cells comprising an ADA inhibitor.

33. A culture medium for expanding a population of pluripotent stem cells comprising a compound of formula (I) as defined in any one of claims 1 to 16.

34. A culture medium according to claim 33, wherein the compound is 3-(6-aminopurin-9-yl)nonan-2-ol.

35. A culture medium according to claim 33, wherein the compound is erythro-3-(3H-imidazo[4,5-b]pyridin-3-yl)nonan-2-ol.

36. A culture medium according to claim 33, wherein the compound is 2-decyl-2H-pyrazolo[3,4-d]pyrimidin-4-amine.

37. A method for preparing a culture medium, comprising the steps of (a) providing a culture medium; and (b) adding an ADA inhibitor to the culture medium.

38. A method for preparing a culture medium, comprising the steps of (a) providing a culture medium; and (b) adding a compound of formula (I) as defined in any one of claims 1 to 16 to the culture medium.

39. A method according to claim 38, wherein the compound is 3-(6-aminopurin-9-yl)nonan-2-ol.

40. A method according to claim 38, wherein the compound is erythro-3-(3H-imidazo[4,5-b]pyridin-3-yl)nonan-2-ol.

41. A method according to claim 38, wherein the compound is 2-decyl-2H-pyrazolo[3,4-d]pyrimidin-4-amine.

42. A culture medium supplement that comprises an ADA inhibitor.

43. A culture medium supplement that comprises a compound of formula (I) as defined in any one of claims 1 to 16.

44. A culture medium supplement according to claim 43, wherein the compound is 3-(6-aminopurin-9-yl)nonan-2-ol.

45. A culture medium supplement according to claim 43, wherein the compound is erythro-3-(3H-imidazo[4,5-b]pyridin-3-yl)nonan-2-ol.

46. A culture medium supplement according to claim 43, wherein the compound is 2-decyl-2H-pyrazolo[3,4-d]pyrimidin-4-amine.

47. A composition comprising an ADA inhibitor and stem cells.

48. A composition comprising a compound of formula (I) as defined in any one of claims 1 to 16 and stem cells.

49. A composition according to claim 48, wherein the compound is 3-(6-aminopurin-9-yl)nonan-2-ol.

50. A composition according to claim 48, wherein the compound is erythro-3-(3H-imidazo[4,5-b]pyridin-3-yl)nonan-2-ol.

51. A composition according to claim 48, wherein the compound is 2-decyl-2H-pyrazolo[3,4-d]pyrimidin-4-amine.


SEQUENCE LISTING (18 sequences)

SEQ ID NO: 1 - DNA, 29 bp, Artificial Sequence
  Probe
cagctacaaacaggtgaagacctggttcc

SEQ ID NO: 2 - DNA, 25 bp, Artificial Sequence
  Probe
ctgtgacaaccagaaaggatgcctc

SEQ ID NO: 3 - DNA, 35 bp, Artificial Sequence
  Probe
catggcgggacacctggcttcagattttgccttct

SEQ ID NO: 4 - DNA, 24 bp, Artificial Sequence
  Probe
catggagaaaacccggtacgctca

SEQ ID NO: 5 - DNA, 28 bp, Artificial Sequence
  Probe
taagcccaggcaaggcaagtcaagccaa

SEQ ID NO: 6 - DNA, 24 bp, Artificial Sequence
  Probe
tggcattgccctcaacgaccactt

SEQ ID NO: 7 - DNA, 21 bp, Artificial Sequence
  PCR primer
gaactctccaacatcctgaac

SEQ ID NO: 8 - DNA, 21 bp, Artificial Sequence
  PCR primer
aaccccaaccaaacaaaactc

SEQ ID NO: 9 - DNA, 18 bp, Artificial Sequence
  PCR primer
cgcaagccctcatttcac

SEQ ID NO: 10 - DNA, 19 bp, Artificial Sequence
  PCR primer
aatgggaggggtgcaaaag

SEQ ID NO: 11 - DNA, 22 bp, Artificial Sequence
  PCR primer
gcaaagacaagacaccagaaag

SEQ ID NO: 12 - DNA, 20 bp, Artificial Sequence
  PCR primer
aggtggtctcctctgacttc

SEQ ID NO: 13 - DNA, 22 bp, Artificial Sequence
  PCR primer
cgtcacaccattgctattcttc

SEQ ID NO: 14 - DNA, 21 bp, Artificial Sequence
  PCR primer
gcgcccctagttaaagtcttc

SEQ ID NO: 15 - DNA, 18 bp, Artificial Sequence
  PCR primer
ccaggtccgaggatcaac

SEQ ID NO: 16 - DNA, 19 bp, Artificial Sequence
  PCR primer
tgagtgtggatgggattgg

SEQ ID NO: 17 - DNA, 21 bp, Artificial Sequence
  PCR primer
catagcacacatagccatcac

SEQ ID NO: 18 - DNA, 22 bp, Artificial Sequence
  PCR primer
cgttgtcataccaggaaatgag